(19) United States
(12) Patent Application Publication
(10) Pub. No.: US 2018/0289655 A1
(43) Pub. Date: Oct. 11, 2018

(54) Methods and Compositions for the Intravenous Administration of Fumarates for the Treatment of Neurological Diseases

(71) Applicant: Biogen MA Inc., Cambridge, MA (US)

(72) Inventors: Ajay Verma, Needham, MA (US); Robert Scannevin, Hopkinton, MA (US); Shyam Karki, Hillsborough, NJ (US); Fengmei Zheng, Quincy, MA (US)

(73) Assignee: Biogen MA Inc., Cambridge, MA (US)

(21) Appl. No.: 15/559,265

(22) PCT Filed: Mar. 18, 2016
(86) PCT No.: PCT/US16/23021

§ 371 Date: Sep. 18, 2017

Related U.S. Application Data
(60) Provisional application No. 62/136,431, filed on Mar. 20, 2015.

(51) Int. Cl. A61K 31/225; A61K 47/40; A61K 9/00

(52) U.S. Cl. CPC A61K 31/225; A61K 2300/00; A61K 9/0019; A61K 47/40

(57) ABSTRACT

Disclosed herein are methods and compositions for the intravenous administration of fumarates for the treatment of neurological diseases, such as stroke, amyotrophic lateral sclerosis, Huntington's disease, Alzheimer's disease, Parkinson's disease, and Multiple Sclerosis.

### CROSS-REFERENCES TO RELATED APPLICATIONS

This application is a national stage of International Patent Application No. PCT/US2016/023021, filed Mar. 18, 2016, which claims the benefit of U.S. Provisional Patent Application No. 62/136,431, filed Mar. 20, 2015, which is incorporated herein by reference in its entirety.

### 1. FIELD OF INVENTION

Disclosed herein are methods and compositions for the intravenous administration of fumarates for the treatment of neurological diseases, such as stroke, amyotrophic lateral sclerosis, Huntington's disease, Alzheimer's disease, Parkinson's disease, and Multiple Sclerosis.

### 2. BACKGROUND

Neurological diseases generally affect neurons in the central nervous system, i.e., the brain and the spinal cord. Treatment of these diseases with safe and effective compounds is desirable.

Stroke

Stroke is the fourth-leading cause of death in the United States. Stroke can be caused by clots in blood vessels that block blood flow to the brain (ischemic stroke) or by a blood vessel rupturing and preventing blood flow to the brain (hemorrhagic stroke). A third type of stroke is a transient ischemic attack, colloquially referred to as a “mini stroke,” which is caused by a temporary blood clot. Ischemic strokes account for the majority of strokes that occur in humans.

Disruption in blood flow to the brain results in cell death in the affected region due to lack of glucose and oxygen. Recovery from stroke is often partial and survivors suffer from long-term or permanent motor, sensory and cognitive impairments. Often, stroke survivors suffer permanent neurological damage and sensorimotor impairments, with an estimated 15-30% of stroke survivors becoming permanently disabled (Roger et al., Circulation 2012; 125:22-e220).

To date, the direct pharmaceutical management of ischemic stroke is confined to drugs administered in the acute phase following a stroke, that is, from the time of onset of the injury to approximately six hours post-injury. Currently, there are no known drugs for the treatment of hemorrhagic stroke.

Presently there is no therapy approved in the U.S. for the treatment of stroke other than tissue plasminogen activator (tPA) and other surgical methods that are employed during the acute phase of stroke. After the available treatments, patients often remain with some level of dysfunction. Patients have to go through physical therapy in an effort to regain the lost sensorimotor functions, with varying degrees of success in rehabilitation (Sun et al., 2014, Ann. Transl. Med., 2(8): 80).

Most drugs currently being investigated for the treatment of stroke are focused on reducing acute cell death, inflammation, and apoptosis and must, therefore, be delivered within hours after the ischemic event (Prakash et al., 2013 Pharmacology, 92:324-334).

Amyotrophic Lateral Sclerosis (ALS)

Amyotrophic lateral sclerosis (ALS) is an adult-onset neurodegenerative disease. ALS is fatal and has a short disease course, resulting in death within approximately five years of diagnosis in most cases (Mitchell et al., 2007, Lancet 369: 2031-41). The onset of disease occurs generally between age 40 and age 70. According to the ALS CARE Database, 60% of ALS patients in the Database are men and 93% of ALS patients in the Database are Caucasian.

ALS is characterized by the progressive degeneration of upper and lower motor neurons in the motor cortex, spinal cord, and brainstem. This leads to an inability to control and initiate muscle movement. Death is often caused by respiratory failure because the diaphragm and intercostal muscles are eventually disabled.

The etiology of ALS is not well-understood. It is known that the disease occurs in one of two forms; a sporadic form, which affects approximately 90% of the patients, or a familial form, which affects approximately 10% of ALS patients, the latter of which is linked to specific genetic mutations. To date, ALS has been linked to mutations in the C9ORF72, Superoxide Dismutase 1 (SOD1), TAR DNA binding protein 43 (TDP-43), and Fused in Sarcoma (FUS) genes (Baloh et al., 2013, Neurol. Clin. 31:4). The sporadic and familial forms of the disease exhibit similar clinical presentations.

Currently, there is no known cure for ALS, and attempts at slowing the progression of the disease have been minimally successful.

Huntington's Disease

Huntington's disease is an inherited neurodegenerative disorder, caused by a genetic mutation. Patients with the disease have an abnormal number of CAG trinucleotide repeats in the HTT gene, which encodes the huntingtin protein (Cabouche et al., 2013, Frontiers in Neurology 4:127), and A Physician's Guide to the Management of Huntington's Disease, Lovecky and Trapata (eds.), 3^{rd} Ed., Huntington's Disease Society of America (2011), page 16.

Huntington's disease affects around 1 in 10,000 people in the United States. Currently, around 30,000 people have Huntington's disease and an additional 200,000 are at risk for developing the disease (Shannon, Hersch & Lovecky, Huntington's Disease, A Guide for Families, (2009) Huntington's Disease Soc'y of America, website at hdsa.org/images/content/1/4/14765.pdf). Disease onset begins at around 30 or 40 years of age. Some patients start exhibiting symptoms in their 20s (juvenile Huntington's disease), which is also associated with a faster progression of the disease.

Currently, there are no treatments available to cure Huntington's disease and to prevent the onset of disease, but medications can help with the symptoms of the movement and psychiatric disorders. Therapeutic options include dopamine-depleting agents (e.g., reserpine, tetrabenazine) and dopamine-receptor antagonists (e.g., neuroleptics), but these drugs carry a high risk of adverse effects especially for long-term use (Kori et al., 2010, Global J. Pharmacology 4(1): 06-12). Neuroleptics have been shown to worsen other features of the disease, such as bradykinesia and rigidity, leading to further functional decline (Kori et al., 2010, Global J. Pharmacology 4(1): 06-12).

Some studies have suggested that valproic acid and clonazepam may be effective in the treatment of chorea, while results of other studies have been less conclusive (Kim et al., 2014 J. Mov. Disord. 7(1): 1-6). Tetrabenazine, a dopamine-depleting agent, was approved by the FDA to suppress the involuntary jerking and writhing movements associated with Huntington's disease. It is thought to be more effective than reserpine in the treatment of chorea and less likely to cause hypotension, but a serious side effect of the drug is the worsening or triggering of depression or other psychiatric conditions (Xenazine® Drug Label, available at the website at accessdata.fda.gov/drugsatfda_docs/label/2008/0218941b1.pdf).

Alzheimer's Disease

Alzheimer disease is an increasingly prevalent form of neurodegeneration that accounts for approximately 50%-60% of the overall cases of dementia among people over 65 years of age. Alzheimer's disease currently affects an estimated 15 million people worldwide and owing to the relative increase of elderly people in the population its prevalence is likely to increase over the next 2 to 3 decades. Although the speed of progression can vary, the average life expectancy following diagnosis is approximately seven years. Fewer than 3% of individuals live more than 14 years after diagnosis. Death of pyramidal neurons and loss of neuronal synapses in brains regions associated with higher mental functions results in the typical symptoms, characterized by gross and progressive impairment of cognitive function (Francis et al., 1999, J. Neurol. Neurosurg. Psychiatry 66:137-47). Alzheimer disease is the most common form of both senile and presenile dementia in the world and is recognized clinically as relentlessly progressive dementia that presents with increasing loss of memory, intellectual function and disturbances in speech (Merritt, 1979, A Textbook of Neurology, 6th edition, pp. 484-489 Lea & Febiger, Philadelphia). The disease itself usually has a slow and insidious progress that affects both sexes equally, worldwide. It begins with mildly inappropriate behavior, uncritical statements, irritability, a tendency towards grandiosity, euphoria and deteriorating performance at work; it progresses through deterioration in operational judgment, loss of insight, depression and loss of recent memory; it ends in severe disorientation and confusion, apraxia of gait, generalized rigidity and incontinence (Gilroy & Meyer, 1979, Medical Neurology, pp. 175-179 MacMillan Publishing Co.).

The cause of Alzheimer's disease is unknown. Based on familial incidence, pedigree analysis, monozygotic and dizygotic twin studies and the association of the disease with Down's syndrome, there appears to be a genetic contribution to Alzheimer disease development (for review see Baraitser, 1990, The Genetics of Neurological Disorders, 2nd edition, pp. 85-88). Additional factors, such as elevated concentrations of aluminum in the brain, manganese in the tissues, may also play a role in Alzheimer disease development (Crapper et al., 1976, Brain, 99:67-80, Banta & Markesberg, 1977, Neurology, 27:213-216). It has also been suggested that defects in the transcriptional splicing of mRNA coding for the tau complex of microtubule associated proteins occur (for review see Kosik, 1990, Curr. Opinion Cell Biol., 2:101-104) and/or that inappropriate phosphorylation of these proteins exists (Grundke-Igbak et al., 1986, Proc. Natl. Acad. Sci. USA, 83:4913-4917; Wolozin & Davies, 1987, Ann. Neurol. 22:521-526; Hyman et al., 1988, Ann. Neurol., 23:371-379; Bancher et al., 1989, Brain Res., 477:90-99) may also play a role in the development of Alzheimer disease. In addition, reduction in the enzymes involved in the synthesis of acetylcholine has led to the belief of Alzheimer disease as a cholinergic system failure (Danes & Moloney, 1976, Lancet, ii: 1403-14).

There are currently no proven therapies for Alzheimer disease, and no agents are consistently effective in preventing the progression of the disease. Most therapeutics focus on the management of the symptoms of Alzheimer disease. Current therapies include anti-psychiatric drugs as well as neuroleptic agents and acetylcholinesterase inhibitors. Due to the side effects and unattractive dosing requirements of these drugs, new methods and compounds that are able to treat Alzheimer disease and its symptoms are highly desired.

Parkinson's Disease

Parkinson's disease is a type of motor system disorder, resulting from the loss of dopamine-producing neurons. Parkinson's disease can be characterized by four primary symptoms, including tremor (e.g., trembling in hands, arms, legs, jaw, and face); rigidity (e.g., stiffness of the limbs and trunk; bradykinesia (e.g., slowness of movement); and postural instability (e.g., impaired balance and coordination). As Parkinson's disease progresses, patients may have difficulty walking, talking, or completing other simple tasks. Parkinson's disease usually affects people over the age of 50. In some people, early symptoms of Parkinson's disease can be subtle and occur gradually. In other people, the disease can progress more quickly. As Parkinson's disease progresses and the symptoms grow in severity, symptoms, such as shaking or tremor, may begin to interfere with daily activities. Parkinson's disease symptoms may also include behavioral symptoms, such as depression and other emotional changes. In addition, Parkinson's disease patients, may experience difficulty in swallowing, chewing, and speaking. Additional, Parkinson's disease symptoms include, but are not limited to, urinary problems or constipation; skin problems; and sleep disruptions. See What is Parkinson's Disease?, NINDS Parkinson's Disease Information Page, National Institute of Neurological Disorders and Stroke at ninds.nih.gov.

There is currently no cure for Parkinson's disease, but current therapies provide relief from one or more symptoms. Current therapies may include levodopa combined with carbidopa, anticholinergics, bromocriptine, pramipexole, and ropinirole. Antivirals, such as amantadine, have also been used to treat Parkinson's disease. Although levodopa may help alleviate some symptoms of Parkinson's disease in Parkinson's disease patients, not all symptoms respond equally to the drug. Some symptoms, such as bradykinesia and rigidity, respond better, while other symptoms, such as tremor, may be only marginally reduced. See What is Parkinson's Disease?, NINDS Parkinson's Disease Information Page, National Institute of Neurological Disorders and Stroke at ninds.nih.gov.

In some cases, Parkinson's disease patients, who are unresponsive to current drug therapies, are treated with surgery. Surgery can involve deep brain stimulation (DBS). In DBS, electrodes are implanted into the brain and connected to a small electrical device called a pulse generator that can be externally programmed. DBS requires careful programming of the stimulator device in order to work correctly. See What is Parkinson's Disease?, NINDS Parkinson's Disease Information Page, National Institute of Neurological Disorders and Stroke at ninds.nih.gov.

Multiple Sclerosis

Multiple sclerosis (MS) is an autoimmune disease with the autoimmune activity directed against central nervous system (CNS) antigens. The disease is characterized by inflammation in parts of the CNS, leading to the loss of the myelin sheathing around neuronal axons (demyelination), axonal loss, and the eventual death of neurons, oligodendrocytes and glial cells. For a comprehensive review of MS and current therapies, see, e.g., McAlpine's Multiple Sclerosis, by Alastair Compston et al., 4th edition, Churchill Livingstone Elsevier, 2006.

More than 2.1 million people in the world suffer from MS, with roughly 400,000 of those living in the United States (see, e.g., Hanson et al., Patient Prefer Adherence, 2014, 8:415-422). It is one of the most common diseases of the CNS in young adults. MS is a chronic, progressing, disabling disease, which generally strikes its victims some time after adolescence, with diagnosis generally made between 20 and 40 years of age, although onset may occur earlier. Women are more likely than men to have the disease and MS itself is highly variable with symptoms and severity ranging from patient to patient (see, e.g., Ruggieri et al., Ther. Clin. Risk Manag., 2014, 10:229-239). The disease is not directly hereditary, although genetic susceptibility plays a part in its development. MS is a complex disease with heterogeneous clinical, pathological and immunological phenotype.

There are four major clinical types of MS: 1) relapsing-remitting MS (RR-MS), characterized by clearly defined relapses with full recovery or with sequelae and residual deficit upon recovery; periods between disease relapses characterized by a lack of disease progression; 2) secondary progressive MS (SP-MS), characterized by initial relapsing remitting course followed by progression with or without occasional relapses, minor remissions, and plateaus; 3) primary progressive MS (PP-MS), characterized by disease progression from onset with occasional plateaus and temporary minor improvements allowed; and 4) progressive relapsing MS (PR-MS), characterized by progressive disease onset, with clear acute relapses, with or without full recovery; periods between relapses characterized by continuing progression.

Clinically, the illness most often presents as a relapsing-remitting disease and, to a lesser extent, as steady progression of neurological disability. Relapsing-remitting MS (RR-MS) presents in the form of recurrent attacks of focal or multifocal neurologic dysfunction. Attacks may occur, remit, and recur, seemingly randomly over many years. Remission is often incomplete and as one attack follows another, a stepwise downward progression ensues with increasing permanent neurological deficit. The usual course of RR-MS is characterized by repeated relapses associated, for the majority of patients, with the eventual onset of disease progression. The subsequent course of the disease is unpredictable, although most patients with a relapsing-remitting disease will eventually develop secondary progressive disease. In the relapsing-remitting phase, relapses alternate with periods of clinical inactivity and may or may not be marked by sequelae depending on the presence of neurological deficits between episodes. Periods between relapses during the relapsing-remitting phase are clinically stable. On the other hand, patients with progressive MS exhibit a steady increase in deficits, as defined above and either from onset or after a period of episodes, but this designation does not preclude the further occurrence of new relapses.

MS pathology is, in part, reflected by the formation of focal inflammatory demyelinating lesions in the white matter, which are the hallmarks in patients with acute and relapsing disease. In patients with progressive disease, the brain is affected in a more global sense, with diffuse but widespread (mainly axonal) damage in the normal appearing white matter and massive demyelination also in the grey matter, particularly, in the cortex.

Salts of fumaric acid esters, in combination with dimethyl fumarate (DMF), such as present in FUMADERM®, have been proposed for the treatment of MS (see, e.g., Schimrigk et al., Eur. J. Neurol., 2006, 13(6):604-610; Drugs R&D, 2005, 6(4):229-30; U.S. Pat. No. 6,436,992). FUMADERM® contains dimethyl fumarate, calcium salt of ethyl hydrogen fumarate, magnesium salt of ethyl hydrogen fumarate, and zinc salt of ethyl hydrogen fumarate (see, e.g., Schimrigk et al., Eur. J. Neurol., 2006, 13(6):604-610).

Although currently there is no cure for MS, treatment options are available for patients with the disease. Currently available treatments typically focus on slowing the progression of the disease over time, improving quality of life, and reducing the number and severity of the symptoms of MS. For those patients with relapsing MS, common initial treatments have included interferon-beta (IFN-β) and glatiramer acetate (see, e.g., Fox et al., N. Engl. J. Med., 2012, 367(12):1087-1097; Erratum in: N. Engl. J. Med., 2012, 367(17):1673). Additional treatments have included natalizumab. In the past few years, fingolimod, teriflunomide, and delayed-release DMF were developed as oral treatments, which are expected to improve adherence to treatment (see, e.g., Cree B. A., Neurohospitalist, 2014, 4(2):63-65).

TECFIDERA®, dimethyl fumarate delayed-release capsules for oral use, was approved in 2013 by the U.S. Food and Drug Administration for the treatment of subjects with relapsing forms of multiple sclerosis. TECFIDERA® contains dimethyl fumarate (DMF).

In conclusion, there exists a need in the area of treating neurological diseases, such as stroke, amyotrophic lateral sclerosis, Huntington's disease, Alzheimer's disease, Parkinson's disease, and multiple sclerosis to develop new therapies and more effective treatment regimens.

### 3. BRIEF SUMMARY OF THE INVENTION

Provided herein are methods of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

In one embodiment, the disease is stroke.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

In one embodiment, the disease or disorder is amyotrophic lateral sclerosis.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, rein a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, in the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

In one embodiment, the disease is Huntington's disease.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, ein the pharmaceutical composition is a sterile isotonic solution.

In one embodiment, the disease is Alzheimer's disease.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, wherein the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

In one embodiment, the disease is Parkinson's disease.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, rein the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

In one embodiment, the disease is Multiple Sclerosis.

In one embodiment, the Multiple Sclerosis is a progressive form of Multiple Sclerosis.

In one embodiment, the progressive form of Multiple Sclerosis is Primary Progressive Multiple Sclerosis (PP-MS) or Secondary Progressive Multiple Sclerosis (SP-MS).

In one embodiment, the Multiple Sclerosis is a relapsing form of Multiple Sclerosis.

In one embodiment, the relapsing form of Multiple Sclerosis is relapsing-remitting Multiple Sclerosis (RR-MS).

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the amount of dimethyl fumarate that is administered in said step of administering intravenously is in the range of 1 to 1000 milligrams.

In one embodiment, the amount of dimethyl fumarate that is administered in said step of administering intravenously is in the range of 10 to 750 milligrams.

In one embodiment, the amount of dimethyl fumarate that is administered in said step of administering intravenously is in the range of 48 to 240 milligrams.

In one embodiment, a therapeutically effective amount of dimethyl fumarate is administered in said step of administering intravenously, said amount being less than 480 milligrams.

In one embodiment, the method consists essentially of said administering step.

In one embodiment, the at least one fumarate is the only active agent administered to the patient for said treating.

In one embodiment, the only active agent in the pharmaceutical composition is the at least one fumarate.

In one embodiment, the only active agents in the pharmaceutical composition are dimethyl fumarate and monomethyl fumarate.

In one embodiment, the only active agent in the pharmaceutical composition is one fumarate selected from said group.

In one embodiment, the only active agent in the pharmaceutical composition is dimethyl fumarate and optionally one or more compounds produced by degradation from dimethyl fumarate in said pharmaceutical composition prior to said administering.

In one embodiment, the only active agent in the pharmaceutical composition is dimethyl fumarate.

In one embodiment, the pharmaceutical composition consists essentially of the at least one fumarate.

In one embodiment, the pharmaceutical composition consists essentially of dimethyl fumarate.

In one embodiment, said administering is performed daily.

In one embodiment, said administering is performed once per week.

In one embodiment, said administering is performed every other week.

In one embodiment, said administering is performed once per month.

In one embodiment, the step of administering intravenously is repeated over a time period of at least two weeks.

In one embodiment, the step of administering intravenously is repeated over a time period of at least one month.

In one embodiment, the step of administering intravenously is repeated over a time period of at least six months.

In one embodiment, the step of administering intravenously is repeated over a time period of at least one year.

In one embodiment, said administering is part of a treatment regimen wherein said administering intravenously to the patient alternates with one or more steps of administering the fumarate orally to the patient.

In one embodiment, the fumarate is dimethyl fumarate, and the amount of dimethyl fumarate administered orally is 480 mg daily.

In one embodiment, the patient does not have a known hypersensitivity to the fumarate.

In one embodiment, the patient is not treated simultaneously with a fumarate and any immunosuppressive or immunomodulatory medications or natalizumab.

In one embodiment, the patient is not treated simultaneously with a fumarate and any medications carrying a known risk of causing progressive multifocal leukoencephalopathy (PML).

In one embodiment, the patient has no identified systemic medical condition resulting in a compromised immune system function.

In one embodiment, the pharmaceutical composition is a sterile isotonic solution.

Provided herein are pharmaceutical compositions comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is a nanosuspension.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the concentration of the dimethyl fumarate is about 1 mg/ml to about 150 mg/ml.

In one embodiment, the concentration of the dimethyl fumarate is about 150 mg/ml.

In one embodiment, the pharmaceutical composition further comprises one or more excipients selected from a small molecule stabilizer, a polymeric stabilizer, and a buffer.

In one embodiment, the small molecule stabilizer is sodium dodecyl sulfate.

In one embodiment, the polymeric stabilizer is hydroxy propyl methyl cellulose (HPMC).

In one embodiment, the buffer is a phosphate buffer.

In one embodiment, the pH of the composition is in the range from about 4 to about 7.

In one embodiment, the pH of the composition is about 5.0.

In one embodiment, the fumarate has a mean particle size (D50) of about 100 nm to about 250 nm.

In one embodiment, the D50 is about 180 nm.

In one embodiment, the fumarate is dimethyl fumarate, wherein the pharmaceutical composition further comprises sodium dodecyl sulfate; HPMC, and a phosphate buffer, wherein the pH of the pharmaceutical composition is about 5.0 and the D50 is about 180 nm.

Provided herein are pharmaceutical compositions comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is an aqueous solution, wherein the aqueous solution comprises a cyclodextrin, wherein the cyclodextrin is an alpha cyclodextrin or a substituted beta cyclodextrin.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the concentration of the dimethyl fumarate is about 1 mg/ml to about 16 mg/ml.

In one embodiment, the concentration of the dimethyl fumarate is about 2 mg/ml to about 4 mg/ml.

In one embodiment, the cyclodextrin is a substituted beta cyclodextrin.

In one embodiment, the substituted beta cyclodextrin is present from about 5% (w/v) to about 40% (w/v).

In one embodiment, the substituted beta cyclodextrin is present at about 20% (w/v).

In one embodiment, the substituted beta cyclodextrin is hydroxypropyl beta cyclodextrin or sulfobutylether beta cyclodextrin.

In one embodiment, the substituted beta cyclodextrin is a sulfobutylether beta cyclodextrin.

In one embodiment, the pharmaceutical composition comprises one or more sulfobutylether beta cyclodextrins of Formula XX:

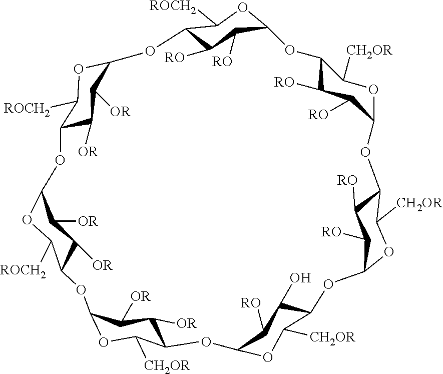

wherein R is independently selected from H or —CH₂CH₂CH₂CH₂SO₃Na, with the proviso that R is H but for 6 or 7 instances where R is —CH₂CH₂CH₂CH₂SO₃Na.

In one embodiment, the pharmaceutical composition comprises CAPTISOL.

In one embodiment, the fumarate is dimethyl fumarate, and wherein the aqueous solution comprises 20% (w/v) CAPTISOL, and the concentration of the DMF is about 2 mg/ml to about 4 mg/ml.

In one embodiment, the pharmaceutical composition is a nanosuspension.

In one embodiment, the pharmaceutical composition is an aqueous solution, wherein the aqueous solution comprises a cyclodextrin, wherein the cyclodextrin is an alpha cyclodextrin or a substituted beta cyclodextrin.

### 3.1 Terminology

In order to provide a clear and consistent understanding of the specification and claims, the following definitions are provided:

The term “alkanediyl,” as used herein refers to divalent linear or branched alkyl chains with, for example 1 to 6 carbon atoms. Representative examples of alkanediyl groups include, but are not limited to —CH₂—, —(CH₂)₂, —CH(CH₃)—, —(CH₂)₃—, —CH₂CH(CH₃)—, —CH(CH₃)CH₂—, —CH(C₂H₅)—, —C(CH₃)₂—, —(CH₂)₄—, —(CH₂)₂CH(CH₃)—, —CH₂CH(CH₃)CH₂—, —CH(CH₃)(CH₂)₂—, —CH(C₂H₅)CH₂—, —CH₂CH(C₂H₅)—, —C(CH₃)₂CH₂—, —CH₂C(CH₃)₂—, —CH(CH₃)CH(CH₃)—, —CH(C₃H₇)—, —(CH₂)₅, —(CH₂)₃CH(CH₃), —(CH₂)₂CH(CH₃)CH₂—, —CH₂CHCH₃(CH₂)₂—, —CH₂C(CH₃)₂CH₂—, —(CH₂)₂C(CH₃)₂—, —(CH₂)₆—, —(CH₂)₄CH(CH₃)—, —(CH₂)₃CH(CH₃)CH₂—, —CH₂CHCH₃(CH₂)₃—, —(CH₂)₃C(CH₃)₂—, and —(CH₂)₂C(CH₃)₂CH₂—.

The term “alkenyl,” as used herein, refers to a monovalent straight or branched chain hydrocarbon having from two to six carbons and at least one carbon-carbon double bond. Representative examples of alkenyl groups include, but are not limited to, —CH═CH₂, —CH═CH—CH₃, —CH₂—CH═CH—CH₃, or —CH(CH₃)—CH═CH—CH₃.

The term “alkyl,” as used herein, refers to a monovalent fully saturated branched or unbranched hydrocarbon moiety. In one embodiment, the alkyl comprises 1 to 20 carbon atoms, 1 to 16 carbon atoms, 1 to 10 carbon atoms, 1 to 6 carbon atoms, or 1 to 4 carbon atoms. Representative examples of alkyl groups include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, or n-decyl.

The term “alkynyl,” as used herein, refers to a monovalent straight or branched chain hydrocarbon having from two to six carbons and at least one carbon-carbon triple bond. Representative examples of alkynyl groups include, but are not limited to, 2-propynyl, 3-butynyl, 2-butynyl, 4-pentynyl, 3-pentynyl.

The term “aryl,” as used herein, refers to monocyclic, bicyclic or tricyclic aromatic hydrocarbon groups having, for example, from 5 to 14 carbon atoms in the ring portion. In one embodiment, the aryl refers to monocyclic and bicyclic aromatic hydrocarbon groups having from 6 to 10 carbon atoms. Representative examples of aryl groups include, but are not limited to, phenyl, naphthyl, fluorenyl, and anthracenyl.

The term “arylalkyl,” as used herein, refers to an acyclic alkyl group in which one of the hydrogen atoms bonded to a carbon atom, typically a terminal or sp³ carbon atom, is replaced with an aryl group. Representative examples of arylalkyl groups include, but are not limited to, benzyl, 2-phenylethan-1-yl, naphthylmethyl, 2-naphthylethan-1-yl, naphthobenzyl, or 2-naphthophenylethan-1-yl. In certain embodiments, an arylalkyl group is C₇₋₃₀ arylalkyl, e.g., the alkyl moiety of the arylalkyl group is C₁₋₁₀ and the aryl moiety is C₆₋₂₀. In certain embodiments, an arylalkyl group is C₆₋₁₈ arylalkyl, e.g., the alkyl moiety of the arylalkyl group is C₁₋₈ and the aryl moiety is C₆₋₁₀. In certain embodiments, the arylalkyl group is C₇₋₁₂ arylalkyl.

The term “alkyl linker,” as used herein, refers to C₁, C₂, C₃, C₄, C₅ or C₆ straight chain (linear) saturated aliphatic hydrocarbon groups and C₃, C₄, C₅ or C₆ branched saturated aliphatic hydrocarbon groups. In one embodiment a C₁₋₆ alkyl linker is a C₁, C₂, C₃, C₄, C₅, or C₆ alkyl linker group. Representative examples of alkyl linkers include, but are not limited to, moieties having from one to six carbon atoms, such as, methyl (—CH₂—), ethyl (—CH₂CH₂—), n-propyl (—CH₂CH₂CH₂—), i-propyl (—CHCH₃CH₂—), n-butyl (—CH₂CH₂CH₂CH₂—), s-butyl (—CHCH₃CH₂CH₂—), i-butyl (—C(CH₃)₂CH₂—), n-pentyl (—CH₂CH₂CH₂CH₂CH₂—), s-pentyl (—CHCH₃CH₂CH₂CH₂—), or n-hexyl (—CH₂CH₂CH₂CH₂CH₂CH₂—). The term “substituted alkyl linker” refers to alkyl linkers having substituents replacing one or more hydrogen atoms on one or more carbons of the hydrocarbon backbone. Such substituents do not alter the sp³-hybridization of the carbon atom to which they are attached and include those substituents listed below in the definition of the term “substituted.”

The term “carbocycle,” as used herein, refers to any stable monocyclic, bicyclic or tricyclic ring having the specified number of carbons, any of which may be saturated or unsaturated. In one embodiment, a C₃₋₁₄ carbocycle is intended to include a monocyclic, bicyclic, tricyclic, or spirocyclic (mono- or polycyclic) ring having 3, 4, 5, 6, 7, 8, 9, 10, 11, 12, 13, or 14 carbon atoms. Representative examples of carbocycles include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, fluorenyl, phenyl, naphthyl, indanyl, adamantly, tetrahydronaphthyl, octahydropentalene, ocatahydro-1H-indene, bicyclo[2.2.2]octane, spiro[3.4]octane, spiro[4.5]decane, spiro[4.5]deca-1,6-diene, and dispiro[2.2.4.2]dodecane. In one embodiment, the bridge linking to non-adjacent carbon atoms to form a tricyclic ring is a C₁ or C₂ bridge. When a ring is bridged, the substituents recited for the ring may also be present on the bridge.

The term “cycloalkyl,” as used herein, refers to a saturated or partially unsaturated cyclic alkyl group. Representative examples of cycloalkyl groups include, but are not limited to, cyclopropane, cyclobutane, cyclopentane, or cyclohexane. In one embodiment, a cycloalkyl group is C₃₋₁₅ cycloalkyl, C₃₋₁₂ cycloalkyl, or C₃₋₈ cycloalkyl.

The term “cycloalkylalkyl,” as used herein, refers to an acyclic alkyl group in which one of the hydrogen atoms bonded to a carbon atom, typically a terminal or sp³ carbon atom, is replaced with a cycloalkyl group. In certain embodiments, a cycloalkylalkyl group is C₄₋₃₀ cycloalkylalkyl, and, for example, the alkyl moiety of the cycloalkylalkyl group is C₁₋₁₀ and the cycloalkyl moiety is C₃₋₂₀. In another embodiment a cycloalkylalkyl group is C₃₋₂₀ cycloalkylalkyl, and, for example, the alkyl moiety of the cycloalkylalkyl group is C₁₋₈ and the cycloalkyl moiety is C₃₋₁₂. In a particular embodiment, a cycloalkylalkyl group is C₄₋₁₂ cycloalkylalkyl.

The term “deuterium enrichment factor”, as used herein, refers to the ratio between the isotopic abundance and the natural abundance of deuterium in a given sample of a compound.

The term “deuterium incorporation percentage,” as used herein, refers to the percentage of the molecules having deuterium at a particular position in a given sample of a compound out of the total amount of the molecules including deuterated and non-deuterated.

The terms “deuterated methyl” and “deuterated ethyl,” as used herein, refer to a methyl group and ethyl group, respectively, that contains at least one deuterium atom. Examples of deuterated methyl include —CDH₂, —CD₂H, and —CD₃. Examples of deuterated ethyl include, but are not limited to, —CHDCH₃, —CD₂CH₃, —CHDCDH₂, —CH₂CD₃.

The term “halogen,” as used herein, refers to fluoro, chloro, bromo, or iodo.

The term “heteroalkyl,” as used herein, by itself or as part of another substituent refers to an alkyl group in which one or more of the carbon atoms (and certain associated hydrogen atoms) are independently replaced with heteroatomic groups. Examples of heteroatomic groups include, but are not limited to, —O—, —S—, —O—O—, —S—S—, —O—S—, —NR′, ═N—N═, —N═N—, —N═N—NR′—, —PR′—, —P(O)₂—, —POR′—, —O—P(O)₂—, —SO—, —SO₂—, and —Sn(R′)₂—, where each R′ is independently hydrogen, C₁₋₆ alkyl, substituted C₁₋₆ alkyl, C₆₋₁₂ aryl, substituted C₆₋₁₂ aryl, C₇₋₁₈ arylalkyl, substituted C₇₋₁₈ arylalkyl, C₃₋₇ cycloalkyl, substituted C₃₋₇ cycloalkyl, C₃₋₇ heterocycloalkyl, substituted C₃₋₇ heterocycloalkyl, C₁₋₆ heteroalkyl, substituted C₁₋₆ heteroalkyl, C₆₋₁₂ heteroaryl, substituted C₆₋₁₂ heteroaryl, C₇₋₁₈ heteroarylalkyl, or substituted C₇₋₁₈ heteroarylalkyl. In one embodiment, a C₁₋₆ heteroalkyl, means, for example, a C₁₋₆ alkyl group in which at least one of the carbon atoms (and certain associated hydrogen atoms) is replaced with a heteroatom. In a particular embodiment, a C₁₋₆ heteroalkyl, for example, includes groups having five carbon atoms and one heteroatom, groups having four carbon atoms and two heteroatoms, etc. In one embodiment, each R′ is independently hydrogen or C₁₋₃ alkyl. In another embodiment, a heteroatomic group is —O—, —S—, —NH—, —N(CH₃)—, or —SO₂—. In a specific embodiment, the heteroatomic group is —O—.

The term “heteroaryl,” as used herein, refers to, for example, a 5-14 membered monocyclic-, bicyclic-, or tricyclic-ring system, having 1 to 10 heteroatoms independently selected from N, O, or S, wherein N and S can be optionally oxidized to various oxidation states, and wherein at least one ring in the ring system is aromatic. In one embodiment, the heteroaryl is monocyclic and has 5 or 6 ring members. Representative examples of monocyclic heteroaryl groups include, but are not limited to, pyridyl, thienyl, furanyl, pyrrolyl, pyrazolyl, imidazoyl, oxazolyl, isoxazolyl, thiazolyl, isothiazolyl, triazolyl, oxadiazolyl, thiadiazolyl and tetrazolyl. In another embodiment, the heteroaryl is bicyclic and has from 8 to 10 ring members. Representative examples of bicyclic heteroaryl groups include indolyl, benzofuranyl, quinolyl, isoquinolyl indazolyl, indolinyl, isoindolyl, indolizinyl, benzamidazolyl, quinolinyl, 5,6,7,8-tetrahydroquinoline, and 6,7-dihydro-5H-pyrrolo[3,2-d]pyrimidine.

The term “heteroarylalkyl,” as used herein, refers to an acyclic alkyl group in which one of the hydrogen atoms bonded to a carbon atom, typically a terminal or sp³ carbon atom, is replaced with a heteroaryl group. In certain embodiments, a heteroarylalkyl group is C₇₋₁₂ heteroarylalkyl, and, for example, the alkyl moiety of the heteroarylalkyl group is C₁₋₂ and the heteroaryl moiety is C₆₋₁₀.

The term “heterocycle,” as used herein, refers to any ring structure (saturated or partially unsaturated) which contains at least one ring heteroatom (e.g., N, O, or S). Examples of heterocycles include, but are not limited to, morpholine, pyrrolidine, tetrahydrothiophene, piperidine, piperazine and tetrahydrofuran.

The term “heterocycloalkyl,” as used herein, refers to a saturated or unsaturated cyclic alkyl group in which one or more carbon atoms (and certain associated hydrogen atoms) are independently replaced with one or more heteroatoms; or to a parent aromatic ring system in which one or more carbon atoms (and certain associated hydrogen atoms) are independently replaced with one or more heteroatoms such that the ring system no longer contains at least one aromatic ring. Representative examples of heteroatoms to replace the carbon atom(s) include, but are not limited to, N, P, O, S, and Si. Representative examples of heterocycloalkyl groups include, but are not limited to, epoxides, azirines, thiuranes, imidazolidine, morpholine, piperazine, piperidine, pyrazolidine, pyrrolidine, and quinuclidine. In one embodiment, a heterocycloalkyl group is C₅₋₁₀ heterocycloalkyl, C₅₋₈ heterocycloalkyl. In a specific embodiment a heterocycloalkyl group is C₅₋₆ heterocycloalkyl.

The term “heterocycloalkylalkyl,” as used herein, refers to an acyclic alkyl group in which one of the hydrogen atoms bonded to a carbon atom, typically a terminal or sp³ carbon atom, is replaced with a heterocycloalkyl group. In certain embodiments, a heterocycloalkylalkyl group is C₇₋₁₂ heterocycloalkylalkyl, and, for example, the alkyl moiety of the heterocycloalkylalkyl group is C₁₋₂ and the heterocycloalkyl moiety is C₆₋₁₀.

The term “isotopologue,” as used herein, refers to an isotopically enriched fumarate.

The term “isotopically enriched,” as used herein, refers to an atom having an isotopic composition other than the natural isotopic composition of that atom. In one embodiment an “isotopically enriched” fumarate contains at least one atom having an isotopic composition other than the natural isotopic composition of that atom.

The term “isotopic composition,” as used herein, refers to the amount of each isotope present for a given atom.

The term “pharmaceutically acceptable salt,” as used herein, refers to a salt prepared from a pharmaceutically acceptable non-toxic acid or base including an inorganic acid and base and an organic acid and base. Suitable pharmaceutically acceptable base addition salts of the fumarates provided herein include, but are not limited to, metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. Others are well known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995).

The term “stereoisomer” as used herein refers to one stereoisomer of a fumarate that is substantially free of other stereoisomers of that fumarate. For example, a “stereomerically pure” fumarate having one chiral center will be substantially free of the opposite enantiomer of the fumarate. A “stereomerically pure” fumarate having two chiral centers will be substantially free of the other diastereomers of the fumarate. A typical “stereomerically pure” fumarate comprises greater than about 80% by weight of one stereoisomer of the fumarate and less than about 20% by weight of other stereoisomers of the fumarate, greater than about 90% by weight of one stereoisomer of the fumarate and less than about 10% by weight of the other stereoisomers of the fumarate, greater than about 95% by weight of one stereoisomer of the fumarate and less than about 5% by weight of the other stereoisomers of the fumarate, or greater than about 97% by weight of one stereoisomer of the fumarate and less than about 3% by weight of the other stereoisomers of the fumarate. The fumarate can have chiral centers and can occur as racemates, individual enantiomers or diastereomers, and mixtures thereof. All such isomeric forms are included within the embodiments disclosed herein, including mixtures thereof. The use of stereomerically pure forms of such fumarates, as well as the use of mixtures of those forms, are encompassed by the embodiments disclosed herein. For example, mixtures comprising equal or unequal amounts of the enantiomers of a particular fumarate may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, N Y, 1962); and Wilen, S. H., Tables of Resolving Agents and Optical Resolutions p. 268 (E. L. Eliel, Ed., Univ. of Notre Dame Press, Notre Dame, Ind., 1972).

The term “substituted,” as used herein, refers to a group in which one or more hydrogen atoms are independently replaced with the same or different substituent group(s). In certain embodiments, each substituent group is independently halogen, —OH, —CN, —CF₃, ═O, —NO₂, benzyl, —C(O)NH₂, —R″, —OR″, —C(O)R″, —COOR″, —S(O)₂R″ or —NR₂″ wherein each R″ is independently hydrogen or C₁₋₆ alkyl. In certain embodiments, each substituent group is independently halogen, —OH, —CN, —CF₃, —NO₂, benzyl, —R″, —OR″, or —NR₂″ wherein each R″ is independently hydrogen or C₁₋₄ alkyl. In certain embodiments, each substituent group is independently halogen, —OH, —CN, —CF₃, ═O, —NO₂, benzyl, —C(O)NR₂″, —R″, —OR″, —C(O)R″, —COOR″, or —NR₂″ wherein each R″ is independently hydrogen or C₁₋₄ alkyl. In certain embodiments, each substituent group is independently —OH, C₁₋₄ alkyl, and —NH₂.

The number of carbon atoms in a group is specified herein by the prefix “Cₓ₋ₓₓ”, wherein x and xx are integers. For example, “C₁₋₄ alkyl” is an alkyl group which has from 1 to 4 carbon atoms; “C₁₋₆ alkyl” is an alkyl group having from 1 to 6 carbon atoms; and “C₆₋₁₀ aryl” is an aryl group which has from 6 to 10 carbon atoms.

### 4. BRIEF DESCRIPTION OF DRAWINGS

FIG. 1 shows sagittal, coronal, and transverse sections from PET imaged (FIG. 1A) and MR imaged (FIG. 1B) mice, as well as a merged image of PET and MR imaging (FIG. 1C) for mice orally administered (¹¹C)-DMF at 0.5 mg/kg.

FIG. 2 shows sagittal, coronal, and transverse sections from PET imaged (FIG. 2A) and MR imaged (FIG. 2B) mice, as well as a merged image of PET and MR imaging (FIG. 2C) for mice orally administered (¹¹C)-DMF at 200 mg/kg.

FIG. 3 shows sagittal, coronal, and transverse sections from PET imaged (FIG. 3A) and MR imaged (FIG. 3B) mice, as well as a merged image of PET and MR imaging (FIG. 3C) for mice intravenously administered (¹¹C)-DMF at 0.5 mg/kg.

FIG. 4 shows the quantified signal in various mouse tissues from PET imaging of mice administered (¹¹C)-DMF at a concentration of 0.5 mg/kg (intravenous).

FIG. 5 shows the quantified signal in various mouse tissues from PET imaging of mice administered (¹¹C)-DMF at a concentration of 0.5 mg/kg (oral).

FIG. 6 shows the quantified signal in various mouse tissues from PET imaging of mice administered (¹¹C)-DMF at a concentration of 200 mg/kg (oral).

FIG. 7 shows the quantified signal in various brain regions from PET imaging of mice administered (¹¹C)-DMF at a concentration of 0.5 mg/kg (intravenous).

FIG. 8 shows the quantified signal in various brain regions from PET imaging of mice administered (¹¹C)-DMF at a concentration of 0.5 mg/kg (oral).

FIG. 9 shows the quantified signal in various brain regions from PET imaging of mice administered (¹¹C)-DMF at a concentration 200 mg/kg (oral).

FIG. 10 shows a time course of PET imaging results for mice administered (¹¹C)-DMF at a concentration of 0.5 mg/kg (intravenous). Grey Scale: 0 to 12% of the % ID/g.

FIG. 11 shows a time course of PET imaging results for mice administered (¹¹C)-DMF at a concentration of 0.5 mg/kg (oral). Grey Scale: 0 to 12% of the % ID/g.

FIG. 12 shows a time course of PET imaging results for mice administered DMF at a concentration of 200 mg/kg (oral). Grey Scale: 0 to 12% of the % ID/g

FIG. 13 shows the results of mice administered (¹⁴C)DMF intravenously at a concentration of 0.5 mg/kg viewed in sagittal section either 10 minutes (FIGS. 13A and B) or 60 minutes (FIGS. 13C and D) after administration.

FIG. 14 shows the results of mice administered (¹⁴C)DMF orally at a concentration of 0.5 mg/kg viewed in sagittal section either 10 minutes (FIGS. 14A and B) or 60 minutes (FIGS. 14C and D) after administration.

FIG. 15 shows box and whisker MMF exposure plots. Plasma (FIG. 15A), jejunum (FIG. 15B), forebrain (FIG. 15C), cerebellum (FIG. 15D), kidney (FIG. 15E), and spleen (FIG. 15F) 10 minutes and 2 hours after DMF dosing. Black bars represent PO dosing (100 mg/kg) and gray bars represent IV dosing (30 mg/kg). FIG. 15G shows tissue to plasma ratios after IV and PO dosing in various tissues (tissue [MMF]/plasma [MMF]*100). For box and whiskers plots: boxes represent 1st and 3rd quartile values, the median is the horizontal line within the box, and bars represent the minimum and maximum value; n=5. Statistical comparisons were performed with Mann-Whitney U Test (*, p<0.05; **, p<0.01; ***, p<0.001; ****, p<0.0001).

FIG. 16 shows the transcriptional changes in forebrain after IV and PO administration of DMF at 2 and 6 hours. NADP (H) dehydrogenase quinone 1 (Ngo1) (FIG. 16A); oxidative stress induced growth inhibitor 1 (Osgin1) (FIG. 16B); aldo-keto reductase family 1, member b8 (Akr1b8) (FIG. 16C); glutamate-cysteine ligase, catalytic subunit (Gclc) (FIG. 16D); heme oxygenase 1 (Hmox1) (FIG. 16E); thioredoxin reductase 1 (Txnrd1) (FIG. 16F). Bars represent mean determinations of fold-change of indicated genes relative to vehicle controls (n=5). Hatch-marked bars represent vehicle control, black bars the 2 hour time point and gray bars the 6 hour time point. Error bars indicate standard deviation. Statistical comparisons were performed using ANOVA with Tukey's multiple comparisons to evaluate differences between animals receiving vehicle or DMF within the same dosing regimen (*, p<0.05; **, p<0.01; ***, p<0.001; ****, p<0.0001).

FIG. 17 shows the transcriptional changes in cerebellum after IV and PO administration of DMF at 2 and 6 hours. NADP (H) dehydrogenase quinone 1 (Ngo1) (FIG. 17A); oxidative stress induced growth inhibitor 1 (Osgin1) (FIG. 17B); aldo-keto reductase family 1, member b8 (Akr1b8) (FIG. 17C); glutamate-cysteine ligase, catalytic subunit (Gclc) (FIG. 17D); heme oxygenase 1 (Hmox1) (FIG. 17E); thioredoxin reductase 1 (Txnrd1) (FIG. 17F). Bars represent mean determinations of fold-change of indicated genes relative to vehicle controls (n=5). Hatch-marked bars represent vehicle control, black bars the 2 hour time point and gray bars the 6 hour time point. Error bars indicate standard deviation. Statistical comparisons were performed using ANOVA with Tukey's multiple comparisons to evaluate differences between animals receiving vehicle or DMF within the same dosing regimen (*, p<0.05; **, p<0.01; ***, p<0.001; ****, p<0.0001).

FIG. 18 shows the transcriptional changes in kidney after IV and PO administration of DMF at 2 and 6 hours. NADP (H) dehydrogenase quinone 1 (Ngo1) (FIG. 18A); oxidative stress induced growth inhibitor 1 (Osgin1) (FIG. 18B); aldo-keto reductase family 1, member b8 (Akr1b8) (FIG. 18C); glutamate-cysteine ligase, catalytic subunit (Gclc) (FIG. 18D); heme oxygenase 1 (Hmox1) (FIG. 18E); thioredoxin reductase 1 (Txnrd1) (FIG. 18F). Bars represent mean determinations of fold-change of indicated genes relative to vehicle controls (n=5). Hatch-marked bars represent vehicle control, black bars the 2 hour time point and gray bars the 6 hour time point. Error bars indicate standard deviation. Statistical comparisons were performed using ANOVA with Tukey's multiple comparisons to evaluate differences between animals receiving vehicle or DMF within the same dosing regimen (*, p<0.05; **, p<0.01; ***, p<0.001; ****, p<0.0001).

FIG. 19 shows the transcriptional changes in spleen after IV and PO administration of DMF at 2 and 6 hours. NADP (H) dehydrogenase quinone 1 (Ngo1) (FIG. 19A); oxidative stress induced growth inhibitor 1 (Osgin1) (FIG. 19B); aldo-keto reductase family 1, member b8 (Akr1b8) (FIG. 19C); glutamate-cysteine ligase, catalytic subunit (Gclc) (FIG. 19D); heme oxygenase 1 (Hmox1) (FIG. 19E); thioredoxin reductase 1 (Txnrd1) (FIG. 19F). Bars represent mean determinations of fold-change of indicated genes relative to vehicle controls (n=5). Hatch-marked bars represent vehicle control, black bars the 2 hour time point and gray bars the 6 hour time point. Error bars indicate standard deviation. Statistical comparisons were performed using ANOVA with Tukey's multiple comparisons to evaluate differences between animals receiving vehicle or DMF within the same dosing regimen (*, p<0.05; **, p<0.01; ***, p<0.001; ****, p<0.0001).

FIG. 20 shows the transcriptional changes in jejunum after IV and PO administration of DMF at 2 and 6 hours. NADP (H) dehydrogenase quinone 1 (Ngo1) (FIG. 20A); oxidative stress induced growth inhibitor 1 (Osgin1) (FIG. 20B); aldo-keto reductase family 1, member b8 (Akr1b8) (FIG. 20C); glutamate-cysteine ligase, catalytic subunit (Gclc) (FIG. 20D); heme oxygenase 1 (Hmox1) (FIG. 20E); thioredoxin reductase 1 (Txnrd1) (FIG. 20F). Bars represent mean determinations of fold-change of indicated genes relative to vehicle controls (n=5). Hatch-marked bars represent vehicle control, black bars the 2 hour time point and gray bars the 6 hour time point. Error bars indicate standard deviation. Statistical comparisons were performed using ANOVA with Tukey's multiple comparisons to evaluate differences between animals receiving vehicle or DMF within the same dosing regimen (*, p<0.05; **, p<0.01; ***, p<0.001; ****, p<0.0001).

FIG. 21 shows an analysis of MMF exposures at 10 minutes (mean±standard deviation, n=5, X-axis) graphed against normalized fold change in pharmacodynamic response at 2 or 6 hours (mean±standard deviation, n=5, Y-axis), comparing DMF administered PO (100 mg/kg, black circles) and IV (30 mg/kg, gray squares). FIG. 21A-C: Exposure-pharmacodynamic relationships in forebrain for Osgin1 at 2 hours (FIG. 21A), Akr1b8 at 6 hours (FIG. 21B), and (FIG. 21C) Hmox1 at 6 hours. FIG. 21D-F: Exposure-pharmacodynamic relationships in kidney for Nqo1 at 6 hours (FIG. 21D), Hmox1 at 2 hours (FIG. 21E), and Txnrd1 at 6 hours (FIG. 21F). FIG. 21G-I: Exposure-pharmacodynamic relationships in spleen for Nqo1 at 6 hours (FIG. 21G), Osgin1 at 2 hours (FIG. 21H), and Akr1b8 at 2 hours (FIG. 21I).

FIG. 22 shows MMF levels measured in plasma (FIG. 22A), brain (FIG. 22B), jejunum (FIG. 22C), and kidney (FIG. 22D) 10 minutes after administration of vehicle or DMF PO (100 mg/kg, black bars) or IV (17.5 mg/kg, open bars, or 30 mg/kg, gray bars). Bars represent mean values (n=4), error bars denote standard deviation. Evaluation of MMF tissue penetration at 10 minutes after dosing DMF PO or IV in brain (FIG. 22E), kidney (FIG. 22F), and jejunum (FIG. 22G). Bars represent mean values of (tissue [MMF]/plasma [MMF]*100), error bars denote standard deviation (n=4). Statistical comparisons were performed using an analysis of variance (ANOVA) with Tukey's multiple comparison test to evaluate changes between routes of administration and between IV dose levels. * p<0.05; **, p<0.01; ****, p<0.0001.

FIG. 23 shows the fold-change of transcript levels of certain genes (Aldo-keto reductase family 1, member b8 (Akr1b8) (FIG. 23A); glutamate-cysteine ligase, catalytic subunit (Gclc) (FIG. 23B); heme oxygenase 1 (Hmox1) (FIG. 23C); NADP(H) dehydrogenase quinone 1 (Ngo1) (FIG. 23D); and oxidative stress induced growth inhibitor 1 (Osgin1) (FIG. 23E)) in the brain two hours after the administration of DMF by oral gavage (PO, 100 mg/kg, black bars) or intravenous infusion (IV, 17.5 mg/kg, open bars, or 30 mg/kg, gray bars) relative to vehicle controls. Hatch-marked bars represent vehicle control levels. Bars represent mean determinations of fold-change of indicated genes relative to vehicle controls (n=4), error bars indicate standard deviation. Statistical comparisons were performed for the PO groups using Student's t-test. IV groups were analyzed ANOVA with Tukey's multiple comparison test to evaluate differences between vehicle, DMF 17.5 mg/kg and DMF 30 mg/kg groups. *, p<0.05; **, p<0.01; ***, p<0.001; ****, p<0.0001.

FIG. 24 shows the fold-change of transcript levels of certain genes (Aldo-keto reductase family 1, member b8 (Akr1b8) (FIG. 24A); glutamate-cysteine ligase, catalytic subunit (Gclc) (FIG. 24B); heme oxygenase 1 (Hmox1) (FIG. 24C); NADP(H) dehydrogenase quinone 1 (Ngo1) (FIG. 24D); and oxidative stress induced growth inhibitor 1 (Osgin1) (FIG. 24E)) in the kidney two hours after the administration of DMF by oral gavage (PO, 100 mg/kg, black bars) or intravenous infusion (IV, 17.5 mg/kg, open bars, or 30 mg/kg, gray bars) relative to vehicle controls. Hatch-marked bars represent vehicle control levels. Bars represent mean determinations of fold-change of indicated genes relative to vehicle controls (n=4), error bars indicate standard deviation. Statistical comparisons were performed for the PO groups using Student's t-test. IV groups were analyzed ANOVA with Tukey's multiple comparison test to evaluate differences between vehicle, DMF 17.5 mg/kg and DMF 30 mg/kg groups. *, p<0.05; **, p<0.01; ***, p<0.001; ****, p<0.0001.

FIG. 25 shows the fold-change of transcript levels of certain genes (Aldo-keto reductase family 1, member b8 (Akr1b8) (FIG. 25A); glutamate-cysteine ligase, catalytic subunit (Gclc) (FIG. 25B); heme oxygenase 1 (Hmox1) (FIG. 25C); NADP(H) dehydrogenase quinone 1 (Ngo1) (FIG. 25D); and oxidative stress induced growth inhibitor 1 (Osgin1) (FIG. 25E)) in the jejunum two hours after the administration of DMF by oral gavage (PO, 100 mg/kg, black bars) or intravenous infusion (IV, 17.5 mg/kg, open bars, or 30 mg/kg, gray bars) relative to vehicle controls. Hatch-marked bars represent vehicle control levels. Bars represent mean determinations of fold-change of indicated genes relative to vehicle controls (n=4), error bars indicate standard deviation. Statistical comparisons were performed for the PO groups using Student's t-test. IV groups were analyzed ANOVA with Tukey's multiple comparison test to evaluate differences between vehicle, DMF 17.5 mg/kg and DMF 30 mg/kg groups. *, p<0.05; **, p<0.01.

FIG. 26 shows the evaluation of mean tissue MMF exposures at 10 minutes (±standard deviation, x-axis) for DMF PO (100 mg/kg, solid black circle) and IV (17.5 mg/kg, open gray square or 30 mg/kg, open gray triangle) graphed against indicated pharmacodynamic transcriptional changes for brain (FIG. 26A, B), kidney (FIG. 26C, D), and jejunum (FIG. 26E, F). Mean fold-changes measured at 2 hours are graphed on the Y-axis±standard deviation for Osgin1 (FIG. 26A), Akr1b8 (FIG. 26B, E), Hmox1 (FIG. 26C, F) and Ngo1 (FIG. 26D). Dashed line at Y=1 represents basal gene expression levels observed in the vehicle controls.

FIG. 27 shows MMF levels that were measured in plasma (FIG. 27A), brain (FIG. 27B), kidney (FIG. 27C), jejunum (FIG. 27D), and spleen (FIG. 27E) 10 minutes after IV administration of DMF (30 mg/kg, black bars) or MMF (27 mg/kg, gray bars). Bars represent mean values (n=4), error bars denote standard deviation. FIG. 27F shows the evaluation of MMF tissue penetration at 10 minutes after dosing IV DMF or MMF in brain, kidney, jejunum and spleen. Bars represent mean values of (tissue [MMF]/plasma [MMF]*100), n=4. Error bars denote standard deviation. Statistical comparisons were performed using t-tests for individual tissues comparing DMF to MMF IV administration. * p<0.05.

FIG. 28 shows the fold-change of transcript levels of certain genes (Aldo-keto reductase family 1, member b8 (Akr1b8) (FIG. 28A); glutamate-cysteine ligase, catalytic subunit (Gclc) (FIG. 28B); heme oxygenase 1 (Hmox1) (FIG. 28C); NADP(H) dehydrogenase quinone 1 (Ngo1) (FIG. 28D); and oxidative stress induced growth inhibitor 1 (Osgin1) (FIG. 28E)) in the brain two or six hours after IV administration of DMF, MMF or vehicle (30 mg/kg, black bars or 27 mg/kg, gray bars, vehicle hatched bars, respectively) relative to vehicle controls. Bars represent mean determinations of fold-change of indicated genes relative to vehicle controls (hatched bars) for matched time points (n=4), error bars indicate standard deviation. Statistical comparisons were performed using ANOVA with Tukey's multiple comparison test to evaluate changes between animals receiving vehicle, DMF or MMF. Two and 6-hour time points were analyzed separately. *, p<0.05; **, p<0.01.

FIG. 29 shows the fold-change of transcript levels of certain genes (Aldo-keto reductase family 1, member b8 (Akr1b8) (FIG. 29A); glutamate-cysteine ligase, catalytic subunit (Gclc) (FIG. 29B); heme oxygenase 1 (Hmox1) (FIG. 29C); NADP(H) dehydrogenase quinone 1 (Ngo1) (FIG. 29D); and oxidative stress induced growth inhibitor 1 (Osgin1) (FIG. 29E)) in the kidney two or six hours after IV administration of DMF, MMF or vehicle (30 mg/kg, black bars or 27 mg/kg, gray bars, vehicle hatched bars, respectively) relative to vehicle controls. Bars represent mean determinations of fold-change of indicated genes relative to vehicle controls (hatched bars) for matched time points (n=4), error bars indicate standard deviation. Statistical comparisons were performed using ANOVA with Tukey's multiple comparison test to evaluate changes between animals receiving vehicle, DMF or MMF. Two and 6-hour time points were analyzed separately. *, p<0.05; **, p<0.01; ***, p<0.001; **** p<0.0001.

FIG. 30 shows the fold-change of transcript levels of certain genes (Aldo-keto reductase family 1, member b8 (Akr1b8) (FIG. 30A); glutamate-cysteine ligase, catalytic subunit (Gclc) (FIG. 30B); heme oxygenase 1 (Hmox1) (FIG. 30C); NADP(H) dehydrogenase quinone 1 (Ngo1) (FIG. 30D); and oxidative stress induced growth inhibitor 1 (Osgin1) (FIG. 30E)) in the jenunum two or six hours after IV administration of DMF, MMF or vehicle (30 mg/kg, black bars or 27 mg/kg, gray bars, vehicle hatched bars, respectively) relative to vehicle controls. Bars represent mean determinations of fold-change of indicated genes relative to vehicle controls (hatched bars) for matched time points (n=4), error bars indicate standard deviation. Statistical comparisons were performed using ANOVA with Tukey's multiple comparison test to evaluate changes between animals receiving vehicle, DMF or MMF. Two and 6-hour time points were analyzed separately. *, p<0.05; **, p<0.01; ***, p<0.001; ****, p<0.0001.

FIG. 31 shows an evaluation of tissue MMF exposures at 10 minutes (mean±standard deviation, n=4, X-axis) for IV DMF (30 mg/kg, solid black circle) and IV MMF (27 mg/kg, gray square) graphed against indicated pharmacodynamic transcriptional changes for brain (FIG. 31A, B), kidney (FIG. 31C, D, E), and jejunum (FIG. 31F, G, H). Mean fold-changes measured at 2 hours are graphed on the Y-axis±standard deviation for Osgin1 (FIG. 31A, E), Akr1b8 (FIG. 31F), Hmox1 (FIG. 31C), and Nqo1 (FIG. 31B, D, G) (n=4). Dashed line at Y=1 represents basal gene expression observed in the vehicle controls.

FIG. 32 shows an analysis of white blood cell counts 10 minutes, 2 hours, and 6 hours (Hr) after IV dosing of DMF (30 mg/kg, black bars), MMF (27 mg/kg, gray bars) or vehicle (20% Captisol, open bars). White blood cells (FIG. 32A), Neutrophils (FIG. 32B), Lymphocytes (FIG. 32C), Monocytes (FIG. 32D), Eosinophils (FIG. 32E) and Basophils (FIG. 32F). Bars represent mean cell counts, error bar indicates standard deviation (n=4, each group).

FIG. 33 shows an analysis of red blood cells and platelets 10 minutes, 2 hours (Hr), and 6 hours after IV dosing of DMF (30 mg/kg, black bars), MMF (27 mg/kg, gray bars) or vehicle (20% Captisol, open bars). Red blood cells (FIG. 33A), hemoglobin levels (FIG. 33B), hematocrit (FIG. 33C), mean corpuscular volume (FIG. 33D), and platelets (FIG. 33E). Bars represent mean cell counts and values, error bar indicates standard deviation (n=4, each group).

FIG. 34 shows the fold-change of transcript levels of certain genes (Aldo-keto reductase family 1, member b8 (Akr1b8) (FIG. 34A); heme oxygenase 1 (Hmox1) (FIG. 34B); NADP(H) dehydrogenase quinone 1 (Ngo1) (FIG. 34C); oxidative stress induced growth inhibitor 1 (Osgin1) (FIG. 34D); and glutamate-cysteine ligase, catalytic subunit (Gclc) (FIG. 34E)) in various tissues two hours after the last (5^{th}) IV dose of DMF 30 mg/kg or vehicle relative to vehicle controls. Gray bars represent mean determinations of DMF-induced fold-change of indicated genes relative to vehicle controls (black bars) for matched time points (n=4 vehicle, n=5 DMF), error bars indicate standard deviation. Student's t-test was utilized to compare vehicle versus DMF within each tissue. * p<0.05, **p<0.01, **** p<0.0001.

FIG. 35 shows an analysis of white blood cell counts 10 minutes after last (5th) IV dose of DMF (30 mg/kg, gray bars) or vehicle (20% Captisol, black bars). Bars represent mean cell counts, error bar indicates standard deviation (n=4 vehicle, n=5 DMF). Statistical comparisons were performed using Student's t-test comparing vehicle to DMF for each indicated cell type. * p<0.05.

FIG. 36 shows the impact of orally administered DMF (100 mg/kg daily) on rotarod performance of SOD1-G93A mice.

FIG. 37 shows the impact of orally administered DMF (p.o. 100 mg/kg daily) on the onset of motor neuron symptoms (FIG. 37A) and survival (FIG. 37B) in the SOD1-G93A mice for the vehicle and DMF groups.

FIG. 38 shows a break point analysis indicating the transition from weight gain to weight loss for vehicle and DMF groups.

FIG. 39 shows the effect of DMF (p.o.) compared to vehicle for Experiment 1 (FIG. 39A) and Experiment 2 (FIG. 39B) in the malonate-induced striatial lesion model.

FIG. 40 shows the effect of DMF (p.o.) on rotational behavior in rats after administration of apomorphine (1.0 mg/kg, s.c.).

FIG. 41 shows representative images of lesioned rat brain sections staining for immunofluorescence (Astrocytes, FIG. 41A, B; Neurons, FIG. 41C, D). Vehicle (FIG. 41A, C) and DMF (p.o., 100 mg/kg) (FIG. 41B, D).

FIG. 42 shows MMF exposure in malonate model 30 min after last oral dose of DMF in mg/kg in plasma, brain, and cerebrospinal fluid (CSF).

FIG. 43 shows an HPLC of the nano suspension of at day 1 (FIG. 43A; t=3.829 min (MMF) and t=7.196 min (DMF) and day 7 (FIG. 43B; t=3.819 min (MMF); t=7.163 min (DMF)).

FIG. 44 shows the particle size distribution of the nano suspension at day 1 (FIG. 44A) and day 7 (FIG. 44B).

### 5. DETAILED DESCRIPTION

Provided herein are methods of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing. In one embodiment, the fumarate is dimethyl fumarate.

In one embodiment, the method consists essentially of said administering step.

In one embodiment, the at least one fumarate is the only active agent administered to the patient for said treating.

In one embodiment, the only active agent in the pharmaceutical composition is the at least one fumarate. In one embodiment, the only active agents in the pharmaceutical composition are dimethyl fumarate and monomethyl fumarate. In one embodiment, the only active agent in the pharmaceutical composition is one fumarate selected from said group. In one embodiment, the only active agent in the pharmaceutical composition is dimethyl fumarate and optionally one or more compounds produced by degradation from dimethyl fumarate in said pharmaceutical composition prior to said administering. In one embodiment, the only active agent in the pharmaceutical composition is dimethyl fumarate. In one embodiment, the pharmaceutical composition consists essentially of the at least one fumarate. In one embodiment, the pharmaceutical composition consists essentially of dimethyl fumarate.

In one embodiment, said administering is part of a treatment regimen wherein said administering intravenously to the patient alternates with one or more steps of administering the fumarate orally to the patient.

All of the various aspects, embodiments, and options disclosed herein can be combined in any and all variations. The compositions and methods provided are exemplary and are not intended to limit the scope of the claimed embodiments.

### 5.1 Fumarates for Use in the Methods Provided Herein

The active agent (i.e., drug) for use in the methods and compositions disclosed herein is at least one fumarate. Such a fumarate can be a dialkyl fumarate (e.g., dimethyl fumarate), a monoalkyl fumarate (e.g., monomethyl fumarate), a combination of dialkyl and monoalkyl fumarates (e.g., dimethyl fumarate and monomethyl fumarate), a prodrug of monoalkyl (e.g., monomethyl) fumarate, a deuterated form of any of the foregoing, or a pharmaceutically acceptable salt, tautomer, or stereoisomer of any of the foregoing, or a combination of any of the foregoing. In one embodiment, the fumarate used in the methods, compositions and products described in this specification is dimethyl fumarate. In a specific embodiment, the fumarate is (i) a monoalkyl fumarate or prodrug thereof, or (ii) a dialkyl fumarate. In one embodiment, the monoalkylfumarate is monomethyl fumarate (“MMF”). In another embodiment, the dialkyl fumarate is dimethyl fumarate (“DMF”).

### 5.1.1 Mono- and Dialkyl Fumarates

In particular, provided herein are mono- and dialkyl fumarates or pharmaceutically acceptable salts, or stereoisomers thereof for use in the methods provided herein.

In one embodiment, the fumarate is a monoalkyl fumarate of Formula (I):

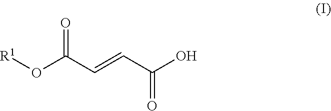

or a pharmaceutically acceptable salt, or stereoisomer thereof, wherein

R¹ is C₁₋₆ alkyl.

In certain embodiments of a compound of Formula (I), R¹ is methyl (monomethyl fumarate, “MMF”).

In one embodiment, the compounds of Formula (I) may be prepared using methods known to those skilled in the art, for example, as disclosed in U.S. Pat. No. 4,959,389.

In another embodiment, the fumarate is a dialkyl fumarate of Formula (II):

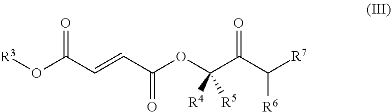

or a pharmaceutically acceptable salt, or stereoisomer thereof, wherein

each R² is independently C₁₋₆ alkyl.

In certain embodiments of a compound of Formula (II), each R² is methyl (dimethyl fumarate, “DMF”).

In one embodiment, the compounds of Formula (II) may be prepared using methods known to those skilled in the art, for example, as disclosed in U.S. Pat. No. 4,959,389.

In one embodiment, the fumarate is dimethyl fumarate and/or monomethyl fumarate.

In one embodiment, the fumarate is dimethyl fumarate.

### 5.1.2 Prodrugs of Monoalkyl Fumarates

Further provided herein are prodrugs of monoalkyl fumarates or pharmaceutically acceptable salts, or stereoisomers thereof for use in the methods provided herein.

In particular, the prodrugs of monoalkyl fumarates are the prodrugs disclosed in WO2013/119677, such as the communds of Formula (BD:

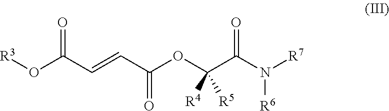

or a pharmaceutically acceptable salt, tautomer, or stereoisomer thereof,
R³ is C₁₋₆ alkyl;
R⁴ and R⁵ are each independently hydrogen, C₁₋₆ alkyl, or substituted C₁₋₆ alkyl;
R⁶ and R⁷ are each independently hydrogen, C₁₋₆ alkyl, substituted C₁₋₆ alkyl, C₁₋₆ heteroalkyl, substituted C₁₋₆ heteroalkyl, C₄₋₁₂ cycloalkylalkyl, substituted C₄₋₁₂ cycloalkylalkyl, C₇₋₁₂ arylalkyl, or substituted C₇₋₁₂ arylalkyl; or R⁶ and R⁷ together with the nitrogen to which they are attached form a ring chosen from C₅₋₁₀ heteroaryl, substituted C₅₋₁₀ heteroaryl, C₅₋₁₀ heterocycloalkyl, and substituted C₅₋₁₀ heterocycloalkyl; and
wherein each substituent is independently halogen, —OH, —CN, —CF₃, ═O, —NO₂, benzyl, —C(O)NR⁸₂, —R⁸, —C(O)R⁸, —COW, or —NR⁸₂ wherein each R⁸ is independently hydrogen or C₁₋₄ alkyl.

In certain embodiments of a compound of Formula (III), when R³ is ethyl; then R⁶ and R⁷ are each independently hydrogen, C₁₋₆ alkyl, or substituted C₁₋₆ alkyl.

In certain embodiments of a compound of Formula (III), each substituent group is independently halogen, —OH, —CN, —CF₃, —R⁸, —OR⁸, or —NR⁸₂ wherein each R⁸ is independently hydrogen or C₁₋₄ alkyl. In certain embodiments, each substituent group is independently —OH or —COOH.

In certain embodiments of a compound of Formula (III), each substituent group is independently ═O, C₁₋₄ alkyl, or —COOR⁸, wherein R⁸ is hydrogen or C₁₋₄ alkyl.

In certain embodiments of a compound of Formula (III), R³ is methyl.

In certain embodiments of a compound of Formula (III), R³ is ethyl.

In certain embodiments of a compound of Formula (III), R³ is C₃₋₆ alkyl.

In certain embodiments of a compound of Formula (III), R³ is methyl, n-propyl, isopropyl, n-butyl, sec-butyl, isobutyl, or tert-butyl.

In certain embodiments of a compound of Formula (III), R³ is methyl, ethyl, n-propyl, isopropyl, n-butyl, sec-butyl, isobutyl, or tert-butyl.

In certain embodiments of a compound of Formula (III), each of R⁴ and R⁵ is hydrogen.

In certain embodiments of a compound of Formula (III), one of R⁴ and R⁵ is hydrogen and the other of R⁴ and R⁵ is C₁₋₄ alkyl.

In certain embodiments of a compound of Formula (III), one of R⁴ and R⁵ is hydrogen and the other of R⁴ and R⁵ is methyl, ethyl, n-propyl, isopropyl, n-butyl, isobutyl, sec-butyl, or tert-butyl.

In certain embodiments of a compound of Formula (III), one of R⁴ and R⁵ is hydrogen and the other of R⁴ and R⁵ is methyl.

In certain embodiments of a compound of Formula (III), R⁶ and R⁷ are each independently hydrogen or C₁₋₆ alkyl.

In certain embodiments of a compound of Formula (III), R⁶ and R⁷ are each independently hydrogen or C₁₋₄ alkyl.

In certain embodiments of a compound of Formula (III), R⁶ and R⁷ are each independently hydrogen, methyl, or ethyl.

In certain embodiments of a compound of Formula (III), R⁶ and R⁷ are each hydrogen; in certain embodiments, R⁶ and R⁷ are each methyl; and in certain embodiments, R⁶ and R⁷ are each ethyl.

In certain embodiments of a compound of Formula (III), R⁶ is hydrogen; and R⁷ is C₁₋₄ alkyl, substituted C₁₋₄ alkyl wherein each substituent independently is ═O, —OR⁸, —COOR⁸, or —NR⁸₂, and wherein each R⁸ is independently hydrogen or C₁₋₄ alkyl.

In certain embodiments of a compound of Formula (III), R⁶ is hydrogen; and R⁷ is C₁₋₄ alkyl, benzyl, 2-methoxyethyl, carboxymethyl, carboxypropyl, 1,3,4-thiadiazolyl, methoxy, —COOCH₃, 2-oxo-1,3-oxazolidinyl, 2-(methylethoxy)ethyl, 2-ethoxyethyl, (tert-butyloxycarbonyl)methyl, (ethoxycarbonyl)methyl, (methylethyl)oxycarbonylmethyl, or ethoxycarbonylmethyl.

In certain embodiments of a compound of Formula (III), R⁶ and R⁷ together with the nitrogen to which they are attached form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (III), R⁶ and R⁷ together with the nitrogen to which they are attached form a ring chosen from a C₅ heterocycloalkyl, substituted C₅ heterocycloalkyl, C₅ heteroaryl, and substituted C₅ heteroaryl ring. In certain embodiments of a compound of Formula (III), R⁶ and R⁷ together with the nitrogen to which they are attached form a ring chosen from a C₆ heterocycloalkyl, substituted C₆ heterocycloalkyl, C₆ heteroaryl, and substituted C₆ heteroaryl ring. In certain embodiments of a compound of Formula (III), R⁶ and R⁷ together with the nitrogen to which they are attached form a ring chosen from piperazine, 1,3-oxazolidinyl, pyrrolidine, and morpholine ring.

In certain embodiments of a compound of Formula (III), R⁶ and R⁷ together with the nitrogen to which they are attached form a C₅₋₁₀ heterocycloalkyl ring.

In certain embodiments of a compound of Formula (III), one of R⁴ and R⁵ is hydrogen and the other of R⁴ and R⁵ is C₁₋₆ alkyl; R⁶ is hydrogen; R⁷ is hydrogen, C₁₋₆ alkyl, or benzyl.

In certain embodiments of a compound of Formula (III), R³ is methyl; one of R⁴ and R⁵ is hydrogen and the other of R⁴ and R⁵ is C₁₋₆ alkyl; R⁶ is hydrogen; and R⁷ is hydrogen, C₁₋₆ alkyl, or benzyl.

In certain embodiments of a compound of Formula (III), one of R⁴ and R⁵ is hydrogen and the other of R⁴ and R⁵ is hydrogen or C₁₋₆ alkyl; and each of R⁶ and R⁷ is C₁₋₆ alkyl.

In certain embodiments of a compound of Formula (III), R³ is methyl; one of R⁴ and R⁵ is hydrogen and the other of R⁴ and R⁵ is hydrogen or C₁₋₆ alkyl; and each of R⁶ and R⁷ is C₁₋₆ alkyl. In certain embodiments of a compound of Formula (III), R⁵ is methyl; each of R⁴ and R⁵ is hydrogen; and each of R⁶ and R⁷ is C₁₋₆ alkyl.

In certain embodiments of a compound of Formula (III), R³ is methyl; one of R⁴ and R⁵ is hydrogen and the other of R⁴ and R⁵ is hydrogen or C₁₋₄ alkyl; R⁶ is hydrogen; and R⁷ is C₁₋₄ alkyl or substituted C₁₋₄ alkyl wherein the substituent group is ═O, —OR⁸, —COOR⁸, or —NR⁸₂, wherein each R⁸ is independently hydrogen or C₁₋₄ alkyl. In certain embodiments of a compound of Formula (III), R³ is methyl; one of R⁴ and R⁵ is hydrogen and the other of R⁴ and R⁵ is methyl; R⁶ is hydrogen; and R⁷ is C₁₋₄ alkyl or substituted C₁₋₄ alkyl wherein the substituent group is ═O, —OR⁸, —COW, or —NR⁸₂, wherein each R⁸ is independently hydrogen or C₁₋₄ alkyl. In certain embodiments of a compound of Formula (III), R³ is methyl; each of R⁴ and R⁵ is hydrogen; R⁶ is hydrogen; and R⁷ is C₁₋₄ alkyl or substituted C₁₋₄ alkyl wherein the substituent group is ═O, —OR¹¹, —COOR¹¹, or —NR¹¹₂, wherein each R¹¹ is independently hydrogen or C₁₋₄ alkyl.

In certain embodiments of a compound of Formula (III), R⁶ and R⁷ together with the nitrogen to which they are attached form a C₅₋₁₀ heterocycloalkyl ring.

In certain embodiments of a compound of Formula (III), R³ is methyl; one of R⁴ and R⁵ is hydrogen and the other of R⁴ and R⁵ is hydrogen or C₁₋₆ alkyl; and R⁶ and R⁷ together with the nitrogen to which they are attached form a ring chosen from C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (III), R³ is methyl; one of R⁴ and R⁵ is hydrogen and the other of R⁴ and R⁵ is methyl; R⁶ and R⁷ together with the nitrogen to which they are attached form a ring chosen from a C₅₋₆ heterocycloalkyl, substituted C₅₋₆ heterocycloalkyl, C₅₋₆ heteroaryl, and substituted C₅₋₆ heteroaryl ring. In certain embodiments of a compound of Formula (III), R³ is methyl; each of R⁴ and R⁵ is hydrogen; and R⁶ and R⁷ together with the nitrogen to which they are attached form a ring chosen from C₅₋₆ heterocycloalkyl, substituted C₅₋₆ heterocycloalkyl, C₅₋₆ heteroaryl, and substituted C₅₋₆ heteroaryl ring.

In certain embodiments of a compound of Formula (III), one of R⁴ and R⁵ is hydrogen and the other of R⁴ and R⁵ is hydrogen or C₁₋₆ alkyl; and R⁶ and R⁷ together with the nitrogen to which they are attached form a ring chosen from morpholine, piperazine, and N-substituted piperazine.

In certain embodiments of a compound of Formula (III), R³ is methyl; one of R⁴ and R⁵ is hydrogen and the other of R⁴ and R⁵ is hydrogen or C₁₋₆ alkyl; and R⁶ and R⁷ together with the nitrogen to which they are attached form a ring chosen from morpholine, piperazine, and N-substituted piperazine.

In certain embodiments of a compound of Formula (III), R³ is not methyl.

In certain embodiments of a compound of Formula (III), R⁴ is hydrogen, and in certain embodiments, R⁵ is hydrogen.

In certain embodiments of a compound of Formula (III), R⁶ and R⁷ are independently hydrogen, C₁₋₆ alkyl, substituted C₁₋₆ alkyl, C₆₋₁₀ aryl, substituted C₆₋₁₀ aryl, C₄₋₁₂ cycloalkylalkyl, substituted C₄₋₁₂ cycloalkylalkyl, C₇₋₁₂ arylalkyl, substituted C₇₋₁₂ arylalkyl, C₁₋₆ heteroalkyl, substituted C₁₋₆ heteroalkyl, C₆₋₁₀ heteroaryl, substituted C₆₋₁₀ heteroaryl, C₄₋₁₂ heterocycloalkylalkyl, substituted C₄₋₁₂ heterocycloalkylalkyl, C₇₋₁₂ heteroarylalkyl, substituted C₇₋₁₂ heteroarylalkyl; or R⁶ and R⁷ together with the nitrogen to which they are attached form a ring chosen from a C₅₋₁₀ heteroaryl, substituted C₅₋₁₀ heteroaryl, C₅₋₁₀ heterocycloalkyl, and substituted C₅₋₁₀ heterocycloalkyl.

In certain embodiments of a compound of Formula (III), the compound is: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; 4-{2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; 4-{2-[(2E)-3-(methoxycarbonyl)prop-2-enoyloxy]acetylamino}butanoic acid, sodium salt; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N[2-(dimethylamino)ethyl]carbamoyl}methyl methyl(2E)but-2ene-1,4 dioate; methyl 2-(4-methylpiperazinyl)-2-oxoethyl(2E)but-2-ene-1,4-dioate; methyl {N-[(propylamino)carbonyl]carbamoyl}methyl(2E)but-2ene-1,4-dioate; 2-(4-acetylpiperazinyl)-2-oxoethyl methyl(2E)but-2ene-1,4-dioate; {N,N-bis[2-(methylethoxy)ethyl]carbamoyl}methyl methyl(2E)but-2-ene-1,4-dioate; methyl 2-(4-benzylpiperazinyl)-2-oxoethyl(2E)but-2-ene-1,4-dioate; [N,N-bis(2-ethoxyethyl)carbamoyl]methyl methyl(2E)but-2-ene-1,4-dioate; 2-{(2S)-2-[(tert-butyl)oxycarbonyl]pyrrolidinyl}-2-oxoethyl methyl(2E)but-2ene-1,4-dioate; 1-{2-[(2E)-3-(methoxycarbonyl)prop-2-enoyloxy]acetyl}(2S)pyrrolidine-2-carboxylic acid; (N-{[(tert-butyl)oxycarbonyl]methyl}-N-methylcarbamoyl)methyl methyl(2E)but-2-ene-1,4-dioate; {N-(ethoxycarbonyl)methyl]-N-methylcarbamoyl}methyl methyl(2E)but-2-ene-1,4-dioate; methyl 1-methyl-2-morpholin-4-yl-2-oxoethyl(2E)but-2-ene-1,4-dioate; [N,N-bis(2-methoxyethyl)carbamoyl]ethyl methyl(2E)but-2-ene-1,4-dioate; (N,N-dimethylcarbamoyl)ethyl methyl(2E)but-2-ene-1,4-dioate; 2-{2-[(2E)-3-(methoxy carbonyl)prop-2-enoyloxyl]-N-methylacetylamino}acetic acid; (N-{[(tert-butyl)oxycarbonyl]methyl}carbamoyl)methyl methyl(2E)but-2-ene-1,4-dioate; (2E)but-methyl-N-{[(methylethyl)oxycarbonyl]methyl}carbamoyl)methyl(2E)but-2-ene-1,4-dioate; {N-[(ethoxycarbonyl)methyl]-N-benzylcarbamoyl}methyl methyl(2E)but-2-ene-1,4-dioate; {N-[(ethoxycarbonyl)methyl]-N-benzylcarbamoyl}ethyl methyl(2E)but-2-ene-1,4-dioate; {N-[(ethoxycarbonyl)methyl]-N-methylcarbamoyl}ethyl methyl(2E)but-2-ene-1,4-dioate; (1S)-1-methyl-2-morpholin-4-yl-2-oxo ethyl methyl(2E)but-2-ene-1,4-dioate; (1S)-1-[N,N-bis(2-methoxyethyl)carbamoyl]ethyl methyl(2E)but-2-ene-1,4-dioate; (1R)-1-(N,N-diethylcarbamoyl)ethyl methyl(2E)but-2-ene-1,4-dioate; or (1S)-1-(N,N-diethylcarbamoyl)ethyl methyl(2E)but-2-ene-1,4-dioate; or a pharmaceutically acceptable salt, tautomer, or stereoisomer thereof.

In certain embodiments of a compound of Formula (III), the compound is:

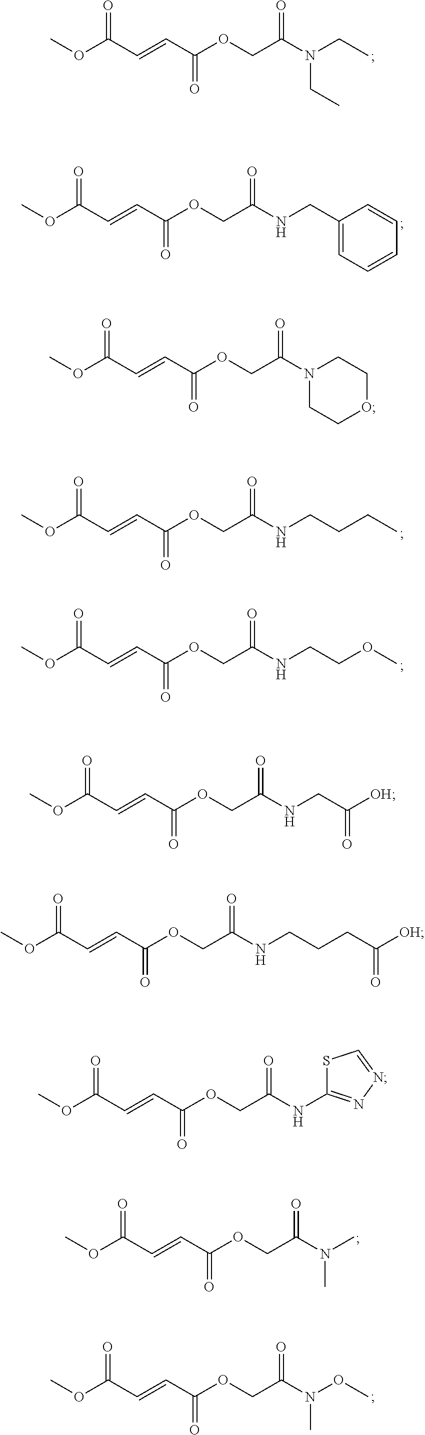

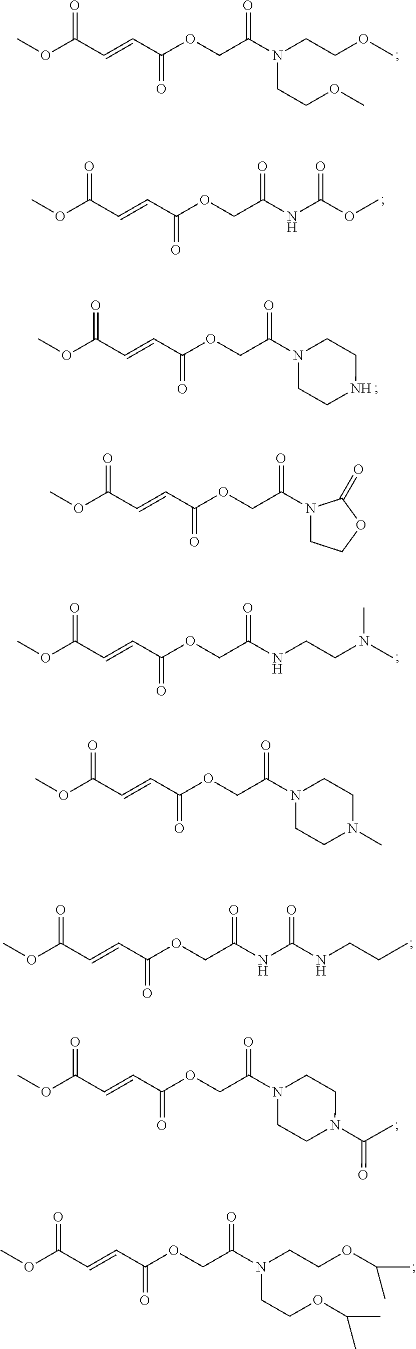

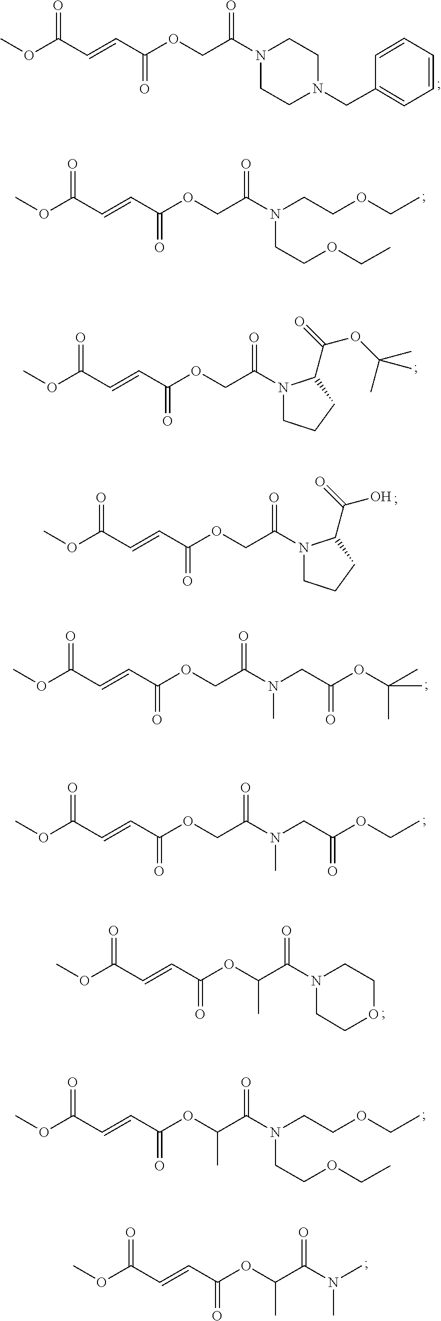

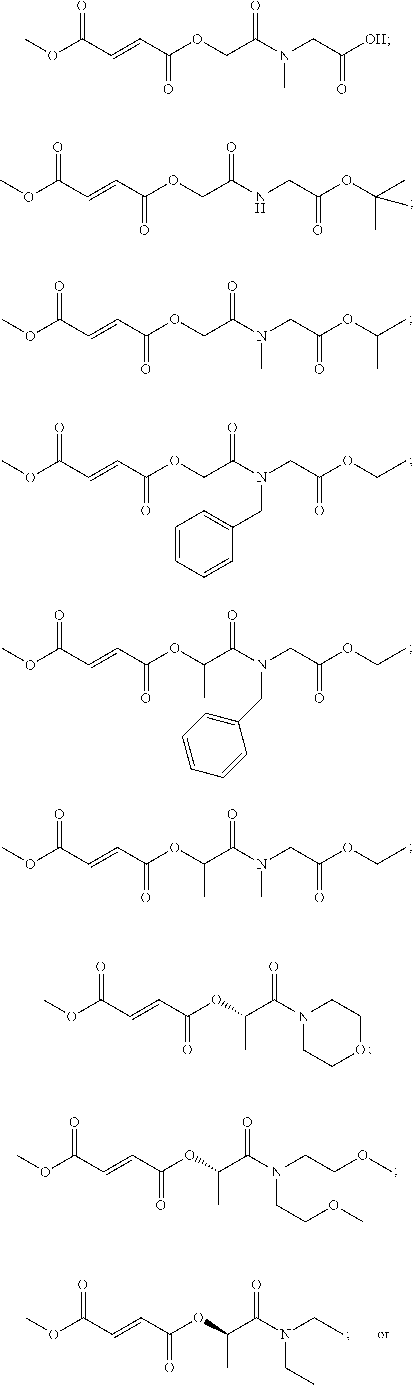

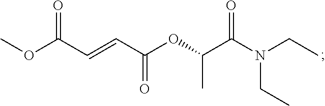 [SMILES: CCN(CC)C(=O)[C@H](C)OC(=O)/C=C/C(=O)OC]

or a pharmaceutically acceptable salt, tautomer, or stereoisomer thereof.

In certain embodiments of a compound of Formula (III), the compound is: (N,N-diethylcarbamoyl)methyl methyl(2E)but-2-ene-1,4-dioate; methyl[N-benzylcarbamoyl]methyl(2E)but-2-ene-1,4-dioate; methyl 2-morpholin-4-yl-2-oxoethyl(2E)but-2-ene-1,4-dioate; (N-butylcarbamoyl)methyl methyl(2E)but-2-ene-1,4-dioate; [N-(2-methoxyethyl)carbamoyl]methyl methyl(2E)but-2-ene-1,4-dioate; 2-{2-[(2E)-3-(methoxycarbonyl)prop-2-enoyloxy]acetylamino}acetic acid; {2-[(2E)-3-(methoxycarbonyl)prop-2-enoyloxy]acetylamino}butanoic acid; methyl(N-(1,3,4-thiadiazol-2-yl)carbamoyl)methyl(2E)but-2ene-1,4-dioate; (N,N-dimethylcarbamoyl)methyl methyl(2E)but-2-ene-1,4-dioate; (N-methoxy-N-methylcarbamoyl)methyl methyl(2E)but-2-ene-1,4-dioate; bis-(2-methoxyethylamino)carbamoyl]methyl methyl(2E)but-2-ene-1,4-dioate; [N-(methoxycarbonyl)carbamoyl]methyl methyl(2E)but-2ene-1,4-dioate; methyl 2-oxo-2-piperazinylethyl(2E)but-2-ene-1,4-dioate; methyl 2-oxo-2-(2-oxo(1,3-oxazolidin-3-yl)ethyl(2E)but-2ene-1,4-dioate; {N[2-(dimethylamino)ethyl]carbamoyl}methyl methyl(2E)but-2ene-1,4-dioate; (N-[(methoxycarbonyl)ethyl]carbamoyl)methyl methyl(2E)but-2-ene-1,4-dioate; or 2-{2-[(2E)-3-(methoxycarbonyl)prop-2-enoyloxy]acetylamino}propanoic acid; or a pharmaceutically acceptable salt, tautomer, or stereoisomer thereof.

In certain embodiments of a compound of Formula (III), the compound is:

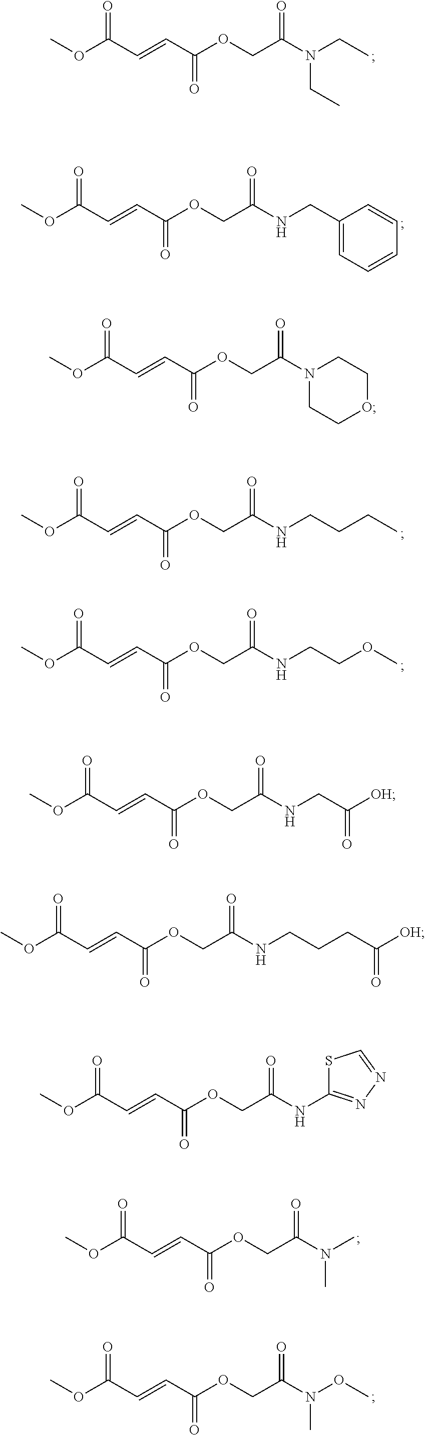

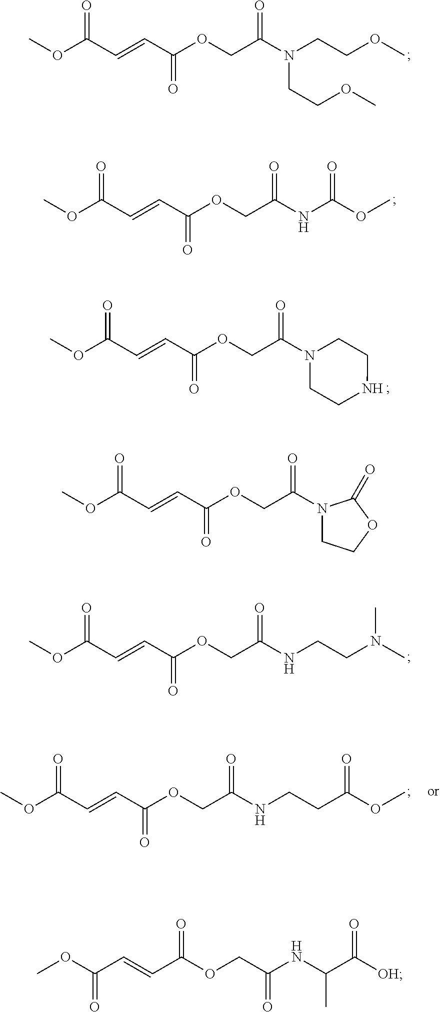

or a pharmaceutically acceptable salt, tautomer, or stereoisomer thereof.

In certain embodiments of a compound of Formula (III), the compound is:

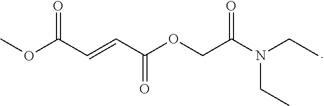 [SMILES: CCN(CC)C(=O)COC(=O)/C=C/C(=O)OC]

See also US 2014-0179778 A1.

In certain embodiments of a compound of Formula (III), the compound is:

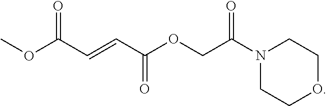 [SMILES: COC(=O)/C=C/C(=O)OCC(=O)N1CCOCC1]

In certain embodiments of a compound of Formula (III), the compound is:

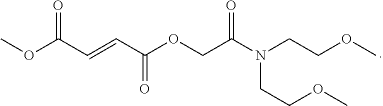

In certain embodiments of a compound of Formula (III), the compound is:

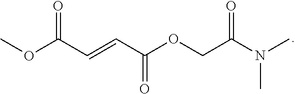

The compounds recited in paragraphs [00411] and [00413] are named using Chemistry 4-D Draw Pro, Version 7.01c (ChemInnovation Software, Inc., San Diego, Calif.).

In one embodiment, the compounds of Formula (III) may be prepared using methods known to those skilled in the art, for example, as disclosed in U.S. Pat. No. 8,148,414 B2.

In one embodiment, the prodrugs of monoalkyl fumarates are the prodrugs disclosed in WO2013/119677, such as the compounds of Formula (IV):

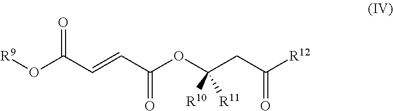

or a pharmaceutically acceptable salt, tautomer, or stereoisomer thereof, wherein
R⁹ is C₁₋₆ alkyl;
R¹⁰ and R¹¹ are each independently hydrogen, C₁₋₆ alkyl, or substituted C₁₋₆ alkyl; and
R¹² is C₁₋₆ alkyl, substituted C₁₋₆ alkyl, C₁₋₆ alkenyl, substituted C₁₋₆ alkenyl, C₁₋₆ heteroalkyl, substituted C₁₋₆ heteroalkyl, C₃₋₈ cycloalkyl, substituted C₃₋₈ cycloalkyl, C₆₋₈ aryl, substituted C₆₋₈ aryl, or
   —OR¹³ wherein R¹³ is C₁₋₆ alkyl, substituted C₁₋₆ alkyl, C₃₋₁₀ cycloalkyl, substituted C₃₋₁₀ cycloalkyl, C₆₋₁₀ aryl, or substituted C₆₋₁₀ aryl;
wherein each substituent is independently halogen, —OH, —CN, —CF₃, ═O, —NO₂, benzyl, —C(O)NR¹⁴₂, —R¹⁴, —OR¹⁴, —C(O)R¹⁴, —COOR¹⁴, or —NR¹⁴₂ wherein each R¹⁴ is independently hydrogen or C₁₋₄ alkyl.

In certain embodiments of a compound of Formula (IV), each substituent is independently halogen, —OH, —CN, —CF₃, —R¹⁴, —OR¹⁴, or —NR¹⁴₂ wherein each R¹⁴ is independently hydrogen or C₁₋₄ alkyl.

In certain embodiments of a compound of Formula (IV), each substituent is independently ═O, C₁₋₄ alkyl, and —COOR¹⁴ wherein R¹⁴ is hydrogen or C₁₋₄ alkyl.

In certain embodiments of a compound of Formula (IV), R⁹ is C₁₋₆ alkyl; in certain embodiments, R⁹ is C₁₋₃ alkyl; and in certain embodiments, R⁹ is methyl or ethyl.

In certain embodiments of a compound of Formula (IV), R⁹ is methyl.

In certain embodiments of a compound of Formula (IV), R⁹ is ethyl, n-propyl, isopropyl, n-butyl, sec-butyl, isobutyl, or tert-butyl.

In certain embodiments of a compound of Formula (IV), R⁹ is methyl, ethyl, n-propyl, isopropyl, n-butyl, isobutyl, or tert-butyl.

In certain embodiments of a compound of Formula (IV), one of R¹⁰ and R¹¹ is hydrogen and the other of R¹⁰ and R¹¹ is C₁₋₆ alkyl. In certain embodiments of a compound of Formula (IV), one of R¹⁰ and R¹¹ is hydrogen and the other of R¹⁰ and R¹¹ is C₁₋₄ alkyl.

In certain embodiments of a compound of Formula (IV), one of R¹⁰ and R¹¹ is hydrogen and the other of R¹⁰ and R¹¹ is methyl, ethyl, n-propyl, or isopropyl. In certain embodiments of a compound of Formula (IV), each of R¹⁰ and R¹¹ is hydrogen.

In certain embodiments of a compound of Formula (IV), R¹² is C₁₋₆ alkyl; one of R¹⁰ and R¹¹ is hydrogen and the other of R¹⁰ and R¹¹ is C₁₋₆ alkyl; and R⁹ is C₁₋₆ alkyl.

In certain embodiments of a compound of Formula (IV), R¹² is —OR¹³.

In certain embodiments of a compound of Formula (IV), R¹³ is C₁₋₄ alkyl, cyclohexyl, or phenyl.

In certain embodiments of a compound of Formula (IV), R¹² is methyl, ethyl, n-propyl, or isopropyl; one of R¹⁰ and R¹¹ is hydrogen and the other of R¹⁰ and R¹¹ is methyl, ethyl, n-propyl, or isopropyl.

In certain embodiments of a compound of Formula (IV), R¹² is substituted C₁₋₂ alkyl, wherein each substituent is independently —COOH, —NHC(O)CH₂NH₂, or —NH₂.

In certain embodiments of a compound of Formula (IV), R¹² is ethoxy, methylethoxy, isopropyl, phenyl, cyclohexyl, cyclohexyloxy, —CH(NH₂)CH₂COOH, —CH₂CH(NH₂)COOH, —CH(NHC(O)CH₂NH₂)—CH₂COOH, or —CH₂CH(NHC(O)CH₂NH₂)—COOH.

In certain embodiments of a compound of Formula (IV), R⁹ is methyl or ethyl; one of R¹⁰ and R¹¹ is hydrogen and the other of R¹⁰ and R¹¹ is hydrogen, methyl, ethyl, n-propyl, or isopropyl; and R¹² is C₁₋₃ alkyl, substituted C₁₋₂ alkyl wherein each substituent group is —COOH, —NHC(O)CH₂NH₂, —NH₂, or —OR¹³ wherein R¹³ is C₁₋₃ alkyl, cyclohexyl, phenyl, or cyclohexyl.

In certain embodiments of a compound of Formula (IV), the compound is: ethoxycarbonyloxyethyl methyl(2E)but-2-ene-1,4-dioate; methyl(methylethoxycarbonyloxy)ethyl(2E)but-2-ene-1,4-dioate; or (cyclohexyloxycarbonyloxy)ethyl methyl(2E)but-2-ene-1,4-dioate; or stereoisomer thereof.

In certain embodiments of a compound of Formula (IV), the compound is:

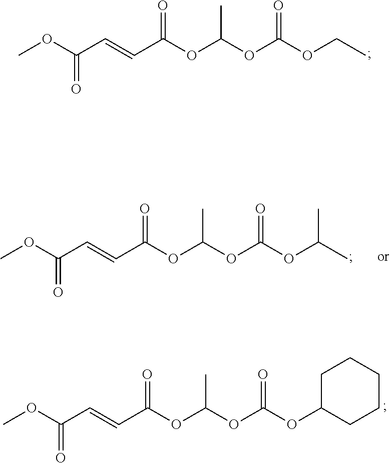

or stereoisomer thereof.

In certain embodiments of a compound of Formula (IV), the compound is: methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; or methyl 2-methyl-1-phenylcarbonyloxypropyl(2E)but-2-ene-1,4-dioate; or stereoisomer thereof.

In certain embodiments of a compound of Formula (IV), the compound is:

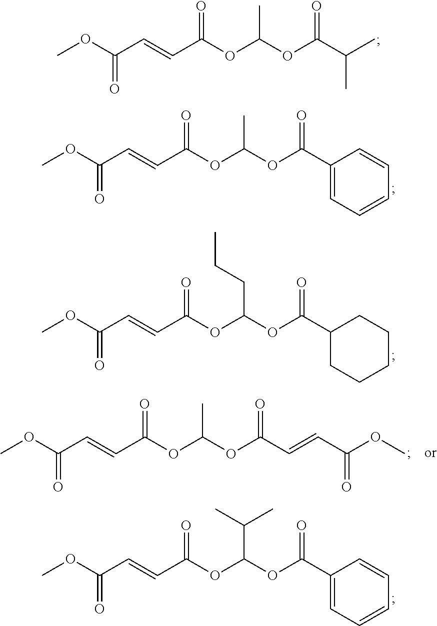

or stereoisomer thereof.

In certain embodiments of a compound of Formula (IV), the compound is: ethoxycarbonyloxyethyl methyl(2E)but-2-ene-1,4-dioate; methyl(methylethoxycarbonyloxy)ethyl(2E)but-2-ene-1,4-dioate; methyl(2-methylpropanoyloxy)ethyl(2E)but-2-ene-1,4-dioate; methyl phenylcarbonyloxyethyl(2E)but-2-ene-1,4-dioate; cyclohexylcarbonyloxybutyl methyl(2E)but-2-ene-1,4-dioate; [(2E)-3-(methoxycarbonyl)prop-2-enoyloxy]ethyl methyl(2E)but-2-ene-1,4-dioate; (cyclohexyloxycarbonyloxy)ethyl methyl(2E)but-2-ene-1,4-dioate; methyl 2-methyl-1-phenylcarbonyloxypropyl(2E)but-2-ene-1,4-dioate; or stereoisomer thereof.

In certain embodiments of a compound of Formula (IV), the compound is: 3-({[(2E)-3-(methoxycarbonyl)prop-2-enoyloxy]methyl}oxycarbonyl)(3 S)-3-aminopropanoic acid, 2,2,2-trifluoroacetic acid; 3-({[(2E)-3-(methoxycarbonyl)prop-2-enoyloxy]methyl}oxycarbonyl)(2 S)-2-aminopropanoic acid, 2,2,2-trifluoroacetic acid; 3-({[(2E)-3-(methoxycarbonyl)prop-2-enoyloxy]methyl}oxycarbonyl)(3 S)-3-(2-aminoacetylamino)propanoic acid, 2,2,2-trifluoroacetic acid; or 3-{[(2E)-3-(methoxycarbonyl)prop-2enoyloxy]ethoxycarbonyloxy}(2 S)-2-aminopropanoic acid, chloride; or stereoisomer thereof.

In certain embodiments of a compound of Formula (IV), the compound is:

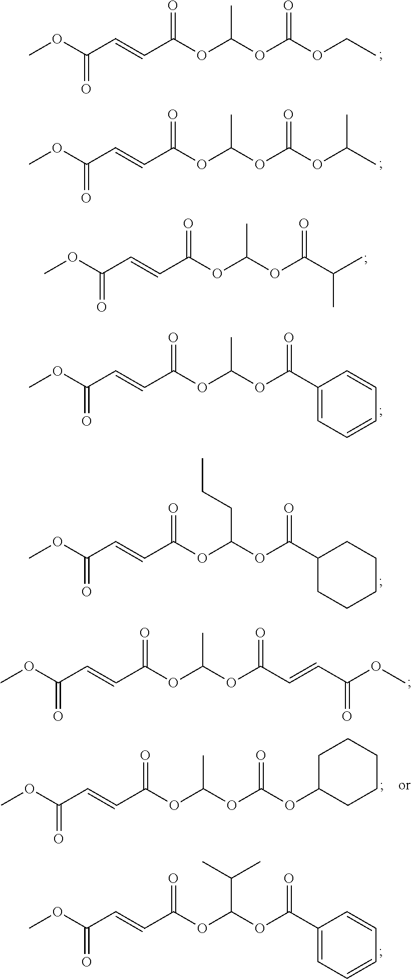

or stereoisomer thereof.

In certain embodiments of a compound of Formula (IV), the compound is:

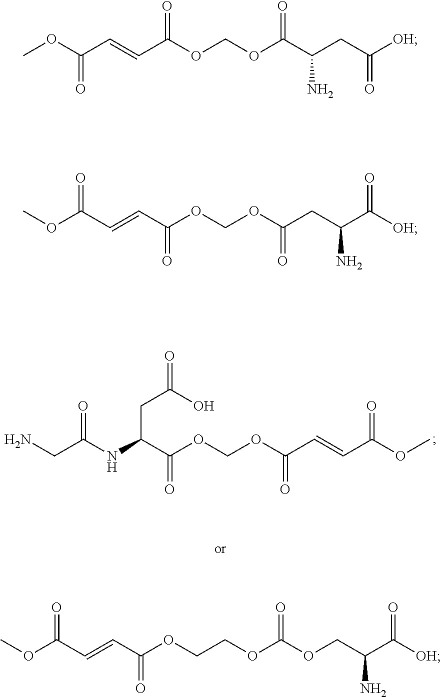

or a pharmaceutically acceptable salt, or stereoisomer thereof.

In certain embodiments of a compound of Formula (IV), the compound is:

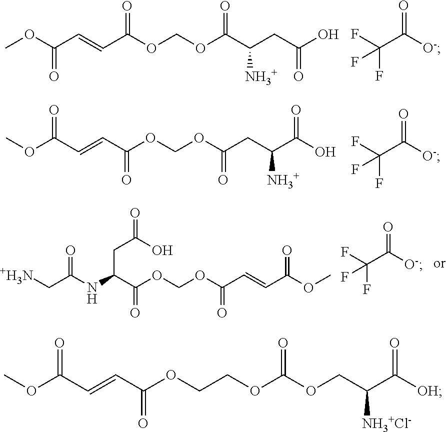

or stereoisomer thereof.

The compounds recited in paragraphs [00437], [00439], [00441], and [00442] are named using Chemistry 4-D Draw Pro, Version 7.01c (ChemInnovation Software, Inc., San Diego, Calif.).

In one embodiment, the compounds of Formula (IV) may be prepared using methods known to those skilled in the art, for example, as disclosed in U.S. Pat. No. 8,148,414 B2.

In one embodiment, the prodrugs of monoalkyl fumarates are the prodrugs disclosed in U.S. Patent Application Publication No. 2014/0057918, such as the compounds of Formula (V):

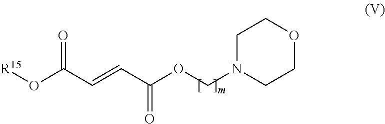

or a pharmaceutically acceptable salt thereof, wherein

R¹⁵ is C₁₋₆ alkyl; and

m is an integer from 2 to 6.

In certain embodiments of a compound of Formula (V), R¹⁵ is methyl.

In certain embodiments of a compound of Formula (V), R¹⁵ is ethyl.

In certain embodiments of a compound of Formula (V), R¹⁵ is C₃₋₆ alkyl.

In certain embodiments of a compound of Formula (V), R¹⁵ is methyl, n-propyl, isopropyl, n-butyl, sec-butyl, isobutyl, or tert-butyl.

In certain embodiments of a compound of Formula (V), R¹⁵ is methyl, ethyl, n-propyl, isopropyl, n-butyl, sec-butyl, isobutyl, or tert-butyl.

In certain embodiments of a compound of Formula (V), the compound is:
methyl (2-morpholinoethyl)fumarate;
methyl (3-morpholinopropyl)fumarate;
methyl (4-morpholinobutyl)fumarate;
methyl (5-morpholinopentyl)fumarate; or
methyl (6-morpholinohexyl)fumarate;
   or a pharmaceutically acceptable salt thereof.

In certain embodiments of a compound of Formula (V), the compound is:

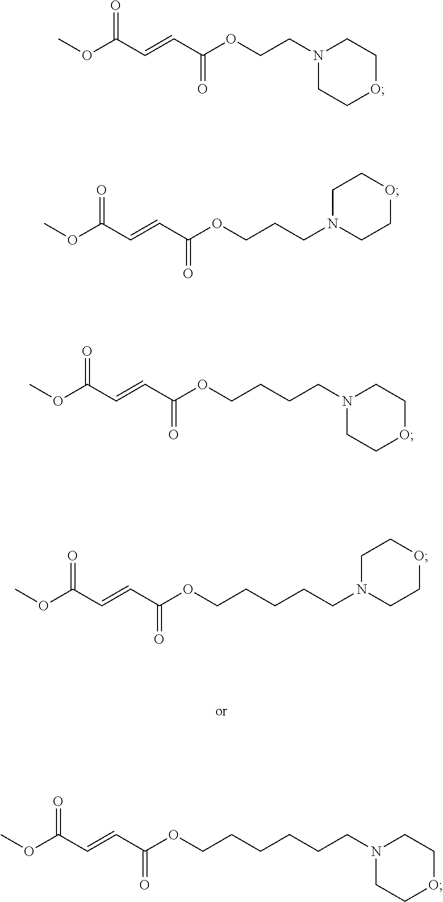 [SMILES: COC(=O)/C=C/C(=O)OCCN1CCOCC1 ; COC(=O)/C=C/C(=O)OCCCN1CCOCC1 ; COC(=O)/C=C/C(=O)OCCCCN1CCOCC1 ; COC(=O)/C=C/C(=O)OCCCCCN1CCOCC1 ; COC(=O)/C=C/C(=O)OCCCCCCN1CCOCC1]

or a pharmaceutically acceptable salt thereof.

The compounds recited in paragraph [00454] are named using Chemistry 4-D Draw Pro, Version 7.01c (ChemInnovation Software, Inc., San Diego, Calif.).

In one embodiment, the compounds of Formula (V) may be prepared using methods known to those skilled in the art, for example, as disclosed in U.S. Patent Application Publication No. 2014/0057918.

In one embodiment, the prodrugs of monoalkyl fumarates are the prodrugs disclosed in WO2013/119677, such as the compounds of Formula (VI):

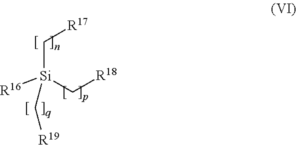

or a pharmaceutically acceptable salt, or stereoisomer thereof, wherein:
R¹⁶ is C₁₋₁₀ alkyl, C₅₋₁₄ aryl, hydroxyl, —O—C₁₋₁₀ alkyl, or —O—C₅₋₁₄ aryl;
each of R¹⁷, R¹⁸, and R¹⁹ independently is C₁₋₁₀ alkyl, C₅₋₁₄ aryl, hydroxyl, alkyl, —O—C₅₋₁₄ aryl, or

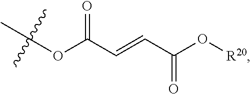

wherein R²⁰ is C₁₋₆ alkyl; each of which can be optionally substituted; and
each of n, p, and q independently is 0-4;
provided that at least one of R¹⁷, R¹⁻⁸, and R¹⁹ is

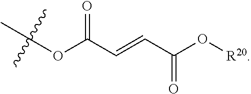

In certain embodiments of a compound of Formula (VI), R²⁰ is optionally substituted C₁₋₆ alkyl. In certain embodiments of a compound of Formula (VI), R²⁰ is optionally substituted methyl, ethyl, or isopropyl. In certain embodiments of a compound of Formula (VI), R²⁰ is methyl.

In certain embodiments of a compound of Formula (VI), R¹⁶ is C₁₋₁₀ alkyl. In certain embodiments of a compound of Formula (VI), R¹⁶ is optionally substituted C₁₋₆ alkyl. In certain embodiments of a compound of Formula (VI), R¹⁶ is optionally substituted methyl, ethyl, or isopropyl. In certain embodiments of a compound of Formula (VI), R¹⁶ is optionally substituted C₅₋₁₅ aryl. In certain embodiments of a compound of Formula (VI), R¹⁶ is optionally substituted C₅-C₁₀ aryl.

In one embodiment, the prodrugs of monoalkyl fumarates are the prodrugs disclosed in WO2013/119677, such as the compounds of Formula (VI′):

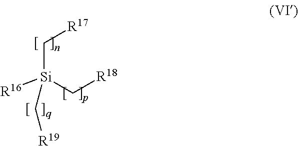

or a pharmaceutically acceptable salt, or stereoisomer thereof, wherein
R¹⁶ is C₁₋₁₀ alkyl, C₆₋₁₀ aryl, hydroxyl, alkyl, or —O—C₆₋₁₀ aryl;
each of R¹⁷, R¹⁸, and R¹⁹ independently is C₁₋₁₀ alkyl, C₆₋₁₀ aryl, hydroxyl, alkyl, —O—C₆₋₁₀ aryl, or

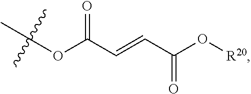

wherein R²⁰ is C₁₋₆ alkyl; each of which can be optionally substituted; and
each of n, p, and q independently is 0-4;
provided that at least one of R¹⁷, R¹⁻⁸, and R¹⁹ is

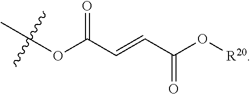

In certain embodiments of a compound of Formula (VI′), R²⁰ is methyl.

In certain embodiments of a compound of Formula (VI) or Formula (VI′), the compound is: (dimethyl silanediyl)dimethyl difumarate; methyl ((trimethoxysilyl)methyl) fumarate; methyl ((trihydroxysilyl)methyl) fumarate; or trimethyl (methylsilanetriyl) trifumarate; or a pharmaceutically acceptable salt thereof.

In certain embodiments of a compound of Formula (VI) or Formula (VI′), the compound is:

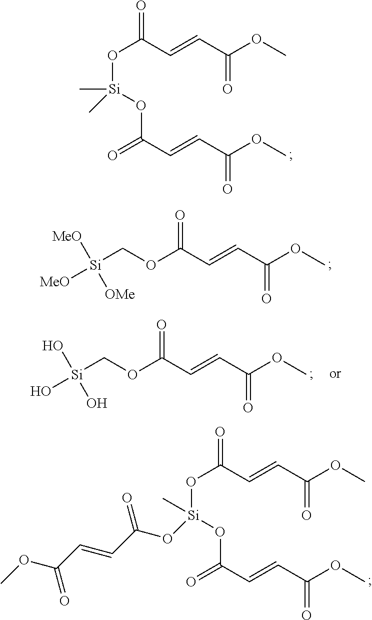

or a pharmaceutically acceptable salt thereof.

In one embodiment, the compounds of Formula (VI) and Formula (VI′) may be prepared using methods known to those skilled in the art, for example, as disclosed in WO2013/119677.

In one embodiment, the prodrugs of monoalkyl fumarates are the prodrugs disclosed in WO2013/119677, such as the compounds of Formula (VII):

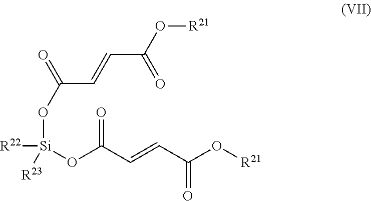

or a pharmaceutically acceptable salt, or stereoisomer thereof, wherein:

wherein R²¹ is C₁₋₆ alkyl; and

each of R²² and R²³ independently is C₁₋₁₀ alkyl or C₅₋₁₄ aryl;

each of which can be optionally substituted.

In certain embodiments of a compound of Formula (VII), R²¹ is optionally substituted C₁₋₆ alkyl. In certain embodiments of a compound of Formula (VII), R²¹ is optionally substituted methyl, ethyl, or isopropyl. In certain embodiments of a compound of Formula (VII), R²¹ is methyl.

In certain embodiments of a compound of Formula (VII), each of R²² and R²³ independently is optionally substituted C₁₋₁₀ alkyl. In certain embodiments of a compound of Formula (VII), each of R²² and R²³ independently is optionally substituted C₁₋₆ alkyl. In certain embodiments of a compound of Formula (VII), each of R²² and R²³ independently is optionally substituted methyl, ethyl, or isopropyl. In certain embodiments of a compound of Formula (VII), each of R²² and R²³ independently is optionally substituted C₅₋₁₄ aryl. In certain embodiments of a compound of Formula (VII), each of R²² and R²³ independently is optionally substituted C₅₋₁₀ aryl.

In one embodiment, the prodrugs of monoalkyl fumarates are the prodrugs disclosed in WO2013/119677, such as the compounds of Formula (VII′):

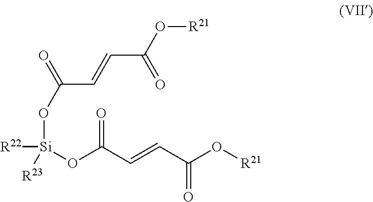

or a pharmaceutically acceptable salt, or stereoisomer thereof, wherein

R²¹ is C₁₋₆ alkyl; and

each of R²² and R²³ independently is C₁₋₁₀ alkyl or C₆₋₁₀ aryl.

In one embodiment, the compounds of Formula (VII) and Formula (VII′) may be prepared using methods known to those skilled in the art, for example, as disclosed in WO2013/119677.

In one embodiment, the prodrugs of monoalkyl fumarates are the prodrugs disclosed in WO2013/119677, such as the compounds of Formula (VIII):

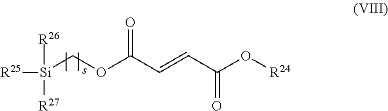

or a pharmaceutically acceptable salt, or stereoisomer thereof, wherein:
R²⁴ is C₁₋₆ alkyl;
each of R²⁵, R²⁶, and R²⁷ independently is hydroxyl, C₁₋₁₀ alkyl, C₅₋₁₄ aryl, alkyl, or —O—C₅₋₁₄ aryl;
each of which can be optionally substituted; and
s is 1 or 2.

In certain embodiments of a compound of Formula (VIII), R²⁴ is optionally substituted C₁-C₆ alkyl. In certain embodiments of a compound of Formula (VIII), R²⁴ is optionally substituted methyl, ethyl, or isopropyl. In certain embodiments of a compound of Formula (VIII), R²⁴ is methyl.

In certain embodiments of a compound of Formula (VIII), each of R²⁵, R²⁶, and R²⁷ is hydroxyl. In certain embodiments of a compound of Formula (VIII), each of R²⁵, R²⁶, and R²⁷ independently is optionally substituted C₁₋₁₀ alkyl. In certain embodiments of a compound of Formula (VIII), each of R²⁵, R²⁶, and R²⁷ independently is optionally substituted C₁₋₆ alkyl. In certain embodiments of a compound of Formula (VIII), each of R²⁵, R²⁶, and R²⁷ independently is optionally substituted methyl, ethyl, or isopropyl. In certain embodiments of a compound of Formula (VIII), each of R²⁵, R²⁶, and R²⁷ independently is optionally substituted C₅₋₁₄ aryl. In certain embodiments of a compound of Formula (VIII), each of R²⁵, R²⁶, and R²⁷ independently is optionally substituted C₅₋₁₀ aryl.

In one embodiment, the prodrugs of monoalkyl fumarates are the prodrugs disclosed in WO2013/119677, such as the compounds of Formula (VIII′):

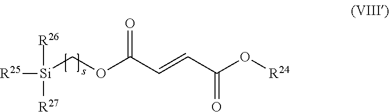

or a pharmaceutically acceptable salt, or stereoisomer thereof, wherein:
R²⁴ is C₁₋₆ alkyl;
each of R²⁵, R²⁶, and R²⁷ independently is hydroxyl, C₁₋₁₀ alkyl, C₆₋₁₀ aryl, —O—C₁₋₁₀ alkyl, or —O—C₆₋₁₀ aryl; and
s is 1 or 2.

In one embodiment, the compounds of Formula (VIII) and Formula (VIII′) may be prepared using methods known to those skilled in the art, for example, as disclosed in WO2013/119677.

In one embodiment, the prodrugs of monoalkyl fumarates are the prodrugs disclosed in WO2013/119677, such as the compounds of Formula (IX):

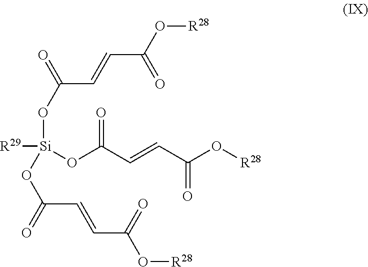

or a pharmaceutically acceptable salt, or stereoisomer thereof, wherein

each of R²⁸ independently is C₁₋₆ alkyl; and

R²⁹ is C₁₋₁₀ alkyl;

each of which can be optionally substituted.

In certain embodiments of a compound of Formula (IX), each of R²⁸ independently is optionally substituted C₁₋₆ alkyl. In certain embodiments of a compound of Formula (IX), each of R²⁸ independently is optionally substituted methyl, ethyl, or isopropyl. In certain embodiments of a compound of Formula (IX), each of R²⁸ is methyl.

In certain embodiments of a compound of Formula (IX), R²⁹ is optionally substituted C₁₋₆ alkyl. In certain embodiments of a compound of Formula (IX), R²⁹ is optionally substituted methyl, ethyl, or isopropyl.

In one embodiment, the prodrugs of monoalkyl fumarates are the prodrugs disclosed in WO2013/119677, such as the compounds of Formula (IX′):

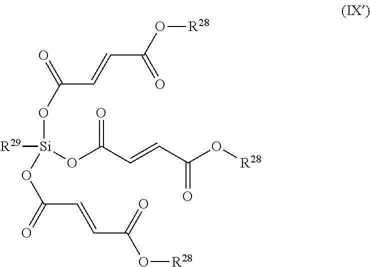

or a pharmaceutically acceptable salt, or stereoisomer thereof, wherein

R²⁸ is C₁₋₆ alkyl; and

R²⁹ is C₁₋₁₀ alkyl.

In one embodiment, the compounds of Formula (IX) and Formula (IX′) may be prepared using methods known to those skilled in the art, for example, as disclosed in WO2013/119677.

In one embodiment, the prodrugs of monoalkyl fumarates are the prodrugs disclosed in U.S. Pat. No. 8,669,281 B1, such as the compounds of Formula (X):

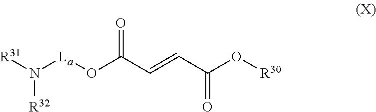

or a pharmaceutically acceptable salt, tautomer, or stereoisomer thereof, wherein
R³⁰ is unsubstituted C₁₋₆ alkyl;
Lₐ is substituted or unsubstituted C₁₋₆ alkyl linker, substituted or unsubstituted C₃₋₁₀ carbocycle, substituted or unsubstituted C₆₋₁₀ aryl, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S; and
R³¹ and R³² are each, independently, hydrogen, substituted or unsubstituted C₁₋₆ alkyl, substituted or unsubstituted C₂₋₆ alkenyl, substituted or unsubstituted C₂₋₆ alkynyl, substituted or unsubstituted C₆₋₁₀ aryl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
or alternatively, R³¹ and R³², together with the nitrogen atom to which they are attached, form a substituted or unsubstituted heteroaryl comprising one or two 5- or 6-member rings and 1-4 heteroatoms selected from N, O, and S or a substituted or unsubstituted heterocycle comprising one or two 5- or 6-member rings and 1-4 heteroatoms selected from N, O, and S.

In certain embodiments of a compound of Formula (X), R³⁰ is methyl. In certain embodiments of a compound of Formula (X), R³⁰ is ethyl.

In certain embodiments of a compound of Formula (X), Lₐ is substituted or unsubstituted C₁₋₆ alkyl linker. In certain embodiments of a compound of Formula (X), Lₐ is substituted or unsubstituted C₁₋₃ alkyl linker. In certain embodiments of a compound of Formula (X), Lₐ is substituted or unsubstituted C₂ alkyl linker. In certain embodiments of a compound of Formula (X), Lₐ is a methyl substituted or unsubstituted C₂ alkyl linker. In certain embodiments of a compound of Formula (X), Lₐ is a di-methyl substituted or unsubstituted C₂ alkyl linker. In certain embodiments of a compound of Formula (X), Lₐ is a methyl or di-methyl substituted C₂ alkyl linker. In certain embodiments of a compound of Formula (X), Lₐ is unsubstituted C₂ alkyl linker.

In certain embodiments of a compound of Formula (X), R³¹ is substituted or unsubstituted C₁₋₆ alkyl. In certain embodiments of a compound of Formula (X), R³¹ is unsubstituted C₁₋₆ alkyl. In certain embodiments of a compound of Formula (X), R³¹ is unsubstituted C₁₋₃ alkyl. In certain embodiments of a compound of Formula (X), R³¹ is unsubstituted C₁₋₂ alkyl.

In certain embodiments of a compound of Formula (X), R³¹ is C(O)ORₐ— substituted C₁₋₆ alkyl, wherein Rₐ is hydrogen or unsubstituted C₁₋₆ alkyl. In certain embodiments of a compound of Formula (X), R³¹ is S(O)(O)R_{b}-substituted C₁₋₆ alkyl, wherein R_{b} is unsubstituted C₁₋₆ alkyl.

In certain embodiments of a compound of Formula (X), R³² is hydrogen. In certain embodiments of a compound of Formula (X), R³² is substituted or unsubstituted C₁₋₆ alkyl. In certain embodiments of a compound of Formula (X), R³² is unsubstituted C₁₋₆ alkyl.

In certain embodiments of a compound of Formula (X), R³¹ and R³², together with the nitrogen atom to which they are attached, form a substituted or unsubstituted heteroaryl comprising one or two 5- or 6-member rings and 1-4 heteroatoms selected from N, O, and S, or a substituted or unsubstituted heterocycle comprising one or two 5- or 6-member rings and 1-4 heteroatoms selected from N, O, and S.

In certain embodiments of a compound of Formula (X), R³¹ and R³², together with the nitrogen atom to which they are attached, form a substituted or unsubstituted heterocycle comprising one or two 5- or 6-member rings and 1-4 heteroatoms selected from N, O, and S.

In certain embodiments of a compound of Formula (X), R³¹ and R³², together with the nitrogen atom to which they are attached, form a substituted or unsubstituted pyrrolidinyl, imidazolidinyl, pyrazolidinyl, oxazolidinyl, isoxazolidinyl, triazolidinyl, tetrahydrofuranyl, piperidinyl, piperazinyl, or morpholinyl ring.

In certain embodiments of a compound of Formula (X), R³¹ and R³², together with the nitrogen atom to which they are attached, form a substituted or unsubstituted piperidinyl ring.

In certain embodiments of a compound of Formula (X), R³¹ and R³², together with the nitrogen atom to which they are attached, form an unsubstituted piperidinyl ring.

In certain embodiments of a compound of Formula (X), R³¹ and R³², together with the nitrogen atom to which they are attached, form a halogen substituted piperidinyl ring. In certain embodiments of a compound of Formula (X), R³¹ and R³², together with the nitrogen atom to which they are attached, form a 4-halogen substituted piperidinyl ring.

In certain embodiments of a compound of Formula (X), R³¹ and R³², together with the nitrogen atom to which they are attached, form an unsubstituted morpholinyl ring.

In certain embodiments of a compound of Formula (X), R³¹ and R³², together with the nitrogen atom to which they are attached, form an unsubstituted pyrrolidinyl ring.

In certain embodiments of a compound of Formula (X), R³¹ and R³², together with the nitrogen atom to which they are attached, form a substituted or unsubstituted heteroaryl comprising one or two 5 or 6-member rings and 1-4 heteroatoms selected from N, O, and S.

In certain embodiments of a compound of Formula (X), R³¹ is substituted or unsubstituted C₆₋₁₀ aryl. In certain embodiments of a compound of Formula (X), R³¹ is unsubstituted C₆-C₁₀ aryl. In certain embodiments of a compound of Formula (X), R³¹ is unsubstituted phenyl. In certain embodiments of a compound of Formula (X), R³¹ is unsubstituted benzyl.

In one embodiment, the compounds of Formula (X) may be prepared using methods known to those skilled in the art, for example, as disclosed in U.S. Pat. No. 8,669,281 B1.

In one embodiment, the prodrugs of monoalkyl fumarates are the prodrugs disclosed in U.S. Pat. No. 8,669,281 B1, such as the compounds of Formula (X′):

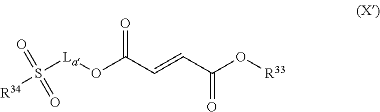

or a pharmaceutically acceptable salt, tautomer, or stereoisomer thereof, wherein
R³³ is unsubstituted C₁₋₆ alkyl;
L_{a′} is substituted or unsubstituted C₁₋₆ alkyl linker, substituted or unsubstituted C₃₋₁₀ carbocycle, substituted or unsubstituted C₆₋₁₀ aryl, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S; and
R³⁴ is hydrogen, substituted or unsubstituted C₁₋₆ alkyl, substituted or unsubstituted C₂₋₆ alkenyl, substituted or unsubstituted C₂₋₆ alkynyl, substituted or unsubstituted C₆₋₁₀ aryl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S.

In certain embodiments of a compound of Formula (X′), R³³ is methyl. In certain embodiments of a compound of Formula (X′), R³³ is ethyl.

In certain embodiments of a compound of Formula (X′), L_{a′} is substituted or unsubstituted C₁₋₆ alkyl linker. In certain embodiments of a compound of Formula (X′), L_{a′} is substituted or unsubstituted C₁₋₃ alkyl linker.

In certain embodiments of a compound of Formula (X′), L_{a′} is substituted or unsubstituted C₂ alkyl linker. In certain embodiments of a compound of Formula (X′), L_{a′} is methyl substituted or unsubstituted C₂ alkyl linker. In certain embodiments of a compound of Formula (X′), L_{a′} is di-methyl substituted or unsubstituted C₂ alkyl linker. In certain embodiments of a compound of Formula (X′), L_{a′} is methyl or di-methyl substituted C₂ alkyl linker. In certain embodiments of a compound of Formula (X′), L_{a′} is unsubstituted C₂ alkyl linker.

In certain embodiments of a compound of Formula (X′), R³⁴ is substituted or unsubstituted C₁₋₆ alkyl. In certain embodiments of a compound of Formula (X′), R³⁴ is unsubstituted C₁₋₆ alkyl. In certain embodiments of a compound of Formula (X′), R³⁴ is methyl. In certain embodiments of a compound of Formula (X′), R³⁴ is unsubstituted C₁₋₃ alkyl. In certain embodiments of a compound of Formula (X′), R³⁴ is unsubstituted C₁₋₂ alkyl.

In certain embodiments of a compound of Formula (X′), R³⁴ is C(O)OR_{a′}-substituted C₁₋₆ alkyl, wherein R_{a′} is H or unsubstituted C₁₋₆ alkyl. In certain embodiments of a compound of Formula (X′), R³⁴ is S(O)(O)R_{b′}-substituted C₁₋₆ alkyl, wherein R_{b} is unsubstituted C₁₋₆ alkyl.

In one embodiment, the compounds of Formula (X′) may be prepared using methods known to those skilled in the art, for example, as disclosed in U.S. Pat. No. 8,669,281 B1.

In one embodiment, the prodrugs of monoalkyl fumarates are the prodrugs disclosed in U.S. Pat. No. 8,669,281 B1, such as the compounds of Formula (X″):

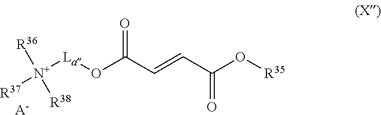

or a tautomer or stereoisomer thereof, wherein
A⁻ is a pharmaceutically acceptable anion;
R³⁵ is unsubstituted C₁₋₆ alkyl;
L_{a″} is substituted or unsubstituted C₁₋₆ alkyl linker, substituted or unsubstituted C₃₋₁₀ carbocycle, substituted or unsubstituted C₆₋₁₀ aryl, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
R³⁶ and R³⁷ are each, independently, hydrogen, substituted or unsubstituted C₁₋₆ alkyl, substituted or unsubstituted C₂₋₆ alkenyl, substituted or unsubstituted C₂-C₆ alkynyl, substituted or unsubstituted C₆₋₁₀ aryl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
or alternatively, R³⁶ and R³⁷, together with the nitrogen atom to which they are attached, form a substituted or unsubstituted heteroaryl comprising one or two 5- or 6-member rings and 1-4 heteroatoms selected from N, O, and S, or a substituted or unsubstituted heterocycle comprising one or two 5- or 6-member rings and 1-4 heteroatoms selected from N, O, and S; and
R³⁸ is substituted or unsubstituted C₁₋₆ alkyl.

In certain embodiments of a compound of Formula (X″), R³⁵ is methyl. In certain embodiments of a compound of Formula (X″), R³⁵ is ethyl.

In certain embodiments of a compound of Formula (X″), L_{a″} is substituted or unsubstituted C₁₋₆ alkyl linker. In certain embodiments of a compound of Formula (X″), L_{a″} is substituted or unsubstituted C₁₋₃ alkyl linker.

In certain embodiments of a compound of Formula (X″), L_{a″} is substituted or unsubstituted C₂ alkyl linker. In certain embodiments of a compound of Formula (X″), L_{a″} is methyl substituted or unsubstituted C₂ alkyl linker. In certain embodiments of a compound of Formula (X″), L_{a″} is di-methyl substituted or unsubstituted C₂ alkyl linker. In certain embodiments of a compound of Formula (X″), L_{a″} is methyl or di-methyl substituted C₂ alkyl linker. In certain embodiments of a compound of Formula (X″), L_{a″} is unsubstituted C₂ alkyl linker.

In certain embodiments of a compound of Formula (X″), R³⁶ is substituted or unsubstituted C₁₋₆ alkyl. In certain embodiments of a compound of Formula (X″), R³⁶ is unsubstituted C₁₋₆ alkyl. In certain embodiments of a compound of Formula (X″), R³⁶ is unsubstituted C₁₋₃ alkyl. In certain embodiments of a compound of Formula (X″), R³⁶ is unsubstituted C₁₋₂ alkyl.

In certain embodiments of a compound of Formula (X″), R³⁶ is C(O)OR_{a″}-substituted C₁₋₆ alkyl, wherein R_{a″} is hydrogen or unsubstituted C₁₋₆ alkyl. In certain embodiments of a compound of Formula (X″), R³⁶ is S(O)(O)R_{b″}-substituted C₁₋₆ alkyl, wherein R_{b″} is unsubstituted C₁₋₆ alkyl.

In certain embodiments of a compound of Formula (X″), R³⁶ and R³⁷, together with the nitrogen atom to which they are attached, form a substituted or unsubstituted heteroaryl comprising one or two 5- or 6-member rings and 1-4 heteroatoms selected from N, O, and S, or a substituted or unsubstituted heterocycle comprising one or two 5- or 6-member rings and 1-4 heteroatoms selected from N, O, and S.

In certain embodiments of a compound of Formula (X″), R³⁶ and R³⁷, together with the nitrogen atom to which they are attached, form a substituted or unsubstituted heterocycle comprising one or two 5- or 6-member rings and 1-4 heteroatoms selected from N, O, and S.

In certain embodiments of a compound of Formula (X″), R³⁶ and R³⁷, together with the nitrogen atom to which they are attached, form a substituted or unsubstituted pyrrolidinyl, imidazolidinyl, pyrazolidinyl, oxazolidinyl, isoxazolidinyl, triazolidinyl, tetrahydrofuranyl, piperidinyl, piperazinyl, or morpholinyl ring.

In certain embodiments of a compound of Formula (X″), R³⁶ and R³⁷, together with the nitrogen atom to which they are attached, form a substituted or unsubstituted piperidinyl ring. In certain embodiments of a compound of Formula (X″), R³⁶ and R³⁷, together with the nitrogen atom to which they are attached, form an unsubstituted piperidinyl ring. In certain embodiments of a compound of Formula (X″), R³⁶ and R³⁷, together with the nitrogen atom to which they are attached, form a halogen substituted piperidinyl ring. In certain embodiments of a compound of Formula (X″), R³⁶ and R³⁷, together with the nitrogen atom to which they are attached, form a 4-halogen substituted piperidinyl ring.

In certain embodiments of a compound of Formula (X″), R³⁶ and R³⁷, together with the nitrogen atom to which they are attached, form an unsubstituted morpholinyl ring.

In certain embodiments of a compound of Formula (X″), R³⁶ and R³⁷, together with the nitrogen atom to which they are attached, form an unsubstituted pyrrolidinyl ring.

In certain embodiments of a compound of Formula (X″), R³⁶ and R³⁷, together with the nitrogen atom to which they are attached, form a substituted or unsubstituted heteroaryl comprising one or two 5- or 6-member rings and 1-4 heteroatoms selected from N, O, and S.

In certain embodiments of a compound of Formula (X″), R³⁶ is substituted or unsubstituted C₆₋₁₀ aryl. In certain embodiments of a compound of Formula (X″), R³⁶ is unsubstituted C₆₋₁₀ aryl. In certain embodiments of a compound of Formula (X″), R³⁶ is unsubstituted phenyl. In certain embodiments of a compound of Formula (X″), R³⁶ is unsubstituted benzyl.

In certain embodiments of a compound of Formula (X″), R³⁷ is hydrogen.

In certain embodiments of a compound of Formula (X″), R³⁷ is substituted or unsubstituted C₁₋₆ alkyl. In certain embodiments of a compound of Formula (X″), R³⁷ is unsubstituted C₁₋₆ alkyl.

In certain embodiments of a compound of Formula (X″), R³⁸ is unsubstituted C₁₋₆ alkyl. In certain embodiments of a compound of Formula (X″), R³⁸ is unsubstituted C₁₋₃ alkyl. In certain embodiments of a compound of Formula (X″), R³⁸ is methyl.

In one embodiment, the compounds of Formula (X″) may be prepared using methods known to those skilled in the art, for example, as disclosed in U.S. Pat. No. 8,669,281 B1.

In one embodiment, the prodrugs of monoalkyl fumarates are the prodrugs disclosed in U.S. Pat. No. 8,669,281 B1, such as the compounds of Formula (XI):

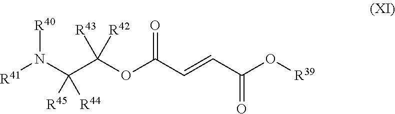

or a pharmaceutically acceptable salt, tautomer, or stereoisomer thereof, wherein
R³⁹ is unsubstituted C₁₋₆ alkyl;
R⁴⁰ and R⁴¹ are each, independently, hydrogen, substituted or unsubstituted C₁₋₆ alkyl, substituted or unsubstituted C₂₋₆ alkenyl, substituted or unsubstituted C₂₋₆ alkynyl, substituted or unsubstituted C₆₋₁₀ aryl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
R⁴², R⁴³, R⁴⁴ and R⁴⁵ are each, independently, hydrogen, substituted or unsubstituted C₁₋₆ alkyl, substituted or unsubstituted C₂₋₆ alkenyl, substituted or unsubstituted C₂₋₆ alkynyl or C(O)OR_{b}; and R_{b} is H or substituted or unsubstituted C₁-C₆ alkyl.

In certain embodiments of a compound of Formula (XI), R³⁹ is methyl. In certain embodiments of a compound of Formula (XI), R³⁹ is ethyl.

In certain embodiments of a compound of Formula (XI), R⁴⁰ is substituted or unsubstituted C₁₋₆ alkyl. In certain embodiments of a compound of Formula (XI), R⁴⁰ is unsubstituted C₁₋₆ alkyl. In certain embodiments of a compound of Formula (XI), R⁴⁰ is unsubstituted C₁₋₃ alkyl. In certain embodiments of a compound of Formula (XI), R⁴⁰ is unsubstituted C₁₋₂ alkyl.

In certain embodiments of a compound of Formula (XI), R⁴⁰ is C(O)OR_{b}-substituted C₁₋₆ alkyl, wherein R_{b} is hydrogen or unsubstituted C₁₋₆ alkyl. In certain embodiments of a compound of Formula (XI), R⁴⁰ is S(O)(O)R_{b}-substituted C₁₋₆ alkyl, wherein R_{b} is unsubstituted C₁₋₆ alkyl.

In certain embodiments of a compound of Formula (XI), R⁴⁰ is substituted or unsubstituted C₆₋₁₀ aryl. In certain embodiments of a compound of Formula (XI), R⁴⁰ is unsubstituted C₆₋₁₀ aryl. In certain embodiments of a compound of Formula (XI), R⁴⁰ is unsubstituted phenyl. In certain embodiments of a compound of Formula (XI), R⁴⁰ is unsubstituted benzyl.

In certain embodiments of a compound of Formula (XI), R⁴¹ is hydrogen.

In certain embodiments of a compound of Formula (XI), R⁴¹ is substituted or unsubstituted C₁₋₆ alkyl. In certain embodiments of a compound of Formula (XI), R⁴¹ is unsubstituted C₁₋₆ alkyl.

In certain embodiments of a compound of Formula (XI), R⁴², R⁴³, R⁴⁴, and R⁴⁵ are each hydrogen.

In certain embodiments of a compound of Formula (XI), R⁴² is substituted or unsubstituted C₁₋₆ alkyl and R⁴³, R⁴⁴, and R⁴⁵ are each hydrogen. In certain embodiments of a compound of Formula (XI), R⁴² is unsubstituted C₁₋₆ alkyl and R⁴³, R⁴⁴, and R⁴⁵ are each hydrogen.

In certain embodiments of a compound of Formula (XI), R⁴⁴ is substituted or unsubstituted C₁₋₆ alkyl and R⁴², R⁴³, and R⁴⁵ are each hydrogen. In certain embodiments of a compound of Formula (XI), R⁴⁴ is unsubstituted C₁₋₆ alkyl and R⁴², R⁴³, and R⁴⁵ are each hydrogen.

In certain embodiments of a compound of Formula (XI), R⁴² and R⁴⁴ are each, independently, substituted or unsubstituted C₁₋₆ alkyl and R⁴³ and R⁴⁵ are each hydrogen. In certain embodiments of a compound of Formula (XI), R⁴² and R⁴⁴ are each, independently, unsubstituted C₁₋₆ alkyl and R⁴³ and R⁴⁵ are each hydrogen.

In certain embodiments of a compound of Formula (XI), R⁴² and R⁴³ are each, independently, substituted or unsubstituted C₁₋₆ alkyl and R⁴⁴ and R⁴⁵ are each hydrogen. In certain embodiments of a compound of Formula (XI), R⁴² and R⁴³ are each, independently, unsubstituted C₁₋₆ alkyl and R⁴⁴ and R⁴⁵ are each hydrogen.

In certain embodiments of a compound of Formula (XI), R⁴⁴ and R⁴⁵ are each, independently, substituted or unsubstituted C₁₋₆ alkyl and R⁴² and R⁴³ are each hydrogen. In certain embodiments of a compound of Formula (XI), R⁴⁴ and R⁴⁵ are each, independently, unsubstituted C₁₋₆ alkyl and R⁴² and R⁴³ are each hydrogen.

In one embodiment, the compounds of Formula (XI) may be prepared using methods known to those skilled in the art, for example, as disclosed in U.S. Pat. No. 8,669,281 B1.

In one embodiment, the prodrugs of monoalkyl fumarates are the prodrugs disclosed in U.S. Pat. No. 8,669,281 B1, such as the compounds of Formula (XII):

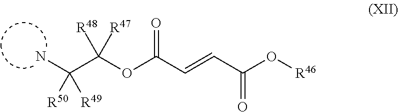

or a pharmaceutically acceptable salt, tautomer, or stereoisomer thereof, wherein
R⁴⁶ is unsubstituted C₁₋₆ alkyl;

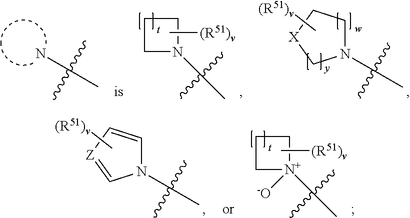

X is N, O, S, or SO₂;
Z is C or N;
t is 0, 1, 2, or 3;
y is 1 or 2;
w is 0, 1, 2, or 3;
v is 0, 1, 2, 3, 4, 5, 6, 7, 8, 9, or 10;
R⁴⁷, R⁴⁸, R⁴⁹ and R⁵⁰ are each, independently, hydrogen, substituted or unsubstituted C₁₋₆ alkyl, substituted or unsubstituted C₂₋₆ alkenyl, substituted or unsubstituted C₂₋₆ alkynyl or C(O)OR⁵²; and
   R⁵² is hydrogen or substituted or unsubstituted C₁₋₆ alkyl; and
each R⁵¹ is, independently, hydrogen, halogen, substituted or unsubstituted C₁₋₆ alkyl, substituted or unsubstituted C₂₋₆ alkenyl, substituted or unsubstituted C₂₋₆ alkynyl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
or, alternatively, two R⁵¹'s attached to the same carbon atom, together with the carbon atom to which they are attached, form a carbonyl, substituted or unsubstituted C₃₋₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S;
or, alternatively, two R⁵¹'s attached to different atoms, together with the atoms to which they are attached, form a substituted or unsubstituted C₃-C₁₀ carbocycle, substituted or unsubstituted heterocycle comprising one or two 5- or 6-member rings and 1-4 heteroatoms selected from N, O, and S, or substituted or unsubstituted heteroaryl comprising one or two 5- or 6-member rings and 1-4 heteroatoms selected from N, O, and S.

In certain embodiments of a compound of Formula (XII), R⁴⁶ is methyl. In certain embodiments of a compound of Formula (XII), R⁴⁶ is ethyl.

In certain embodiments of a compound of Formula (XII),

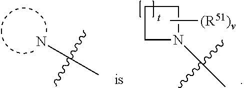

In certain embodiments of a compound of Formula (XII),

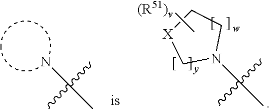

In certain embodiments of a compound of Formula (XII),

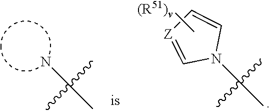

In certain embodiments of a compound of Formula (XII),

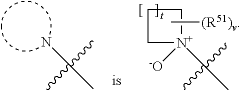

In certain embodiments of a compound of Formula (XII), R⁴⁷ is substituted or unsubstituted C₁₋₆ alkyl and R⁴⁸, R⁴⁹, and R⁵⁰ are each hydrogen. In certain embodiments of a compound of Formula (XII), R⁴⁷ is unsubstituted C₁₋₆ alkyl and R⁴⁸, R⁴⁹, and R⁵⁰ are each hydrogen.

In certain embodiments of a compound of Formula (XII), R⁴⁹ is substituted or unsubstituted C₁₋₆ alkyl and R⁴⁷, R⁴⁸, and R⁵⁰ are each hydrogen. In certain embodiments of a compound of Formula (XII), R⁴⁹ is unsubstituted C₁₋₆ alkyl and R⁴⁷, R⁴⁸, and R⁵⁰ are each hydrogen.

In certain embodiments of a compound of Formula (XII), R⁴⁷ and R⁴⁹ are each, independently, substituted or unsubstituted C₁₋₆ alkyl and R⁴⁸ and R⁴⁹ are each hydrogen. In certain embodiments of a compound of Formula (XII), R⁴⁷ and R⁴⁹ are each, independently, unsubstituted C₁₋₆ alkyl and R⁴⁸ and R⁵⁰ are each hydrogen.

In certain embodiments of a compound of Formula (XII), R⁴⁷ and R⁴⁸ are each, independently, substituted or unsubstituted C₁₋₆ alkyl and R⁴⁹ and R⁵⁰ are each hydrogen. In certain embodiments of a compound of Formula (XII), R⁴⁷ and R⁴⁸ are each, independently, unsubstituted C₁₋₆ alkyl and R⁴⁹ and R⁵⁰ are each hydrogen.

In certain embodiments of a compound of Formula (XII), R⁴⁹ and R⁵⁰ are each, independently, substituted or unsubstituted C₁₋₆ alkyl and R⁴⁷ and R⁴⁸ are each hydrogen. In certain embodiments of a compound of Formula (XII), R⁴⁹ and R⁵⁰ are each, independently, unsubstituted C₁₋₆ alkyl and R⁴⁷ and R⁴⁸ are each hydrogen.

In one embodiment, the compounds of Formula (XII) may be prepared using methods known to those skilled in the art, for example, as disclosed in U.S. Pat. No. 8,669,281 B1.

In certain embodiments of a compound of Formula (X), (X′), (X″), (XI), or (XII), the compound is:

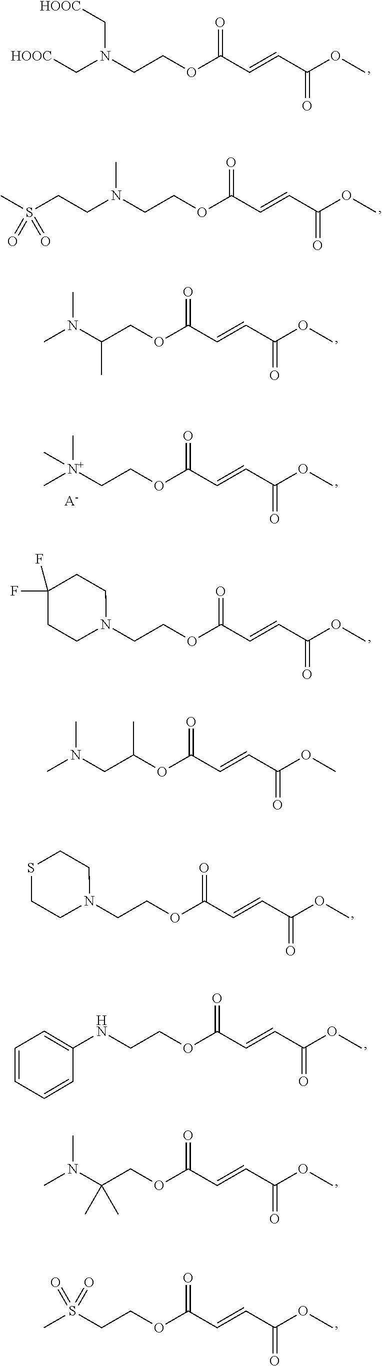

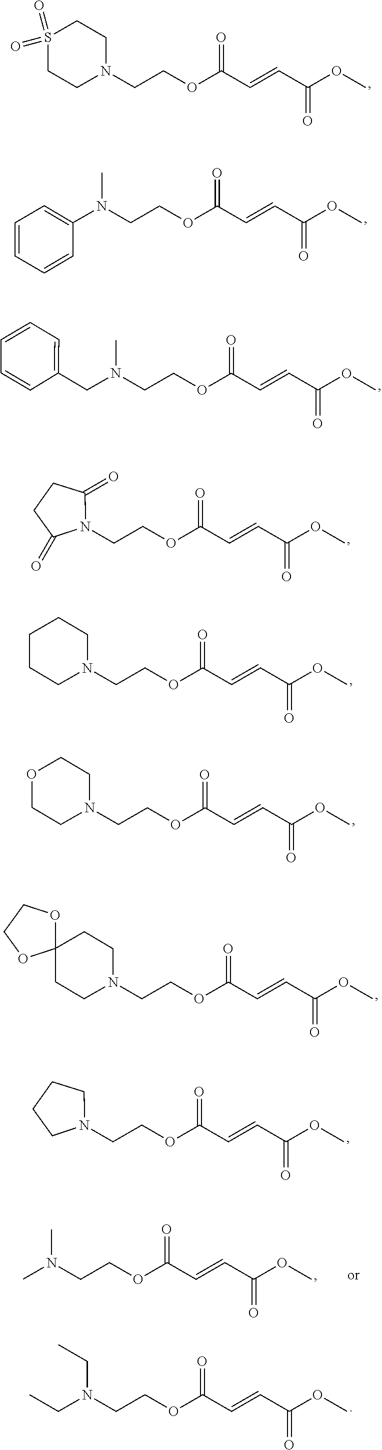

In certain embodiments of a compound of (XII), the compound is

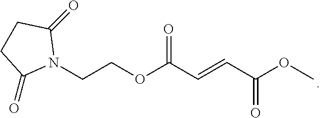 [SMILES: COC(=O)/C=C/C(=O)OCCN1C(=O)CCC1=O]

In one embodiment, the prodrugs of monoalkyl fumarates are the prodrugs disclosed in WO2014/096425, such as the compounds of Formula (XIII):

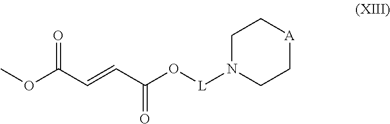

or a pharmaceutically acceptable salt or stereoisomer thereof, wherein

L is is an alkanediyl group with 1 to 6 carbon atoms;

A is SO, SO₂, or NR⁵³, and

R⁵³ is C₁₋₆ alkyl or C₃₋₆ cycloalkyl.

In certain embodiments of a compound of Formula (XIII), L is an alkanediyl group with 2, 3 or 4 carbon atoms, or with 2 or 4 carbon atoms, or with 2 carbons atoms. In certain embodiments of a compound of Formula (XIII), L is —CH₂CH₂—. In certain embodiments of a compound of Formula (XIII), A is SO or SO₂. In certain embodiments of a compound of Formula (XIII), R⁵³ is methyl, ethyl, propyl, isopropyl, butyl, isobutyl, tert-butyl, pentyl, sec-pentyl, or hexyl. In certain embodiments of a compound of Formula (XIII), R⁵³ is cyclopropyl, cyclobutyl, cyclopentyl and cyclohexyl. In certain embodiments of a compound of Formula (XIII), R⁵³ is C₁₋₄ alkyl, C₃ or C₄ or C₅ cycloalkyl. In certain embodiments of a compound of Formula (XIII), R⁵³ is methyl or isopropyl.

In one embodiment, the compounds of Formula (XIII) may be prepared using methods known to those skilled in the art, for example, as disclosed in WO2014/096425.

In one embodiment, the prodrugs of monoalkyl fumarates are the prodrugs disclosed in WO2014/096425, such as the compounds of Formula (XIV):

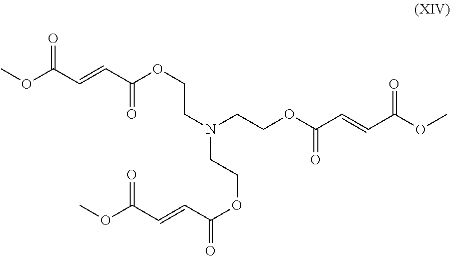

or a pharmaceutical acceptable salt thereof.

In one embodiment, the compounds of Formula (XIV) may be prepared using methods known to those skilled in the art, for example, as disclosed in WO2014/096425.

In one embodiment, the prodrugs of monoalkyl fumarates are the prodrugs disclosed in WO2014/096425, such as the compounds of Formula (XV):

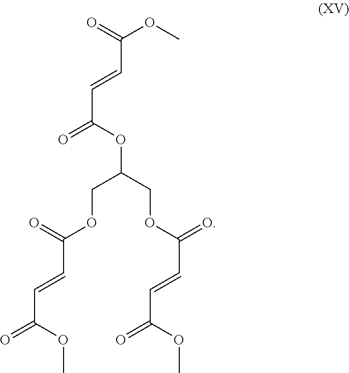 [SMILES: COC(=O)/C=C/C(=O)OCC(COC(=O)/C=C/C(=O)OC)OC(=O)/C=C/C(=O)OC]

In one embodiment, the compounds of Formula (XV) may be prepared using methods known to those skilled in the art, for example, as disclosed in WO2014/096425.

In one embodiment, the prodrugs of monoalkyl fumarates are the prodrugs disclosed in WO2014/096425, such as the compounds of Formula (XVI):

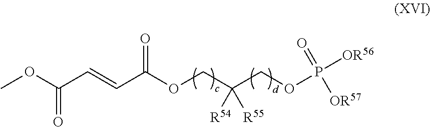

or a stereoisomer thereof, wherein

R⁵⁴ and R⁵⁵ are each, independently, hydrogen, C₁₋₆ alkyl, or C₃₋₆ cycloalkyl;

R⁵⁶ and R⁵⁷ are each, independently, hydrogen or C₁₋₆ alkyl; and

c and d are each, independently, an integer from 0 to 3.

In certain embodiments of a compound of Formula (XVI), R⁵⁴ and R⁵⁵ are each, independently, hydrogen, methyl, or ethyl. In certain embodiments of a compound of Formula (XVI), R⁵⁴ and R⁵⁵ are each, independently, hydrogen or methyl. In certain embodiments of a compound of Formula (XVI), R⁵⁴ and R⁵⁵ are both hydrogen; or R⁵⁴ is hydrogen and R⁵⁵ is methyl. In certain embodiments of a compound of Formula (XVI), c and d each are, independently, 0 or 1. In certain embodiments of a compound of Formula (XVI), c and d are both 0. In certain embodiments of a compound of Formula (XVI), R⁵⁶ and R⁵⁷ are each, independently, C₁₋₅ alkyl or C₁₋₄ alkyl. In certain embodiments of a compound of Formula (XVI), R⁵⁶ and R⁵⁷ are tert-butyl. In certain embodiments of a compound of Formula (XVI), R⁵⁶ and R⁵⁷ are identical.

In one embodiment, the compounds of Formula (XVI) may be prepared using methods known to those skilled in the art, for example, as disclosed in WO2014/096425.

In one embodiment, the prodrugs of monoalkyl fumarates are the prodrugs disclosed in WO2014/096425, such as the compounds of Formula (XVII):

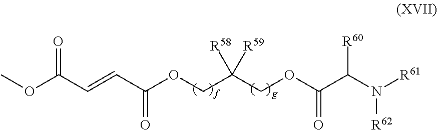

or a pharmaceutically acceptable salt, tautomer, or stereoisomer thereof, wherein
R⁵⁸, R⁵⁹, R⁶¹, and R⁶² are each, independently, hydrogen, C₁₋₆ alkyl, or C₃₋₆ cycloalkyl;
R⁶⁰ is hydrogen, C₃₋₆ cycloalkyl or C₁₋₆ alkyl, wherein the C₁₋₆ alkyl is optionally substituted with or or more of amino, NH—C(NH)NH₂, carboxamide, carboxylic acid, hydroxy, imidazole, indole, mercapto, methylthio, phenyl, hydroxyphenyl, and wherein one of R⁶¹ and R⁶² together with R⁶⁰ optionally belong to a 5 or 6-membered heteroaliphatic ring; and
f and g are each, independently, an integer from 0 to 3, with the proviso that both f and g are not 0.

In certain embodiments of a compound of Formula (XVII), R⁶¹ and R⁶² are each, independently, hydrogen or C₁₋₂ alkyl. In certain embodiments of a compound of Formula (XVII), R⁶¹ and R⁶² are hydrogen. In certain embodiments of a compound of Formula (XVII), R⁶¹ is hydrogen and R⁶² is methyl. In certain embodiments of a compound of Formula (XVII), at least one off and g is 0. In certain embodiments of a compound of Formula (XVII), g is 0.

In certain embodiments of a compound of Formula (XVII), R⁶⁰ is a substituted C₁₋₆ alkyl, wherein the substituent is one or more of the following: halogen, nitro, nitrile, urea, phenyl, aldehyde, sulfate, amino, NH—C(NH)NH₂, carboxamide, carboxylic acid, hydroxy, imidazole, indole, mercapto, methylthio, phenyl, and hydroxyphenyl. In particular embodiments the substituents are one or more of the following: amino, NH—C(NH)NH₂, carboxamide, carboxylic acid, hydroxy, imidazole, indole, mercapto, methylthio, phenyl, and hydroxyphenyl. In certain embodiments of a compound of Formula (XVII), R⁶⁰ is —CH₂—C₆H₅. In certain embodiments of a compound of Formula (XVII), the compound is a compound of Formula XVII′:

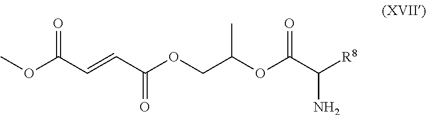

In one embodiment, the compounds of Formula (XVII) or (XVII′) may be prepared using methods known to those skilled in the art, for example, as disclosed in WO2014/096425.

In one embodiment, the prodrugs of monoalkyl fumarates are the prodrugs disclosed in WO2014/096425, such as the compounds of Formula (XVIII):

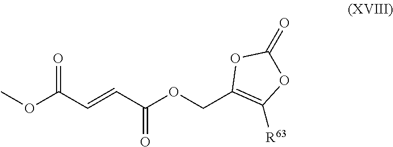

or a pharmaceutically acceptable salt or stereoisomer thereof, wherein
R⁶³ is hydrogen, C₁₋₆ alkyl, C₃₋₆ cycloalkyl, C₂₋₆ alkenyl, halogen, cyano, hydroxy, amino, carboxy, mercapto, 5 or 6-membered aryl or hetero aryl optionally substituted with one of or more of methyl, tert-butyl, hydroxy, methoxy, halogen, nitro, nitrile, amine, and carboxamide.

In certain embodiments of a compound of Formula (XVIII), R⁶³ is hydrogen, C₁₋₂ alkyl, halogen, cyano, amino, or hydroxy. In certain embodiments of a compound of Formula (XVIII), R⁶³ is hydrogen, hydroxyl, or methyl. In certain embodiments of a compound of Formula (XVIII), R⁶³ is methyl.

In one embodiment, the compounds of Formula (XVIII) may be prepared using methods known to those skilled in the art, for example, as disclosed in WO2014/096425.

In certain embodiments of a compound of Formula (XIII), (XVI), (XVII), or (XVIII), the compound is:

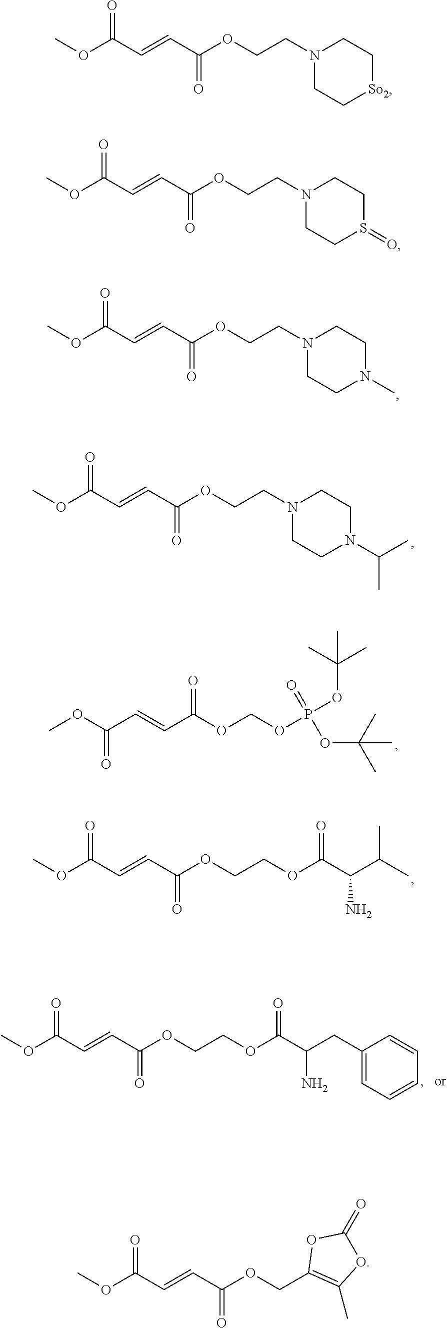

### 5.1.3 Deuterated Fumarates

In one embodiment, a deuterated fumarate is a compound disclosed in U.S. patent application publication number US 2014-0179779 A1, such as a compound of Formula (XIX):

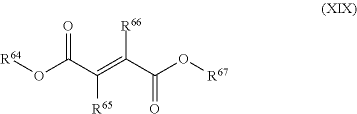

or a pharmaceutically acceptable salt, tautomer, or stereoisomer thereof, wherein
R⁶⁴ and R⁶⁷ are each independently hydrogen, deuterium, deuterated methyl, deuterated ethyl, C₁₋₆ alkyl, phenyl, 3-7 membered saturated or partially unsaturated monocyclic carbocyclic ring, 3-7 membered saturated or partially unsaturated monocyclic heterocyclic ring having 1-3 heteroatoms independently selected from nitrogen, oxygen, and sulfur, or a 5-6 membered heteroaryl ring having 1-3 heteroatoms independently selected from nitrogen, oxygen, and sulfur; and
R⁶⁵ and R⁶⁶ are each independently hydrogen or deuterium, provided that the compound of Formula (XIX) contains at least one deuterium atom and that R⁶⁴ and R⁶⁷ are not hydrogen or deuterium at the same time.

In particular, fumarate isotopologues are the compounds disclosed in US patent application publication number US 2014-0179779 A1, such as the compounds of Formula (XIX′):

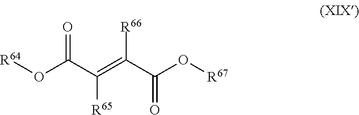

or a pharmaceutically acceptable salt or stereoisomer thereof, wherein
R⁶⁴ and R⁶⁷ are each independently hydrogen, deuterium, deuterated methyl, deuterated ethyl, or C₁₋₆ aliphatic, and
R⁶⁵ and R⁶⁶ are each independently hydrogen or deuterium, provided that the compound of formula (XIX′) contains at least one deuterium atom and that R⁶⁴ and R⁶⁷ are not hydrogen or deuterium at the same time.

In certain embodiments of a compound of Formula (XIX) or Formula (XIX′), R⁶⁴ is hydrogen or —CH₃. In certain embodiments of a compound of Formula (XIX) or Formula (XIX′), R⁶⁴ is —CD₃. In certain embodiments of a compound of Formula (XIX) or Formula (XIX′), R⁶⁴ is —CD₂CD₃.

In certain embodiments of a compound of Formula (XIX) or Formula (XIX′), R⁶⁷ is —CH₂D, —CHD₂, or —CD₃. In certain embodiments of a compound of Formula (XIX) or Formula (XIX′), R⁶⁷ is H, —CH₃, —CH₂D, —CHD₂, or —CD₃.

In certain embodiments of a compound of Formula (XIX) or Formula (XIX′), R⁶⁴ is hydrogen or —CH₃ and R⁶⁷ is —CH₂D, —CHD₂, or —CD₃.

In certain embodiments of a compound of Formula (XIX) or Formula (XIX′), R⁶⁴ is —CD₃ and R⁶⁷ is —CH₂D, —CHD₂, or —CD₃.

In certain embodiments of a compound of Formula (XIX) or Formula (XIX′), at least one of R⁶⁵ and R⁶⁶ is deuterium. In certain embodiments of a compound of Formula (XIX) or Formula (XIX′), both of R⁶⁵ and R⁶⁶ are deuterium.

In certain embodiments of a compound of Formula (XIX) or Formula (XIX′), at least one of R⁶⁵ and R⁶⁶ is deuterium and R⁶⁷ is hydrogen, —CH₃, —CH₂D, —CHD₂, or —CD₃. In certain embodiments of a compound of Formula (XIX) or Formula (XIX′), both of R⁶⁵ and R⁶⁶ are deuterium and R⁶⁷ is hydrogen, —CH₃, —CH₂D, —CHD₂, or —CD₃.

In certain embodiments of a compound of Formula (XIX) or Formula (XIX′), R⁶⁴ is —CD₂CD₃ and R⁶⁷ is H, —CH₃, —CH₂D, —CHD₂, or —CD₃

In certain embodiments of a compound of Formula (XIX) or Formula (XIX′), the compound is (²H₆)dimethyl fumaric acid ester, (²H₃)methyl fumaric acid ester, (²H₃)dimethyl fumaric acid ester, dimethyl fumaric(2,3-²H₂) acid ester, methyl fumaric(2,3-²H₂) acid ester, ethyl fumaric(2,3-²H₂) acid ester, (²H₃)methyl fumaric(2,3-²H₂) acid ester, (²H₆)dimethyl fumaric(2,3-²H₂) acid ester, methyl (2-morpholino-2-oxoethyl) fumaric(2,3-²H₂) acid ester, methyl (4-morpholino-1-butyl) fumaric(2,3-²H₂) acid ester, 2-(benzoyloxy)ethyl methyl fumaric(2,3-²H₂) acid ester, 2-(benzoyloxy)ethyl (²H₃)methyl fumaric acid ester, (S)-2-((2-amino-3-phenylpropanoyl)oxy)ethyl methyl fumaric(2,3-²H₂) acid ester, or (S)-2-((2-amino-3-phenylpropanoyl)oxy)ethyl (²H₃)methyl fumaric acid ester; or a pharmaceutically acceptable salt or stereoisomer thereof.

In certain embodiments of a compound of Formula (XIX) or Formula (XIX′), the compound is:

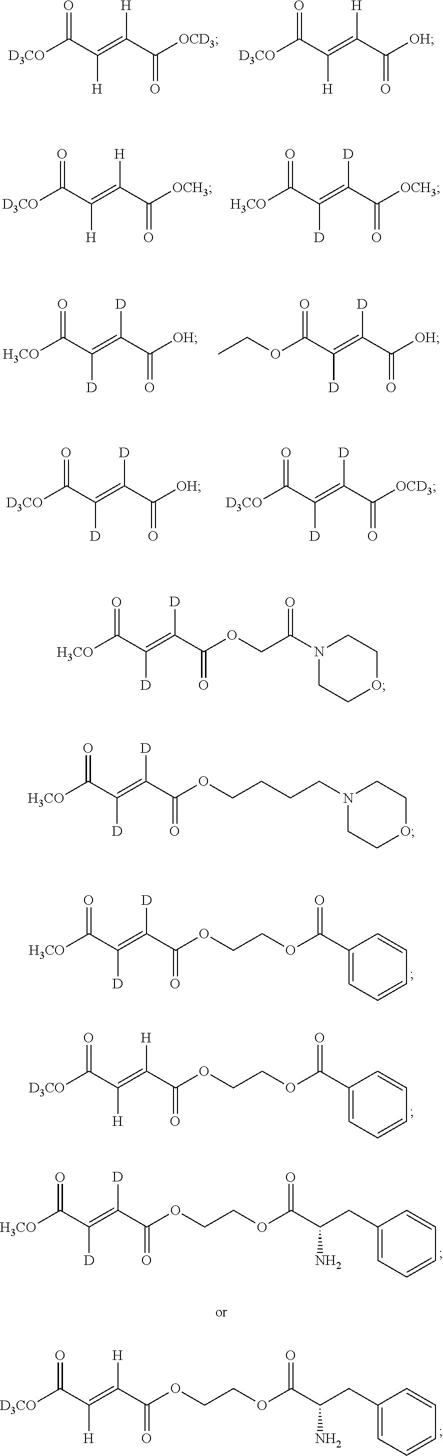

or a pharmaceutically acceptable salt or stereoisomer thereof.

In one embodiment, the compounds of Formula (XIX) and (XIX′) may be prepared using methods known to those skilled in the art, for example, as disclosed in US patent application publication number US 2014-0179779 A1.

Deuterated fumarates are useful as active agents for the methods provided herein, e.g., treating a neurological disease or treating an impairment associated with a neurological disease.

In one embodiment, when a particular position in a fumarate is designated as having deuterium, it is understood that the abundance of deuterium at that position is substantially greater than the natural abundance of deuterium, which is 0.015%. A position designated as having deuterium typically has a minimum deuterium enrichment factor of at least 3340 (50.1% deuterium incorporation) at each atom designated as deuterium in said compound.

In other embodiments, a fumarate provided herein has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

### 5.1.4 Salts

In particular aspects, included within the scope of the fumarates described herein are the non-toxic pharmaceutically acceptable salts of the fumarates described hereinabove (wherein the fumarate is a dialkyl fumarate, a monoalkyl fumarate, a combination of a dialkyl fumarate and a monoalkyl fumarate, a prodrug of monoalkyl fumarate, a deuterated form of any of the foregoing, or a tautomer, or stereoisomer of any of the foregoing, or a combination of any of the foregoing). Acid addition salts are formed by mixing a solution of a fumarate with a solution of a pharmaceutically acceptable non-toxic acid such as hydrochloride, hydrobromide, hydroiodide, nitrate, sulfate, bisulfate, phosphate, acid phosphate, isonicotinate, acetate, lactate, salicylate, citrate, tartrate, pantothenate, bitartrate, ascorbate, succinate, maleate, gentisinate, gluconate, glucaronate, saccharate, formate, benzoate, glutamate, methanesulfonate, ethanesulfonate, benzenesulfonate, p-toluenesulfonate, and pamoate. Acceptable base salts include aluminum, calcium, lithium, magnesium, potassium, sodium, zinc, and diethanolamine salts.

### 5.2 Neurological Diseases and Impairments Treated in Accordance with the Methods Provided Herein

Provided herein are methods of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

Provided herein are methods of treating neurological diseases, for example, impairments associated with neurological diseases, comprising administering intravenously to a patient in need thereof at least one fumarate disclosed herein. In one embodiment, the neurological disease is a disease that can be treated by upregulating the Nrf2/ARE pathway. In a specific embodiment, the neurological disease is stroke. In a specific embodiment, the neurological disease is amyotrophic lateral sclerosis. In a specific embodiment, the neurological disease is Huntington's disease. In a specific embodiment, the neurological disease is Alzheimer's disease. In a specific embodiment, the neurological disease is Parkinson's disease. In a specific embodiment, the neurological disease is Multiple Sclerosis.

In one embodiment, the neurological disease is a disease involving white matter. In another embodiment, the neurological disease is a disease involving demyelination. In a specific embodiment, the disease is not multiple sclerosis.

In specific embodiments, the terms “treating” and “treatment” can include improving, ameliorating, curing, or lessening one or more symptoms or impairments of, maintaining remission of, or inhibiting progression of, a disease or disorder.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat the disease, for example, an impairment associated with the disease.

In a specific embodiment, the patient is a human.

In certain embodiments, intravenous administration of a fumarate is more effective at treating neurological diseases than oral administration of the fumarate. In certain embodiments, intravenous administration of a fumarate is more effective at having the fumarate or its in vivo conversion product (e.g., dimethyl fumarate and monomethyl fumarate, respectively) reach the brain than oral administration of the fumarate; that is, greater amounts in the brain are achieved upon intravenous administration relative to oral administration. In one embodiment, the in vivo conversion product is, e.g., a fumarate conjugated to a second compound, wherein the second compound is, e.g., gluthathione, cysteine, or a protein. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with a neurological disease in a patient. This can be demonstrated by an improved readout in one or more methods, which are known in the art and which may be used to assess the impairment associated with a neurological disease, over periods of at least or more than: 2 weeks, 1 month, 1 year, or 2 years.

In certain embodiments, at least one fumarate is administered to a patient repeatedly for at least or more than: 1 day, 2 days, 5 days, 1 week, 2 weeks, 1 month, 1 year, or 2 years.

In specific embodiments, the impairment associated with a neurological disease is assessed by one or more methods known in the art. In other specific embodiments, the methods described herein further comprise assessing the impairment associated with a neurological disease before and/or after the administering step, wherein the impairment is assessed by one or more methods known in the art. In one embodiment, the methods described herein further comprise assessing the level of said impairment after repeated administration of a fumarate described herein.

In a specific embodiment, treatment of a neurological disease, for example, the improvement of an impairment associated with the neurological disease, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by a neurological disease, or to eliminate an impairment associated with a neurological disease.

In certain embodiments, treating a patient by administering an amount of a fumarate is effective to inhibit progression of, or to inhibit development of, an impairment associated with a neurological disease.

In one embodiment, the fumarate is administered in a therapeutically effective amount to the patient. In a particular embodiment, the administration of the fumarate in a therapeutically effective amount improves the impairment associated with a neurological disease in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform a particular task. In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

### 5.2.1 Stroke

Provided herein are methods of treating stroke, for example, one or more impairments associated with stroke, comprising administering intravenously to a patient in need thereof at least one fumarate disclosed herein.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat stroke, for example, an impairment associated with stroke.

In a specific embodiment, the patient is a human.

In certain embodiments, intravenous administration of a fumarate is more effective at treating stroke than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with stroke in a patient. This can be demonstrated by an improved readout in one or more methods, which are known in the art and which may be used to assess the impairment associated with stroke, over periods of at least or more than: 2 weeks, 1 month, 1 year, or 2 years.

In certain embodiments, at least one fumarate is administered to a patient repeatedly for at least or more than: 1 day, 2 days, 5 days, 1 week, 2 weeks, 1 month, 1 year, or 2 years.

In certain embodiments, at least one fumarate is administered to a patient within at least 1 hour, 3 hours, 5 hours, 12 hours, 1 day, 2 days, 5 days, 1 week, 2 weeks, or 1 month from when the stroke occurred.

In specific embodiments, the impairment associated with stroke is assessed by one or more methods known in the art. In other specific embodiments, the methods described herein further comprise assessing the impairment associated with stroke before and/or after the administering step, wherein the impairment is assessed by one or more methods known in the art.

In one embodiment, the methods described herein further comprise assessing the level of said impairment after repeated administration of a fumarate described herein.

In a specific embodiment, treatment of stroke, for example, the improvement of an impairment associated with stroke, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by stroke, or to eliminate an impairment associated with stroke.

In certain embodiments, treating a patient by administering an amount of a fumarate is effective to inhibit progression of, or to inhibit development of, an impairment associated with stroke.

In one embodiment, the fumarate is administered in a therapeutically effective amount to the patient. In a particular embodiment, the administration of the fumarate in a therapeutically effective amount improves the impairment associated with stroke in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform particular task. In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

In specific embodiments, provided herein are methods of treating stroke for improvement of an impairment associated with stroke, wherein the impairment is a sensorimotor impairment, upper limb spasticity, impairment in walking, impairment in global body control, proprioception, impairment in reflexes, impairment in dexterity, limb paralysis, impairment in endurance, impairment in hand strength, impairment in manual dexterity, fine hand coordination loss, hyperreflexia, muscle weakness, impairment in muscle tone, impairment in gait, impairment in range of motion, impairment in speech, ataxia, weakness or fatigue, tremor, impairment in limb function and mobility, impairment in coordination or balance, impairment in chewing or swallowing, impairment of visual function, impairment in hand function, facial paralysis, or impairment in upper and lower extremity motor function.

The methods disclosed below for assessing stroke-associated impairments are discussed in Compendium of Instructions for Outcome Measure, StrokEDGE Taskforce (2011), American Physical Therapy Association, Neurology Section.

### 5.2.1.1. Impairment of Visual Function

In one embodiment, the impairment associated with stroke and treated according to the methods described herein is an impairment of visual function. In a specific embodiment, the impairment in visual function can be assessed (before and/or after administration of a fumarate) using one or more methods known in the art.

In one embodiment, the visual function impairment associated with stroke in a human patient can be assessed (before and/or after administration of a fumarate) by Contrast Sensitivity Testing.

### 5.2.1.2. Facial Paralysis

In one embodiment, the impairment associated with stroke and treated according to the methods described herein is facial paralysis. In a specific embodiment, facial paralysis can be assessed (before and/or after administration of a fumarate) using one or more methods known in the art.

### 5.2.1.3. Proprioception

In one embodiment, the impairment associated with stroke and treated according to the methods described herein is a proprioception. In a specific embodiment, proprioception can be assessed (before and/or after administration of a fumarate) using one or more methods known in the art.

### 5.2.1.4. Global Body Control Impairments

In one embodiment, the impairment associated with stroke and treated according to the methods described herein is an impairment in global body control. In a specific embodiment, the impairment in global body control can be assessed before and/or after administration of a fumarate) using one or more methods known in the art.

In one embodiment, the impairment in global body control associated with stroke in a human patient can be assessed (before and/or after administration of a fumarate) by the Functional Independence Measure (FIM™).

In one embodiment, an improvement in the impairment in global body control associated with stroke is assessed by administering the FIM™, which contains 13 motor tasks and 5 cognitive tasks, rated on a 7 point ordinal scale ranging from total assistance (or complete dependence) to complete independence.

### 5.2.1.5. Coordination or Balance Impairments

In one embodiment, the impairment associated with stroke and treated according to the methods described herein is an impairment in coordination or balance. In a specific embodiment, the impairment in coordination or balance can be assessed (before and/or after administration of a fumarate) using one or more methods known in the art.

In one embodiment, the impairment in balance associated with stroke in a human patient can be assessed (before and/or after administration of a fumarate) by the Berg Balance Scale.

### 5.2.1.6. Impairment in Gait

In one embodiment, the impairment associated with stroke and treated according to the methods described herein is an impairment in gait. In a specific embodiment, the impairment in gait can be assessed (before and/or after administration of a fumarate) using one or more methods known in the art.

In one embodiment, the impairment in gait associated with stroke in a human patient can be assessed (before and/or after administration of a fumarate) via the Timed 10-Meter Gait Test.

In one embodiment, an improvement in the impairment in gait associated with stroke is assessed by making the subject walk 10 meters (32.8 feet) without assistance and measuring the time it takes for the patient to walk the intermediate 6 meters (19.7 feet). Timing begins when the patient reaches meter 2 and stops when the patient's toes reach meter 8, to allow for acceleration and deceleration. The scores are expressed in meters covered per second. Timing a 10-Meter walk, which provides a snapshot of gait velocity, is considered a scientifically reliable and valid test that provides an accurate measurement of a patient's ambulatory capacity.

### 5.2.1.7. Impairment in Endurance

In one embodiment, the impairment associated with stroke and treated according to the methods described herein is an impairment in endurance. In a specific embodiment, the impairment in endurance can be assessed (before and/or after administration of a fumarate) using one or more methods known in the art.

In one embodiment, the impairment in endurance associated with stroke in a human patient can be assessed (before and/or after administration of a fumarate) by the 6 Minute Walk Test. In one embodiment, an improvement in the impairment in endurance associated with stroke is assessed by comparing the distance walked in six minutes, before and after treatment.

### 5.2.1.8. Ataxia

In one embodiment, the impairment associated with stroke and treated according to the methods described herein is ataxia. In a specific embodiment, ataxia can be assessed before and/or after administration of a fumarate using one or more methods known in the art.

In one embodiment, the ataxia associated with stroke in a human patient can be assessed (before and/or after administration of a fumarate) by the Finger-to-Nose Test. In another embodiment, the ataxia associated with stroke in a human patient can be assessed (before and/or after administration of a fumarate) by the Heel-To-Shin Test.

### 5.2.1.9. Walking Impairment

In one embodiment, the impairment associated with stroke and treated according to the methods described herein is an impairment in walking. In a specific embodiment, the impairment in walking can be assessed (before and/or after administration of a fumarate or a pharmaceutically acceptable salt thereof) using one or more methods known in the art.

In one embodiment, the impairment in walking associated with stroke in a human patient can be assessed (before and/or after administration of a fumarate or) by the Timed 25-Foot Walk. In one embodiment, an improvement in the impairment in walking associated with stroke is assessed by measuring the time it takes for the patient to complete a 25-foot walk. The Time 25-Foot Walk is a well-known method for assessing walking impairment. It is composed of directing the patient to one end of a clearly marked 25-foot course and instructing the patient to walk 25 feet as quickly as possible, but safely. The time is calculated from the initiation of the instruction to start to when the patient has reached the 25-foot mark. The task is immediately administered again by having the patient walk back the same distance. The score for the test is the average of the two completed trials.

### 5.2.1.10. Impairment in Dexterity

In one embodiment, the impairment associated with stroke and treated according to the methods described herein is an impairment in dexterity. In a specific embodiment, the impairment in dexterity can be assessed (before and/or after administration of a fumarate) using one or more methods known in the art.

### 5.2.1.11. Impairment in Hand Function

In one embodiment, the impairment associated with stroke and treated according to the methods described herein is impairment in hand function. In a specific embodiment, impairment in hand function can be assessed (before and/or after administration of a fumarate) using one or more methods known in the art.

In one embodiment, impairment in hand function associated with stroke in a human patient can be assessed (before and/or after administration of a fumarate) by the Jebsen-Taylor Hand Function test. In one embodiment, an improvement in the impairment in hand function associated with stroke is assessed by comparing the time taken to complete each of the Jebsen-Taylor test's seven tasks before and after treatment.

The Jebsen-Taylor Hand Function test is a commonly used test of unilateral hand function in adults with stable hand impairments. The test measures the amount of time a subject takes to complete each of the following tasks: (1) writing (copying) a 24-letter sentence, (2) turning over 3″×5″ cards (simulated page turning), (3) picking up small common objects (e.g., a paper clip, bottle cap, and coin) (4) simulated feeding using a teaspoon and five kidney beans, (5) stacking checkers, (6) picking up large light objects (e.g., empty tin can) and (7) picking up large heavy objects (full tin can weighing 1 pound). The non-dominant hand is tested first; then the dominant hand is tested.

### 5.2.1.12. Impairment in Reflexes

In one embodiment, the impairment associated with stroke and treated according to the methods described herein is an impairment in reflexes. In a specific embodiment, the impairment in reflexes can be assessed (before and/or after administration of a fumarate) using one or more methods known in the art.

### 5.2.1.13. Impairment in Hand Strength

In one embodiment, the impairment associated with stroke and treated according to the methods described herein is impairment in hand strength. In a specific embodiment, impairment in hand strength can be assessed (before and/or after administration of a fumarate) using one or more methods known in the art.

In one embodiment, impairment in hand strength associated with stroke in a human patient can be assessed (before and/or after administration of a fumarate) by the Grip test. In one embodiment, an improvement in the impairment in hand strength associated with stroke is assessed by using a dynamometer, before and after treatment. The Grip test is a simple, valid and reliable measure to identify hand strength and to detect the change that may result from a course of treatment. Hand strength on each hand is measured using a dynamometer.

In one embodiment, impairment in hand strength associated with stroke in a human patient can be assessed (before and/or after administration of a fumarate) by the Pinch test. In one embodiment, an improvement in the impairment in hand strength associated with stroke is assessed by comparing pinch strength before and after treatment. The Pinch tests are simple, valid and reliable measures to identify hand strength and to detect the change that may result from a course of treatment. Hand strength on each hand is measured using a dynamometer. The tests comprise three components: the tip, key, and palmar pinch. Pinch strength is measured using a pinch gauge.

### 5.2.1.14. Hyperreflexia

In one embodiment, the impairment associated with stroke and treated according to the methods described herein is hyperreflexia. In a specific embodiment, hyperreflexia can be assessed (before and/or after administration of a fumarate) using one or more methods known in the art.

### 5.2.1.15. Impairment in Manual Dexterity

In one embodiment, the impairment associated with stroke and treated according to the methods described herein is an impairment in manual dexterity. In a specific embodiment, the impairment in manual dexterity can be assessed (before and/or after administration of a fumarate) using one or more methods known in the art.

In one embodiment, the impairment in manual dexterity associated with stroke in a human patient can be assessed (before and/or after administration of a fumarate) by the Box and Block test. In one embodiment, an improvement in the impairment in manual dexterity associated with stroke is assessed by comparing the number of blocks moved, one at a time, from one side of a partition to another in one minute, before and after treatment. The Box and Block test is the standard test of manual dexterity. It measures how many blocks a subject can move from one side of a box to another, over a partition in the middle of the box, in one minute. The subject is instructed to move only one block at a time.

### 5.2.1.16. Fine Hand Coordination Loss

In one embodiment, the impairment associated with stroke and treated according to the methods described herein is fine hand coordination loss. In a specific embodiment, fine hand coordination loss can be assessed (before and/or after administration of a fumarate) using one or more methods known in the art.

### 5.2.1.17. Muscle Tone Impairment

In one embodiment, the impairment associated with stroke and treated according to the methods described herein is an impairment in muscle tone. In a specific embodiment, the impairment in muscle tone can be assessed (before and/or after administration of a fumarate) using one or more methods known in the art.

### 5.2.1.18. Range of Motion Impairment

In one embodiment, the impairment associated with stroke and treated according to the methods described herein is an impairment in range of motion. In a specific embodiment, range of motion impairment can be assessed (before and/or after administration of a fumarate) using one or more methods known in the art.

### 5.2.1.19. Weakness or Fatigue

In one embodiment, the impairment associated with stroke and treated according to the methods described herein is weakness or fatigue. In a specific embodiment, weakness or fatigue can be assessed (before and/or after administration of a fumarate) using one or more methods or known in the art.

### 5.2.1.20. Muscle Weakness

In one embodiment, the impairment associated with stroke and treated according to the methods described herein is muscle weakness. In a specific embodiment, muscle weakness can be assessed before and/or after administration of a fumarate) using one or more methods known in the art.

In one embodiment, the muscle weakness associated with stroke in a human patient can be assessed (before and/or after administration of a fumarate) by the Five Times Sit-to-Stand Test. In one embodiment, an improvement in the muscle strength associated with stroke is assessed by comparing the time taken to complete the Five Times Sit-to-Stand Test, before and after treatment. The Five Times Sit-to-Stand test provides a measure of functional lower limb muscle strength. The patient sits arms with arms folded across chest and with his or her back against the chair. The patient is instructed to stand and sit five times as quickly as possible without touching the back of the chair.

### 5.2.1.21. Upper Limb Spasticity

In one embodiment, the impairment associated with stroke and treated according to the methods described herein is upper limb spasticity. In a specific embodiment, upper limb spasticity can be assessed (before and/or after administration of a fumarate) using one or more methods known in the art.

In one embodiment, the upper limb spasticity associated with stroke in a human patient can be assessed (before and/or after administration of a fumarate) by the Disability Assessment Scale.

In one embodiment, the upper limb spasticity associated with stroke in a human patient can be assessed (before and/or after administration of a fumarate) by the Modified Ashworth Scale. In one embodiment, an improvement in the upper limb spasticity associated with stroke is assessed by comparing the subjective rating of the amount of resistance or tone perceived by the examiner as the limb is moved through its full range of motion, before and after treatment. The Modified Ashworth Scale is a widespread method routinely used to measure spasticity. It measures resistance during passive soft-tissue stretching.

### 5.2.1.22. Tremors

In one embodiment, the impairment associated with stroke and treated according to the methods described herein is tremor. In a specific embodiment, tremor can be assessed (before and/or after administration of a fumarate) using one or more methods known in the art.

### 5.2.1.23. Impairment in Limb Function and Mobility

In one embodiment, the impairment associated with stroke and treated according to the methods described herein is an impairment in limb function and mobility. In a specific embodiment, the impairment in limb function and mobility can be assessed (before and/or after administration of a fumarate) using one or more methods known in the art.

In one embodiment, the impairment in limb function and mobility associated with stroke in a human patient can be assessed (before and/or after administration of a fumarate) by the Wolf Function Motor Test. In one embodiment, an improvement in the impairment in limb function and mobility associated with stroke is assessed by administering the Wolf Function Mobility Test, before and after treatment. The Wolf Motor Function Test quantifies upper extremity motor ability through timed and functional tasks. It consists of 17 items or tasks. Tasks are arranged in order of complexity and progress from proximal to distal joint involvement. Tasks are assessed for performance time and quality of movement and function. While each task is timed, excessive performance time is typically truncated to 120 seconds. Summary score for performance time assessment is the median time recorded over all tasks.

### 5.2.1.24. Limb Paralysis

In one embodiment, the impairment associated with stroke and treated according to the methods described herein is limb paralysis. In a specific embodiment, limb paralysis can be assessed (before and/or after administration of a fumarate) using one or more methods known in the art.

### 5.2.1.25. Speech Impairments (e.g., Dystharia, Apraxia, or Dysphonia)

In one embodiment, the impairment associated with stroke and treated according to the methods described herein is an impairment in speech. In a specific embodiment, the impairment in speech is, Dystharia, Apraxia, or Dysphonia. In a certain embodiments, the impairment in speech can be assessed (before and/or after administration of a fumarate) using one or more methods known in the art.

### 5.2.1.26. Chewing or Swallowing Impairments

In one embodiment, the impairment associated with stroke and treated according to the methods described herein is an impairment in chewing or swallowing. In a specific embodiment, the impairment in chewing or swallowing is dysphagia. In a specific embodiment, the impairment in chewing or swallowing can be assessed (before and/or after administration of a fumarate) using one or more methods known in the art.

In one embodiment, the impairment in chewing or swallowing associated with stroke in a human patient can be assessed (before and/or after administration of a fumarate or a pharmaceutically acceptable salt thereof) by an X-ray with a contrast material, such as a Barium X-ray. In one embodiment, an improvement in the impairment in chewing or swallowing associated with stroke is assessed by administering a Barium X-ray, before and after treatment. The Barium X-ray is a well-known method in the art. The patient swallows a barium solution that coats the esophagus, allowing the physician to see changes in the shape of your esophagus and to assess the muscular activity.

In one embodiment, the impairment in swallowing associated with stroke in a human patient can be assessed (before and/or after administration of a fumarate) by the Dynamic Swallowing Study. In one embodiment, an improvement in the impairment in swallowing associated with stroke is assessed by the Dynamic Swallowing Study, before and after treatment. The Dynamic Swallowing Study is a well-established method in the art. The patient swallows barium-coated foods of different consistencies. This test provides an image of these foods as they travel through the mouth and down the throat.

In one embodiment, the impairment in swallowing associated with stroke in a human patient can be assessed (before and/or after administration of a) by esophageal muscle test (manometry). In one embodiment, an improvement in the impairment in swallowing associated with stroke is assessed by administering the esophageal muscle test, before and after treatment. Manometry is a known method in the art. A small tube is inserted into the patient's esophagus and connected to a pressure recorder to measure the muscle contractions of the esophagus as the patient swallows.

### 5.2.1.27. Upper and Lower Extremity Motor Function Impairment

In one embodiment, the impairment associated with stroke and treated according to the methods described herein is an impairment in upper and lower extremity motor function. In a specific embodiment, the impairment in upper and lower extremity motor function can be assessed (before and/or after administration of a fumarate) using one or more methods known in the art.

In one embodiment, the impairment in upper and lower extremity motor function associated with stroke in a human patient can be assessed (before and/or after administration of a fumarate) by the Fugl-Meyer Assessment.

### 5.2.1.28. Various Other Tests for Measuring Sensorimotor Impairments

In other embodiments, an impairment associated with stroke described herein or known in the art, including an impairment of motor functions, can be assessed, without limitation, using: the 2 minute walk test, Six Spot Step Test, the Manual Muscle test for lower extremity function, Lower Extremity Manual Muscle Test (LEMMT), the Ashworth score, 9-hole peg test, fine finger movement, rapid alternating fingers for upper extremity function, or functional system scoring for sensory function. In specific embodiments, a 2 minute walk test can be used to measure walking, LEMMT can be used to measure lower extremity muscle strength, and/or the Modified Ashworth Scale can be used to measure spasticity. GAITRite™ technology (e.g., 26 foot GAITRite™) can be used to measure gait, e.g., stride length and velocity. The NeuroCom SMART Balance Mastelx can be used to measure gait and balance parameters such as step length. A Step Watch® accelerometer can be used to measure gait. Other known upper extremity function assessments include, without limitation, performance scale-self-report measures, hand-held dynamometry, and Upper Extremity Index (UEI). Other assessment tests that can be used to measure motor functions include but are not limited to: Kela Coordination Test, Postural Stability Test, Shoulder Tug Test, Maximal isometric force of the knee extensors, muscle endurance tests, passive straight leg raise, TEMP A (upper extremity performance test for the elderly), The Disabilities of the Arm, Shoulder and Hand (DASH) Questionnaire, and Manual Ability Measure-36 (MAM-36). Such assessments can be performed before and after administration of a fumarate to a patient in accordance with the methods disclosed herein.

### 5.2.2 Amyotrophic Lateral Sclerosis

Provided herein are methods of treating Amyotrophic Lateral Sclerosis (“ALS”) or Lou Gehrig's Disease, for example, one or more impairments associated with ALS, comprising administering intravenously to a patient in need thereof at least one fumarate disclosed herein.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat ALS, for example, an impairment associated with ALS.

In a specific embodiment, the patient is a human.

In certain embodiments, intravenous administration of a fumarate is more effective at treating ALS than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with ALS in a patient. This can be demonstrated by an improved readout in one or more methods, which are known in the art and which may be used to assess the impairment associated with ALS, over periods of at least or more than: 2 weeks, 1 month, 1 year, or 2 years.

In certain embodiments, at least one fumarate is administered to a patient repeatedly for at least or more than: 2 weeks, 1 month, 1 year, or 2 years.

In specific embodiments, the impairment associated with ALS is assessed by one or more methods known in the art. In other specific embodiments, the methods described herein further comprise assessing the impairment associated with ALS before and/or after the administering step, wherein the impairment is assessed by one or more methods known in the art. In one embodiment, the methods described herein further comprise assessing the level of said impairment after repeated administration of a fumarate described herein.

In a specific embodiment, treatment of ALS, for example, the improvement of an impairment associated with ALS, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by ALS, or to eliminate an impairment associated with ALS.

In certain embodiments, treating a patient by administering an amount of a fumarate is effective to inhibit progression of, or to inhibit development of, an impairment associated with ALS.

In one embodiment, the fumarate is administered in a therapeutically effective amount to the patient. In a particular embodiment, the administration of the fumarate in a therapeutically effective amount improves the impairment associated with ALS in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform particular task. In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

In specific embodiments, provided herein are methods of treating ALS for improvement of an impairment associated with ALS, wherein the impairment is a sensorimotor impairment, upper limb spasticity, impairment in walking, impairment in global body control, proprioception, impairment in reflexes, impairment in dexterity, limb paralysis, impairment in endurance, impairment in hand strength, impairment in manual dexterity, fine hand coordination loss, hyperreflexia, muscle weakness, impairment in muscle tone, impairment in gait, impairment in range of motion, impairment in speech, ataxia, weakness or fatigue, tremor, impairment in limb function and mobility, impairment in coordination or balance, impairment in chewing or swallowing, impairment of visual function, impairment in hand function, facial paralysis, or impairment in upper or lower extremity motor function.

### 5.2.2.1. Lower Motor Neuron Function Impairments

In one embodiment, the impairment associated with ALS and treated according to a method described herein is a lower motor neuron function impairment, such as muscle weakness, muscle wasting and fasciculation, or muscle twitching.

### 5.2.2.2. Upper Motor Neuron Function Impairments

In another embodiment, the impairment associated with ALS and treated according to a method described here is an upper motor neuron function impairment, such as spasticity in the lower limbs, face, or jaw; severe walking impairment; heaviness, fatigue, stiffness, or lack of coordination of any affected limb; an impairment in reflexes such as brisk or exaggerated reflexes.

### 5.2.2.3. Bulbar ALS Impairments

In another embodiment, the impairment associated with ALS and treated according to a method described herein is caused by a degeneration of the motor neurons in the brainstem (bulbar ALS), such as an impairment in the ability to speak loudly and clearly (dysarthria), or a complete inability to vocalize. Other bulbar ALS impairments include nasal speech quality; difficulty pronouncing words due to impairments in speech muscles; and reduced breath control(Wijesekera et al., 2009, Orphanet J Rare Dis. (2) 4:3).

In another embodiment, the impairment associated with ALS and treated according to a method described herein is difficulty chewing and swallowing (dysphagia), or outbursts of laughter or crying with minimal provocation.

### 5.2.2.4. Spinal ALS Impairments

In another embodiment, the impairment associated with ALS and treated according to a method described herein is caused when motor neurons in the spinal cord are affected (spinal ALS). Such an impairment can be awkwardness and stumbling when walking or running (or an eventual inability to walk or stand); difficulty in lifting objects; an impairment in manual dexterity, and an inability to perform activities of daily living (Wijesekera et al., 2009, Orphanet J Rare Dis. (2) 4:3).

### 5.2.2.5. Tests for the Impairments Associated with ALS

The symptoms and impairments associated with ALS and treated according to the methods described herein can be assessed using one or more of the following methods described below or known in the art.

TUFTS Quantitative Neuromuscular Examination (TQNE)

In a particular embodiment, impairment in muscle strength and function associated with ALS in a human patient can be assessed (before and/or after administration of a fumarate) by the TUFTS Quantitative Neuromuscular Examination (TQNE).

The TQNE is a standardized test to measure strength and function in ALS. The test involves measurement of maximum voluntary isometric contraction (MVIC) of 8 muscle groups in the arms using a strain gauge tensiometer. This measurement is a standard for clinical trials in ALS (Ross et al., 1996, Neurology May; 46 (5):1442-4).

ALS Functional Rating Scale (ALSFRS)

In a particular embodiment, an impairment in muscle strength and function associated with ALS in a human patient can be assessed (before and/or after administration of a fumarate or a pharmaceutically acceptable salt thereof) by the ALS Functional Rating Scale (ALSFRS). The ALSFRS is an ordinal rating scale used to determine patients' assessment of their ability in various functional activities (Cedarbaum et al. 1999, J. Neurological Sciences 169: 13-21).

Forced Vital Capacity (FVC)

In a particular embodiment, an impairment in respiration associated with ALS in a human patient can be assessed (before and/or after administration of a fumarate or a pharmaceutically acceptable salt thereof) by measuring the Forced Vital Capacity (FVC).

FVC is a measure of total amount of air that can be moved in or out of the lung, measured by instructing the patient to exhale into a spirometer. The FVC is easy to perform and is a meaningful indicator of respiratory status (Czaplinski et al., 2006, Journal of Neurology, Neurosurgery, and Psychiatry 77.3: 390-392).

Other Assessment Methods

In other embodiments, an impairment associated with ALS described herein or known in the art, including an impairment of motor functions, can be assessed, without limitation, using: the Timed 25-Foot Walk Test, 6 Minute Gait Test, 2 minute walk test, Six Spot Step Test, the Manual Muscle test for lower extremity function, Lower Extremity Manual Muscle Test (LEMMT), the Ashworth score, Modified Ashworth Scale, 9-hole peg test, fine finger movement, rapid alternating fingers for upper extremity function, or functional system scoring for sensory function. In particular, the Timed 25-Foot Walk, 6 Minute Gait Test, and/or 2 Minute Walk Test can be used to measure walking, LEMMT can be used to measure lower extremity muscle strength, and/or the Modified Ashworth Scale can be used to measure spasticity. GAITRite™ technology (e.g., 26 foot GAITRite™) can be used to measure gait, e.g., stride length and velocity. The NeuroCom SMART Balance Mastel® can be used to measure gait and balance parameters such as step length. A Step Watch® accelerometer can be used to measure gait. Other known upper extremity function assessments include, without limitation, performance scale-self-report measures, hand-held dynamometry, and Upper Extremity Index (UEI). Other assessment tests that can be used to measure motor functions include but are not limited to: Berg Balance Test, Kela Coordination Test, Postural Stability Test, Shoulder Tug Test, Maximal isometric force of the knee extensors, muscle endurance tests, passive straight leg raise, TEMP-A (upper extremity performance test for the elderly), The Disabilities of the Arm, Shoulder and Hand (DASH) Questionnaire, Timed Up-and-Go Test, and Manual Ability Measure-36 (MAM-36). Such assessments can be performed before and after administration of a fumarate to a patient in accordance with the methods disclosed herein.

### 5.2.3 Huntington's Disease

Provided herein are methods of treating Huntington's disease, for example, one or more impairments associated with Huntington's disease, comprising administering intravenously to a patient in need thereof at least one fumarate disclosed herein.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat Huntington's disease, for example, an impairment associated with Huntington's disease.

In a specific embodiment, the patient is a human.

In certain embodiments, intravenous administration of a fumarate is more effective at treating Huntington's disease than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with Huntington's disease in a patient. This can be demonstrated by an improved readout in one or more methods, which are known in the art and which may be used to assess the impairment associated with Huntington's disease, over periods of at least or more than: 2 weeks, 1 month, 1 year, or 2 years.

In certain embodiments, at least one fumarate is administered to a patient repeatedly for at least or more than: 2 weeks, 1 month, 1 year, or 2 years.

In specific embodiments, the impairment associated with Huntington's disease is assessed by one or more methods known in the art. In other specific embodiments, the methods described herein further comprise assessing the impairment associated with Huntington's disease before and/or after the administering step, wherein the impairment is assessed by one or more methods known in the art. In one embodiment, the methods described herein further comprise assessing the level of said impairment after repeated administration of a fumarate described herein.

In a specific embodiment, treatment of Huntington's disease, for example, the improvement of an impairment associated with Huntington's disease, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by Huntington's disease, or to eliminate an impairment associated with Huntington's disease.

In certain embodiments, treating a patient by administering an amount of a fumarate is effective to inhibit progression of, or to inhibit development of, an impairment associated with Huntington's disease.

In one embodiment, the fumarate is administered in a therapeutically effective amount to the patient. In a particular embodiment, the administration of the fumarate in a therapeutically effective amount improves the impairment associated with Huntington's disease in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform particular task. In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

In one embodiment, the severity of Huntington's disease or the severity of one or more impairments associated with Huntington's disease are assessed using the Unified Huntington's Disease Rating Scale (UHDRS). The UHDRS is a method developed by the Huntington Study Group (“HSG”) to provide an assessment of the clinical features and course of Huntington's disease. The UHDRS has been used as a major outcome measure in controlled clinical trials. The components of the UHDRS are:

1. Motor Assessment

2. Cognitive Assessment

3. Behavioral Assessment

4. Independence Scale

5. Functional Assessment

6. Total Functional Capacity (TFC)

See Huntington Study Group (Kieburtz K, primary author). The Unified Huntington's Disease Rating Scale: Reliability and Consistency. Mov. Dis. 1996; 11:136-142. The Motor Section of the UHDRS is a supplement to the following Movement Disorders Journal publication: Volume 11, Issues 1-3, The Unified Huntington's Disease Rating Scale: Reliability and Consistency. Mov. Dis. 1996; 11:136-142, Supplemental Tape.

In specific embodiments, provided herein are methods of treating Huntington's disease for improvement of an impairment associated with Huntington's disease, wherein the impairment is an impairment in movement, cognitive impairment, or psychiatric impairment, or an impairment described in A Physician's Guide to the Management of Huntington's Disease, Lovecky and Trapata (eds.), 3^{rd} Ed., Huntington's disease Society of America (2011).

### 5.2.3.1. Impairment in Movement

In one embodiment, the impairment associated with Huntington's disease and treated according to the methods described herein is an impairment in movement. In a particular embodiment, the impairment in movement is an emergence of involuntary movements (chorea) and/or the impairment of voluntary movements, which may result in one or more of the following: reduced manual dexterity, hand coordination, slurred speech, swallowing difficulties, problems with balance, and falls.

In one embodiment, the impairment in movement is an impairment described in A Physician's Guide to the Management of Huntington's Disease, Lovecky and Trapata (eds.), 3rd Ed., Huntington's disease Society of America (2011), pp. 39-50.

In one embodiment, the impairment in movement is dystonia, which is characterized, for example, by a repetitive, abnormal pattern of muscle contraction frequently associated with a twisting quality. In specific embodiments, dystonia may include, for example, one or more of the following: dystonic arm elevation while walking, tilting of the trunk, bruxism, and elevation and adduction of the foot while walking.

In one embodiment, the impairment in movement is bradykinesia, which implies slowing of automatic or voluntary movements. In one embodiment, bradykinesia may include, for example, one or more of the following: loss of facial expressivity, absence of arm swing, difficulty with finger tapping and rapid alternating movements and gait slowness.

In one embodiment, the impairment in movement is tics (sudden, brief, intermittent movements, gestures, or vocalizations that mimic fragments of normal behavior), myoclonus (sudden, brief, shock-like involuntary movements), tremor (rhythmic oscillating movement present at rest, with posture, or with voluntary movements), or rigidity (increase in muscle tone and a reduction of passive range of motion).

In one embodiment, the impairment in movement is loss of voluntary motor control, such as slow initiation and velocity of saccadic eye movements, difficulty with finger and manual dexterity, slowness in finger tapping and rapid alternating movements of the hands.

In one embodiment, the impairment in movement is motor impersistence, i.e., the inability to maintain voluntary motor contraction, as evidenced, for example, by the “milk-maid's grip” or uneven pressure on the gas pedal while driving. In a particular embodiment, motor impersistence may be assayed assessing sustained maximum eyelid closure or tongue protrusion.

In one embodiment, the impairment in movement is and impairment in gait. In a particular embodiment, the gait is slower and more wide-based.

In one embodiment, the impairment in movement is dysarthria (slurred or slow speech). In another embodiment, the impairment in movement is dysphagia (difficulty in swallowing). In another embodiment, the impairment in movement is bladder and bowel incontinence. In certain embodiments, the impairment in movement is caused by an epileptic seizure.

In a specific embodiment, the impairment in movement can be assessed before and/or after administration of a fumarate or a pharmaceutically acceptable salt thereof using one or more methods described below or known in the art.

In one embodiment, the severity of chorea is assessed using the Unified Huntington's Disease Rating Scale (UHDRS). A Physician's Guide to the Management of Huntington's Disease, Lovecky and Trapata (eds.), 3^{rd} Ed., Huntington's Disease Society of America (2011), pp. 40-41. The UHDRS includes a subscale for assessing motor disorders. Chorea is rated in one of seven body regions. The total chorea score is the sum of the scores of each body region and can range from 0-28.

### 5.2.3.2. Cognitive Impairment

In one embodiment, the impairment associated with Huntington's disease and treated according to the methods described herein is a cognitive impairment. In a particular embodiment, the cognitive impairment is a reduction of speed and flexibility in mental processing and accumulation of cognitive losses.

In one embodiment, the cognitive impairment is an impairment described in A Physician's Guide to the Management of Huntington's Disease, Lovecky and Trapata (eds.), 3rd Ed., Huntington's Disease Society of America (2011), pp. 51-62.

In one embodiment, the cognitive impairment is an impairment in memory. The patient has difficulties in learning new information and retrieving previously learned information due to, for example, slower processing speeds and an impaired ability to organize information.

In one embodiment, the cognitive impairment is an impairment in the ability to perceive information. In certain embodiments, this impairment is characterized by one or more of the following impairments: impairment in emotional recognition (e.g., ability to accurately identify which emotions being communicated un a facial expression), perception of time (e.g., difficulty with the estimation of time), smell identification (e.g., ability to detect smell, but impaired ability to identify smell), spatial perception (e.g., impaired judgment of where the body is in relation so walls, corners or tables, resulting in accidents or falls), and unawareness (e.g., of one's own action and feelings, inability to recognize own disability and behavior).

In one embodiment, the cognitive impairment is an impairment in executive efficiency. Executive processes are universally and significantly impacted in Huntington's disease. Executive functions involve fundamental abilities that regulate the primary cognitive processes in the brain. In certain embodiments, these fundamental abilities include, but are not limited to, speed of cognitive processing, attention (e.g., capacity to do two things at once), planning and organization (e.g., sequencing and prioritization), initiation (e.g., ability to initiate or start an activity, conversation, or behavior), perseveration (e.g., patients may get fixed on a specific thought or action), impulse control (e.g., patients may experience difficulties in impulse control and problem behaviors, such as irritability, temper outbursts, acting without thinking and inappropriate sexual behavior), and other regulatory processes impacting cognition.

In one embodiment, the cognitive impairment is an impairment in communication, such as speaking clearly (articulation), starting a conversation (initiation) and organizing (e.g., what information is coming in and what information is going out).

In a specific embodiment, the cognitive impairment can be assessed before and/or after administration of a fumarate or a pharmaceutically acceptable salt thereof using one or more methods described below or known in the art.

In one embodiment, the cognitive impairment is assessed using the Unified Huntington's Disease Rating Scale (UHDRS). A Physician's Guide to the Management of Huntington's Disease, Lovecky and Trapata (eds.), 3^{rd} Ed., Huntington's Disease Society of America (2011), pp. 61-62. To measure cognition, the UHDRS uses three tasks:
1) Symbol Digit Modalities test: The test requires a patient to match as many symbols and numbers as quickly as possible in 90 seconds.
2) Stroop Color Word test: The test requires a patient to name the colors of boxes, read words and name the colors of ink in a word. Each task is allowed 45 seconds and the score is the number of items correctly read aloud.
3) Verbal Fluency test: The test requires a patient to say aloud as many words that begin with a specified letter in 60 seconds.

### 5.2.3.3. Psychiatric Impairment

In one embodiment, the impairment associated with Huntington's disease and treated according to the methods described herein is a psychiatric impairment. In a particular embodiment, the psychiatric impairment is depression, mania, obsessive compulsive disorder, psychosis, hypofrontal or dysexecutive syndrome. In a specific embodiment, the hypofrontal or dysexecutive syndrome is characterized by one or more of the following: apathy, irritability, impulsivity, and obsessionality. The syndrome may have severe consequences form the patient's marital, social and economic well-being.

In one embodiment, the psychiatric impairment is an impairment described in A Physician's Guide to the Management of Huntington's Disease, Lovecky and Trapata (eds.), 3^{rd} Ed., Huntington's Disease Society of America (2011), pp. 63-82.

In one embodiment, the psychiatric impairment is major depression, mania, obsessive compulsive disorder, delusional disorder, or psychotic disorders. In another embodiment, the psychiatric impairment is organic personality syndrome (e.g., behavioral and personality changes, which may include apathy, irritability, disinhibition, perseveration, jocularity, obsessiveness and impaired judgment), which is also known as frontal lobe syndrome or dysexecutive syndrome. In yet another embodiment, the psychiatric impairment is delirium, agitation, or a sexual disorder.

### 5.2.4 Alzheimer's Disease

Provided herein are methods of treating Alzheimer's disease, for example, one or more impairments associated with Alzheimer's disease, comprising administering intravenously to a patient in need thereof at least one fumarate disclosed herein.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat Alzheimer's disease, for example, an impairment associated with Alzheimer's disease.

In a specific embodiment, the patient is a human.

In certain embodiments, intravenous administration of a fumarate is more effective at treating Alzheimer's disease than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with Alzheimer's disease in a patient. This can be demonstrated by an improved readout in one or more methods, which are known in the art and which may be used to assess the impairment associated with Alzheimer's disease, over periods of at least or more than: 2 weeks, 1 month, 1 year, or 2 years.

In certain embodiments, at least one fumarate is administered to a patient repeatedly for at least or more than: 2 weeks, 1 month, 1 year, or 2 years.

In specific embodiments, the impairment associated with Alzheimer's disease is assessed by one or more methods known in the art. In other specific embodiments, the methods described herein further comprise assessing the impairment associated with Alzheimer's disease before and/or after the administering step, wherein the impairment is assessed by one or more methods known in the art. In one embodiment, the methods described herein further comprise assessing the level of said impairment after repeated administration of a fumarate described herein.

In a specific embodiment, treatment of Alzheimer's disease, for example, the improvement of an impairment associated with Alzheimer's disease, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by Alzheimer's disease, or to eliminate an impairment associated with Alzheimer's disease.

In certain embodiments, treating a patient by administering an amount of a fumarate is effective to inhibit progression of, or to inhibit development of, an impairment associated with Alzheimer's disease.

In one embodiment, the fumarate is administered in a therapeutically effective amount to the patient. In a particular embodiment, the administration of the fumarate in a therapeutically effective amount improves the impairment associated with Alzheimer's disease in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform particular task In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

In specific embodiments, provided herein are methods of treating Alzheimer's disease for improvement of an impairment associated with Alzheimer's disease. The impairment can be an impairment in cognition, an impairment in functional capacity, a change in behavior, an impairment in general physical health, reduced quality of life, or any impairment associated with Alzheimer's disease described below or known in the art, or assayed in the methods of assessing an impairment associated with Alzheimer's disease described below.

### 5.2.4.1. Impairments Associated with Alzheimer's Disease

In one embodiment, the impairment associated with Alzheimer's disease and treated according to the methods described herein is an impairment in cognition, impairment in functional capacity, change in behavior, impairment in general physical health, or reduced quality of life. In some embodiments, the impairment in cognition is a memory impairment or thinking impairment. In some embodiments, a memory impairment can be memory problems in immediate recall, short-term memory, or long-term memory. In some embodiments, a thinking impairment can be an impairment in expressing or comprehending language, identifying familiar objects through the senses, poor coordination, gait or muscle function, or an executive function (e.g., planning, ordering, or making judgments).

In a specific embodiment, an impairment is assessed before and/or after administration of a fumarate or a pharmaceutically acceptable salt thereof) using one or more methods described below or known in the art. Alzheimer's Disease Fact Sheet, NIH Publication No. 11-6423, July 2011 and Understanding Alzheimer's Disease: What you need to know, NIH Publication No. 11-5441, June 2011.

In one embodiment, an impairment associated with Alzheimer's disease is assessed (before and/or after administration of a fumarate), by one or more of Cognitive assessments (such as Alzheimer's Disease Assessment Scale, cognitive subsection (ADAS-cog), Blessed Information-Memory-Concentration Test (BIMC), Blessed Orientation Memory Concentration instrument, short test of mental status (STMS), Clinical Dementia Rating Scale (CDR), Mini-Mental State Examination (MMSE)), Functional assessments (such as Functional Activities Questionnaire (FAQ), Instrumental Activities of Daily Living (IADL), Physical Self-Maintenance Scale (PSMS), and Progressive Deterioration Scale (PDS)), and Global assessments (such as Clinical Global Impression of Change (CGIC), Clinical Interview-Based Impression (CIBI), and Global Deterioration Scale (GDS)), and Caregiver-based assessments (such as Behavioral Pathology in Alzheimer's Disease Rating Scale (BEHAVE-AD) and Neuropsychiatric Inventory (NPI)). Robert P et al., Review of Alzheimer's disease scales: is there a need for a new multi-domain scale for therapy evaluation in medical practice?, Alzheimers Res. Ther. 2010 Aug. 26; 2(4):24, Adelman and Daly, Initial evaluation of the patient with suspected dementia, Am. Fam. Physician. 2005 May 1; 71(9):1745-50, and Boustani Metal., Screening for Dementia, Systematic Evidence Reviews, No. 20, 2003.

In one embodiment, an impairment associated with Alzheimer's disease is assessed (before and/or after administration of a fumarate), by the ADAS-Cog subscale test. The ADAS-Cog subscale can be used in differentiating people with normal thinking processes from those with impaired thinking. It can also assess the extent of decline in the thinking abilities in individuals. The ADAS-Cog subscale can determine incremental improvements or declines in thinking processes of the subject. ADAS-Cog subscale contains eleven areas, including word recall, naming objects and fingers, following commands, constructional (drawing abilities) praxis, ideational (thinking process) praxis, orientation, word recognition, remembering test directions, spoken language, comprehension, and word-finding difficulty before and after treatment. In the word recall portion, a subject is given three chances to recall as many words as possible from a list of ten words that they were shown. In naming objects and fingers, several real objects are shown to the subject, such as a flower, pencil and a comb, and the subject is asked to name them. The subject is then asked to state the name of each of the fingers on the hand, such as pinky, thumb, etc. In following commands, the subject is asked to follow a series of sometimes multi-step but simple directions, such as, “make a fist” and “place the pencil on top of the card.” In constructional (drawing abilities) praxis, the task involves showing the person four different shapes, progressively more difficult such as overlapping rectangles, and asking them to draw each one. In ideational (thinking process) praxis, the test administrator asks the subject to pretend the subject has written a letter to himself, fold it, place it in the envelop, seal the envelop, address it and demonstrate where to place the stamp. In orientation, the subject's orientation is measured by asking him what his last and first name are, the day of the week, date, month, year, season, time of day, and location. In word recognition, the subject is asked to read and try to remember a list of twelve words. The subject is presented with those words along with several other words and asked if each word is one that she saw earlier or not. In remembering test directions, the individual's ability to remember directions without reminders or with a limited amount of reminders is assessed. In spoken language, the subject's ability to use language to make herself understood is evaluated throughout the test. In comprehension, the subject's ability to understand words and language over the course of the test is assessed by the test administrator. In word-finding difficulty, throughout the test, the test administrator assesses the subject's word-finding ability throughout spontaneous conversation. Doraiswamy P M et al., Memory, language, and praxis in Alzheimer's disease: norms for outpatient clinical trial populations, Psychopharmacol. Bull. 1997; 33(1):123-8; What is the Alzheimer's Disease Assessment Scale-Cognitive Subscale at alzheimers.about.com.

In one embodiment, an impairment associated with Alzheimer's disease is assessed (before and/or after administration of a fumarate), by Blessed Information-Memory-Concentration Test (BIMC). The Blessed Information Memory Concentration (BIMC) instrument primarily assesses orientation, memory, and concentration (counting forward and backward, and naming the months of the year in reverse order). Errors are counted and can total from zero to 28. Making more than 10 errors indicates cognitive impairment. Adelman and Daly, Initial evaluation of the patient with suspected dementia, Am. Fam. Physician. 2005 May 1; 71(9):1745-50.

In one embodiment, an impairment associated with Alzheimer's disease is assessed (before and/or after administration of a fumarate), by the Blessed Orientation Memory Concentration instrument. The Blessed Orientation Memory Concentration instrument is a shortened version of the BIMC with six questions assessing orientation to time, recall of a short phrase, counting backward, and reciting the months in reverse order. A weighted score of errors is calculated. As with the BIMC, making more than 10 errors is indicative of cognitive impairment. Adelman and Daly, Initial evaluation of the patient with suspected dementia, Am. Fam. Physician. 2005 May 1; 71(9):1745-50.

In one embodiment, an impairment associated with Alzheimer's disease is assessed (before and/or after administration of a fumarate), by the Short Test of Mental Status (STMS). The Short Test of Mental Status (STMS) assesses orientation, attention, recall, calculation, abstraction, clock drawing, and copying. The STMS has a total score of 38. A score of 29 or lower indicates impaired cognitive function. Adelman and Daly, Initial evaluation of the patient with suspected dementia, Am. Fam. Physician. 2005 May 1; 71(9):1745-50, Barclay L, Short Test of Mental Status Helpful in Diagnosing Dementia, Medscape Medical News, 2003 at medscape.com, Kokmen E et al., A Short Test of Mental Status: Description and Preliminary Results, Mayo Clin Proc, 1987 April; 62(4):281-8, and Tang-Wai D F et al., Comparison of the short test of mental status and the mini-mental state examination in mild cognitive impairment, Arch. Neurol., 2003 December; 60(12):1777-81.

In one embodiment, an impairment associated with Alzheimer's disease is assessed (before and/or after administration of a fumarate), by Clinical Dementia Rating Scale (CDR). The CDR is a 5-point scale used to characterize six domains of cognitive and functional performance applicable to Alzheimer disease and related dementias: Memory, Orientation, Judgment & Problem Solving, Community Affairs, Home & Hobbies, and Personal Care. The necessary information to make each rating is obtained through a semi-structured interview of the patient and a reliable informant or collateral source (e.g., family member). The CDR table provides descriptive anchors that guide the clinician in making appropriate ratings based on interview data and clinical judgment. In addition to ratings for each domain, an overall CDR score may be calculated through the use of an algorithm. This score is useful for characterizing and tracking a patient's level of impairment/dementia:

0=Normal

0.5=Very Mild Dementia

1=Mild Dementia

2=Moderate Dementia

3=Severe Dementia

Berg L. Clinical Dementia Rating (CDR). Psychopharmacol. Bull. 1988; 24:637-639.

In one embodiment, an impairment associated with Alzheimer's disease is assessed (before and/or after administration of a fumarate or a pharmaceutically acceptable salt thereof), by Mini-Mental State Examination (MMSE). The most frequently used mental state examination in North America is the Mini-Mental State Examination (MMSE). The MMSE measures many areas of cognitive functioning including memory, orientation to place and time, naming, reading, copying (visuospatial orientation), writing, and the ability to follow a three-stage command. It can be administered in five to 10 minutes and is scored from zero to 30 points. A score of fewer than 24 points signifies cognitive impairment, although the test can be adjusted for educational level. The MMSE can be more specific but less sensitive (i.e., gives more false negatives but fewer false positives) in highly educated individuals. Adelman and Daly, Initial evaluation of the patient with suspected dementia, Am. Fam. Physician. 2005 May 1; 71(9):1745-50 and Folstein et al., “Mini-Mental State” a Practical Method for Grading the Cognitive State of Patients for the Clinician. Journal of Psychiatric Research, 12(3); 189-198.

In one embodiment, an impairment associated with Alzheimer's disease is assessed (before and/or after administration of a fumarate or a pharmaceutically acceptable salt thereof), by Functional Activities Questionnaire (FAQ). The Functional Activities Questionnaire (FAQ) measures functional activities that may be impaired by dementia (e.g., ability to shop, cook, pay bills). The FAQ is answered by a family member or friend who knows and has observed the patient. The “informant” is asked to rate the performance of the patient in 10 activities as someone who is dependent, requires assistance, or has difficulty but does independently. Scores range from zero to 30 with a cutoff of 9 (i.e., dependent in three or more activities) signifying impairment. This information may be useful in a clinical context, but the patient's cognitive function still needs to be evaluated. Adelman and Daly, Initial evaluation of the patient with suspected dementia, Am. Fam. Physician. 2005 May 1; 71(9):1745-50.

In one embodiment, an impairment associated with Alzheimer's disease is assessed (before and/or after administration of a fumarate or a pharmaceutically acceptable salt thereof), by Instrumental Activities of Daily Living (IADL). The Lawton Instrumental Activities of Daily Living (IADL) Scale assesses a person's ability to perform activities that people do once they are up, dressed, put together. These activities include, but are not limited to, cooking, driving, using a telephone or computer, shopping, keeping track of finances, and managing medication. Measuring eight domains, IADL can be administered in 10 to 15 minutes. The scale may provide an early warning of functional decline or signal the need for further assessment. Wiener J M et al., Measuring the activities of daily living: comparisons across national surveys, J. Gerontol. 1990 November; 45(6):5229-37 and Robert P et al., Review of Alzheimer's disease scales: is there a need for a new multi-domain scale for therapy evaluation in medical practice?, Alzheimers Res. Ther. 2010 Aug. 26; 2(4):24.

In one embodiment, an impairment associated with Alzheimer's disease is assessed (before and/or after administration of a fumarate or a pharmaceutically acceptable salt thereof), by Physical Self-Maintenance Scale (PSMS). The Physical Self-Maintenance Scale was developed to gauge disability in an elderly people currently in a community or institution for use in planning and assessing treatment. Items in the scale specifically target observable behaviors. The format the PSMS is first a six item based on the ADL and then eight-items based on the IADL scale. A 5-point scale for responses ranges from total independence to total dependence. Ages recommended for the test are 60 and over. There is a rating version of instrument and a self-administered version. Physical Self-Maintenance Scale (PSMS). Original observer-rated version; Psychopharmacol Bull. 1988; 24(4):793-4; Physical Self-Maintenance Scale (PSMS). Self-rated version. Incorporated in the Philadelphia Geriatric Center. Multilevel Assessment Instrument (MAI). Psychopharmacol. Bull. 1988; 24(4):795-7.

In one embodiment, an impairment associated with Alzheimer's disease is assessed (before and/or after administration of a fumarate), by Progressive Deterioration Scale (PDS). The Progressive Deterioration Scale (PDS) contains 27 quality-of-life factors and is a self-administered scale for caregivers that examines the ability of patients to accomplish basic ADLs and IADLs in 11 areas. Each item is scored using a 100 mm bipolar visual analogue scale, then a total score range from 0 to 100 is derived from the average across the items. DeJong R et al., Measurement of quality-of-life changes in patients with Alzheimer's disease. Clin Ther. 1989; 11:545-54.

In one embodiment, an impairment associated with Alzheimer's disease is assessed (before and/or after administration of a fumarate), by Clinical Global Impression (CGI). The Clinical Global Impression rating scales are commonly used measures of symptom severity, treatment response and the efficacy of treatments in treatment studies of patients with mental disorders. The Clinical Global Impression-Severity scale (CGI-S) is a 7-point scale that requires the clinician to rate the severity of the patient's illness at the time of assessment, relative to the clinician's past experience with patients who have the same diagnosis. Considering total clinical experience, a patient is assessed on severity of mental illness at the time of rating 1, normal, not at all ill; 2, borderline mentally ill; 3, mildly ill; 4, moderately ill; 5, markedly ill; 6, severely ill; or 7, extremely ill. The Clinical Global Impression-Improvement scale (CGI-I) is a 7 point scale that requires the clinician to assess how much the patient's illness has improved or worsened relative to a baseline state at the beginning of the intervention, and rated as: 1, very much improved; 2, much improved; 3, minimally improved; 4, no change; 5, minimally worse; 6, much worse; or 7, very much worse. The Clinical Global Impression-Efficacy Index is a 4 point×4 point rating scale that assesses the therapeutic effect of the treatment as 1, unchanged to worse; 2, minimal; 3, moderate; 4, marked by side effects rated as none, do not significantly interfere with patient's functioning, significantly interferes with patient's functioning and outweighs therapeutic effect. Robert P et al., Review of Alzheimer's disease scales: is there a need for a new multi-domain scale for therapy evaluation in medical practice?, Alzheimers Res. Ther. 2010 Aug. 26; 2(4):24.

In one embodiment, an impairment associated with Alzheimer's disease is assessed (before and/or after administration of a fumarate), by Clinical Interview-Based Impression (CIBI). The CIBI is a semi-structured interview based, in part, upon the ADCS Global Impression of Change instrument. It identifies four major categories for evaluation: General, Mental/Cognitive State, Behavior, and Activities of Daily Living. Each of these four categories is subdivided into domains as shown in Table 1 below

TABLE 1

| | | | Activities of |
|---|---|---|---|
| General | Mental/Cognitive | Behavior | Daily Living |
| Relevant | Arousal/Alertness/ | Thought Content | Basic and |
| History | Attention/ | Hallucinations/ | Complex |
| Observation/ | Concentration | Delusions/ | (instrumental |
| Evaluation | Orientation | Illusions | activities) |
| | Memory | Behavior/Mood | Social |
| | Language/Speech | Sleep/Appetite | Function |
| | Praxis | Neurological/ | |
| | Judgment/ | Psychomotor | |
| | Problem Solving/ | Activity | |
| | Insight | | |

Each domain is assessed by the use of probes. For all domains, some suggested probes are provided. The Interviewer is encouraged to choose additional probes, as necessary, to enhance the comprehensiveness of the interview. Clinician Interview Based Impression of Severity, morethanmedication.com.au and Knopman D S, Knapp M J, Gracon S I, Davis C S: The Clinician Interview-Based Impression (CIBI): A clinician's global change rating scale in Alzheimer's disease, Neurology 1994, 44:2315-2321.

In one embodiment, an impairment associated with Alzheimer's disease is assessed (before and/or after administration of a fumarate), by Global Deterioration Scale (GDS). The Global Deterioration Scale (GDS) provides caregivers an overview of the stages of cognitive function for those suffering from a primary degenerative dementia such as Alzheimer's disease. It is broken down into 7 different stages. Stages 1-3 are the pre-dementia stages. Stages 4-7 are the dementia stages. Beginning in stage 5, an individual can no longer survive without assistance. Within the GDS, each stage is numbered (1-7), given a short title (i.e., Forgetfulness, Early Confusional, etc. followed by a brief listing of the characteristics for that stage. Caregivers can get a rough idea of where an individual is at in the disease process by observing that individual's behavioral characteristics and comparing them to the GDS. The Global Deterioration Scale for Assessment of Primary Degenerative Dementia, www.fhca.org and Reisberg, B. et al., The global deterioration scale for assessment of primary degenerative dementia. American Journal of Psychiatry, 1982, 139: 1136-1139.

In one embodiment, an impairment associated with Alzheimer's disease is assessed (before and/or after administration of a fumarate or a pharmaceutically acceptable salt thereof), by Behavioral Pathology in Alzheimer's Disease Rating Scale (BEHAVE-AD). BEHAVE-AD is a neurological testing instrument used to assess patients with Alzheimer's disease, which provides a global rating of non-cognitive symptoms. It can be used to benchmark the efficacy of clinical drugs. Robert P et. al., Review of Alzheimer's disease scales: is there a need for a new multi-domain scale for therapy evaluation in medical practice?, Alzheimers Res. Ther. 2010 Aug. 26; 2(4):24, Auer et al., The Empirical Behavioral Pathology in Alzheimer's Disease (E-BEHAVE-AD) Rating Scale, Int. Psychogeriatr. 1996 Summer; 8(2):247-66 and Reisberg et al., Behavioral pathology in Alzheimer's disease (BEHAVE-AD) rating scale, Int. Psychogeriatr., 1996; 8 Suppl. 3:301-8; discussion 351-4.

In one embodiment, an impairment associated with Alzheimer's disease is assessed (before and/or after administration of a fumarate), by Neuropsychiatric Inventory (NPI). The Neuropsychiatric Inventory (NPI) assesses 10 behavioral disturbances occurring in dementia patients: delusions, hallucinations, dysphoria, anxiety, agitation/aggression, euphoria, disinhibition, irritability/lability, apathy, and aberrant motor activity. The NPI uses a screening strategy to minimize administration time, examining and scoring only those behavioral domains with positive responses to screening questions. Both the frequency and the severity of each behavior are determined. Each item on the NPI is scored on a 1- to 4-point frequency scale and a 1- to 3-point severity scale. The severity score is then multiplied by the frequency score, resulting in a total score ranging from 10 to 120 points. Information for the NPI is obtained from a caregiver familiar with the patient's behavior. Cummings J L et al., The Neuropsychiatric Inventory: comprehensive assessment of psychopathology in dementia, Neurology. 1994 December; 44(12):2308-14 and Boustani M et al., Screening for Dementia, Appendix C. Detailed Description of Standard Scales Used, Systematic Evidence Reviews, No. 20, 2003.

In one embodiment, an impairment associated with Alzheimer's disease is assessed (before and/or after administration of a fumarate or a pharmaceutically acceptable salt thereof), by Alzheimer's Disease Functional Assessment of Change Scale (ADFACS). The ADFACS is a 16-item functional assessment instrument based on both basic ADLs and IADLs. A trained clinician or research assistant obtains information directly from both the patient and the caregiver. Each of the basic ADL items is scored on a scale of 0 (no impairment) to 4 (severe impairment) and each IADL item is scored on a scale ranging from 0 (no impairment) to 3 (severe impairment). The total score for the 16-item scale ranges from 0 to 54. Boustani M et al., Screening for Dementia, Appendix C. Detailed Description of Standard Scales Used, Systematic Evidence Reviews, No. 20, 2003.

In one embodiment, an impairment associated with Alzheimer's disease is assessed (before and/or after administration of a fumarate), by Gottfries-Brane-Steen Scale (GBS). The Gottfries-Brane-Steen (GBS) scale is a 27-item global scale for rating dementia symptoms based on a semi-structured interview by the clinician, with both the patient and the caregiver. The GBS assesses 4 domains: intellectual impairment (orientation, memory, concentration [12 items]), self-care motor function (6 items), emotional reaction (3 items), and behavioral symptoms (6 items). A 7-point scoring system from 0 to 6 is used for each of the 27 items of this scale, giving a total score range of 0 to 162 points, with an increase in score representing clinical deterioration. Boustani M et al., Screening for Dementia, Appendix C. Detailed Description of Standard Scales Used, Systematic Evidence Reviews, No. 20, 2003, Gottfries C G et al., A new rating scale for dementia syndromes, Arch Gerontol Geriatr 1982, 1:311-330, and Bråne G et al., The Gottfries-Bråne-Steen scale: validity, reliability and application in anti-dementia drug trials, Dement. Geriatr. Cogn. Disord. 2001, 12:1-14.

In one embodiment, an impairment associated with Alzheimer's disease is assessed (before and/or after administration of a fumarate), by Interview for Deterioration in Daily living in Dementia Scale (IDDD). This scale assesses functional disability in basic ADLs (16 items) and IADLs (17 items) of patients living in the community. The caregiver assesses patients' severity of impairment in each item on a 7-point scale, where 1 to 2 points denotes no or slight impairment, 3 to 4 points denotes mild impairment, 5 to 6 points denotes moderate impairment, and 7 points denotes severe impairment. The total score range is 33 to 231 points. Boustani M et al., Screening for Dementia, Appendix C. Detailed Description of Standard Scales Used, Systematic Evidence Reviews, No. 20, 2003, Katz S et al., Studies of illness in the aged. The Index of ADL: a standardized measure of biological and psychosocial function, JAMA 1963, 185:914-919, Lawton M P and Brody E M: Assessment of older people: self-maintaining and instrumental activities of daily living, Gerontologist 1969, 9:179-186, and Robert P et. al., Review of Alzheimer's disease scales: is there a need for a new multi-domain scale for therapy evaluation in medical practice?, Alzheimers Res. Ther. 2010 Aug. 26; 2(4):24.

In one embodiment, an impairment associated with Alzheimer's disease is assessed (before and/or after administration of a fumarate or a pharmaceutically acceptable salt thereof), by Resource Utilization in Dementia Questionnaire Scale (RUD). The RUD scale is completed by caregivers and compiles data on the use of social services, frequency and duration of hospitalizations, unscheduled contacts with health care professionals, use of concomitant medications by both the caregiver and the patient, amount of time the caregiver spends caring for the patient and missing work, and patients' use of study medication. Boustani M et al., Screening for Dementia, Appendix C. Detailed Description of Standard Scales Used, Systematic Evidence Reviews, No. 20, 2003.

In one embodiment, an impairment in functional capacity associated with Alzheimer's disease in a human patient is assessed (before and/or after administration of a fumarate) using a functional assessment. In some embodiments, an impairment in functional capacity associated with Alzheimer's disease in a human patient is assessed (before and/or after administration of a) by a Functional Assessment Questionnaire (FAQ). In some embodiments, an impairment in functional capacity associated with Alzheimer's disease in a human patient is assessed (before and/or after administration of a fumarate) by Instrumental Activities of Daily Living (IADL) test. In some embodiments, an impairment in functional capacity associated with Alzheimer's disease in a human patient is assessed (before and/or after administration of a fumarate) by Physical Self-Maintenance Scale (PSMS) test. In some embodiments, an impairment in functional capacity associated with Alzheimer's disease in a human patient is assessed (before and/or after administration of a fumarate) by Progressive Deterioration Scale (PDS) test.

In one embodiment, an impairment in cognition associated with Alzheimer's disease is assessed (before and/or after administration of a fumarate) using a cognitive assessment. In some embodiments, an impairment in cognition associated with Alzheimer's disease in a human patient is assessed (before and/or after administration of a fumarate) by Alzheimer's disease Assessment Scale, cognitive subsection (ADAS-cog). In some embodiments, an impairment in cognition associated with Alzheimer's disease in a human patient is assessed (before and/or after administration of a fumarate) by Blessed Information-Memory-Concentration Test (BIMC). In some embodiments, an impairment in cognition associated with Alzheimer's disease in a human patient is assessed (before and/or after administration of a fumarate) by Clinical Dementia Rating Scale (CDR). In some embodiments, an impairment in cognition associated with Alzheimer's disease in a human patient is assessed (before and/or after administration of a fumarate) by Mini-Mental State Examination (MMSE).

In one embodiment, the cognitive impairment associated with Alzheimer's disease can be assessed (before and/or after administration of a fumarate or a pharmaceutically acceptable salt thereof) using Alzheimer Disease Assessment Scale-cognitive (ADAS-Cog) subscale. Alzheimer Disease Assessment Scale-cognitive (ADAS-Cog) subscale helps evaluate thinking processes and differentiates between normal thinking processes and impaired thinking functioning. It is especially useful for determining the extent of decline of the thinking processes and can help evaluate which stage of dementia a person is in, based on the answers and score.

In one embodiment, an improvement in the cognitive impairment associated with Alzheimer's disease is assessed by ADAS-Cog subscale. ADAS-Cog subscale evaluates the subject's cognitive abilities and memory over eleven areas, including word recall, naming objects and fingers, following commands, constructional (drawing abilities) praxis, ideational (thinking process) praxis, orientation, word recognition, remembering test directions, spoken language, comprehension, and word-finding difficulty before and after treatment. Points for each section of the ADAS-Cog subscale are added up for a for a total score. The greater the dysfunction in thinking, the greater the score. Doraiswamy P M et al., Memory, language, and praxis in Alzheimer's disease: norms for outpatient clinical trial populations, Psychopharmacol. Bull. 1997; 33(1):123-8; What is the Alzheimer's Disease Assessment Scale-Cognitive Subscale at alzheimers.about.com.

In one embodiment, the cognitive impairment associated with Alzheimer's disease can be assessed (before and/or after administration of a fumarate), or assayable, using the cognitive abilities screening test (CASI).

In one embodiment, an improvement in the cognitive impairment associated with Alzheimer's disease is assessed by CASI. The CASI provides quantitative assessment on attention, concentration, orientation, short-term memory, long-term memory, language abilities, visual construction, list-generating fluency, abstraction, and judgment. Teng E L, et al., The Cognitive Abilities Screening Instrument (CASI): a practical test for cross-cultural epidemiological studies of dementia, Int. Psychogeriatr. 1994; 6:45-58.

In one embodiment, the cognitive impairment associated with Alzheimer's disease can be assessed (before and/or after administration of a fumarate) using the Mini-Mental State Examination (MMSE).

In one embodiment, an improvement in the cognitive impairment associated with Alzheimer's disease is assessed by MMSE. The MMSE is a tool that can be used to systematically and thoroughly assess mental status. The MMSE is effective as a screening instrument to separate patients with cognitive impairment from those without it. In addition, when used repeatedly the instrument is able to measure changes in cognitive status that may benefit from intervention. The MMSE is an 11-question measure that tests five areas of cognitive function: orientation, registration, attention and calculation, recall, and language. The total score ranges from 0 to 30, with the higher score indicating a better cognitive state. In some embodiments, a score of 23 or lower is indicative of cognitive impairment. Folstein et al., “Mini-Mental State” a Practical Method for Grading the Cognitive State of Patients for the Clinician. Journal of Psychiatric Research, 12(3); 189-198.

In one embodiment, the cognitive impairment associated with Alzheimer's disease can be assessed (before and/or after administration of a fumarate) using the Computerized Memory Battery Test (CMBT).

In one embodiment, an improvement in the cognitive impairment associated with Alzheimer's disease is assessed by CMBT. The Computerized Memory Battery Test (CMBT) includes several tests of cognition such as Facial Recognition, First and Last Name Total Acquisition and Name-Face Association Delayed Recall subscales. It is the computerized version of the Memory Assessment Clinical Battery, which simulates critical cognitive tasks of everyday life. Sanches de Oliveira R et al., Use of computerized tests to assess the cognitive impact of interventions in the elderly, Dement Neuropsychol, 2014 June; 8(2):107-111 and Seltzer B et al. Efficacy of donepezil in early-stage Alzheimer disease: a randomized placebo-controlled trial, Arch Neurol. 2004 December; 61(12):1852-6.

In one embodiment, the cognitive impairment associated with Alzheimer's disease can be assessed (before and/or after administration of a fumarate or a pharmaceutically acceptable salt thereof) using the Clinical Dementia Rating Scale-Sum of the Boxes (CDR-SOB). O'Bryant S E et al., Staging Dementia Using Clinical Dementia Rating Scale Sum of Boxes Scores, Arch. Neurol., 65(8): 1091-1095.

### 5.2.5 Parkinson's Disease

Provided herein are methods of treating Parkinson's disease, for example, one or more impairments associated with Parkinson's disease, comprising administering intravenously to a patient in need thereof at least one fumarate disclosed herein.

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat Parkinson's disease, for example, an impairment associated with Parkinson's disease.

In a specific embodiment, the patient is a human.

In certain embodiments, intravenous administration of a fumarate is more effective at treating Parkinson's disease than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with Parkinson's disease in a patient. This can be demonstrated by an improved readout in one or more methods, which are known in the art and which may be used to assess the impairment associated with Parkinson's disease, over periods of at least or more than: 2 weeks, 1 month, 1 year, or 2 years.

In certain embodiments, at least one fumarate is administered repeatedly to a patient for at least or more than: 2 weeks, 1 month, 1 year, or 2 years.

In specific embodiments, the impairment associated with Parkinson's disease is assessed by one or more methods known in the art. In other specific embodiments, the methods described herein further comprise assessing the impairment associated with Parkinson's disease before and/or after the administering step, wherein the impairment is assessed by one or more methods known in the art. In one embodiment, the methods described herein further comprise assessing the level of said impairment after repeated administration of a fumarate described herein.

In a specific embodiment, treatment of Parkinson's disease, for example, the improvement of an impairment associated with Parkinson's disease, is assessed in accordance with the methods described herein at one or more time points during the treatment period of at least 2 weeks, 1 month, 1 year, 2 years.

In another specific embodiment, treating a patient by administering an amount of a fumarate is effective to restore or regain or improve the function impaired by Parkinson's disease, or to eliminate an impairment associated with Parkinson's disease.

In certain embodiments, treating a patient by administering an amount of a fumarate is effective to inhibit progression of, or to inhibit development of, an impairment associated with Parkinson's disease.

In one embodiment, the fumarate is administered in a therapeutically effective amount to the patient. In a particular embodiment, the administration of the fumarate in a therapeutically effective amount improves the impairment associated with Parkinson's disease in a patient by at least about 5%, 10%, 20%, 30%, 40%, or 50% compared to untreated patients, as assessed by methods known in the art, such as the methods described below. These methods may include objective and subjective measurements that assign values to the ability of a patient or a group of patients to perform particular task. In some embodiments, treatment in accordance with the methods provided herein results in an improvement of an impairment associated with a neurological disease that is statistically significant compared to a control value. In one embodiment, the control value may be a baseline value for the impairment, in the patient or a group of patients assessed performing the particular task before the treatment begins. In one embodiment, the control value may be a value for patients given a placebo, assessed performing the particular task. In certain embodiments, the statistical significance of an improvement of an impairment associated with a neurological disease is determined by methods known in the art.

In specific embodiments, provided herein are methods of treating Parkinson's disease for improvement of an impairment associated with Parkinson's disease, wherein the impairment is resting tremor, bradykinesia, rigidity, postural instability, freezing of gait, micrographia, mast-like expression, unwanted accelerations, stooped posture, a tendency to lean forward, dystonia, impaired fine motor dexterity and motor coordination, impaired gross motor coordination, poverty of movement (decreased arm swing, akathisia, speech problems (such as softness of voice or slurred speech caused by lack of muscle control), difficulty swallowing, sexual dysfunction, cramping, drooling, loss of sense of smell, constipation, REM behavior disorder (a sleep disorder), mood disorder, orthostatic hypotension (low blood pressure when standing up), sleep disturbances, constipation, bladder problem, sexual problem, excessive saliva, weight loss or gain, vision or dental problem, fatigue or loss of energy, depression, fear or anxiety, skin problem, cognitive issue, such as memory difficulty, slowed thinking, confusion, dementia, or any other impairment associated with Parkinson's disease that is known in the art or described below.

### 5.2.5.1. Impairments Associated with Parkinson's Disease

The methods disclosed herein provide for treatment of patients who have Parkinson's disease. In particular, the methods provide for treatment of one or more impairments associated with Parkinson's disease in a patient with Parkinson's disease. Parkinson's disease impairments include motor symptoms and nonmotor symptoms. Parkinson's disease motor symptoms can be divided into primary motor symptoms and secondary motor symptoms. In some embodiments, the methods disclosed herein provide treatment for a primary motor symptom associated with Parkinson's disease. In specific embodiments, the primary motor symptom can be resting tremor, bradykinesia, rigidity, or postural instability. In some embodiments, the methods disclosed herein provide treatment for a secondary motor symptom associated with Parkinson's disease. In specific embodiments, the secondary motor symptom can be freezing of gait, micrographia, mast-like expression, or unwanted accelerations. In some embodiments, the secondary motor symptom is stooped posture, a tendency to lean forward, dystonia, impaired fine motor dexterity and motor coordination, impaired gross motor coordination, poverty of movement (decreased arm swing, akathisia, speech problems (such as softness of voice or slurred speech caused by lack of muscle control), difficulty swallowing, sexual dysfunction, cramping, or drooling. In some embodiments, the methods disclosed herein provide treatment for a nonmotor symptom associated with Parkinson's disease. In some embodiments, the nonmotor is loss of sense of smell, constipation, REM behavior disorder (a sleep disorder), mood disorder, orthostatic hypotension (low blood pressure when standing up), sleep disturbance, constipation, bladder problem, sexual problem, excessive saliva, weight loss or gain, vision or dental problem, fatigue or loss of energy, depression, fear or anxiety, skin problem, cognitive issue, such as memory difficulty, slowed thinking, confusion, or dementia. In some embodiments, the impairment associated with Parkinson's disease is a non-motor impairment, tremor, badykinesia (slowness in movement) rigidity, postural instability, impaired balance, gait disturbance (such as freezing of gait), speech impairment, swallowing impairment, voice disorder, or rapid shuffling in walking. The impairment associated with Parkinson's disease can be any described below or known in the art.

In some embodiments, an impairment can be assessed using any of Global Assessment Scale for Wilson's Disease, Global Dystonia Scale, Modified Bradykinesia Rating Scale, Non-Motor Symptoms Scale (NMSS)+(Includes NMSQ), Quality of Life Essential Tremor Questionnaire, Rating Scale for Psychogenic Movement Disorders, Rush Dyskinesia Rating Scale, Rush Videobased Tic Rating Scale, UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), Unified Parkinson's Disease Rating Scale (MDS-UPDRS), 3D Gait analysis, Timed Up and Go Test (TUG), Timed 25-foot Walk Test (T25FW) and/or Freezing of Gait Questionnaire (FOGQ). Naismith S L and Lewis S J, “DASH” symptoms in patients with Parkinson's disease: red flags for early cognitive decline, J Clin Neurosci. 2011 March; 18(3):352-5, Morris M et al., Three-dimensional gait biomechanics in Parkinson's disease: evidence for a centrally mediated amplitude regulation disorder, Mov Disord. 2005 January; 20(1):40-50, Bonnet A M et al., Nonmotor Symptoms in Parkinson's Disease in 2012: Relevant Clinical Aspects, Parkinson Dis. 2012; 2012:198316, Martinez-Martin Petal., Assessing the non-motor symptoms of Parkinson's disease: MDS-UPDRS and NMS Scale, Eur J Neurol. 2013 Apr. 22, and MDS Rating Scales at movementdisorders.org.

In some embodiments, an impairment is assessed using UPDRS. The current UPDRS includes four subscales. Subscale 1 covers mentation, behavior, and mood. Subscale 2 rates activities of daily living. Subscale 3 is a clinician rating of the motor manifestations of PD. Subscale 4 covers complications of therapy. Data for subscales 1,2, and 4 are elicited from patients and caregivers, whereas data for subscale 3 is examination-based. There are training tapes for the UPDRS subscales 2 and 3, and reviewing these can improve the reliability of the measures. However, reliability of the other subscales depends on patient reporting in addition to examiner skills, but there is a training tape for the activities of daily living component subscale 2. The total UPDRS score and the UPDRS subscale scores are not interval scales, which means that there are not quantified, equal distances between values on these scales. For example, a score of 4 is greater than 2 but does not necessarily indicate twice the degree of severity. Each part of the rating is a rank order measure rather than a precise interval change. Perlmutter J S, Assessment of Parkinson disease manifestation, Curr Protoc Neurosci. 2009 October; Chapter 10, Goetz C G, LeWitt P A, Weidenman M. Standardized training tools for the UPDRS activities of daily living scale: Newly available teaching program. Mov. Disord. 2003; 18:1455-1458, Louis E D, Lynch T, Marder K, Fahn S. Reliability of patient completion of the historical section of the Unified Parkinson's Disease Rating Scale. Mov. Disord. 1996; 11:185-192, Goetz C G, Stebbins G T. Assuring interrater reliability for the UPDRS motor section: Utility of the UPDRS teaching tape. Mov. Disord. 2004; 19:1453-1456, and Goetz C G, Stebbins G T, Chmura T A, Fahn S, Klawans H L, Marsden C D. Teaching tape for the motor section of the unified Parkinson's disease rating scale. Mov. Disord. 1995; 10:263-266.

In some embodiments, a motor impairment or general mobility impairment treated in accordance with the methods described herein is an impairment in walking. Walking impairments include, but are not limited to, an impairment in walking speed, unwanted acceleration in walking, impairment in stride time variability, and impairment in double limb support variability. In some embodiments, a general mobility impairment or walking impairment treated in accordance with the methods described herein is assessed (before or after administration of a fumarate or a pharmaceutically acceptable salt thereof) using Global Assessment Scale for Wilson's Disease, Global Dystonia Scale, Modified Bradykinesia Rating Scale, Non-Motor Symptoms Scale (NMSS)+(Includes NMSQ), Quality of Life Essential Tremor Questionnaire, Rating Scale for Psychogenic Movement Disorders, Rush Dyskinesia Rating Scale, Rush Videobased Tic Rating Scale, UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), Unified Parkinson's Disease Rating Scale (MDS-UPDRS), 3D Gait analysis, Timed Up and Go Test (TUG), Timed 25-foot Walk Test (T25FW) and/or Freezing of Gait Questionnaire (FOGQ).

In some embodiments, a motor impairment or a general mobility impairment treated in accordance with the methods described herein is an impairment in gait. In certain embodiments, the impairment treated in accordance with the methods described herein is an abnormal gait or abnormal walking gait in a patient with PD. In some embodiments, the gait impairment treated in accordance with the methods described herein is assessed (before or after administration of a fumarate or a pharmaceutically acceptable salt thereof), using Global Assessment Scale for Wilson's Disease, Global Dystonia Scale, Modified Bradykinesia Rating Scale, Non-Motor Symptoms Scale (NMSS)+(Includes NMSQ), Quality of Life Essential Tremor Questionnaire, Rating Scale for Psychogenic Movement Disorders, Rush Dyskinesia Rating Scale, Rush Videobased Tic Rating Scale, UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), Unified Parkinson's Disease Rating Scale (MDS-UPDRS), 3D Gait analysis, Timed Up and Go Test (TUG), Timed 25-foot Walk Test (T25FW) and/or Freezing of Gait Questionnaire (FOGQ).

In some embodiments, a motor impairment associated with Parkinson's disease is dyskinesia, dystonia and/or a motor fluctuation. Generally, motor fluctuations are the oscillations, or variations, in the control of motor symptoms associated with the long term use of the medication, such as levodopa. In one embodiment, the motor impairment treated in accordance with the methods described herein is dyskinesia and/or dystonia. In some embodiments, a motor impairment (e.g., dyskinesia, dystonia or fluctuation) treated in accordance with the methods described herein is assessed (before or after administration of a fumarate or a pharmaceutically acceptable salt thereof), using Global Assessment Scale for Wilson's Disease, Global Dystonia Scale, Modified Bradykinesia Rating Scale, Non-Motor Symptoms Scale (NMSS)+(Includes NMSQ), Quality of Life Essential Tremor Questionnaire, Rating Scale for Psychogenic Movement Disorders, Rush Dyskinesia Rating Scale, Rush Videobased Tic Rating Scale, UFMG Sydenham's Chorea Rating Scale (USCRS), Unified Dyskinesia Rating Scale (UDysRS), Unified Dystonia Rating Scale (UDRS), Unified Multiple System Atrophy Rating Scale (UMSARS), Unified Parkinson's Disease Rating Scale (MDS-UPDRS), 3D Gait analysis, Timed Up and Go Test (TUG), Timed 25-foot Walk Test (T25FW) and/or Freezing of Gait Questionnaire (FOGQ).

In some embodiments, a motor impairment associated with Parkinson's disease is an impairment in vision (e.g., eye problem or eye difficulty). In some embodiments, impairments in vision associated Parkinson's disease include, but are not limited to, double vision, involuntary closure of the eyelids, deterioration in visuo-spatial orientation, hallucinations and illusions, glaucoma, excessive watering of the eyes, tired eyes, and color vision and contrast sensitivity. In some embodiments, the impairment in vision (e.g, eye problem or eye difficulty) treated in accordance with the methods described herein is assessed (before or after administration of a fumarate or a pharmaceutically acceptable salt thereof), using PD Vision Questionnaire. The PD Vision Questionnaire comprises three sections: Visual and Visuospatial symptoms, performance of visually mediated Activities of Daily Living, and Motor symptoms. Amick M M et al., Web-Based Assessment of Visual and Visuospatial Symptoms in Parkinson's Disease, Parkinsons Dis. 2012; 2012:564812.

In some embodiments, a motor impairment associated with Parkinson's disease is restless leg syndrome. In some embodiments, the motor impairment (e.g., restless syndrome) treated in accordance with the methods described herein is assessed (before or after administration of a fumarate or a pharmaceutically acceptable salt thereof), using IRLS rating scale (IRLS), the Clinical Global Impression (CGI) scale, the Patient Global Impression (PGI), the Sleep Questionnaire Form A, Quality of Life (QoL) for RLS, the Augmentation Severity Rating Scale (ASRS), Visual Analog Scales (VAS), and the Medical Outcomes Study sleep scale (MOS). A. The IRLS consists of a 10-question assessment of RLS in a format of 0 to 4, 0 being “never” or “none,” and 4 being “very severe” or “very often.” The severity of RLS is rated as: 1-10 mild; 11-20 moderate; 21-30 severe; and 31-40 very severe. The CGI has 3 sections: (1) Severity of illness; (2) Global improvement (CGII) or change (CGIC), and (3) Efficacy index. Most, if not all, studies document the proportion of patients with an investigator-rated score of “much improved” (2) or “very much improved” (1) on the CGI-I (or —C) scale (defined as a “response” on this 7-point overall global improvement scale, a non-disease specific outcome measure in which 1=very much improved and 7=very much worse). Aurora R N et al., The Treatment of Restless Legs Syndrome and Periodic Limb Movement Disorder in Adults—An Update for 2012: Practice Parameters with an Evidence-Based Systematic Review and Meta-Analyses: an American Academy of Sleep Medicine Clinical Practice Guideline, Sleep, 2012 Aug. 1; 35(8):1039-62.

In some embodiments, a motor impairment associated with Parkinson's disease is freezing, e.g. freezing of gait. Freezing is a temporary, involuntary inability to initiate or continue movement lasting just a few seconds or, on some occasions, several minutes. It happens suddenly, particularly when walking, as if the feet have become stuck to the ground and speech, writing or opening and closing the eyes can also be affected. In some embodiments, the motor impairment (e.g., freezing) treated in accordance with the methods described herein is assessed (before or after administration of a fumarate or a pharmaceutically acceptable salt thereof), using Unified Parkinson's Disease Rating Scale (MDS-UPDRS), 3D Gait analysis, Timed 25-foot Walk Test (T25FW) and/or Freezing of Gait Questionnaire (FOGQ).

In some embodiments, a motor impairment associated with Parkinson's disease is increased falls. Some people with Parkinson's find their gait becomes impaired, they may walk slowly, shuffle or suffer from freezing. All of these can compromise balance and falls become common, increasingly so as the condition progresses. Falls typically begin between five to 10 years after onset of the first symptoms. In some embodiments, the motor impairment (e.g., falling) treated in accordance with the methods described herein is assessed (before or after administration of a fumarate or a pharmaceutically acceptable salt thereof), using Balance assessments including the Tinetti, Berg Balance Scale (BBS), the Timed Up and Go (TUG), the Functional Gait Assessment (FGA), and/or Balance Evaluation Systems Test (BESTest). Duncan R P et al., Accuracy of fall prediction in Parkinson disease: six-month and 12-month prospective analyses, Parkinson Dis, 2012; 2012:237673.

In some embodiments, an impairment associated with Parkinson's disease is an impairment in cognition. In some embodiments, the impairment in cognition treated in accordance with the methods described herein is assessed (before or after administration of a fumarate or a pharmaceutically acceptable salt thereof), using SCales for Outcomes of PArkinson's disease-cognition (SCOPA-COG), Mini-Mental State Examination (MMSE), and/or Cambridge Cognitive Examination (CAMCOG). SCOPA-COG consists of 10 items with a maximum score of 43, with higher scores reflecting better performance. Marinus J et al., Assessment of cognition in Parkinson's disease, Neurology. 2003 Nov. 11; 61(9):1222-8.

### 5.2.6 Multiple Sclerosis

Provided herein are methods of treating Multiple Sclerosis, comprising administering intravenously to a patient in need thereof at least one fumarate disclosed herein. In a specific embodiment, the Multiple Sclerosis is a progressive form of Multiple Sclerosis. In a specific embodiment, the progressive form of Multiple Sclerosis is Primary Progressive Multiple Sclerosis (PP-MS). In another specific embodiment, the progressive form of Multiple Sclerosis is Secondary Progressive Multiple Sclerosis (SP-MS). In another specific embodiment, the Multiple Sclerosis is a relapsing form of Multiple Sclerosis. In a specific embodiment, the relapsing form of Multiple Sclerosis is relapse-remitting Multiple Sclerosis (RR-MS).

In one aspect, a therapeutically effective amount of a fumarate disclosed herein is intravenously administered to a patient in need thereof. In another specific embodiment, the patient is intravenously administered the fumarate or a composition comprising the fumarate in an amount and for a time sufficient to treat Multiple Sclerosis, for example, an impairment associated with Multiple Sclerosis.

In a specific embodiment, the patient is a human.

In certain embodiments, intravenous administration of a fumarate is more effective at treating Multiple Sclerosis than oral administration of the fumarate. In certain embodiments, intravenous administration of a fumarate is more effective at having the fumarate or its in vivo conversion product (e.g, dimethyl fumarate and monomethyl fumarate, respectively) reach the brain than oral administration of the fumarate; that is, greater amounts in the brain are achieved upon intravenous administration relative to oral administration. In specific embodiments, the fumarate is administered both orally and intravenously. In a specific embodiment, the fumarate is dimethyl fumarate.

In one embodiment, the treatment in accordance with the methods provided herein is to improve, decrease the duration of, maintain an improvement of, or inhibit progression of an impairment associated with Multiple Sclerosis in a patient. This can be demonstrated by an improved readout in one or more methods, which are known in the art and which may be used to assess the impairment associated with Multiple Sclerosis, over periods of at least or more than: 1 week, 2 weeks, 1 month, 1 year, or 2 years.

In certain embodiments, at least one fumarate is administered repeatedly to a patient for at least or more than: 1 week, 2 weeks, 1 month, 1 year, or 2 years.

In specific embodiments, the impairment associated with or severity of Multiple Sclerosis is assessed by one or more methods known in the art. In other specific embodiments, the methods described herein further comprise assessing the impairment associated with or severity of Multiple Sclerosis before and/or after the administering step, wherein the impairment is assessed by one or more methods known in the art. In one embodiment, the methods described herein further comprise assessing the level of said impairment or severity after repeated administration of a fumarate described herein.

In certain embodiments, treating a patient by administering an amount of a fumarate is effective to inhibit progression of, or to inhibit development of, one or more impairments associated with Multiple Sclerosis.

In specific embodiments, provided herein are methods of treating Multiple Sclerosis to achieve one or more of the following endpoints (a) reduced frequency of relapse in the subject; (b) reduced probability of relapse in the subject; (c) reduced annualized relapse rate in the subject; (d) reduced risk of disability progression in the subject; (e) reduced number of new or newly enlarging T2 lesions in the subject; (f) reduced number of new non-enhancing T1 hypointense lesions in the subject; or (g) reduced number of Gd+ lesions in the subject; wherein the changes (a)-(g) are relative to a subject receiving placebo or not being treated.

In one embodiment, the fumarate is administered in a therapeutically effective amount to the patient. In a particular embodiment, the administration of the fumarate in a therapeutically effective amount improves the endpoint associated with Multiple Sclerosis in a patient by at least about 5%, 10%, 20%, 30%, 40%, or 50% compared to untreated or placebo-treated patients, as assessed by any method known in the art.

### 5.3 Patient Populations

Provided herein are methods of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

In one embodiment, the patient does not have a known hypersensitivity to the fumarate. In one embodiment, the patient is not treated simultaneously with a fumarate and any immunosuppressive or immunomodulatory medications or natalizumab. In one embodiment, the patient is not treated simultaneously with a fumarate and any medications carrying a known risk of causing progressive multifocal leukoencephalopathy (PML). In one embodiment, the patient has no identified systemic medical condition resulting in a compromised immune system function.

As used herein, the terms “patient” and “subject” can be used interchangeably. The fumarate as described herein is administered to a subject in need thereof, a subject having a neurological disease. In a specific embodiment, said subject has been diagnosed as having a neurological disease by a medical practitioner.

In a specific embodiment, the human patient is an adult. In one embodiment, the human patient is 18 to 55 years old. In a specific embodiment, the human patient is a female. In yet another specific embodiment, the human patient is a male.

In one embodiment, the patient is not pregnant. In another embodiment, the patient is not a nursing mother. In one embodiment, if the patient is pregnant, the methods provided herein further comprise a step of encouraging the patient to enroll in a pregnancy registry, which monitors pregnancy outcomes in women exposed to the fumarate during pregnancy.

In one embodiment, the patient has no hypersensitivity to a fumarate, such as dimethyl fumarate, administered in the methods described herein. In a further embodiment, the patient has no hypersensitivity to the fumarate, such as dimethyl fumarate, or does have a known hypersensitivity to the fumarate. In certain embodiments, the method provided herein further comprise after the step of intravenously administering a fumarate a step of monitoring the patient for development of an allergic reaction to said fumarate. In specific embodiments, the allergic reaction is, for example, development of hives, angiodema and/or difficulty of breathing.

In one embodiment, the patient is not treated simultaneously with both one or more fumarates (e.g., dimethyl fumarate) and any immunosuppressive or antineoplastic medication. In certain embodiments, the patient is not treated simultaneously with a fumarate (e.g., dimethyl fumarate) and any immunosuppressive or immunomodulatory medications or natalizumab. In certain embodiments, the patient is not treated simultaneously with a fumarate described herein (e.g., dimethyl fumarate) and any medications carrying a known risk of causing progressive multifocal leukoencephalopathy (PML).

In one embodiment, the patient has never been treated with a fumarate, e.g., dimethyl fumarate, prior to commencement of therapy in accordance with the methods disclosed herein. In another embodiment, the patient has not been treated with a fumarate, e.g., dimethyl fumarate, 1, 2, 3, 4, 6, 8, 10, or 12 months or 1, 2, 3, 5, 10, 20, 30, 40, or 50 years, prior to commencement of therapy in accordance with the methods disclosed herein.

In one embodiment, the patient has never been treated with any immunosuppressive or antineoplastic medication prior to commencement of therapy in accordance with the methods disclosed herein. In a further embodiment, the patient has not been treated with any immunosuppressive or antineoplastic medication 1, 2, 3, 4, 6, 8, 10, or 12 months or 1, 2, 3, 5, 10, 20, 30, 40, 50 years, prior to commencement of therapy in accordance with the methods disclosed herein. In another embodiment, the patient has never been treated with any immunosuppressive or immunomodulatory medications or natalizumab prior to commencement of therapy in accordance with the methods disclosed herein. In yet another embodiment, the patient has not been treated with any immunosuppressive or immunomodulatory medications or natalizumab 1, 2, 3, 4, 6, 8, 10, or 12 months or 1, 2, 3, 5, 10, 20, 30, 40, or 50 years, prior to commencement of therapy in accordance with the methods disclosed herein. In another embodiment, the patient has never been treated with any medications carrying a known risk of causing PML prior to commencement of therapy in accordance with the methods disclosed herein. In yet another embodiment, the patient has not been treated with any medications carrying a know risk of causing PML 1, 2, 3, 4, 6, 8, 10, or 12 months or 1, 2, 3, 5, 10, 20, 30, 40, or 50 years, prior to commencement of therapy in accordance with the methods disclosed herein.

In one embodiment, the immunosuppressive or antineoplastic medication is selected from one or more of: chlorambucil, melphalan, 6-mercaptopurine, thiotepa, ifodfamide, dacarbazine, procarbazine, temozolomide, hexamethylmelamine, doxorubicine, daunarubicine, idarubicin, epirubicin, irinotecan, methotrexate, etoposide, vincristine, vinblastine, vinorelbine, cytarabine, busulfan, amonifide, 5-fluorouracil, topotecan, mustargen, bleomycin, lomustine, semustine, mitomycin C, mutamycin, cisplatin, carboplatin, oxaliplatin, methotrexate, trimetrexate, raltitrexid, flurorodeoxyuridine, capecitabine, ftorafur, 5-ethynyluracil, 6-thioguanine, cladribine, pentostatin, teniposide, mitoxantrone, losoxantrone, actinomycin D, vindesine, docetaxel, amifostine, interferon alpha, tamoxefen, edroxyprogesterone, megestrol, raloxifene, letrozole, anastrzole, flutamide, bicalutamide, retinoic acids, arsenic trioxide, rituximab, CAMP ATH-1, mylotarg, mycophenolic acid, tacrolimus, glucocorticoids, sulfasalazine, glatiramer, fumarate, laquinimod, FTY-720, interferon tau, daclizumab, infliximab, ILlO, anti-IL2 receptor antibody, anti-IL-12 antibody, anti-IL6 receptor antibody, CDP-571, adalimumab, entaneracept, Ieflunomide, anti-interferon gamma antibody, abatacept, fludarabine, cyclophosphamide, azathioprine, cyclosporine, intravenous immunoglobulin, 5-ASA (mesalamine), and a β-interferon.

In one embodiment, the immunosuppressive or immunomodulatory medication is selected from one or more of: calcinerurin inhibitors, corticosteroids, cytostatics, nitrosoureas, protein synthesis inhibitors, dactinomycin, anthracyclines, mithramycin, polyclonal antibodies such as atgum and thymoglobulin, monoclonal antibodies such as muromonab-CD3, and basiliximab, cyclosporin, sirolimus, rapamycin, γ-interferon, opioids, TNF binding proteins, TNF-α binding proteins, etanercept, mycophenolate, fingolimode, and myriocin.

In one embodiment, the patient being treated in accordance with the methods described herein has no identified systemic medical condition resulting in a compromised immune system function.

In one embodiment, the patient has been free of an immunosuppressant or immunomodulatory therapy for the patient's lifetime, or since diagnosis with the neurological disease.

In one embodiment, a patient treated in accordance with the methods described herein does not have multiple sclerosis.

### 5.3.1 Stroke

In one embodiment, the methods provided herein further comprises a step of selecting, identifying, or diagnosing a patient with stroke prior to the administering step.

### 5.3.2 Amyotrophic Lateral Sclerosis (“ALS”)

In one embodiment, the methods provided herein further comprises a step of selecting, identifying, or diagnosing a patient with ALS prior to the administering step.

In one embodiment, the patient is diagnosed with familial ALS. In another embodiment, said patient is diagnosed with sporadic ALS.

### 5.3.3 Huntington's Disease

In one embodiment, the methods provided herein further comprises a step of selecting, identifying, or diagnosing a patient with Huntington's disease prior to the administering step.

In one embodiment, the patient is diagnosed with juvenile onset Huntington's disease. Huntington's disease with onset in childhood has somewhat different features. Chorea is a much less prominent feature, and may be absent altogether. Initial symptoms usually include attentional deficits, behavioral disorders, school failure, dystonia, bradykinesia, and sometimes tremor. Seizures, rarely found in adults, may occur in this juvenile form. Juvenile onset HD tends to follow a more rapid course, with survival less than 15 years. A Physician's Guide to the Management of Huntington's Disease, Lovecky and Trapata (eds.), 3^{rd} Ed., Huntington's Disease Society of America (2011), page 10.

In one embodiment, the patent has early stage, middle stage, or late stage Huntington's disease.

In early stage Huntington's disease, patients are largely functional and may continue to do, e.g., work, drive, handle money, and live independently. Impairments may include, but are not limited to, one or more of minor involuntary movements, subtle loss of coordination, difficulty thinking through complex problems, some depression, irritability, and disinhibition.

In middle stage Huntington's disease, patients lose the ability to do, for example, one or more of the following: work, drive, manage their own finances, and perform their own household chores, but will be able to do, for example, one or more of the following: eat, dress, and attend to personal hygiene with assistance. In one embodiment, chorea may be prominent, and patients have increasing difficulty, for example, with voluntary motor tasks. The patient may have problems with, for example, one or more of the following: swallowing, balance, falls, and weight loss. In specific embodiments, problem solving becomes more difficult for the patient, because individuals cannot sequence, organize, or prioritize information.

In late stage Huntington's disease, patients require assistance in all activities of daily living. In one embodiment patients are often nonverbal and bedridden in late stage Huntington's disease but may retain some comprehension. In certain embodiments, chorea may be severe. In some embodiments, late stage impairments are, for example, one or more of the following: rigidity, dystonia, and bradykinesia. In another embodiment, psychiatric symptoms may occur in late stage Huntington's disease, but are harder to recognize and treat because of communication difficulties the patient may experience. A Physician's Guide to the Management of Huntington's Disease, Lovecky and Trapata (eds.), 3^{rd} Ed., Huntington's Disease Society of America (2011), page 7.

In one embodiment, the patient is has a Total Functional Capacity Rating Scale Total Score of 11-13 (Stage I), 7-10 (Stage II), 3-6 (Stage III), 1-2 (Stage IV), or 0 (Stage V). A Physician's Guide to the Management of Huntington's Disease, Lovecky and Trapata (eds.), 3rd Ed., Huntington's Disease Society of America (2011), page 8; Shoulson et al., Assessment of functional capacity in neurodegenerative movement disorders: Huntington's disease as a prototype, in Munsat (ed): Quantification of Neurological Deficit. Boston: Butterworth, 1989, pp 271-283; The Huntington Study Group. Unified Huntington's Disease Rating Scale: reliability and consistency, Mov. Disord. 11, pp. 136-142 (1996).

In one embodiment, the patient has been determined to have more than 40 CAG repeats in the gene encoding the huntingtin protein. In another embodiment, the patient has been determined to have 36-39 CAG repeats. In certain embodiments, the number of CAG repeats is determined using any method known in the art, such as genetic testing methods.

In one embodiment, the patient has no family history of Huntington's disease. In another embodiment, the patient is not aware of a family history of Huntington's disease.

### 5.3.4 Alzheimer's Disease

In specific embodiments, the methods provided herein further comprises a step of selecting, identifying, or diagnosing a patient with Alzheimer's disease prior to the administering step.

In one embodiment, the patient treated according to the methods provided herein has Alzheimer's disease. In one embodiment, the patient treated according to the methods provided herein has had neuroimaging (computed tomography [CT] or Mill) performed after symptom onset consistent with Alzheimer's disease diagnosis.

In one embodiment, the patient treated according to the methods provided herein has dementia of mild to moderate severity defined as mini-mental state examination (MMSE) 16-26 inclusive at the time of screening.

In one embodiment, the patient treated according to the methods provided herein has been on stable doses of regulatory authority approved Alzheimer's disease medication(s) for at least 3 months prior to screening.

In one embodiment, the patient treated according to the methods provided herein has received psychoactive medications (e.g. antidepressants other than monoamine oxidase inhibitors (MAOIs) and most tricyclics, antipsychotics, anxiolytics, anticonvulsants, mood stabilizers, etc). In one embodiment, the patient treated according to the methods provided herein has been on stable doses of psychoactive medications for at least 6 weeks.

In one embodiment, the patient treated according to the methods provided herein has stage 1 Alzheimer's disease. Patients in stage 1 typically have no impairment (e.g., normal function). The person does not experience any memory problems. In some embodiment, an interview with a medical professional does not show any evidence of symptoms of dementia. See Alzheimer's Association at alz.org.

In one embodiment, the patient treated according to the methods provided herein has stage 2 Alzheimer's disease. Patients in stage 2 typically have very mild cognitive decline (may be normal age-related changes or earliest signs of Alzheimer's disease). The person may feel as if he or she is having memory lapses, e.g., forgetting familiar words or the location of everyday objects. But no symptoms of dementia can be detected during a medical examination or by friends, family or co-workers. See Alzheimer's Association at alz.org.

In one embodiment, the patient treated according to the methods provided herein has stage 3 Alzheimer's disease. Patients in stage 3 typically have mild cognitive decline (early-stage Alzheimer's can be diagnosed in some, but not all, individuals with these symptoms). Friends, family or co-workers begin to notice difficulties. During a detailed medical interview, doctors may be able to detect problems in memory or concentration. Common stage 3 difficulties include: noticeable problems coming up with the right word or name, trouble remembering names when introduced to new people, having noticeably greater difficulty performing tasks in social or work settings, forgetting material that one has just read, losing or misplacing a valuable object, and increasing trouble with planning or organizing. See Alzheimer's Association at alz.org.

In one embodiment, the patient treated according to the methods provided herein has stage 4 Alzheimer's disease. Patients in stage 4 typically have moderate cognitive decline (mild or early-stage Alzheimer's disease). At this point, a careful medical interview should be able to detect clear-cut symptoms in several areas: forgetfulness of recent events, impaired ability to perform challenging mental arithmetic—for example, counting backward from 100 by 7s, greater difficulty performing complex tasks, such as planning dinner for guests, paying bills or managing finances, forgetfulness about one's own personal history, and becoming moody or withdrawn, especially in socially or mentally challenging situations. See Alzheimer's Association at alz.org.

In one embodiment, the patient treated according to the methods provided herein has stage 5 Alzheimer's disease. Patients in stage 5 typically have moderately severe cognitive decline (moderate or mid-stage Alzheimer's disease). Gaps in memory and thinking are noticeable, and individuals begin to need help with day-to-day activities. At this stage, those with Alzheimer's may be unable to recall their own address or telephone number or the high school or college from which they graduated, become confused about where they are or what day it is, have trouble with less challenging mental arithmetic; such as counting backward from 40 by subtracting 4s or from 20 by 2s, need help choosing proper clothing for the season or the occasion, still remember significant details about themselves and their family, and still require no assistance with eating or using the toilet. See Alzheimer's Association at alz.org.

In one embodiment, the patient treated according to the methods provided herein has stage 6 Alzheimer's disease. Patients in stage 6 typically have severe cognitive decline (moderately severe or mid-stage Alzheimer's disease). Memory continues to worsen, personality changes may take place and individuals need extensive help with daily activities. At this stage, individuals may lose awareness of recent experiences as well as of their surroundings, remember their own name but have difficulty with their personal history, distinguish familiar and unfamiliar faces but have trouble remembering the name of a spouse or caregiver, need help dressing properly and may, without supervision, make mistakes such as putting pajamas over daytime clothes or shoes on the wrong feet, experience major changes in sleep patterns, e.g., sleeping during the day and becoming restless at night, need help handling details of toileting (for example, flushing the toilet, wiping or disposing of tissue properly), have increasingly frequent trouble controlling their bladder or bowels, experience major personality and behavioral changes, including suspiciousness and delusions (such as believing that their caregiver is an impostor) or compulsive, repetitive behavior like hand-wringing or tissue shredding, and tend to wander or become lost. See Alzheimer's Association at alz.org.

In one embodiment, the patient treated according to the methods provided herein has stage 7 Alzheimer's disease. Patients in stage 7 typically have very severe cognitive decline (severe or late-stage Alzheimer's disease). In the final stage of this disease, individuals lose the ability to respond to their environment, to carry on a conversation and, eventually, to control movement. They may still say words or phrases. At this stage, individuals need help with much of their daily personal care, including eating or using the toilet. They may also lose the ability to smile, to sit without support and to hold their heads up. Their reflexes become abnormal, muscles grow rigid, and swallowing is impaired. See Alzheimer's Association at alz.org.

### 5.3.5 Parkinson's Disease

In specific embodiments, the methods provided herein further comprises a step of selecting, identifying, or diagnosing a patient with Parkinson's disease, in particular, idiopathic Parkinson's disease, prior to the administering step.

The Hoehn and Yahr scale is a system commonly used for describing, in broad terms, how Parkinson's symptoms progress and the relative level of disability. It was originally published in 1967 in the journal Neurology by Melvin Yahr and Margaret Hoehn, and included stages one to five. Since then, stage 0 has been added and stages 1.5 and 2.5 have been proposed and are widely used.
Stage 0—no signs of disease
Stage 1—symptoms on one side only (unilateral)
Stage 1.5—symptoms unilateral and also involving the neck and spine
Stage 2—symptoms on both sides (bilateral) but no impairment of balance
Stage 2.5—mild bilateral symptoms with recovery when the ‘pull’ test is given (the doctor stands behind the person and asks them to maintain their balance when pulled backwards)
Stage 3—balance impairment. Mild to moderate disease. Physically independent
Stage 4—severe disability, but still able to walk or stand unassisted
Stage 5—needing a wheelchair or bedridden unless assisted.

In one embodiment, the patient treated in accordance with the methods disclosed herein is in Stage 1 of the Hoehn and Yahr scale. In one embodiment, the patient treated in accordance with the methods disclosed herein is in Stage 1.5 of the Hoehn and Yahr scale. In one embodiment, the patient treated in accordance with the methods disclosed herein is in Stage 2 of the Hoehn and Yahr scale. In one embodiment, the patient treated in accordance with the methods disclosed herein is in Stage 2.5 of the Hoehn and Yahr scale. In one embodiment, the patient treated in accordance with the methods disclosed herein is in Stage 3 of the Hoehn and Yahr scale. In one embodiment, the patient treated in accordance with the methods disclosed herein is in Stage 4 of the Hoehn and Yahr scale. In one embodiment, the patient treated in accordance with the methods disclosed herein is in Stage 5 of the Hoehn and Yahr scale.

### 5.3.6 Multiple Sclerosis

In one embodiment, the methods provided herein further comprises a step of selecting, identifying, or diagnosing a patient with multiple sclerosis prior to the administering step. In some embodiments, the form of the multiple sclerosis is relapsing remitting, secondary progressive, primary progressive, or progressive-relapsing multiple sclerosis. In one embodiment, the patient with multiple sclerosis is a patient with a relapsing form of MS. In a specific embodiment, the patient has relapsing-remitting MS (RR-MS). In another embodiment, the patient with multiple sclerosis is a patient with a progressive form of MS. In a specific embodiment, the patient has secondary-progressive MS (SP-MS). In another specific embodiment, the patient has primary-progressive MS (SP-MS). In yet another specific embodiment, the patient has progressive-relapsing MS (PR-MS).

### 5.4 Dosing Regimens

This disclosure provides dosing regimens for use in the methods of treatment described herein.

Provided herein are methods of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

In one embodiment, the amount of dimethyl fumarate that is administered in said step of administering intravenously is in the range of 1 to 1000 milligrams. In one embodiment, the amount of dimethyl fumarate that is administered in said step of administering intravenously is in the range of 10 to 750 milligrams. In one embodiment, the amount of dimethyl fumarate that is administered in said step of administering intravenously is in the range of 48 to 240 milligrams. In one embodiment, a therapeutically effective amount of dimethyl fumarate is administered in said step of administering intravenously, said amount being less than 480 milligrams.

In one embodiment, said administering is performed daily. In one embodiment, said administering is performed once per week. In one embodiment, said administering is performed every other week. In one embodiment, said administering is performed once per month.

In one embodiment, the step of administering intravenously is repeated over a time period of at least two weeks. In one embodiment, the step of administering intravenously is repeated over a time period of at least one month. In one embodiment, the step of administering intravenously is repeated over a time period of at least six months. In one embodiment, the step of administering intravenously is repeated over a time period of at least one year.

In one embodiment, said administering is part of a treatment regimen wherein said administering intravenously to the patient alternates with one or more steps of administering the fumarate orally to the patient. In one embodiment, the fumarate is dimethyl fumarate, and the amount of dimethyl fumarate administered orally is 480 mg daily.

The fumarate can be administered to a subject in need thereof at a defined frequency and dosage amount. The amount of the fumarate and pharmaceutical compositions described herein may be intravenously administered once a day or in separate administrations of 2, 3, 4, 5, or 6 doses per day. In one specific embodiment, said fumarate is administered intravenously in doses each day, every two days, every three days, every four days, every five days, every six days or every seven days. In another specific embodiment, said fumarate is administered intravenously every two weeks, every three weeks, every four weeks or every five weeks. In another specific embodiment, said fumarate is administered intravenously every month, every two months, every three months, every four months, every five months or every six months. In specific embodiments, said administrations are in equal dosages. In another specific embodiment, said administration is not in equal dosages (e.g., a subject is treated at a particular dosage that increases during subsequent treatments). In one embodiment, the fumarate is only administered once during a treatment period.

In certain embodiments, the treatment of a subject with the fumarate or compositions comprising the fumarate described herein is repeated over a time period of at least one week. In another specific embodiment, said treatment is repeated over a time period of at least one month. In another specific embodiment, said treatment is repeated over a time period of at least two months. In another specific embodiment, said treatment is repeated over a time period of at least six months. In another specific embodiment, said treatment is repeated over a time period of at least one year.

In one particular aspect, at least one fumarate or compositions comprising the same described herein are administered to a subject in need thereof through multiple routes of administration, which include intravenous administration, or the at least one fumarate can be administered in combination with other agents (i.e., drugs) (see Section 5.6).

The amount of fumarate that is used to produce a single dosage form will vary depending upon the particular mode of administration. It should be understood, however, that a specific dosage and treatment regimen for any particular subject will depend upon a variety of factors, including the activity of the specific compound employed, the age, body weight, general health, sex, diet, time of administration, rate of excretion, drug combination, and the judgment of the treating physician and the severity of the particular disease being treated. The amount of active fumarate can also depend upon the therapeutic or prophylactic agent, if any, with which the fumarate is co-administered.

In a specific embodiment, the fumarate is dimethyl fumarate.

In a specific embodiment, the fumarates or compositions described herein are administered intravenously at a constant rate over the course of the dosing regimen. In another specific embodiment, the fumarate or the compositions comprising a fumarate are administered intravenously at different rates during the course of the dosing regimen (e.g., the initial dosage is at a fixed rate that is increased or decreased during subsequent doses). In another embodiment, the fumarate or compositions comprising a fumarate described herein are administered at a rate of about 10 to 40 mL/kg body weight/hour or at a rate of about 20 to 30 mL/kg body weight/hour.

In specific embodiments, the fumarates or compositions described herein are intravenously administered to a patient in a total volume of 1 mL, 2 mL, 3 mL, 4 mL, 5 mL, 6 mL, 7 mL, 8 mL, 9 mL, or 10 mL. In another specific embodiment, said fumarates or compositions are intravenously administered to a patient in a total volume of 10 mL, 20 mL, 30 mL, 40 mL, 50 mL, 60 mL, 70 mL, 80 mL, 90 mL, or 100 mL. In yet another specific embodiment, the fumarate or compositions comprising a fumarate are intravenously delivered to a patient in a total volume of 100 mL, 200 mL, 300 mL, 400 mL, 500 mL, 600 mL, 700 mL, 800 mL, 900 mL, or 1000 mL.

In some embodiments, the fumarate is administered intravenously in an amount ranging from about 1 mg to about 1000 mg, about 10 mg to about 750 mg, or about 48 mg to about 240 mg. In a particular embodiment, said fumarates or compositions are administered intravenously in an amount that is less than 480 mg or about 160 mg or less. In a particular embodiment, said fumarates or compositions are administered intravenously in an amount that is about 1,120 mg or less once a week, 2,240 mg or less once in two weeks, or 4,800 mg or less once a month.

The amount of the compounds and pharmaceutical compositions described herein administered will also vary, as recognized by those skilled in the art, depending on route of administration, excipient usage, and the possibility of co-usage with other therapeutic treatments including use of other therapeutic agents (i.e., drugs).

In some embodiments, a fumarate is administered intravenously daily (e.g., at a dose of 1.11 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously every other day (e.g., at a dose of 2.22 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously three times a week (e.g., in a dose of 2.59 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously twice a week (e.g., in a dose of 3.89 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once a week (e.g., in a dose of 7.77 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every other week (e.g., in a dose of 15.54 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every three weeks (e.g., in a dose of 23.31 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every four weeks (e.g., in a dose of 31.08 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once a month (e.g., in a dose of 33.30 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every other month (e.g., in a dose of 66.61 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every third month (e.g., in a dose of 99.91 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day). In some embodiments, a fumarate is administered intravenously once every fourth month (e.g., in a dose of 133.21 mmol or less) in combination with an oral dose of a fumarate given daily (e.g., 5.00 mmol/day, 4.16 mmol/day, 3.33 mmol/day, 250 mmol/day, 1.67 mmol/day, or 0.833 mmol/day).

In some embodiments, dimethyl fumarate is administered intravenously daily (e.g., at a dose of 160 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously every other day (e.g., at a dose of 320 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously three times a week (e.g., in a dose of 374 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously twice a week (e.g., in a dose of 560 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once a week (e.g., in a dose of 1,120 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every other week (e.g., in a dose of 2,240 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every three weeks (e.g., in a dose of 3,360 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every four weeks (e.g., in a dose of 4,480 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once a month (e.g., in a dose of 4,800 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every other month (e.g., in a dose of 9,600 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every third month (e.g., in a dose of 14,400 mg or less) in combination with an oral dose of dimethyl fumarate given daily (e.g., 720 mg/day, 480 mg/day, 360 mg/day, 240 mg/day, or 120 mg/day). In some embodiments, dimethyl fumarate is administered intravenously once every fourth month (e.g., in a dose of 19,200 mg or less) in combination with an oral dose of dimethyl fumarate given daily (e.g., 720 mg/day, 480 mg/day, 360 mg/day, 240 mg/day, or 120 mg/day).

The intravenous administration of the fumarates or pharmaceutically acceptable salts, tautomers, or stereoisomers thereof described herein may provide higher levels of said fumarates in the circulatory system of a subject as compared to the oral administration of the same amount of the same fumarates. Thus, the intravenous administration of a fumarate described herein (e.g., dimethyl fumarate) is expected to be administered at a lower dose than oral administration to achieve the same clinical effect. In a specific embodiment, intravenous administration of a fumarate described herein is administered at a dose at least two-fold, at least 10-fold, at least 20-fold, at least 30-fold, at least 40-fold, at least 50-fold, at least 100-fold, at least 200-fold, at least 300-fold, at least 400-fold or at least 500-fold lower than oral administration of the fumarate to achieve a useful clinical or pharmacodynamic effect. In specific embodiments, the clinical or pharmacodynamic effect is determined by the concentration of the fumarate (e.g., dimethyl fumarate or monomethyl fumarate) in the blood or plasma of the patient. In another embodiment, the clinical or pharmacodynamic effect is determined by the concentration or amount of fumarate (e.g., dimethyl fumarate or monomethyl fumarate) in one or more tissues (e.g., brain) of the patient. In yet another embodiment, said clinical or pharmacodynamic effect is the treatment of a neurological disease, for example, the impairment associated with a neurological disease, as described herein (see Section 5.2).

### 5.5 Pharmaceutical Compositions

The at least one fumarate as described herein can be formulated into a pharmaceutically acceptable composition for the intravenous administration to a subject in need thereof. Suitable formulations for intravenous administration are well known. In a preferred embodiment, said fumarates are formulated in a solution for intravenous administration, also known as IV administration or IV drip.

In a specific embodiment, the pharmaceutical composition consists essentially of at least one fumarate as described herein. In another specific embodiment, the fumarate is dimethyl fumarate. In another specific embodiment, the fumarate is monomethyl fumarate or a prodrug thereof. In another embodiment, the only drug in the pharmaceutical composition is dimethyl fumarate and optionally one or more compounds produced by degradation from dimethyl fumarate in said pharmaceutical composition. In another specific embodiment, said additional compound produced by ex vivo degradation is monomethyl fumarate.

The at least one fumarate described herein is formulated in a composition suitable for intravenous administration. Compositions suitable for intravenous formulations are known in the art and include solutions (e.g., aqueous solutions). In specific embodiments, the intravenous compositions described herein comprise volume expanding solutions including but not limited to normal saline, lactated Ringer's solution, Hartmann's Solution, glucose, hydroxyethyl starch, gelofusine and the like. In another specific embodiment, the compositions described herein contain one or more buffering agents including but not limited to sodium bicarbonate, sodium phosphate, sodium biphosphate, citric acid, boric acid, Sorenson's phosphate buffer and all pharmaceutically acceptable salts of said buffering agents. In one specific embodiment, said composition comprises a fumarate described herein (e.g., dimethyl fumarate) formulated in a sterile solution. In certain embodiments, said solution is isotonic to blood. In another specific embodiment, said fumarate is a dimethyl fumarate, monomethyl fumarate or a prodrug thereof, or a deuterated form of the same.

Pharmaceutical compositions suitable for intravenous administration are also described in Sections 5.5.1 and 5.5.2 below.

The pharmaceutical compositions described herein can be administered to a subject in need thereof in any intravenous delivery format that is pharmaceutically acceptable. In a specific embodiment, said composition (e.g., a solution) is delivered through a syringe. In another specific embodiment, said composition (e.g., a solution) is delivered through an infusion bag. In another specific embodiments, said composition (e.g., a solution) is delivered using a hypodermic needle, a peripheral cannula, a central venous catheter, a peripherally inserted line, a tunneled line or implantable port.

In one embodiment, the pharmaceutical composition contains different fumarates from those fumarates in FUMADERM®. FUMADERM® comprises a combination of dimethyl fumarate, calcium salt of ethyl hydrogen fumarate, magnesium salt of ethyl hydrogen fumarate, and zinc salt of ethyl hydrogen fumarate.

In a specific embodiment, the pharmaceutical composition comprises a fumarate; wherein the fumarate is a dialkyl fumarate, a monoalkyl fumarate, a combination of a dialkyl fumarate and a monoalkyl fumarate, a prodrug of monoalkyl fumarate, or a deuterated form of any of the foregoing, or a tautomer, or stereoisomer of any of the foregoing, or a combination of any of the foregoing; with the proviso that a fumarate salt is not present in the pharmaceutical composition.

In a specific embodiment, the pharmaceutical composition comprises a fumarate; wherein the fumarate is a dialkyl fumarate, a monoalkyl fumarate, a combination of a dialkyl fumarate and a monoalkyl fumarate, a prodrug of monoalkyl fumarate, or a deuterated form of any of the foregoing, or a pharmaceutically acceptable salt, tautomer, or stereoisomer of any of the foregoing, or a combination of any of the foregoing; with the proviso that an ethyl hydrogen fumarate salt is not present in the pharmaceutical composition.

In a specific embodiment, the pharmaceutical composition comprises a fumarate; wherein the fumarate is a dialkyl fumarate, a monoalkyl fumarate, a combination of a dialkyl fumarate and a monoalkyl fumarate, a prodrug of monoalkyl fumarate, or a deuterated form of any of the foregoing, or a pharmaceutically acceptable salt, tautomer, or stereoisomer of any of the foregoing, or a combination of any of the foregoing; with the proviso that ethyl hydrogen fumarate calcium salt, ethyl hydrogen fumarate magnesium salt, ethyl hydrogen fumarate zinc salt, and ethyl hydrogen fumarate copper salt are not present in the pharmaceutical composition.

In a specific embodiment, the pharmaceutical composition comprises a fumarate; wherein the fumarate is a dialkyl fumarate, a monoalkyl fumarate, a combination of a dialkyl fumarate and a monoalkyl fumarate, or a deuterated form of any of the foregoing, or a tautomer, or stereoisomer of any of the foregoing, or a combination of any of the foregoing; with the proviso that a fumarate salt is not present in the pharmaceutical composition.

In a specific embodiment, the pharmaceutical composition comprises a fumarate; wherein the fumarate is a dialkyl fumarate, a monoalkyl fumarate, a combination of a dialkyl fumarate and a monoalkyl fumarate, or a deuterated form of any of the foregoing, or a pharmaceutically acceptable salt, tautomer, or stereoisomer of any of the foregoing, or a combination of any of the foregoing; with the proviso that an ethyl hydrogen fumarate salt is not present in the pharmaceutical composition.

In a specific embodiment, the pharmaceutical composition comprises at least one fumarate; wherein the fumarate is a dialkyl fumarate, a monoalkyl fumarate, a combination of a dialkyl fumarate and a monoalkyl fumarate, or a deuterated form of any of the foregoing, or a pharmaceutically acceptable salt, tautomer, or stereoisomer of any of the foregoing, or a combination of any of the foregoing; with the proviso that ethyl hydrogen fumarate calcium salt, ethyl hydrogen fumarate magnesium salt, ethyl hydrogen fumarate zinc salt, and ethyl hydrogen fumarate copper salt are not present in the pharmaceutical composition.

In a specific embodiment, the at least one fumarate described herein is administered as a monotherapy; thus, it is not administered in combination with one or more active pharmaceutical agents (i.e., drugs). In a particular embodiment, the at least one fumarate described herein is not administered in combination with any one or more or all of the following active pharmaceutical agents: an angiotensin-converting enzyme inhibitor (e.g., the inhibitors disclosed in WO2013/022882A1); amino-adamantane-derived NMDA receptor antagonist (e.g., memantine, rimantadine, and amantadine (see, e.g., US2008/0089861); a multiple sclerosis drug, peroxisome proliferator-activated receptor (PPAR) gamma agonist (e.g., the agonists disclosed in US2013/0158077A1); glatiramer acetate or related copolymers (e.g., as disclosed in WO2011/100589A1); interferon-beta (see, e.g., WO2011/100589A1); a drug that reduces or eliminates flushing, such as a prostaglandin modulator, a dietary fiber, a composition comprising inhibitors of mast cell activation and secretion of inflammatory biochemicals, a mineral salt, or a drug as described in, e.g., WO2007/042035A1; guanabenz or guanabenz derivative (e.g., the guanabenz or guanabenz derivatives disclosed in WO2014/138298A1); S-adenosyl methionine decarboxylase (SAMDC) inhibitor, polyamine analog, or polyamine biosynthesis inhibitor (see, e.g., the drugs disclosed in WO2014/110154A1); VLA4 binding antibody (e.g., the antibodies disclosed in EP2783701A2); and angiopoietin-2 (ANG-2) inhibitor (e.g., the inhibitors disclosed in EP2307456B1).

### 5.5.1 Nanosuspensions

Provided herein are pharmaceutical compositions comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is a nanosuspension. In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate.

In one embodiment, the concentration of the dimethyl fumarate is about 1 mg/ml to about 150 mg/ml. In one embodiment, the concentration of the dimethyl fumarate is about 150 mg/ml.

In one embodiment, the pharmaceutical composition further comprises one or more excipients selected from a small molecule stabilizer, a polymeric stabilizer, and a buffer.

In one embodiment, the small molecule stabilizer is sodium dodecyl sulfate. In one embodiment, the polymeric stabilizer is hydroxy propyl methyl cellulose (HPMC). In one embodiment, the buffer is a phosphate buffer.

In one embodiment, the pH of the composition is in the range from about 4 to about 7. In one embodiment, the pH of the composition is about 5.0.

In one embodiment, the fumarate has a mean particle size (D50) of about 100 nm to about 250 nm. In one embodiment, the D50 is about 180 nm.

In one embodiment, the fumarate is dimethyl fumarate, wherein the pharmaceutical composition further comprises sodium dodecyl sulfate; HPMC, and a phosphate buffer, wherein the pH of the pharmaceutical composition is about 5.0 and the D50 is about 180 nm.

The pharmaceutical compositions provided in this section can be used in any of the methods provided herein.

### 5.5.2 Formulations Comprising Cylcodextrins

Provided herein are pharmaceutical compositions comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is an aqueous solution, wherein the aqueous solution comprises a cyclodextrin, wherein the cyclodextrin is an alpha cyclodextrin or a substituted beta cyclodextrin. In one embodiment, the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

In one embodiment, the fumarate is dimethyl fumarate. In one embodiment, the concentration of the dimethyl fumarate is about 1 mg/ml to about 16 mg/ml. In one embodiment, the concentration of the dimethyl fumarate is about 2 mg/ml to about 4 mg/ml.

In one embodiment, the cyclodextrin is a substituted beta cyclodextrin.

In one embodiment, the substituted beta cyclodextrin is present from about 5% (w/v) to about 40% (w/v). In one embodiment, the substituted beta cyclodextrin is present at about 20% (w/v).

In one embodiment, the substituted beta cyclodextrin is hydroxypropyl beta cyclodextrin or sulfobutylether beta cyclodextrin. In one embodiment, the substituted beta cyclodextrin is a sulfobutylether beta cyclodextrin.

In one embodiment, the pharmaceutical composition comprises one or more sulfobutylether beta cyclodextrins of Formula XX:

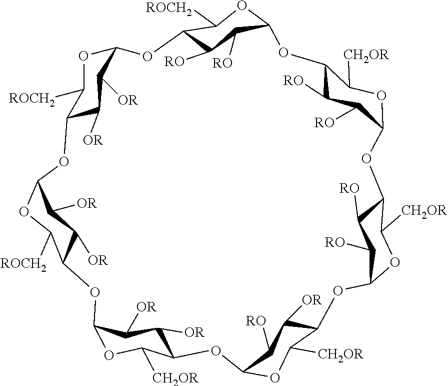

wherein R is independently selected from H or —CH₂CH₂CH₂CH₂SO₃Na, with the proviso that R is H but for 6 or 7 instances where R is —CH₂CH₂CH₂CH₂SO₃Na. In one embodiment, the pharmaceutical composition comprises CAPTISOL.

In one embodiment, the fumarate is dimethyl fumarate, and wherein the aqueous solution comprises 20% (w/v) CAPTISOL, and the concentration of the DMF is about 2 mg/ml to about 4 mg/ml.

The pharmaceutical compositions provided in this section can be used in any of the methods provided herein.

### 5.6 Combination Treatments

In specific embodiments, the at least one fumarate described herein is administered in combination with one or more additional compounds indicated for treatment of the neurological disease which the patient has. The additional compound(s) can be in the same or separate compositions from the at least one fumarate, and can be delivered intravenously or by a different route of administration (e.g., oral). In a specific embodiment, said one or more additional compounds are administered sequentially, prior to, after, or concurrently with the fumarate described herein. In a specific embodiment wherein a fumarate described herein (for example, dimethyl fumate) is administered both intravenously and orally, compositions and dosing regimens for oral fumarates can be used that are known in the art (see, e.g., TECFIDERA® Prescribing Information, March 2013; WO 2008/097596; WO 2010/126605).

### 6. EXAMPLES

### 6.1 Example 1: Intravenous and Oral Administration of Radiolabeled Dimethyl Fumarate

This Example describes the oral and intravenous administration of radiolabeled DMF to mice and the resulting tissue distribution as assessed by imaging.

### 6.1.1 Method of Making (¹¹C) Dimethyl Fumarate

An exemplary method for making (¹¹C) dimethyl fumarate is provided herein below.

Fumaric acid and iron (II) chloride were dissolved in thionyl chloride and refluxed for 24 hours. Fractional distillation was used to purify the fumaroyl dichloride. Unreacted thionyl chloride distilled over at 74° C. and accounted for 45% of the reaction mixture. Any volatile byproducts distilled over between 80° C. and 150° C. The product, fumaroyl dichloride distilled over at 158° C., and was collected as a pungent yellow liquid. The fumaroyl dichloride accounted for ˜45-50% of the reaction mixture. Unreacted fumaric acid and iron (II) chloride remained in the reaction vessel. The fumaroyl dichloride was flushed with argon and stored at −20° C. The fumaroyl dichloride was found to be stable and reactive for up to 7 days when stored under inert atmosphere.

¹¹C-carbon dioxide was trapped in lithium aluminum hydride (10 μmol) to form ¹¹C-methanol, with which fumaroyl dichloride (1 mg) was allowed to react at room temperature for 5-10 minutes. The reaction mixture was quenched with 10 μL of water and 100 μL of methanol to produce ¹¹C-dimethyl fumarate (yield 5-10%, decay corrected). The reaction mixture was quenched with 100 μL of water to produce ¹¹C-monomethyl fumarate (yield 1-3%, decay corrected). The radiolabeled product was diluted to 50 mL using water, and passed over a C-18 Sep-pak (pretreated by rinsing with 10 mL ethanol and 10 mL water). Sep-pak was rinsed with 10 mL water. Radiolabeled products were eluted with 0.1-0.5 mL ethanol, and added to sterile saline (0.9%) to provide a final formulation of 10% ethanol in sterile saline (0.9%). For oral dosing, 400 mg/kg of non-radioactive drug was dissolved in 0.2 mL of 0.8% methocell. The radiolabeled drug, synthesized, for example, as described above, was added to the mixture, and given via oral gavage.

### 6.1.2 (¹⁴C) Dimethyl Fumarate

[2,3-¹⁴C] DMF, which was used in Example 1, was obtained from ViTrax Co., Placentia, Calif., USA (Lot #155-038-000, specific activity 54 mCi/mmol).

### 6.1.3 Results of Intravenous Administration of Dimethyl Fumarate Compared to Oral Administration

To determine the localization and retention of DMF administered by different routes in vivo, mice were administered isotopically labeled DMF either orally or through tail vein injections. To track DMF localization after administration, animals were imaged using microPET Focus220™ (Siemens) for positron emission tomography (PET) and a BioSpin 7T (Bruker) for magnetic resonance imaging (MR). Acquired images from PET experiments were analyzed using inviCRO software.

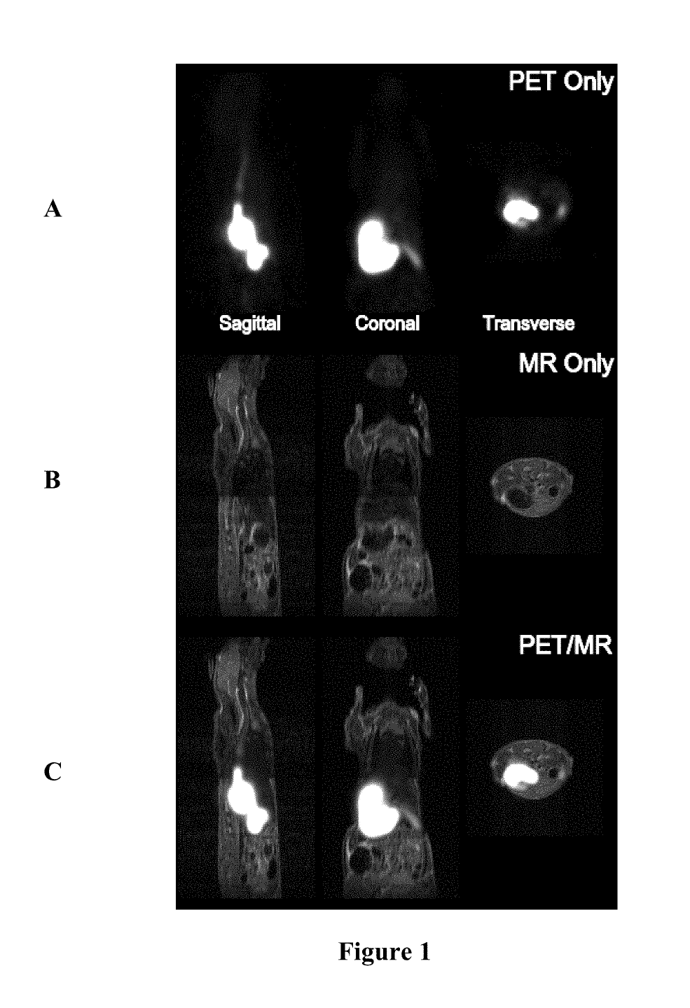

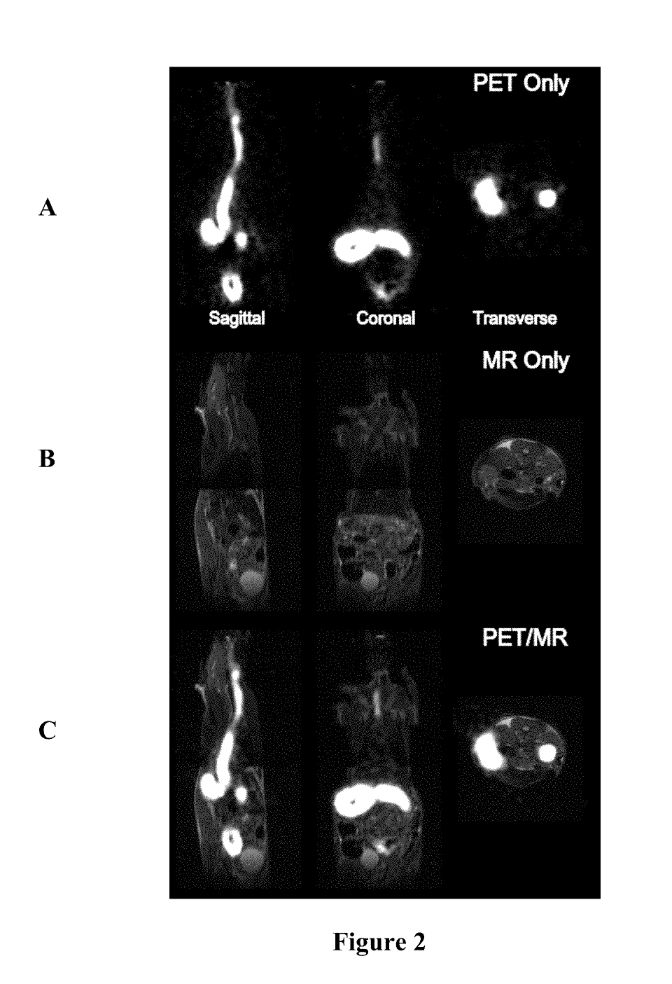

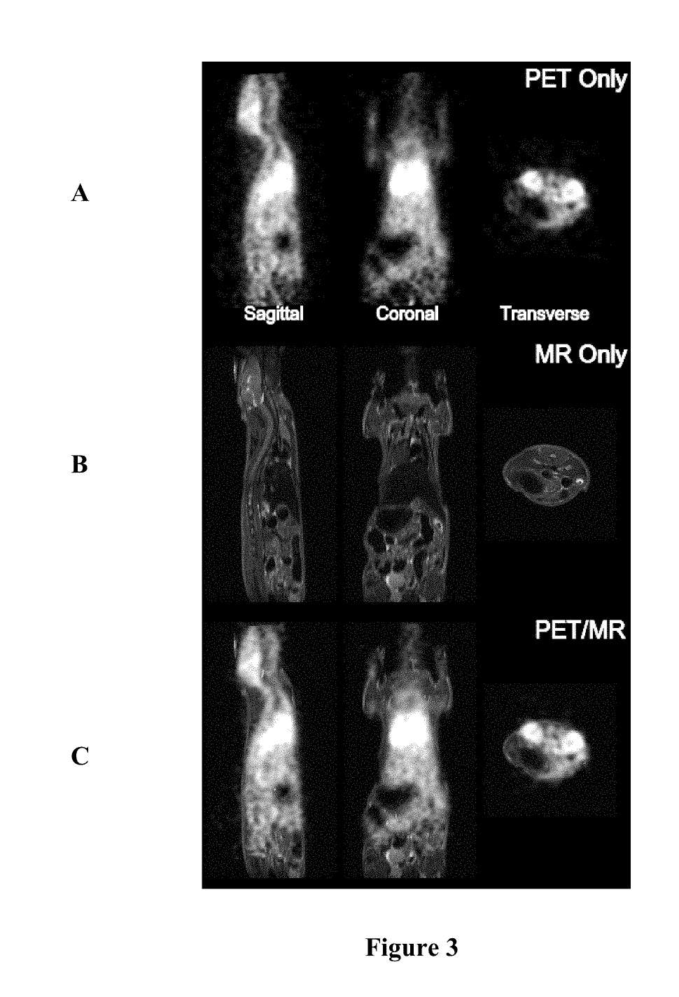

Naïve male CD₁ mice were administered isotopically labeled (¹¹C) dimethyl fumarate (DMF) (see Section 6.1.1) orally (PO) at either 0.5 mg/kg (N=2) or 200 mg/kg (N=3), or intravenously (IV) at 0.5 mg/kg (N=3) and imaged by positive emission tomography (PET) or magnetic resonance (MR). All animals were anaesthetized during image acquisition. Treated mice were imaged at various time points up to 90 minutes after administration of (¹¹C)DMF. Mice treated with PO (¹¹C)DMF at either 0.5 mg/kg or 200 mg/kg displayed positive signal primarily in the digestive tract and the kidneys (FIGS. 1 and 2, respectively). Conversely, animals administered IV (¹¹C)DMF displayed positive signal throughout various tissues of the body including the brain and heart (FIG. 3). These findings indicate that intravenously delivered DMF is localized in various tissues in vivo as compared to orally administered DMF, even when the orally delivered composition is at a much higher concentration (i.e. 0.5 mg/kg IV administration vs. 200 mg/kg PO administration). The signals displayed may derive from (¹¹C)DMF or any in vivo conversion product thereof.

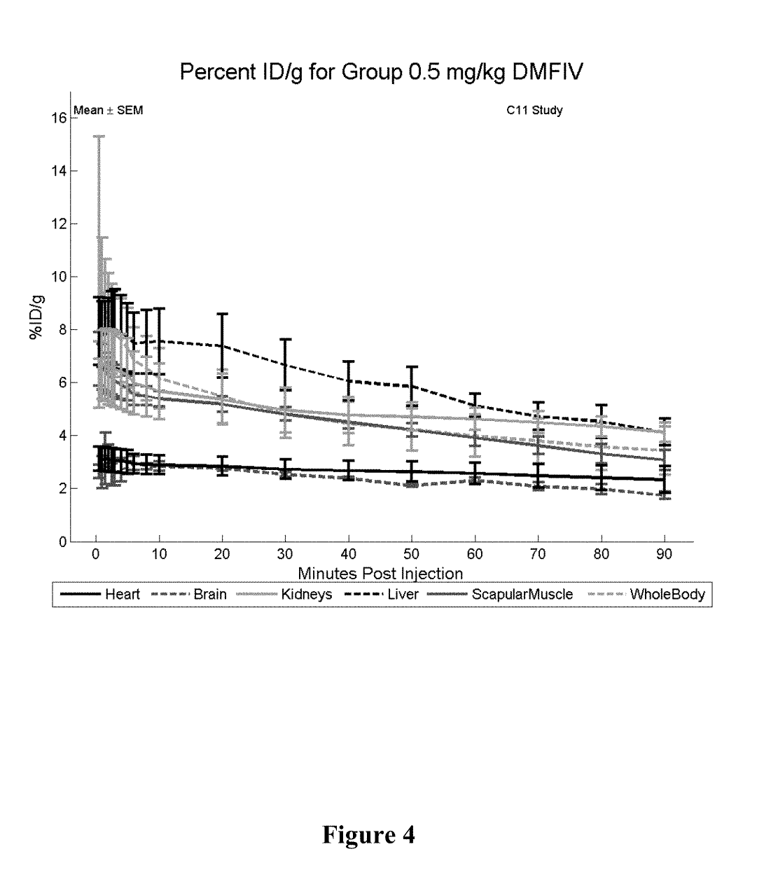

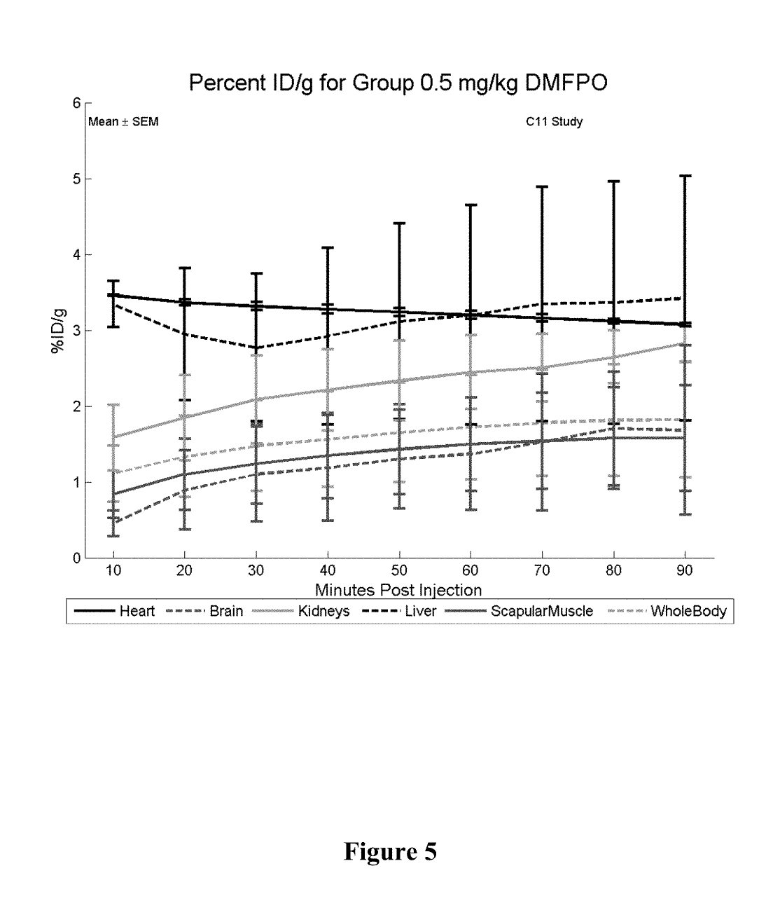

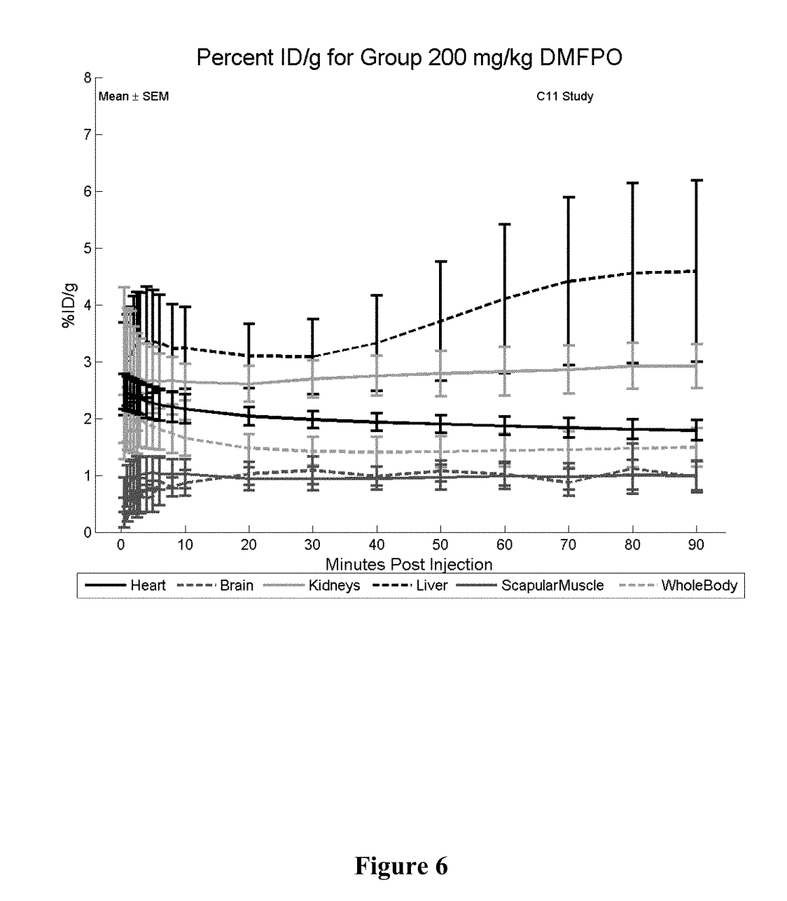

The imaging data from the three groups of treated animals were quantified according to tissue type using inviCRO image analysis software. Regions of Interest (ROIs) for the brain, heart, liver, kidneys, scapular muscle, and whole body were defined by fitting ellipsoids of fixed volume to the regions. The scapular muscle ROI was hand-drawn and is not representative of all mouse muscle, but is composed of only a small region of its respective ROI. In 0.5 mg/kg IV treated animals, signal was higher in most tissues (FIG. 4) as compared to PO treated animals treated at either 0.5 mg/kg (FIG. 5) or 200 mg/kg (¹¹C)DMF (FIG. 6).

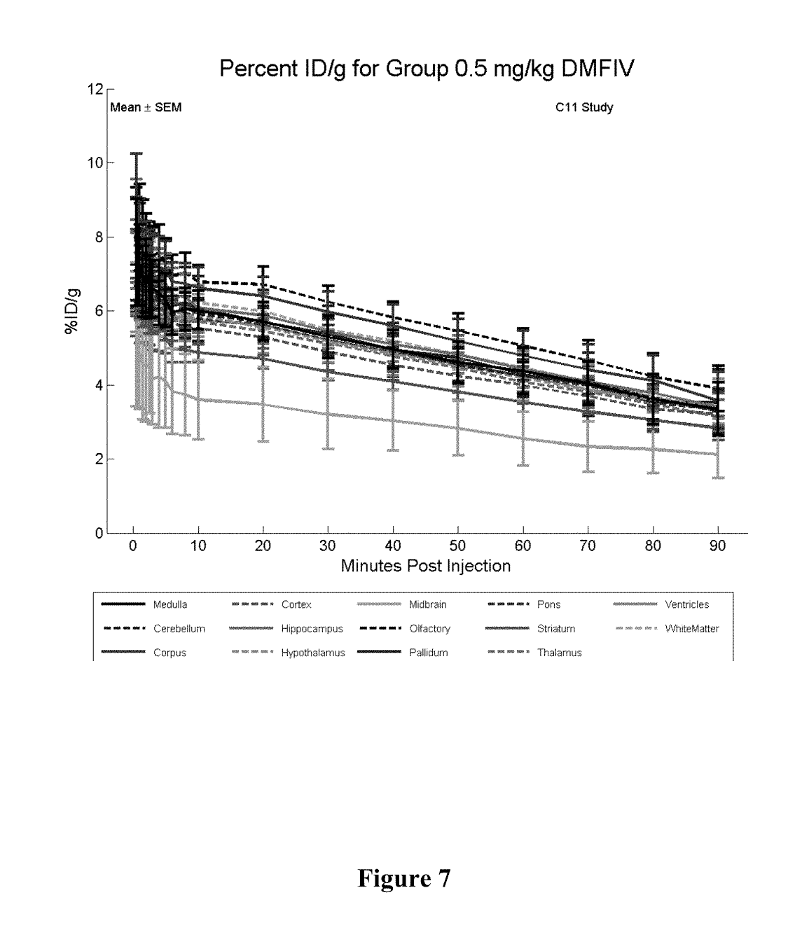

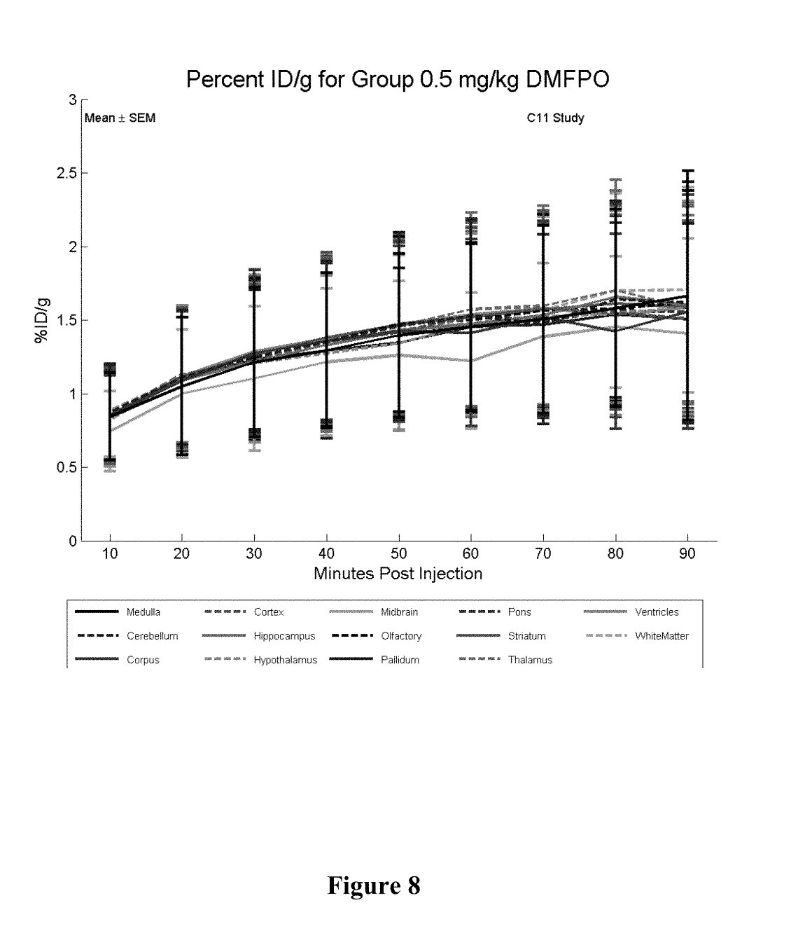

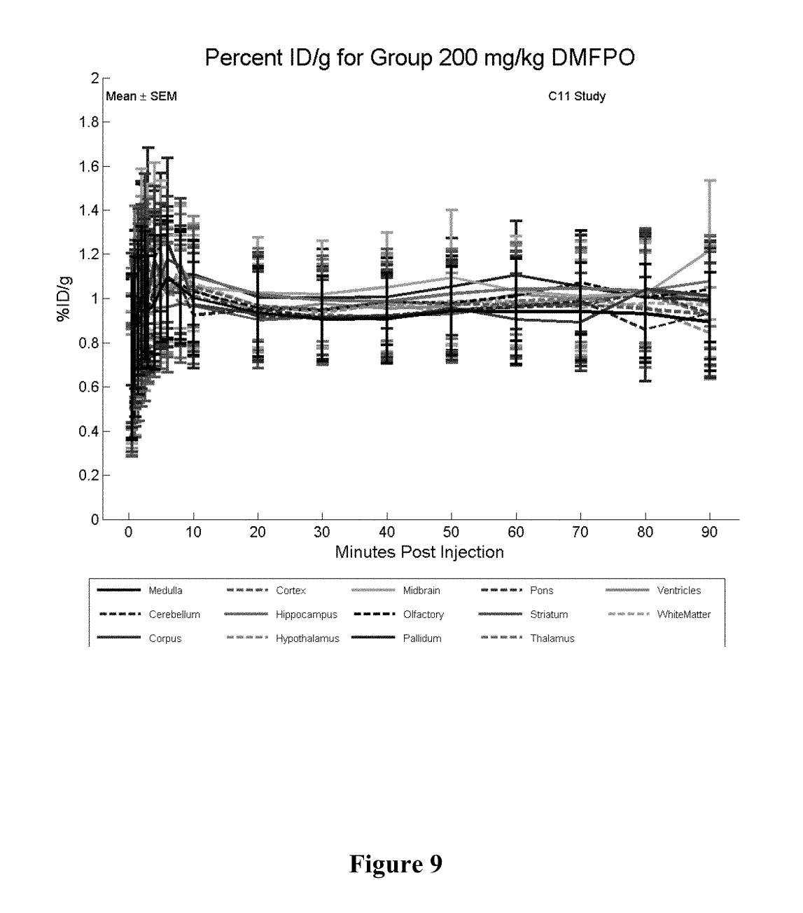

Notably, the signal in the brains of IV treated animals (FIG. 7) was much greater than in the brains of PO treated animals at low or high concentrations (FIGS. 8 and 9). To quantitatively examine signal from various regions of the brain, ROIs for the Medulla, Cerebellum, Midbrain, Pons, Cortex, Hippocampus, Thalamus, Hypothalamus, Striatum, Pallidum, Olfactory, Corpus Callosum, White Matter, and Ventricles were acquired by fitting a proprietary 14-region mouse brain atlas to the brain regions of each animal. Indeed, when signals from these specific regions of the brain were quantified, almost no signal was detected in any region for PO treated animals (FIGS. 8 and 9). IV treated animals, however, displayed high levels of signal in all regions of the brain throughout the observation period (FIG. 7). These results show that IV administration of DMF surprisingly leads to higher concentrations of DMF in the brain than when administering DMF orally.

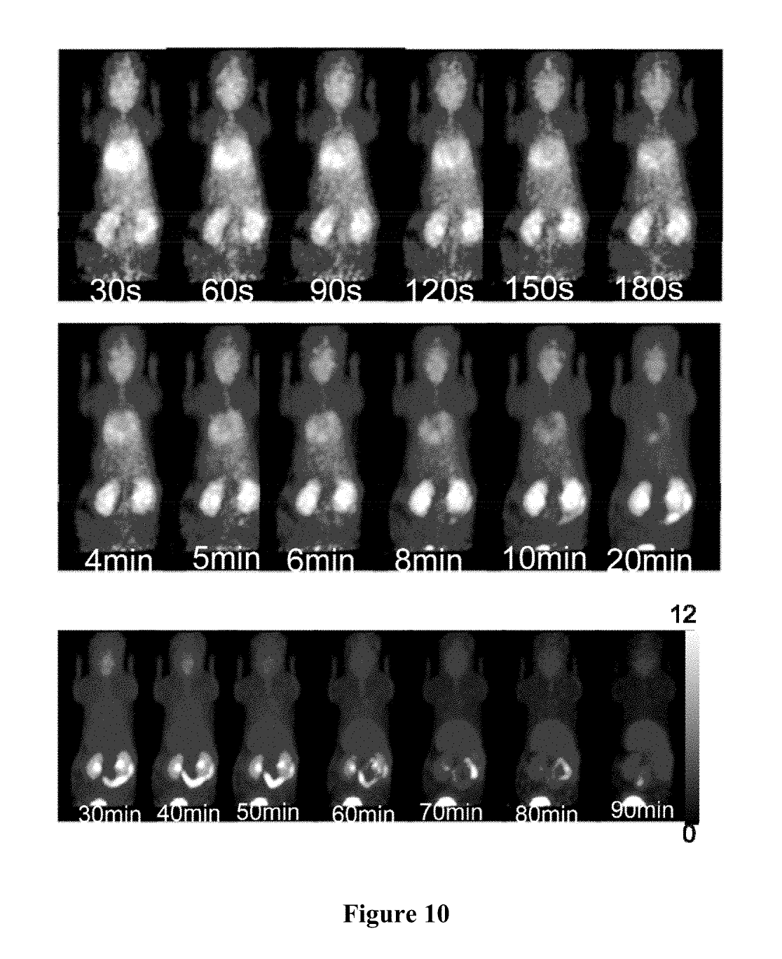

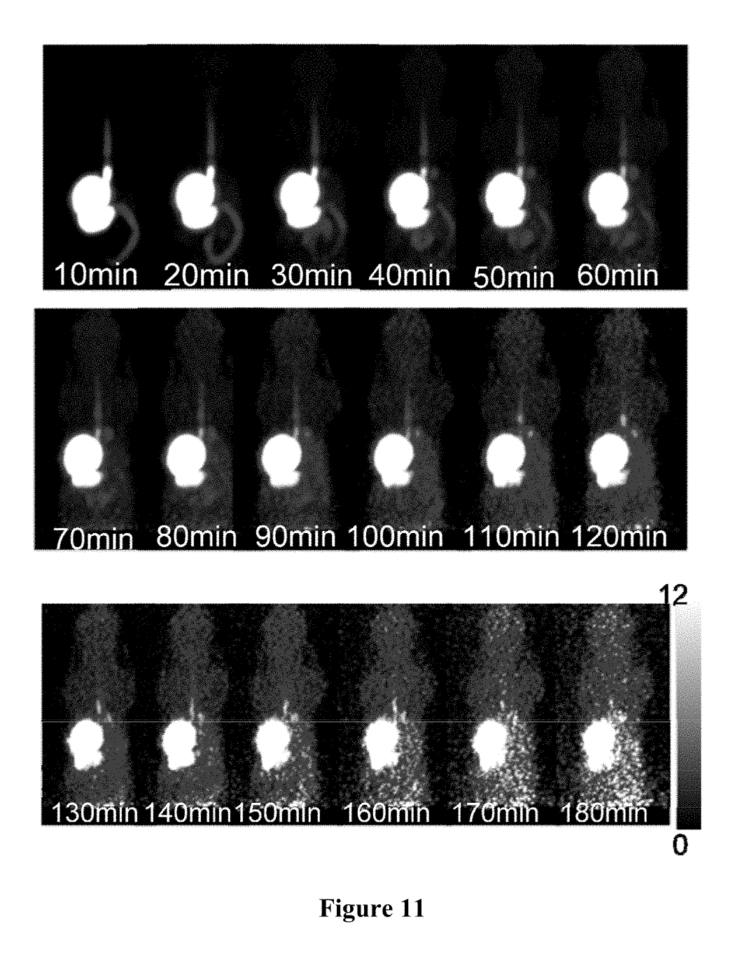

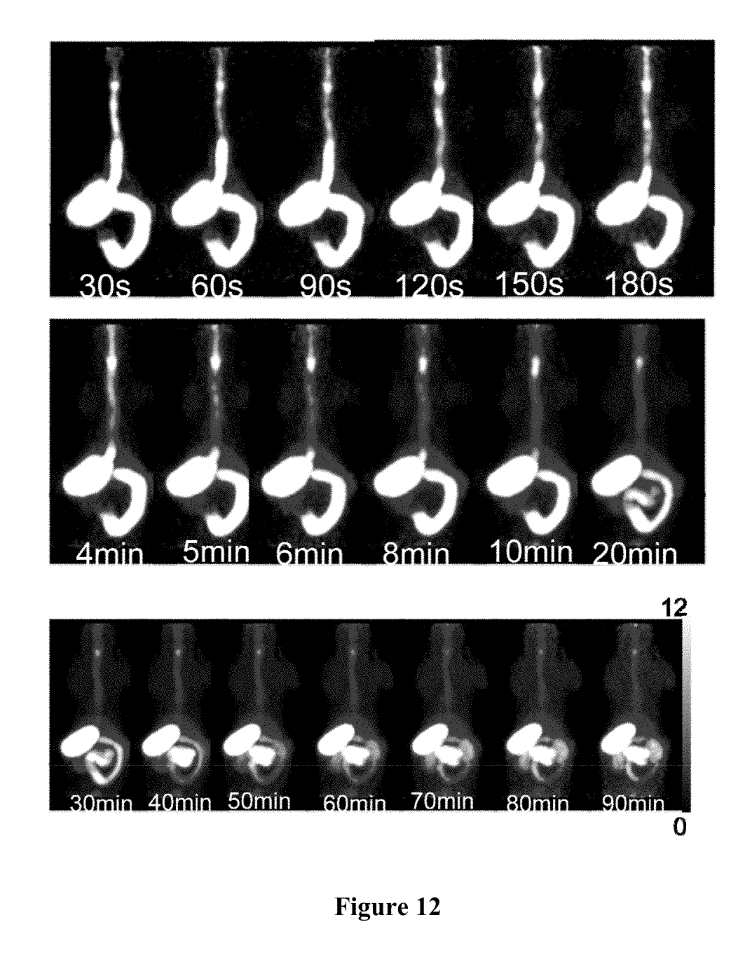

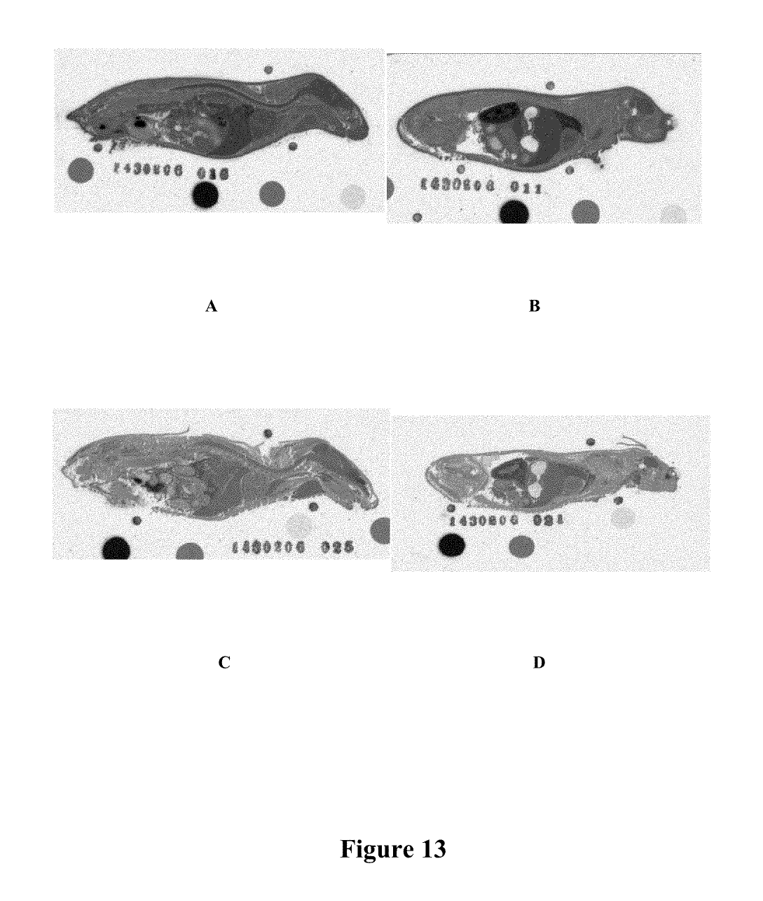

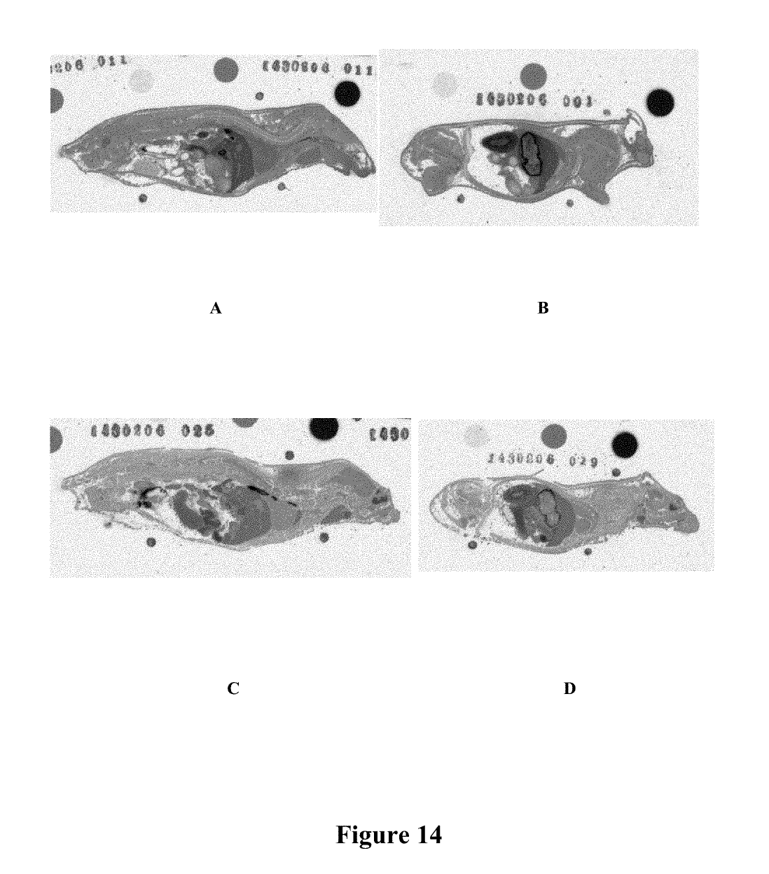

To confirm the effects observed for IV DMF localization in the brain, mice treated with (¹¹C)DMF were compared to mice treated with (¹⁴C)DMF (see Section 6.1.2). As shown in FIG. 10, signal from animals treated with 0.5 mg/kg IV (¹¹C)DMF was localized in numerous tissues, including the brain, as early as 30 seconds after administration. Animals treated with either 0.5 mg/kg PO (¹¹C)DMF (FIG. 11) or 200 mg/kg PO (¹¹C)DMF (FIG. 12) displayed signal almost exclusively in the digestive tract throughout the course of the treatment. Similarly, animals treated with 0.5 mg/kg IV (¹⁴C)DMF (FIG. 13) displayed signal in the kidneys and brain at both 10 minutes (FIG. 13A, B) and 60 minutes (FIG. 13C, D) after administration when viewed in sagittal section, whereas animals treated with 0.5 mg/kg PO (¹⁴C)DMF (FIG. 14) had less signal in the brain 10 minutes (FIGS. 14A and B) and 60 minutes (FIGS. 14C and D) after (¹⁴C)DMF administration. These results indicate that (¹⁴C)DMF administered intravenously surprisingly quickly accumulates in the brain and other tissues compared to (¹⁴C)DMF administered orally, which results (¹⁴C)DMF largely being absent from the brains of treated animals.

Examining the localization of (¹¹C)DMF to particular regions of the brain in treated mice indicated that specific regions emit a higher signal than other regions (FIG. 7). Notably, many regions known to to be relevant to neurological diseases emitted a strong signal after intravenous treatment with (¹¹C)DMF; for example the striatum (Huntington's Disease, stroke), cortex and hippocampus (Alzheimer's Disease, stroke), substantia nigra and brain stem (Parkinson's Disease, stroke), cerebral cortex (stroke) and spinal cord (ALS, stroke) and cerebellum (stroke) all emitted a strong signal as compared to olfactory regions after intravenous treatment with (¹¹C)DMF (FIG. 7). Since oral administration of (¹¹C)DMF at either 0.5 mg/kg or 200 mg/kg mostly displayed significantly lower levels of signal throughout the brain, these results indicate that, surprisingly, intravenous administration of DMF may specifically localize to certain neural structures involved in particular neurological diseases.

### 6.2 Example 2: Characterization of Pharmacokinetics and Pharmacodynamics of Dimethyl Fumarate Administered Orally and Intravenously

This Example compares the pharmacodynamic response of prototypical Nrf2 response genes in rats and mice to oral and intravenous administration of DMF.

### 6.2.1 DMF Administered Orally and Intravenously to Sprague Dawley Rats

### List of Abbreviations

TABLE 2

| | |
|---|---|
| TBD | To be determined |
| PK | Pharmacokinetics |
| PD | Pharmacodynamics |
| IV | Intravenous |
| PO | Per os (oral dosing) |
| PET | Positron emission tomography |
| QWBA | Quantitative whole body autoradiography |
| DMF | Dimethyl fumarate |
| MMF | Monomethyl fumarate |
| HPMC | Hydroxypropyl methylcellulose |
| Akr1b8 | Aldo-keto reductase family 1, member b8 |
| Gclc | Glutamate cysteine ligase, catalytic subunit |
| Hmox1 | Hemeoxygenase 1 |
| Nqo1 | NADP(H) dehydrogenase quinone 1 |
| Osgin1 | Oxidative stress induced growth inhibitor 1 |
| Txnrd1 | Thioredoxin reductase 1 |
| MS | Multiple Sclerosis |
| CNS | Central nervous system |
| qRT-PCR | Quantitative real-time polymerase chain reaction |
| ANOVA | Analysis of variance |

Introduction

The mouse PET and QWBA studies described in Example 1 demonstrate that administering ¹¹C or ¹⁴C-labeled DMF IV resulted in a rapid and selective partitioning of radioactivity into the CNS, whereas PO dosing produced a more peripheral distribution confined predominately to the gastrointestinal tract (GI). These imaging data are able to characterize the anatomical location of radioactivity, but the molecular identity of what is being imaged is unknown and could be DMF itself, MMF or any number of different DMF conjugates or metabolites.

To determine if the observed imaging results correlated with meaningful biological effects (e.g., Nrf2 activation) after DMF IV delivery, pharmacokinetics and transcriptional pharmacodynamic effects were assessed in the plasma, CNS (forebrain and cerebellum) and in peripheral tissues Gejunum, kidney, and spleen) in Sprague-Dawley rats dosed (IV or PO) once daily for 5 consecutive days. Tissues were analyzed by qRT-PCR to assess transcriptional changes in 6 putative Nrf2-target genes that have been previously characterized to respond to DMF/MMF treatment. Scannevin R H, Chollate S, Jung M Y, Shackett M, Patel H, Bista P, Zeng W, RyanS, Yamamoto M, Lukashev M, Rhodes K J. Fumarates promote cytoprotection of central nervous system cells against oxidative stress via the nuclear factor (erythroid-derived 2)-like 2 pathway. J Pharmacal Exp Ther. 2012 April; 341(1):274-84. This study was done to complement the study described further below, which was conducted in wild-type mice (see Section 6.2.2), and to enable future pharmacokinetic, pharmacodynamic and efficacy studies utilizing the IV dosing paradigm.

Summary

The in vivo positron emission tomography (PET) and quantitative whole body autoradiography (QWBA) studies described in Example 1 have demonstrated that intravenous (IV) delivery of dimethyl fumarate (DMF) resulted in a selective distribution of DMF-derived radioactivity into the central nervous system (CNS), whereas per os (PO, oral administration) resulted in distribution predominately localized to the gastrointestinal tract. To evaluate the biological effects of differential biodistribution with IV dosing, pharmacokinetics and pharmacodynamics in the brain and periphery were evaluated comparing DMF administered via IV infusion versus PO in Sprague Dawley rats.

Administering DMF IV (30 mg/kg) resulted in broad distribution of the primary DMF metabolite monomethyl fumarate (MMF) in the tested tissues (forebrain, cerebellum, kidney, spleen, jejunum) when assessed 10 minutes after dosing. Similar broad MMF distribution was observed with DMF administered PO (100 mg/kg) in the same tissues. Interestingly, despite the lower administered dose with IV, the absolute levels of MMF were significantly higher in the brain as compared to PO dosing. This resulted in significantly higher brain-to-plasma ratios with IV as compared to PO dosing. Other tissues did not exhibit this preferential distribution.

In all tested tissues, there was significant modulation of gene expression as assessed by quantitative real-time polymerase chain reaction (qRT-PCR) at 2 and 6 hours after dosing. For individual tested genes, there was differentiation in the temporal aspects of the response (2 versus 6 hours) as well as differentiation in responses from tissue to tissue.

In evaluating the relationship between exposure (measured at 10 minutes) and pharmacodynamic responses (at 2 or 6 hours), there was evidence for a positive correlation in all tested tissues. Thus, administering DMF IV at 30 mg/kg resulted in lower peripheral exposures and pharmacodynamic responses in the periphery as compared to DMF PO at 100 mg/kg. In the brain, the opposite occurred: DMF IV had both higher absolute MMF exposures and transcriptional pharmacodynamic responses as compared to DMF PO.

Material and Methods

All procedures involving animals were performed in accordance with standards established in the Guide for the Care and Use of Laboratory Animals as adopted by the U.S. National Institutes of Health. All animal protocols were approved by the Biogen Idec Inc. Institutional Animal Care and Use Committee; Biogen is accredited by the Association for Assessment and Accreditation of Laboratory Animal Care International. Male Sprague-Dawley rats 8-12 weeks of age were purchased from Charles River Lab in Wilmington, Mass. and given standard water and chow ad libitum throughout the duration of the experiment. Rats were acclimated in the Biogen Idec vivarium for at least 5 days before study initiation.

Compound Formulation and Animal Procedures

Intravenous Formulation and Delivery:

For all IV dosing, a volume of 10 mL/kg was utilized.

Vehicle:

20% (w/v) Captisol solution in H₂O. 1L of vehicle was made by adding 200 g Captisol to 800 mL deionized H₂O (dH₂O).

DMF (30.0 mg/kg):

Source; Cilag Lot 112JS4184; 6 Dec. 2013. DMF was solubilized in vehicle at 3.0 mg/mL. Material was first stirred into the vehicle solution on a stir plate and then sonicated in a room temperature water bath for 5 minutes.

Oral Formulation and Delivery:

For all PO dosing, a volume of 10 mL/kg was utilized.

Vehicle:

0.8% hydroxypropyl methylcellulose (HPMC; Vehicle) E4M Grade in H₂O. 8 g of HPMC powder was added to dH₂O to create 1 L of vehicle solution. Specifically, 500 mL of hot dH₂O (85-90° C.) was added to 8 g of HPMC and stirred with a flat, metal homogenizer until a clear, colorless solution was achieved. The solution was allowed to cool to room temperature, and then the remaining volume of dH₂O added to a final volume of 1 L. Once again, the solution was stirred with a flat, metal homogenizer until homogeneous.

DMF (100 mg/kg):

Source; Cilag Lot 112JS4184; 6 Dec. 2013. DMF was suspended in 0.8% HPMC at 10 mg/mL by agitation with a stir bar, followed by sonication in a room temperature water bath for 5 minutes. The suspension was stirred at 4° C. for the duration of the experiment.

Experimental Design, Dosing Regimen and Tissue Collection

To evaluate and compare the effects of DMF IV and PO dosing, DMF was administered to rats via IV tail vein infusion (n=15) or by oral gavage (n=15) as described below once a day for 5 consecutive days.

| | | |
|---|---|---|
| Group: A, E & I: | PO DMF 100 mg/kg | (n = 15 total, 5/group) |
| Group B, F & J: | IV DMF 30 mg/kg | (n = 15 total, 5/group) |
| Group C & G: | PO Vehicle, 0.8% HPMC | (n = 10 total, 5/group) |
| Group D & H: | IV Vehicle, 20% Captisol only | (n = 10 total, 5/group) |

Experimental Timeline Day 5:

TABLE 3

| Collection | | | Pharmacodynamics |
|---|---|---|---|
| Time | Groups | MMF Exposure | (qRT-PCR) |
| 10 minutes | A, B | plasma, forebrain, cerebellum, | not done |
| | | jejunum, spleen, kidney | |
| 2 hours | C, D, E, F | plasma, forebrain, cerebellum, | forebrain, jejunum, |
| | | jejunum, spleen, kidney | spleen, kidney |
| 6 hours | G, H, I, J | plasma, forebrain, cerebellum, | forebrain, jejunum, |
| | | jejunum, spleen, kidney | spleen, kidney |

On Day 5 of the study 3 time points were evaluated: 10 minutes, 2 hours, and 6 hours after dosing, as illustrated in the timeline. At each time point, 5 animals from each group were sacrificed. At the 2 and 6 hour time points, the tissue samples from groups E, F, I, and J from each animal were split, with half of the tissue being processed for MMF exposure, and half processed for qRT-PCR. Note vehicle groups (C, D, G, and H) were not processed for MMF measurement and only used for qRT-PCR.

Evaluating MMF Exposure:

At 10 minutes, 2 and 6 hours, plasma and tissues (forebrain, cerebellum, jejunum, kidney and spleen) from cohorts of animals (Groups A, B, E, F, I, J) were collected to measure the concentration of MMF in the sample. MMF was quantitated using a non-GLP, but validated LC/MS/MS assay. Statistical comparisons were performed using the non-parametric Mann-Whitney U test.

Evaluating Transcriptional Pharmacodynamic Changes:

Two and 6 hours after dosing, cohorts of animals from Groups C, D, E and F and G, H, I and J, respectively, were sacrificed and tissues (forebrain, cerebellum, jejunum, kidney, and spleen) were collected for RNA analysis by qRT-PCR for expression of prototypical Nrf2 response genes.

MMF Quantification in Plasma and Tissues

As mentioned previously, Sprague-Dawley rats were sacrificed and terminal plasma and tissue samples were collected 10 minutes, 2 and 6 hours after dosing and MMF exposure in plasma, brain, cerebellum, jejunum and kidney was determined as follows. Tissues were snap frozen on dry ice after dissection. Immediately prior to blood collection, 4 μL of 250 mg/mL stock mixture of sodium fluoride (NaF) in water was added to each 100 μL of blood collected in a lithium heparin tube. The stock NaF mixture was prepared on the day of use and kept as a homogenous suspension through continuous stirring on a stir plate. Whole blood was added to the tube, and then samples were inverted several times and stored on wet ice (2° C. to 8° C.). All samples were centrifuged within 30 minutes of collection at 4° C. for 15 minutes at 1500×g (4200 RPM). Plasma was then transferred into pre-chilled tubes, immediately frozen on dry ice and maintained frozen (≤−80° C.) until analysis.

Aliquots (50 μL) of either plasma or tissue homogenate samples were extracted by protein precipitation with acetonitrile containing methylethyl fumarate or −4C₁₃-MMF as the internal standard. For tissue samples, aliquots of homogenization solution (‘blank’ plasma with 12.5 mg/mL of NaF) were added to the tissue samples. The tissue samples were homogenized at 6.5 m/s for 60 seconds on a Fast Prep Tissue Homogenizer prior to protein precipitation. Concentrations of MMF in plasma, forebrain, cerebellum, jejunum, spleen and kidney samples were determined using qualified LC-MS/MS assays in the respective matrices. Data collection and integration was accomplished using an API 5500 triple quadrupole mass spectrometer with a turbo ion spray interface (AB Sciex, Foster City, Calif.), and Analyst software (version 1.6.1). The peak area ratios of MMF relative to its internal standard were used to construct a standard curve using a quadratic regression with a 1/x² weighting. The lower limit of quantitation (LLOQ) was 10 ng/mL for all four assays. The performances of the assays were monitored by the precision and accuracy of the standards and quality control samples.

RNA Extraction and qRT-PCT

Tissue RNA Extraction:

For RNA preparation, frozen tissues were placed in 2 mL RNAse-free 96-well blocks with 1.5 mL QIAzol Lysis Reagent (QIAgen) and a 3.2 mm stainless steel bead (BioSpec Products, Bartlesville, Okla.). Tissues were disrupted for four cycles of 45 seconds in a Mini-Beadbeater (Biospec Products). RNA was extracted in chloroform and the aqueous phase was mixed with an equal volume of 70% ethanol. Extracted RNA was applied to RNeasy 96 plates and purified by the spin method according to the manufacturer's protocol (RNeasy 96 Universal Tissue Protocol, QIAgen, Hilden Germany).

qRT-PCR:

Samples were reverse-transcribed into cDNA according to the manufacturer's protocols (Life Technologies, Carlsbad, Calif.) and analyzed by real-time polymerase chain reaction (qPCR). Rat target gene primers and 6-FAM™ dye-labeled TaqMan® MGB™ probes (Life Technologies) were used. Reactions containing 100 ng of cDNA, 900 nM of each primer, and 250 nM TaqMan probes were cycled on a QuantStudio 12 k-flex system (Life Technologies) once for 10 minutes at 95° C., followed by 40 cycles of 95° C. for 10 seconds and 60° C. for 1 minute. All samples were measured in triplicate with Gapdh as a normalizing gene. Taqman primer/probe sets from Life Technologies included: Akr1b8 (Rn00756513_m1); Gclc (Rn00689046_m1); Hmox1 (Rn01536933_m1); Nqo1 (Rn00566528_m1); Txnrd1 (Rn01503798_m1); Osgin1 (Rn00593303_m1) and Gapdh (Rn01775763_g1). Final analysis was performed using the comparative CT method to calculate fold changes and samples were normalized relative to vehicle controls at each time point. In all graphs, the mean fold-change (±standard deviation) is depicted. Statistical comparisons were performed using ANOVA with Tukey's Multiple Comparisons Test to evaluate differences between vehicle and DMF treated rats at 2 or 6 hours within a given route of administration.

Results

Plasma and Tissue Exposures

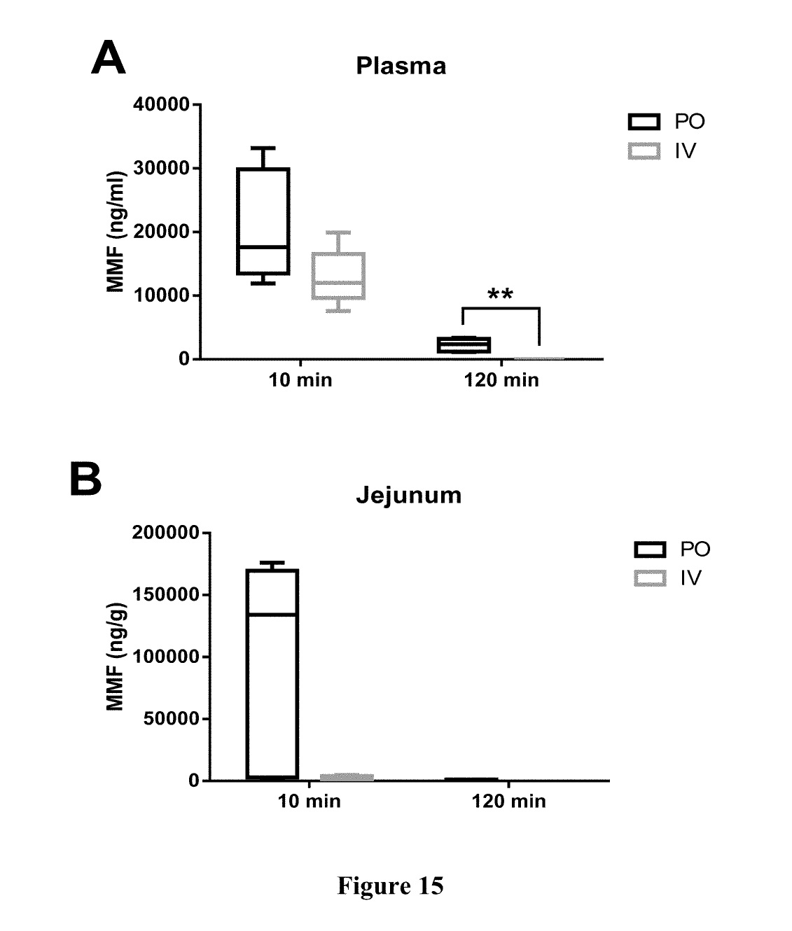

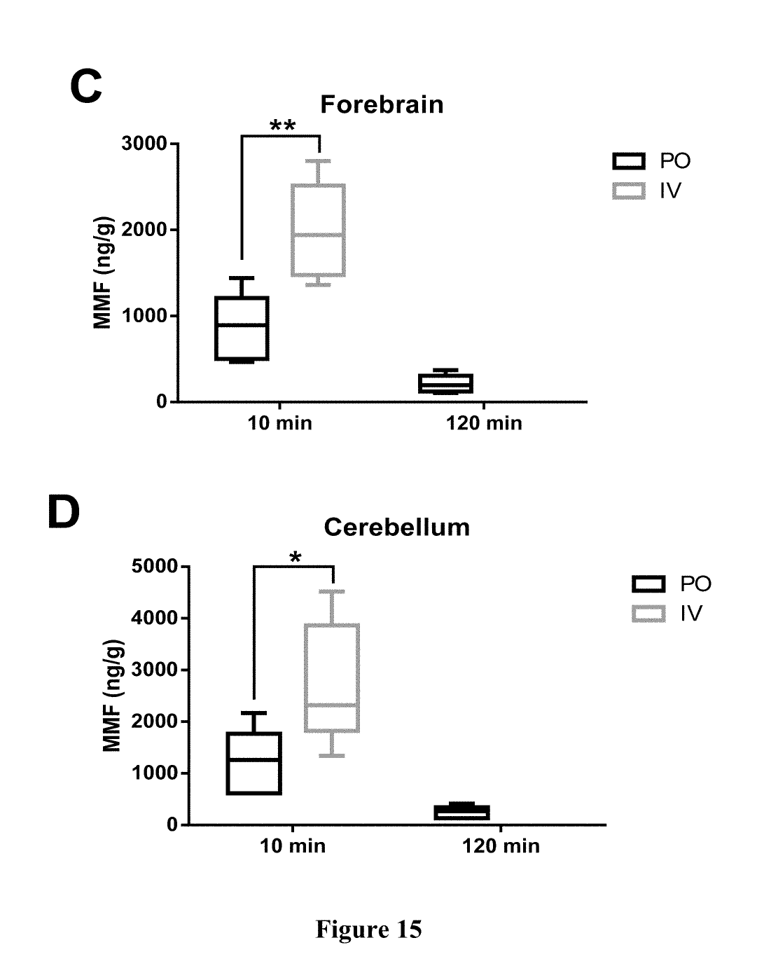

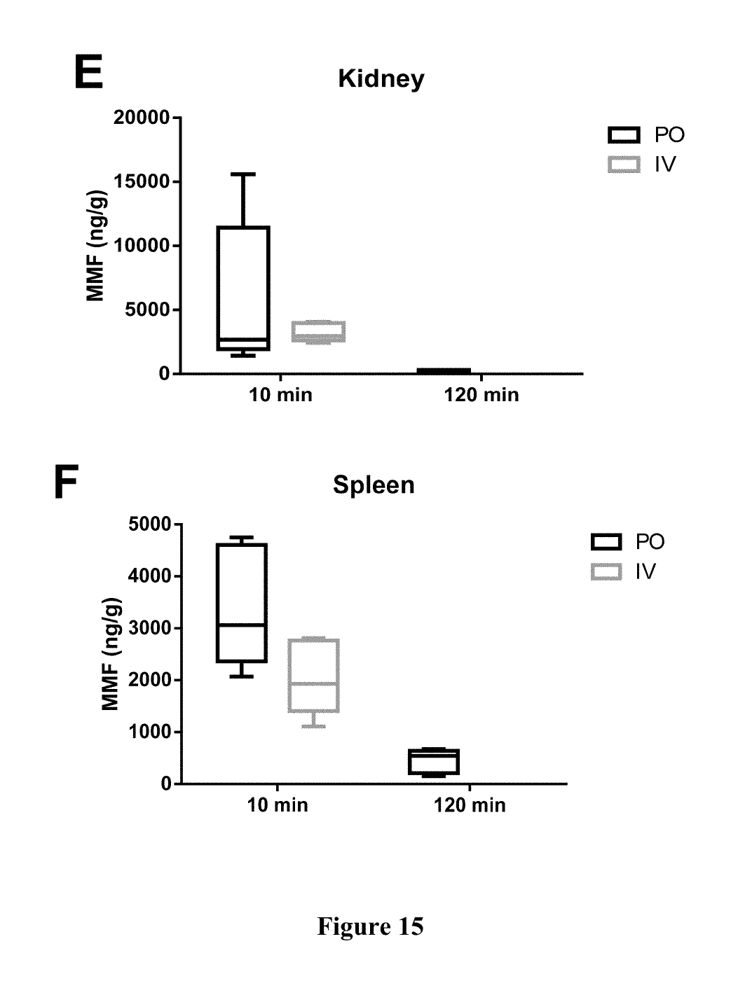

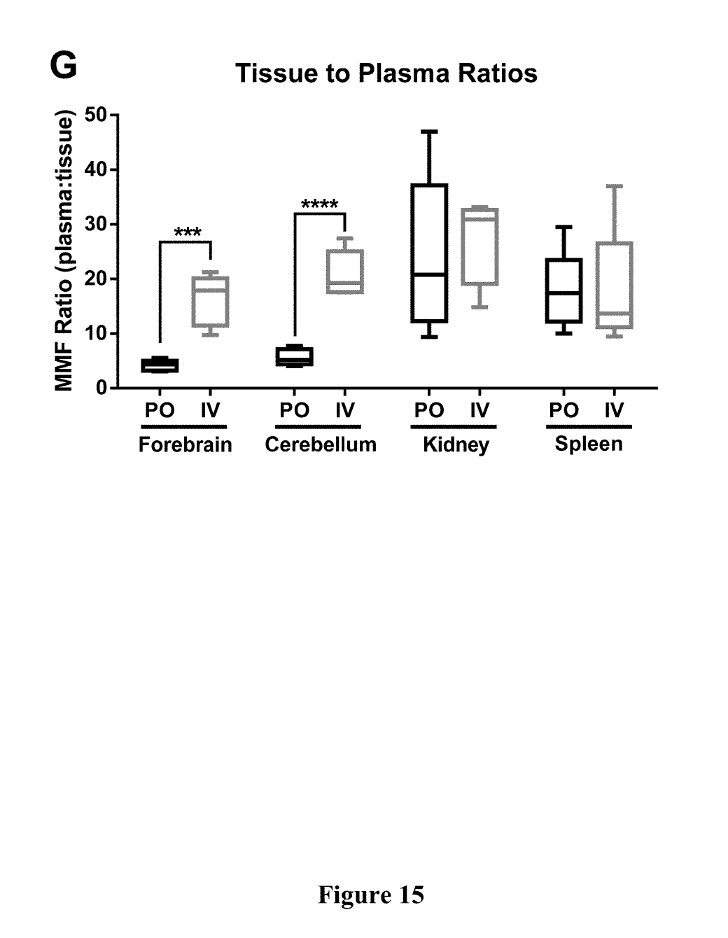

As shown in FIG. 15, 10 minutes after dosing there was robust exposure of MMF in plasma in animals administered DMF by PO (100 mg/kg; 20860±8777 ng/mL, 156.8±66 μM) or IV (30 mg/kg; 12880±4456 ng/mL, 96.8±33.5 μM). Mean plasma MMF levels after IV dosing were 38% lower than those achieved with PO dosing, but this difference was not significant (FIG. 15A). At 2 hours, plasma MMF levels had substantially decreased in animals receiving DMF PO (2248±997 ng/mL, 16.9±7.5 μM) and IV (7.8±2.7 ng/mL, 0.1±0.0 μM), and at this time point the levels after PO dosing were significantly higher than those in animals dosed IV. At the 6 hour time point MMF levels in plasma after DMF PO dosing were detectable in 3 out of 5 animals (30±31 ng/mL, 0.2±0.2 μM); levels in the remaining 2 rats were below the level of quantitation. At the 6 hour time point MMF was not detectable in the plasma samples from rats administered DMF IV.

In forebrain (FIG. 15C), 10 minutes after dosing, MMF levels were significantly higher in animals dosed IV (1984±564 ng/mL, 14.9±4.2 μM) as compared to those dosed PO (862±391 ng/mL, 6.5±2.9 μM). Similar results were observed in the cerebellum (FIG. 15D), with DMF IV achieving significantly higher MMF levels (2740±1195 ng/mL, 20.6±9.0 μM) as compared to DMF PO dosing (1206±644 ng/mL, 9.1±4.8 μM). However, by 2 hours, brain MMF levels from IV dosed animals were below the level of quantitation, whereas those dosed PO still had measurable levels of MMF (forebrain: 211±103 ng/mL, 1.6±0.8 04; cerebellum: 243±118 ng/mL, 1.8±0.9 μM). All exposures provided in text as mean values±standard deviation (n=5). Six hours after dosing, MMF was undetectable in forebrain or cerebellum.

In jejunum, kidney, and spleen at 10 minutes, there were numerical differences in median MMF levels, however these differences were not significant when comparing PO and IV DMF dosing routes (FIG. 15B, E, F). Two hours after DMF PO dosing, MMF levels were markedly decreased, but detectable in the kidney, jejunum and spleen. However, no MMF was detectable in any tissue 2 hours after IV DMF dosing. Six hours after IV or PO DMF dosing, there was no detectable MMF in any tissue.

In comparing the ratio of tissue to plasma MMF exposure, differences were observed between PO and IV dosing and also between tissues. In forebrain and cerebellum, significantly lower penetration of MMF was observed in rats dosed PO as compared to those dosed IV (FIG. 15G, p<0.005 and p<0.0001, respectively). In kidney, the median ratio was numerically higher following IV dosing relative to PO dosing, but this difference was not significant. There were no significant differences in tissue penetration ratios in jejunum, kidney and spleen; however, it should be noted that these samples exhibited considerable variability (jejunum ratio data not shown).

Pharmacodynamic Effects in the CNS and Peripheral Tissues

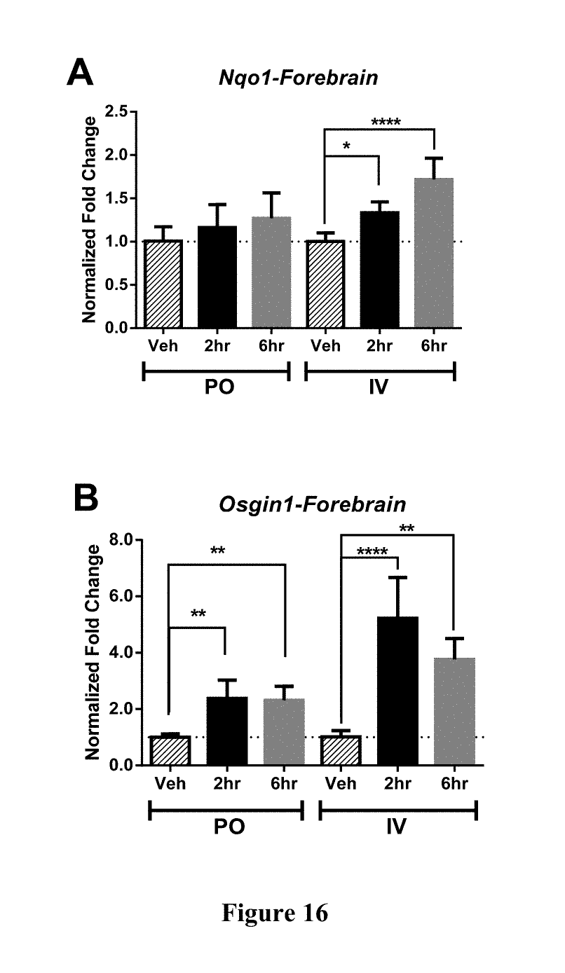

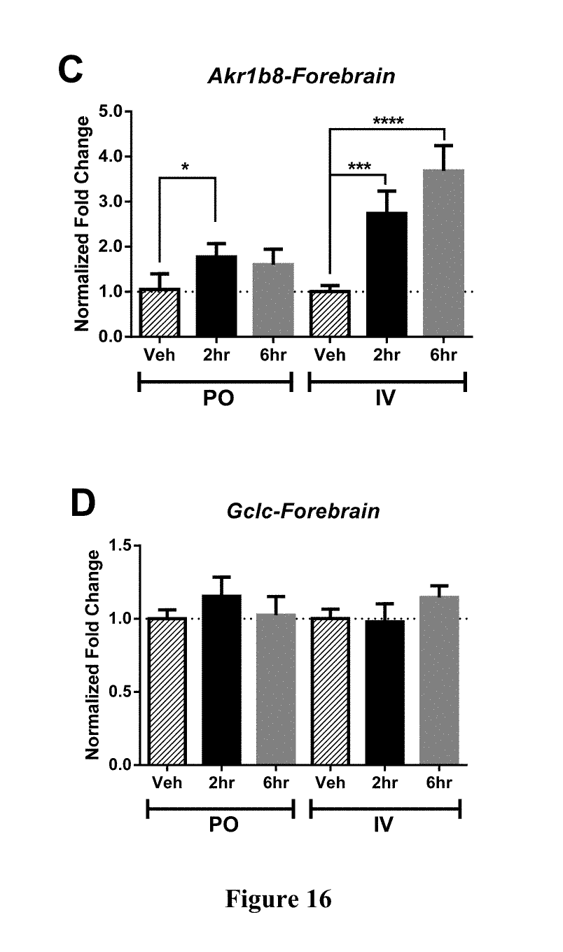

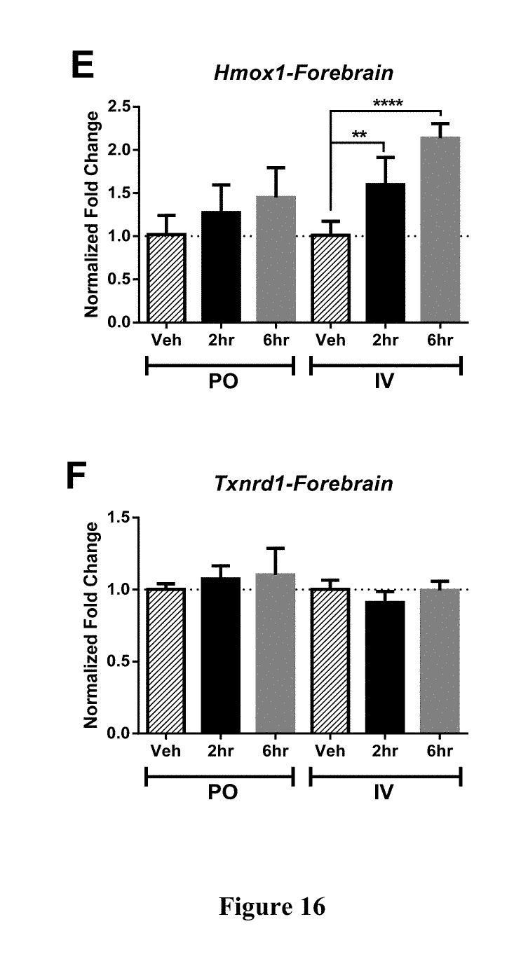

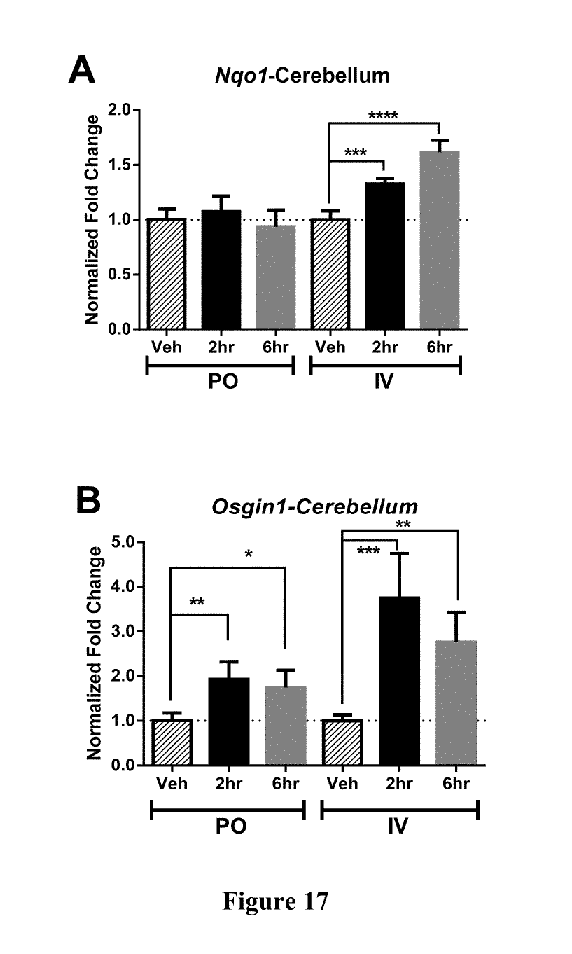

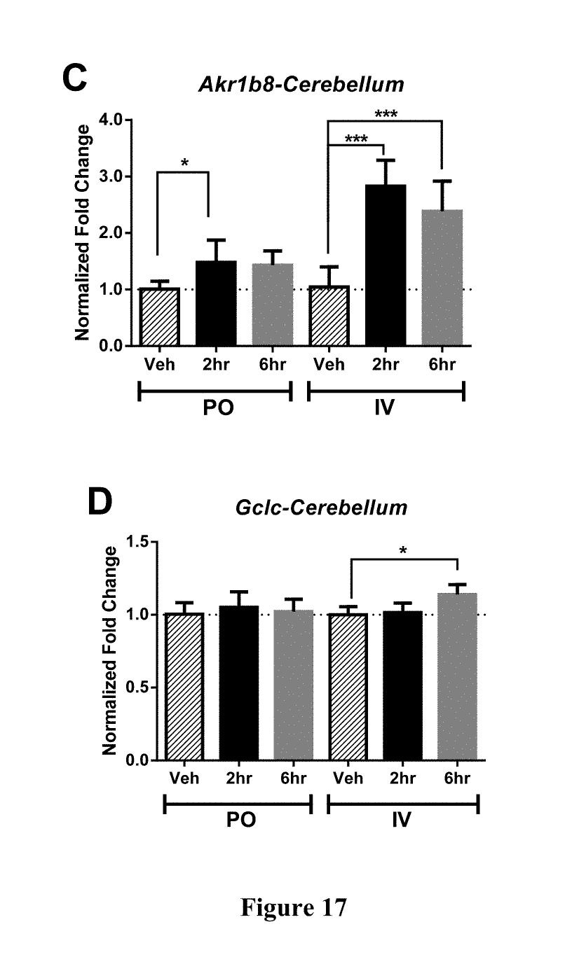

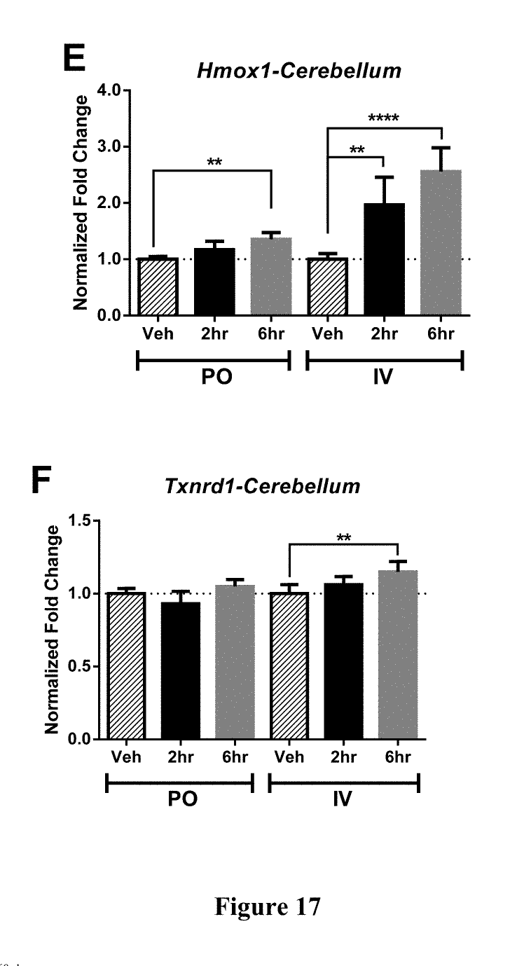

Evaluating Nrf2-dependent transcriptional changes at the 2- and 6-hour time points revealed differences in the qualitative responses between animals treated with DMF PO (100 mg/kg) as compared to those treated with DMF IV (30 mg/kg). Moreover, within the 2 dosing groups there were differences among the collected tissues. In forebrain, IV DMF dosing resulted in significant modulation of Nqo1, Osgin1, Akr1b8, and Hmox1 at both 2 and 6 hours after dosing (FIG. 16). DMF PO dosing led to modulation of only two genes: Osgin1 at 2 and 6 hours and Akr1b8 only at 2 hours. Transcriptional responses in the cerebellum were similar to forebrain (FIG. 17). Following IV DMF dosing, significant modulation was observed for Nqo1, Osgin1, Akr1b8, and Hmox1 at both 2 and 6 hours after dosing. There was also a significant increase in the transcriptional responses of Gclc and Txnrd1 in the cerebellum, but only at the later 6-hour time point. DMF PO dosing led to significant modulation of 3 genes in cerebellar tissue: Osgin1 at 2 and 6 hours, Akr1b8 at 2 hours, and Hmox1 at the 6 hour time point.

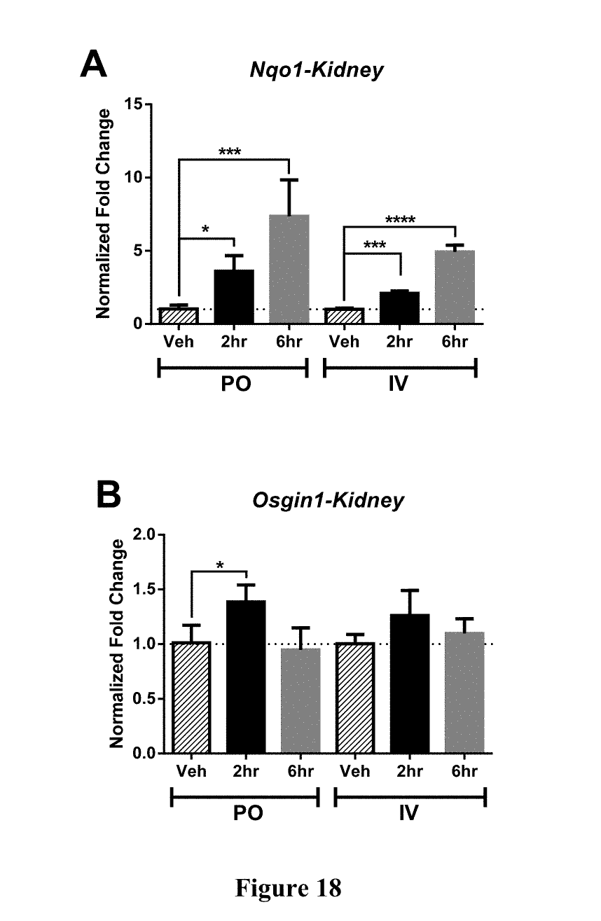

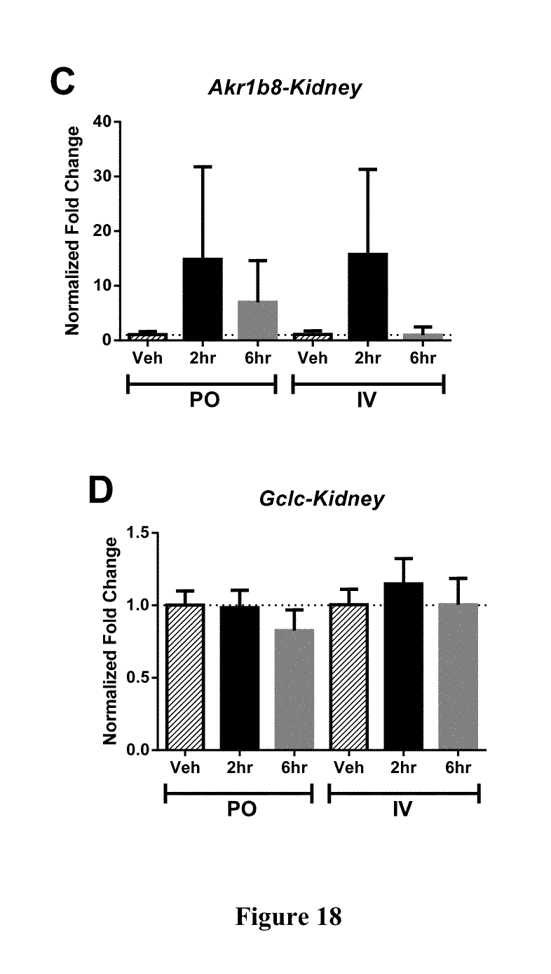

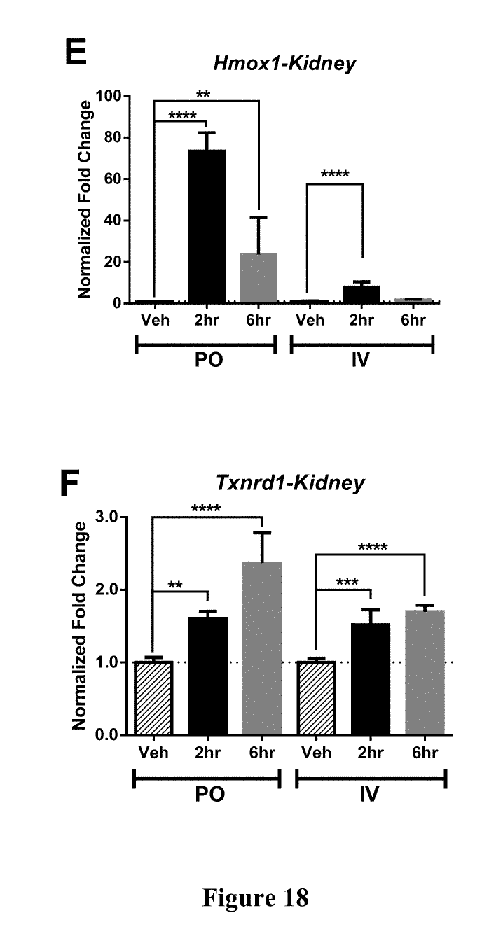

In kidney, DMF IV dosing resulted in significant modulation of Nqo1 and Txnrd1 at 2 and 6 hours, and Hmox1 only at 2 hours (FIG. 18). Following PO dosing of DMF there were significant changes at both 2 and 6 hour time points for Nqo1, Hmox1, and Txnrd1 relative to vehicle controls. Osgin1 was significantly modulated only at the 2 hour time point.

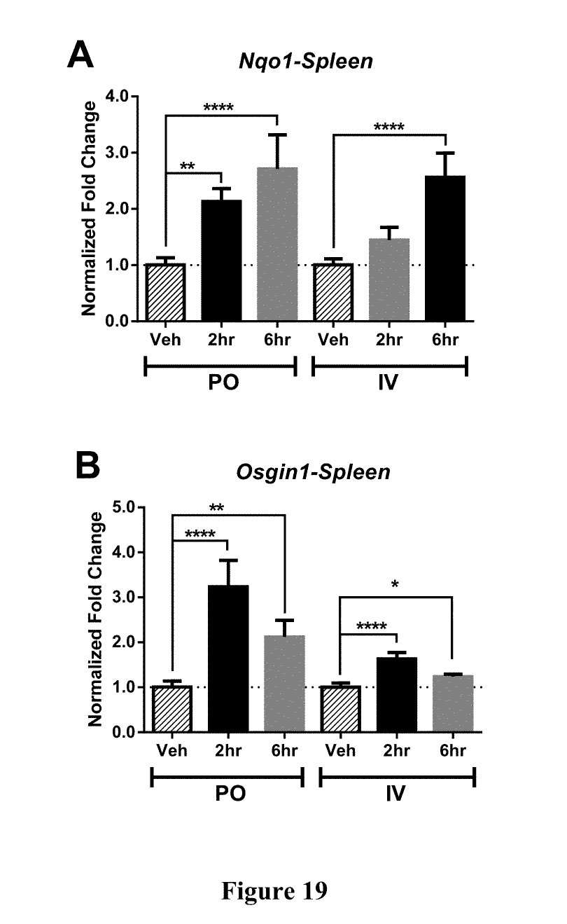

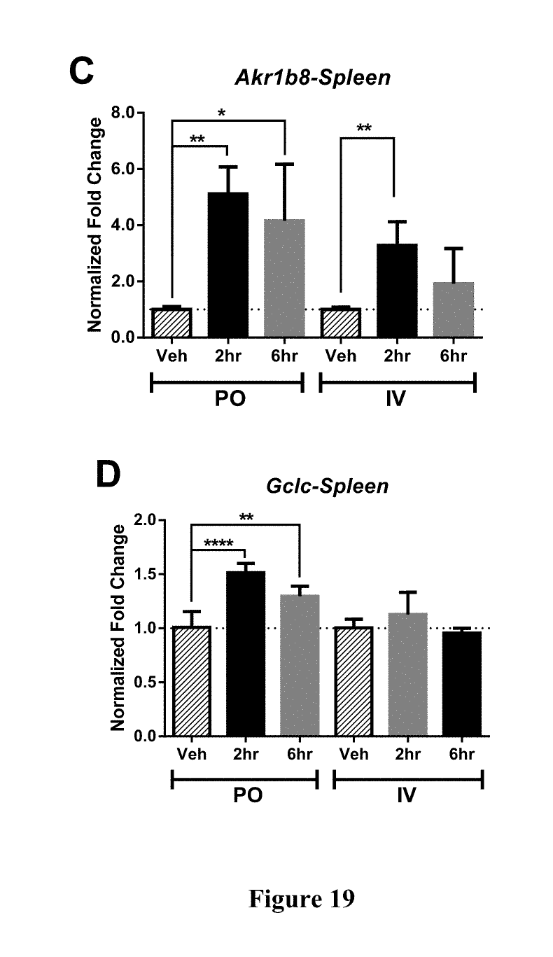

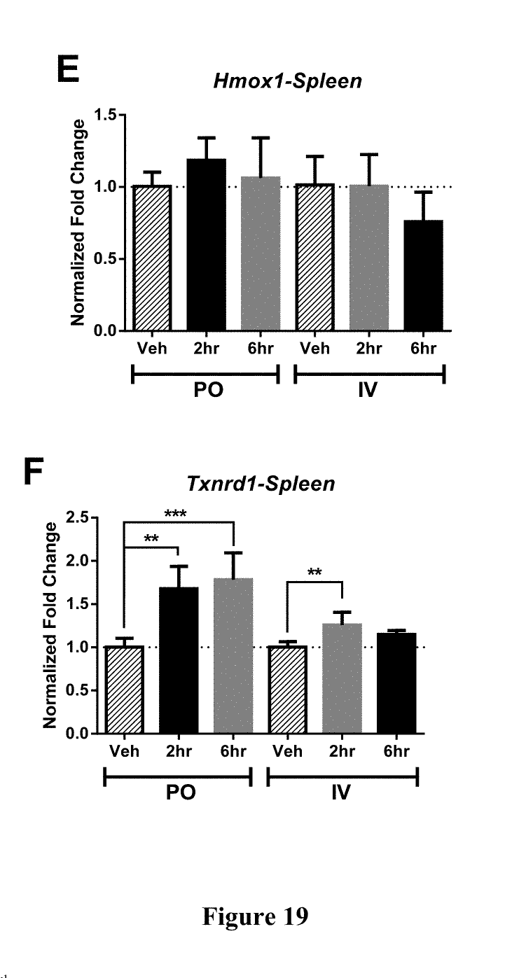

Transcriptional changes in the spleen were similar to changes observed in the kidney, with significant increases in Nqo1, Osgin1, Akr1b8, Gclc, and Txnrd1 at both 2 and 6 hours after DMF PO dosing (FIG. 19). After IV dosing of DMF in the spleen significant transcriptional increases were noted for Osgin1 at both 2 and 6 hour time points, and significant changes in Akr1b8 and Txnrd1 were observed only at the 2 hour time point. A significant change in Nqo1 was also found 6 hours after DMF IV dosing. No changes were observed in Hmox1 expression in spleen after either dosing paradigm.

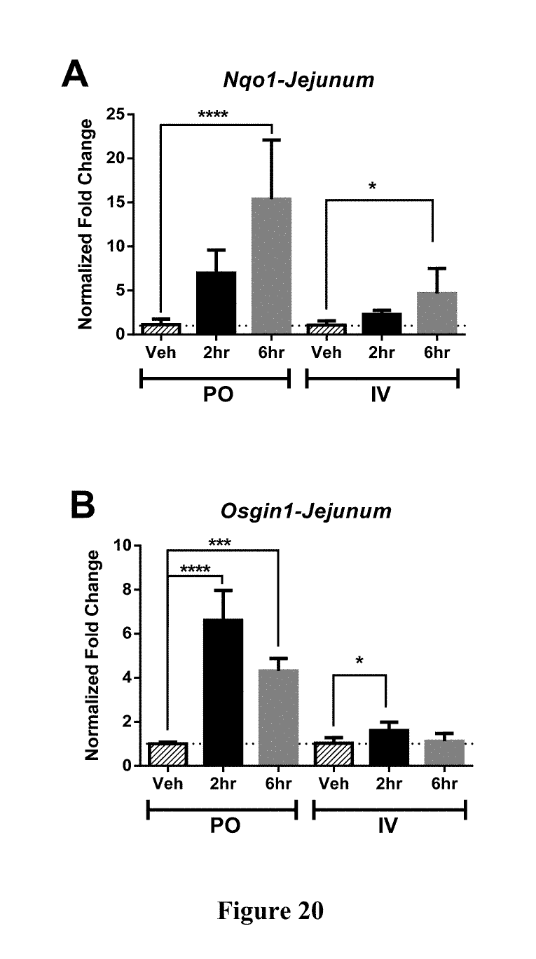

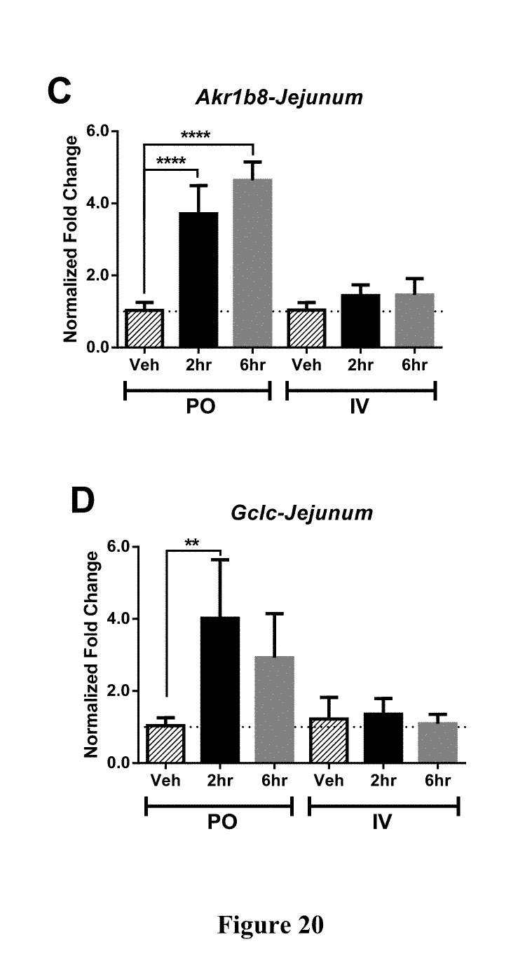

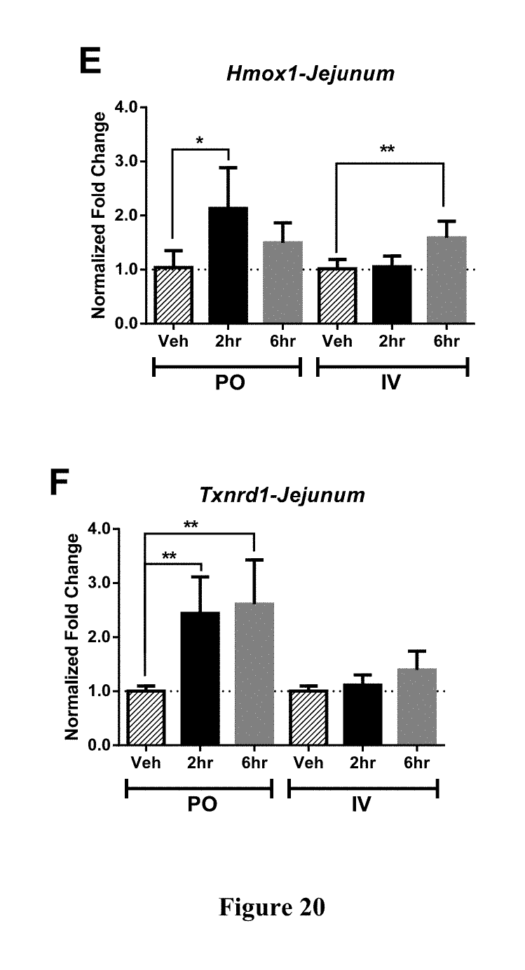

Changes in expression levels in jejunum were qualitatively similar to other peripheral tissues. After DMF IV dosing, significant increases were observed for Nqo1 and Hmox1 at 6 hours, and in Osgin1 expression at 2 hours (FIG. 20). DMF PO dosing resulted in significant increases in Osgin1, Akr1b8, and Txnrd1 at both 2 and 6 hours after dosing. Nqo1 expression was increased after PO DMF dosing only at the 6-hour time point, whereas significant increases in Gclc and Hmox1 were observed at the 2-hour time point.

Exposure-Pharmacodynamic Response Relationships

To evaluate potential relationships between exposure and pharmacodynamic responses, the absolute MMF exposure in several tissues were graphed against fold-changes in gene expression. This approach has inherent limitations; due to the short half-life of MMF and the time required to develop pharmacodynamic responses, these two properties cannot be measured in the same animal. For this analysis the single point exposure at 10 minutes was compared against the pharmacodynamic responses measured in a separate cohort of animals at 2 or 6 hours after dosing.

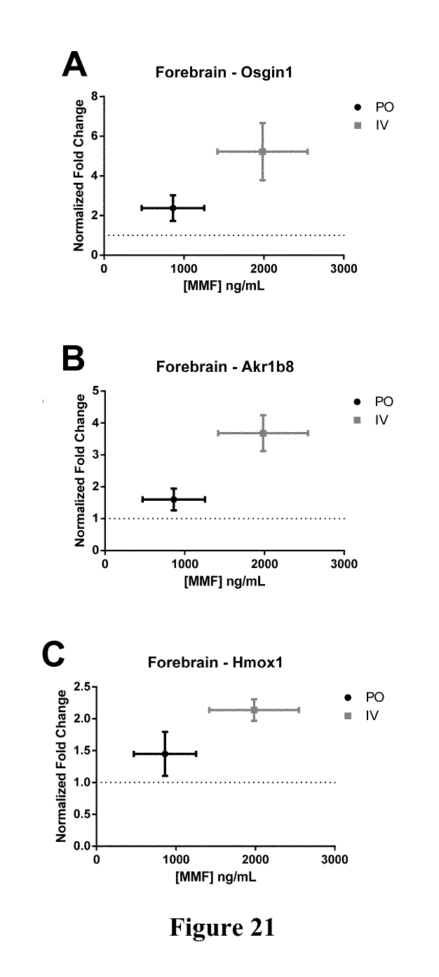

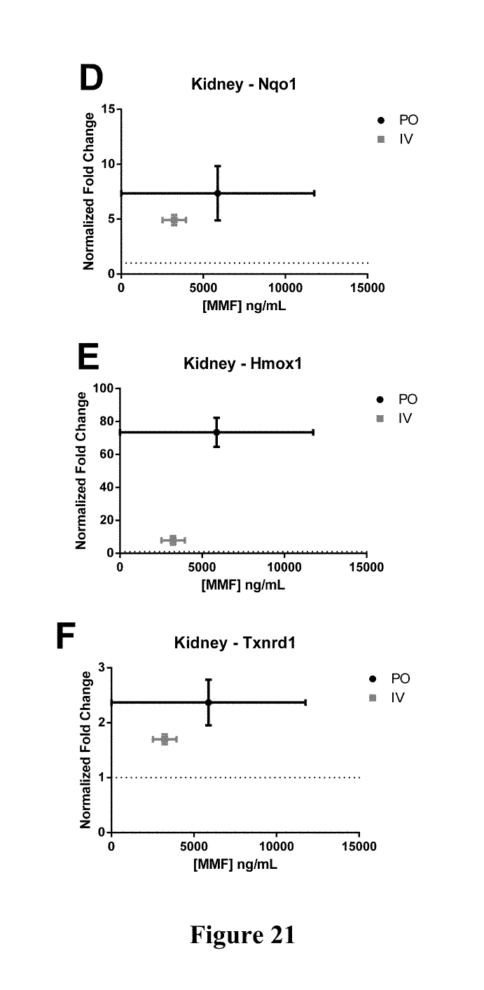

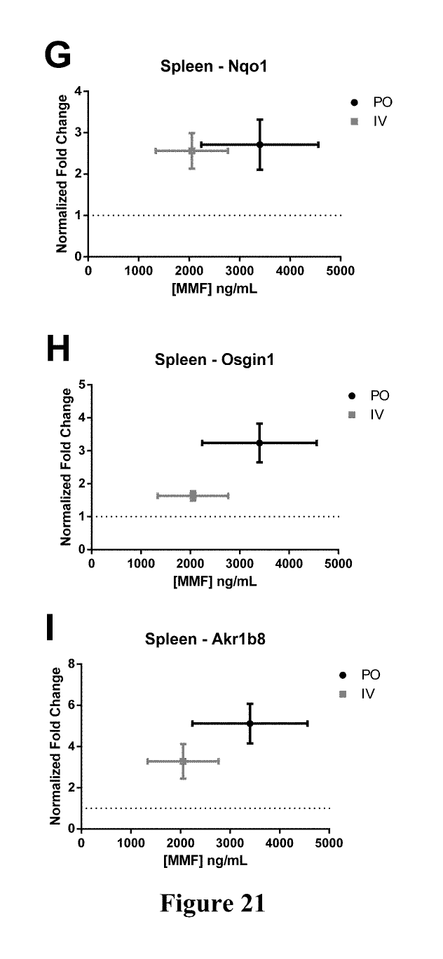

For most evaluated tissues (forebrain, kidney, and spleen) there was a clear positive correlation in MMF exposure and pharmacodynamic response (FIG. 21A-I). Thus in the brain of rats, where dosing of DMF IV resulted in higher exposures, there was a larger magnitude transcriptional response as compared to that seen in animals following DMF PO dosing. In the periphery the opposite was observed: animals given DMF PO had higher exposures and transcriptional changes compared to rats given DMF IV. One exception was in the spleen and the effects on Nqo1 expression (FIG. 21G), where the higher MMF exposure after PO dosing did not result in a larger transcriptional response. Finally, due to the highly variable exposures measured in the jejunum (FIG. 15B), these data were not evaluated for an exposure:response relationship.

Conclusions

Several neurodegenerative diseases have inflammation and oxidative stress as central pathological components. Oral DMF has been shown to activate the Nrf2 pathway in preclinical and clinical studies, and this may mediate, at least in part, the therapeutic effects of treatment in MS (Linker R A, Lee D H, Ryan S, van Dam A M, Conrad R, Bista P, Zeng W, Hronowsky X, Buko A, Chollate S, Ellrichmann G, Bruck W, Dawson K, Goelz S, Wiese S, Scannevin R H, Lukashev M, Gold R. Fumaric acid esters exert neuroprotective effects in neuroinflammation via activation of the Nrf2 antioxidant pathway. Brain. 2011 March; 134(Pt 3):678-92; Scannevin R H, Chollate S, Jung M Y, Shackett M, Patel H, Bista P, Zeng W, Ryan S, Yamamoto M, Lukashev M, Rhodes K J. Fumarates promote cytoprotection of central nervous system cells against oxidative stress via the nuclear factor (erythroid-derived 2)-like 2 pathway. J Pharmacol Exp Ther. 2012 April; 341(1):274-84; L. Amaravadi, S. Gopal, R. Gold, R. J. Fox, A. Mikulskis, M. Lukashev, J. Kong, M. Stephan, K. T. Dawson. Effects of BG-12 on a marker of Nrf2 pathway activation: pharmacodynamic results from the phase 3 DEFINE and CONFIRM studies. Thursday, Oct. 11, 2012, 15:30-17:00. ECTRIMS 2012, Lyon France). Preclinical evidence indicates that increasing MMF exposure (in the periphery and CNS) leads to higher efficacy in neurodegenerative models; however, human dosing cannot be substantially increased beyond current levels due to dose-limiting tolerability with oral administration of the current formulation. Thus, if a mechanism existed to selectively increase relative CNS exposure while maintaining the profile associated with existing peripheral exposures, this might drive enhanced efficacy in neurodegenerative disease through increased CNS cellular resistance to toxic oxidative and inflammatory stress.

The rat imaging data presented in Example 1 demonstrates that DMF administered IV resulted in selective partitioning of DMF or DMF in vivo conversion products into the CNS, while oral delivery produced a distribution more restricted to the GI tract. The results from the studies described hereinabove confirm that the administration of DMF IV resulted in a greater relative partitioning of biologically active MMF into the brain, which resulted in comparable to increased pharmacodynamic responses in the CNS that were achieved at lower total DMF doses, relative to PO dosing, and therefore at lower plasma and peripheral exposure levels.

Preliminary safety of DMF IV dosing was also investigated, and after 5 days of repeated once daily dosing via tail vein injections there were no treatment-related histopathology findings in the tail (data not shown).

### 6.2.2 DMF Administered Orally and Intravenously to C57BL/6 Mice

### List of Abbreviations

TABLE 4

| | |
|---|---|
| MS | Multiple Sclerosis |
| CNS | Central nervous system |
| Nrf2 | Nuclear factor (erythroid-derived 2)-like 2 |
| PK | Pharmacokinetics |
| PD | Pharmacodynamics |
| IV | Intravenous |
| PO | Per os (oral dosing) |
| DMF | Dimethyl fumarate |
| MMF | Monomethyl fumarate |
| HPMC | Hydroxypropyl methylcellulose |
| Akr1b8 | Aldo-keto reductase family 1, member b8 |
| Gclc | Glutamate cysteine ligase, catalytic subunit |
| Hmox1 | Heme oxygenase 1 |
| Nqo1 | NADP(H) dehydrogenase quinone 1 |
| Osgin1 | Oxidative stress induced growth inhibitor 1 |
| CBC | Complete blood count |
| ANOVA | Analysis of variance |
| dH₂O | Deionized water |

Introduction

The PET and QWBA studies in mouse of Example 1 show that administering ¹¹C or ¹⁴C-labeled DMF intravenously (IV) resulted in a rapid and selective partitioning of radioactivity into the CNS, whereas PO dosing produced a more peripheral distribution confined predominately to the gastrointestinal (GI) tract. These imaging data are able to characterize the anatomical location of radioactivity, but the molecular identity of the radioactive species is unknown and could be DMF, MMF or other fumarate conjugates or metabolites. To determine if the observed imaging results correlated with meaningful biological effects (e.g., Nrf2 activation) after DMF IV delivery, pharmacokinetics and transcriptional pharmacodynamic effects were assessed in plasma, CNS and peripheral tissues.

Excessive oxidative stress in the CNS has been identified as a pathological factor in several neurodegenerative disorders, and therapeutic strategies to neutralize this toxic stress could have utility in a number of diseases. If DMF delivered IV could enhance Nrf2-related transcriptional responses and subsequent pro-survival pathways in the CNS beyond that which can be achieved with oral administration, the IV route of administration could provide enhanced beneficial effects in CNS-related diseases.

Summary

The in vivo positron emission tomography (PET) and quantitative whole body autoradiography (QWBA) studies presented in Example 1 demonstrate that intravenous (IV) delivery of dimethyl fumarate (DMF) resulted in a selective distribution of DMF-derived radioactivity into the central nervous system (CNS), whereas per os (PO, oral administration) resulted in distribution predominately localized within the gastrointestinal tract. To evaluate the potential biological effects of differential biodistribution with IV dosing, pharmacokinetic and pharmacodynamic properties in plasma, brain and peripheral tissues were evaluated comparing DMF administered IV versus PO in mice.

Both PO and IV routes of administration resulted in robust monomethyl fumarate (MMF, the bioactive primary metabolite of DMF) exposures in the plasma, brain and peripheral tissues. The higher administered dose of DMF PO (100 mg/kg) resulted in significantly higher plasma and tissue concentrations of MMF relative to DMF administered IV (17.5 or 30 mg/kg). A focused comparison of the two IV doses revealed an exposure dose response in plasma, kidney, jejunum and spleen, but not in the brain. Comparing tissue penetration, the brain-to-plasma ratio of MMF was significantly enhanced with IV dosing as compared to PO, which was consistent with the findings from imaging studies.

Significant pharmacodynamic transcriptional effects were observed in the brain and peripheral tissues after both PO and IV routes of DMF administration. Despite the lower absolute MMF levels in the brain after IV as compared to PO DMF dosing, transcriptional responses were of similar magnitude, suggesting the pharmacodynamic responses in brain were not simply correlated to MMF levels. There was a positive correlation in peripheral tissues between MMF exposure and transcriptional responses.

In comparing the effects of IV DMF administration as compared to IV MMF administration, the MMF brain-to-plasma ratio was significantly higher after IV DMF as compared to IV MMF administration, and furthermore only IV DMF dosing produced significant increases in pharmacodynamic responses in the brain. Both DMF and MMF produced significant transcriptional pharmacodynamic changes in peripheral tissues.

The persistence of pharmacodynamic changes was also evaluated, and the responses after 5 consecutive days of once daily IV DMF dosing were similar to the responses observed after a single dose. Pilot histopathology was also performed in sections of the tail after 5 consecutive days of dosing, and no frank pathology was identified (data not presented).

Blood cell profiles were evaluated after single and 5 once-daily repeated IV doses to assess changes in circulating cell populations. Significant effects on several parameters were observed after a single dose, but effects of vehicle alone were similar to groups receiving DMF. After 5 once-daily IV DMF doses, there was a decrease in lymphocytes and monocytes that was significantly different from mice receiving vehicle.

In summary, DMF when administered via IV infusion produced significant pharmacodynamic effects in the brain. These transcriptional changes were similar to effects that were induced after PO dosing; however, the PO dose was 3.3-fold higher in total level, which resulted in higher relative exposures and pharmacodynamic effects in peripheral tissues such as kidney and jejunum. If one postulates a maximum safe systemic exposure of DMF/MMF, similar to that which is currently being used for multiple sclerosis (MS), if a similar systemic exposure is achieved by IV infusion, there is the potential for enhanced exposure and transcriptional effects in the brain which may confer an added benefit in neurodegenerative disease.

Materials and Methods

All procedures involving animals were performed in accordance with standards established in the Guide for the Care and Use of Laboratory Animals as adopted by the U.S. National Institutes of Health. All animal protocols were approved by the Biogen Idec Inc. Institutional Animal Care and Use Committee, which is accredited by the Association for Assessment and Accreditation of Laboratory Animal Care International. Female C57BL/6 mice at 11-13 weeks of age were purchased from Jackson Laboratories (Bar Harbor, Me.), and provided standard water and chow ad libitum throughout the duration of the experiment. All mice were acclimated at least one week in the Biogen Idec vivarium before study initiation.

Compound Formulation and Animal Procedures

Intravenous Formulation and Delivery:

Vehicle:

20% Captisol °: Every 1 L of vehicle was made with 200 grams (g) Captisol in deionized water (dH₂O). Dose volume was 10 mL/kg into tail vein.

DMF (17.5 mg/kg and 30 mg/kg):

DMF (Cilag Lot I12JS4184; 6 Dec. 2013) was solubilized at 1.75 mg/mL or 3 mg/mL via stirring with a stir bar, followed by sonication in a room temperature water bath for 5-10 minutes. The solutions were stirred with a stir bar at room temperature for the duration of the experiment. Dose volume was 10 mL/kg into tail vein.

MMF (27.08 mg/kg):

MMF (Sigma 651419) was solubilized at 2.708 mg/mL through stirring with a stir bar, followed by sonication in a water bath at room temperature for 5 minutes. The solution was stirred with a stir bar at room temperature for the duration of the experiment. MMF dose was adjusted to compensate for a lower molecular weight as compared with DMF to ensure equivalent “fumarate” was delivered between the two groups. Dose volume was 10 mL/kg into tail vein.

Oral Formulation and Delivery:

Vehicle:

0.8% Hydroxypropyl Methylcellulose (HPMC) E4M Grade: 8 g of HPMC powder was added to each liter of dH₂O. Initially, 500 mL of hot dH₂O (85-90° C.) were added to 8 g of HPMC and was stirred with a flat, metal homogenizer until a clear, colorless solution was achieved. The solution was allowed to cool to room temperature, and then dH₂O (room temperature) was added until a final volume of 1 L was achieved. Once again, the solution was stirred with a flat, metal homogenizer until homogeneous. Dose volume was 10 mL/kg.

DMF (100 mg/kg):

DMF (Cilag Lot I12JS4184; 6 Dec. 2013) was suspended in 0.8% HPMC at 10 mg/mL through stirring with a stir bar, followed by sonication in a room temperature water bath for 5-10 minutes. The suspension was stirred with stir bar at 4° C. for the duration of the experiment. Dose volume was 10 mL/kg.

MMF Quantification in Plasma and Tissues

Aliquots (25 μL) of either plasma or tissue homogenate samples were extracted by protein precipitation with acetonitrile containing 4C₁₃-MMF as the internal standard. For tissue samples, aliquots of homogenization solution (plasma with 12.5 mg/mL of NaF) were added to the tissue samples. The tissue samples were homogenized at 6.5 m/s for 60 seconds on a Fast Prep Tissue Homogenizer prior to protein precipitation. Concentrations of MMF in plasma, brain, jejunum and kidney samples were determined using qualified LC-MS/MS assays in the respective matrices. Data collections and integrations were accomplished using an API 5500 triple quadrupole mass spectrometer with a turbo ion spray interface (AB Sciex, Foster City, Calif.), and Analyst software (version 1.6.1). The peak area ratios of MMF relative to its internal standard were used to construct a standard curve using a quadratic regression with a 1/x² weighting. The lower limits of quantitation (LLOQ) were 1 ng/mL for the three tissue assays and 10 ng/mL for the plasma assay, respectively. The performances of the assays were monitored by the precision and accuracy of the standards and quality control samples.

RNA Extraction and Quantitative PCR

Tissue RNA Extraction:

For RNA preparation, frozen tissues were placed in 2 mL RNAse-free 96-well blocks with 1.5 mL QIAzol Lysis Reagent (QIAgen) and a 3.2 mm stainless steel bead (BioSpec Products, Bartlesville, Okla.). Tissues were disrupted for four cycles of 45 seconds in a Mini-Beadbeater (Biospec Products). RNA was extracted with chloroform and the aqueous phase was mixed with an equal volume of 70% ethanol. Extracted RNA was applied to RNeasy 96 plates and purified by the spin method according to the manufacturer's protocol (RNeasy 96 Universal Tissue Protocol, QIAgen, Hilden Germany).

Quantitative Real-Time Polymerase Chain Reaction (qRT-PCR):

Samples were reverse-transcribed into cDNA according to the manufacturer protocols using the High Capacity cDNA Reverse Transcription Kit (Life Technologies, Carlsbad, Calif.) and analyzed by qRT-PCR. Mouse target gene primers and 6-FAM™ dye-labeled TaqMan® MGB™ probes (Life Technologies) were used. Reactions containing 100 ng of cDNA, 900 nM of each primer, and 250 nM TaqMan probes were cycled on a QuantStudio 12 k-flex system (Life Technologies) once for 10 minutes at 95° C., followed by 40 cycles of 95° C. for 10 seconds and 60° C. for 1 minute. All samples were measured in triplicate with Gapdh as a normalizing gene. Taqman primer/probe sets from Life Technologies included: Akr1b8 (Mm00484314_m1); Gapdh (Mm03302249_g1); Gclc (Mm00802655_m1); Hmox1 (Mm00516005_m1); Nqo1 (Mm01253561_m1); and Osgin1 (Mm00660947_m1). Comparative C_{T} method was used to calculate fold changes and samples were normalized relative to time-matched vehicle control.

Complete Blood Count (CBC)

Whole blood samples (250 μL) were collected in EDTA tubes at indicated time points after dosing, and were maintained at 4° C. until analysis for CBC with differential at Charles River Laboratories (analyzed within 24-36 hours from time of collection).

Tail Histopathology for DMF IV Multi-Dose Tolerability

Mouse tails were collected from DMF and vehicle groups upon study termination (Day 5) for investigation of potential injection site reaction. Tissue samples were preserved in 10% neutral buffered formalin (NBF) and then transferred to the Translational Pathology Laboratory for processing into paraffin blocks, slide preparation, and hematoxylin and eosin staining. Histopathology evaluation of the stained slides was performed by the Biogen Idec Comparative Pathology department (data not provided herein).

Results

Pharmacokinetics and Pharmacodynamics after Intravenous Administration of DMF (Study 1)

To evaluate the effects of DMF IV administration, DMF was formulated in 20% Captisol and infused into mice via tail vein injection and compared to mice receiving DMF with PO administration. MMF exposure levels were evaluated 10 minutes and 2 hours after dosing, while pharmacodynamic transcriptional responses were analyzed at 2 hours post-dose. The PO dose was selected based on previous studies demonstrating 100 mg/kg is an efficacious dose in multiple mouse models, and the high dose for the IV group (30 mg/kg) was selected based on maximum feasible dose given the solubility of DMF in the vehicle. DMF IV (17.5 mg/kg) was selected as a lower IV dose.

| | |
|---|---|
| Group 1: IV Vehicle | (total n = 8, 4/time point) |
| Group 2: IV DMF 17.5 mg/kg | (total n = 8, 4/time point) |
| Group 3: IV DMF 30 mg/kg | (total n = 8, 4/time point) |
| Group 4: PO Vehicle | (total n = 8, 4/time point) |
| Group 5: PO DMF 100 mg/kg | (total n = 8, 4/time point) |

TABLE 5

| Time of | | |
|---|---|---|
| Collection | MMF Exposure | Pharmacodynamics |
| 10 minutes | plasma, brain, jejunum, kidney | not done |
| 2 hours | plasma, brain, jejunum, kidney | brain, jejunum, kidney |
| | | (qRT-PCR) |

At the 10-minute time point, 4 animals from each group were sacrificed, with plasma and tissues (brain, jejunum and kidney) collected to determine MMF exposure. Two hours after dosing, the remaining 4 animals were sacrificed, and plasma and tissues were collected. Tissue samples from the 2-hour time point were divided, with half of each sample used for MMF exposure analysis, and half for RNA extraction and qRT-PCR to evaluate the expression of Nrf2 target genes.

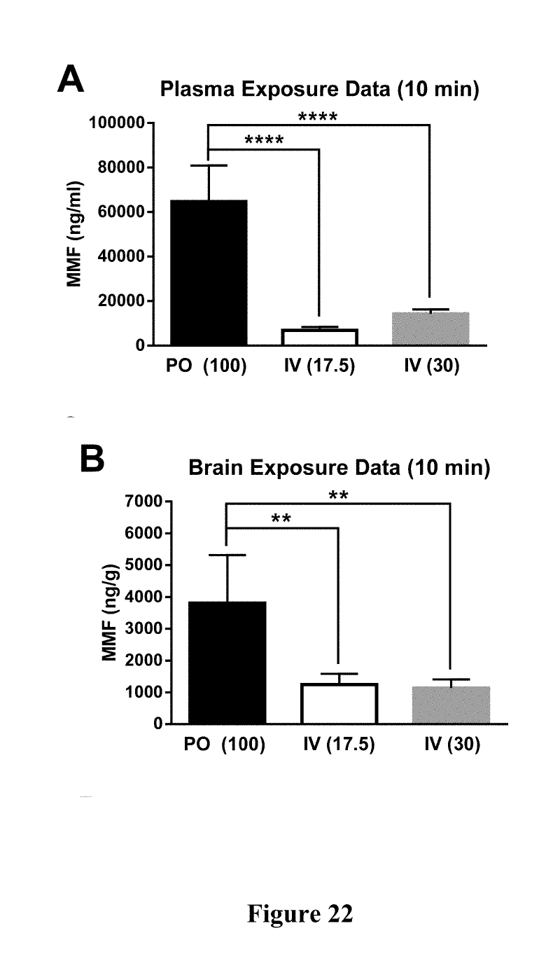

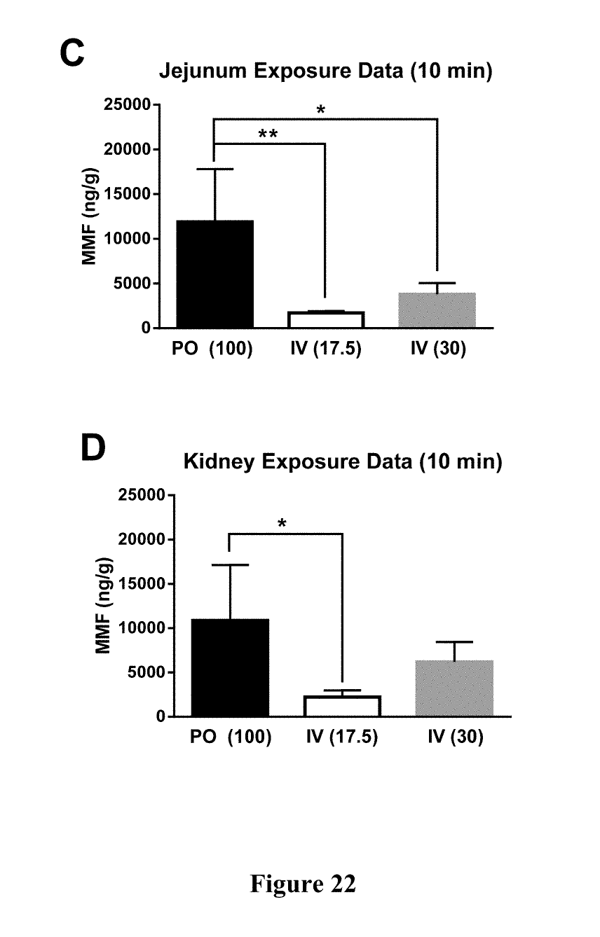

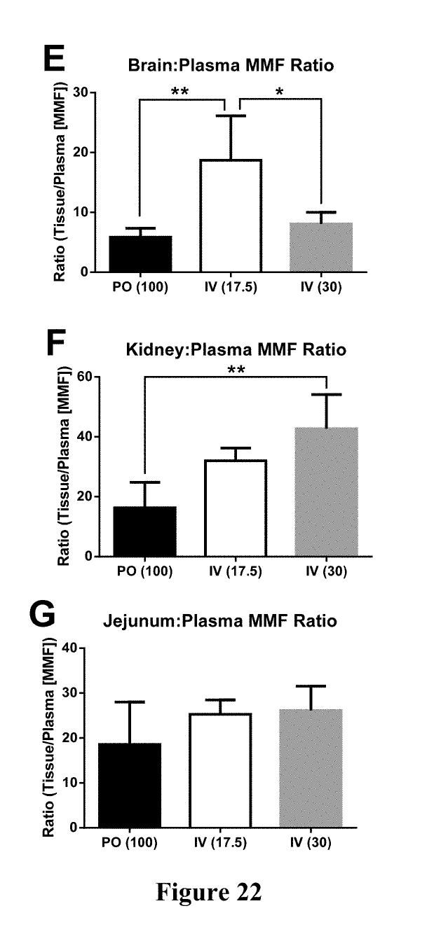

At 10 minutes post-dosing, a robust exposure of MMF in plasma was measured after DMF 100 mg/kg PO administration (64725±16147 ng/mL; 498±124 μM), whereas the plasma levels of MMF after IV dosing of DMF at 17.5 mg/kg (6910±1459 ng/mL; 53±11 μM) or 30 mg/kg (14275±1994 ng/mL; 110±15 μM) were significantly lower (FIG. 22A). In brain, kidney, and jejunum, MMF levels were also significantly higher with PO dosing as compared to DMF IV (17.5) (FIG. 22B, C, D). MMF levels were also significantly higher in the DMF PO group as compared to DMF IV (30 mg/kg) in brain and jejunum, but not kidney. There were no significant differences in plasma or any tissue between DMF IV groups. At 2 hours, there was no detectable MMF in plasma from mice that had received IV DMF, and the plasma MMF levels in the mice receiving PO DMF had markedly decreased (307±431 ng/ml; 2±3 μM). There was no detectable MMF in any tissue at 2 hours.

In the plasma, jejunum and kidney, MMF levels were on average 43% lower in the IV 17.5 group as compared to the IV 30 mg/kg group, however, as denoted above these differences were not significant based on the pre-specified ANOVA multiple comparison analysis, which included the plasma group. An alternative post-hoc analysis directly comparing only the two IV dose groups (t-test) revealed these differences were significant (p<0.05), which was consistent with the difference in the two dose levels and indicated there was an exposure dose-response in these tissues after IV dosing. However, there were no significant differences in MMF levels in brain between the two DMF IV dose groups, which were within 10% of each other. This may indicate there was a saturable mechanism for MMF trafficking into the brain, but it is important to note that this analysis only measures a single DMF metabolite, MMF, at a single time point, and other DMF-derived fumarate species may be present at concentrations that vary between the IV dose levels and possibly with different pharmacokinetics.

In comparing the ratio of tissue to plasma MMF exposure, differences were observed between PO and IV dosing, IV dose levels, and also between tissues. In brain, significantly higher penetration was observed with IV dosing at 17.5 mg/kg as compared to PO dosing (18.7±7.4 versus 5.9±1.5, respectively; p<0.01) (FIG. 22E). However, the ratio for the 30 mg/kg IV dose (8.1±1.9) was not significantly different than the PO dose. In kidney, the ratio was higher for both IV doses (17.5 mg/kg, 32.0±4.3 and 30 mg/kg, 42.7±11.4) as compared to PO (16.3±8.5), and was significantly different (p<0.05) for the higher IV dose (FIG. 22F). There were no significant differences in tissue penetration ratios amongst dose groups in jejunum (FIG. 22G).

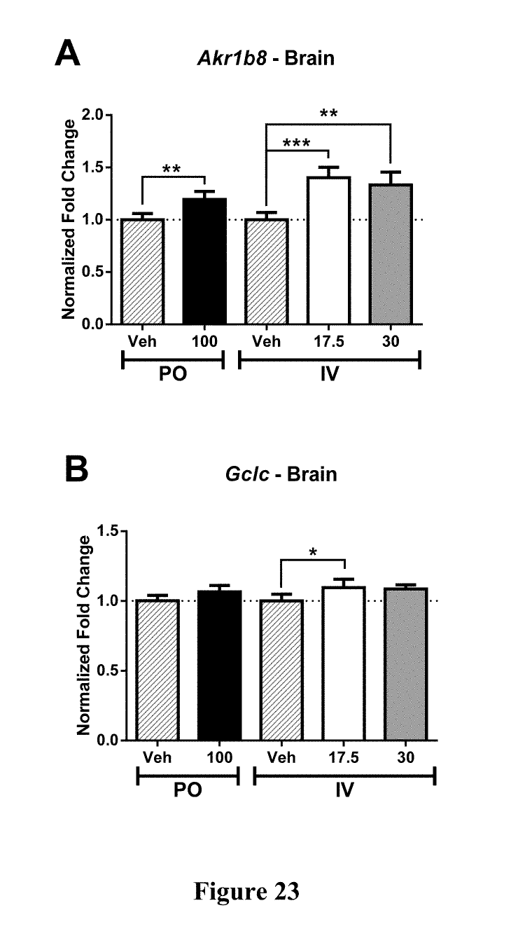

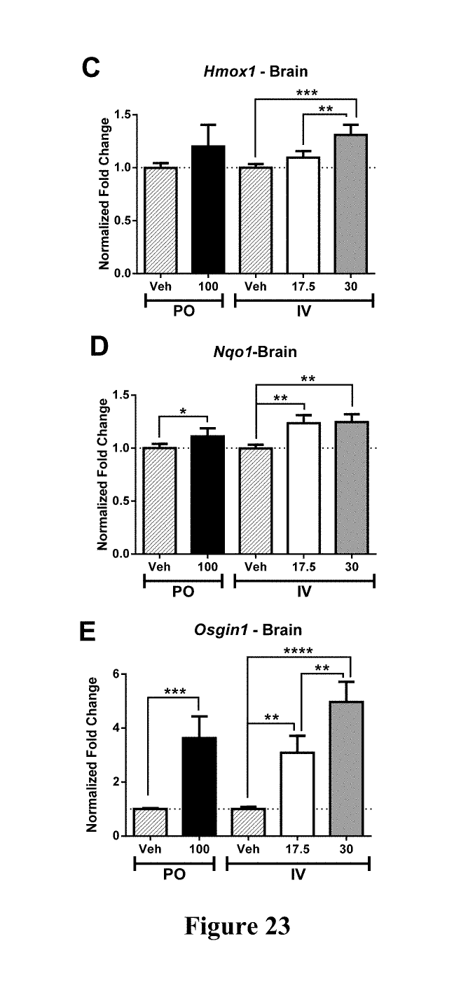

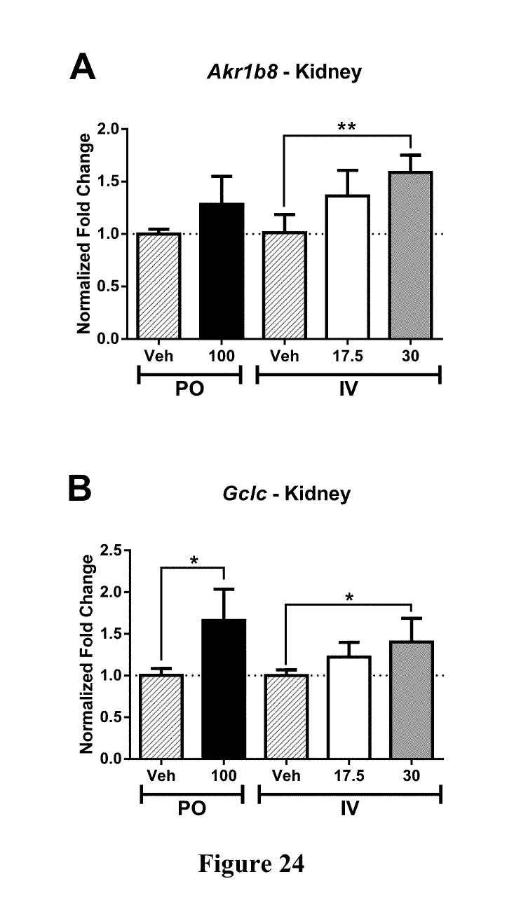

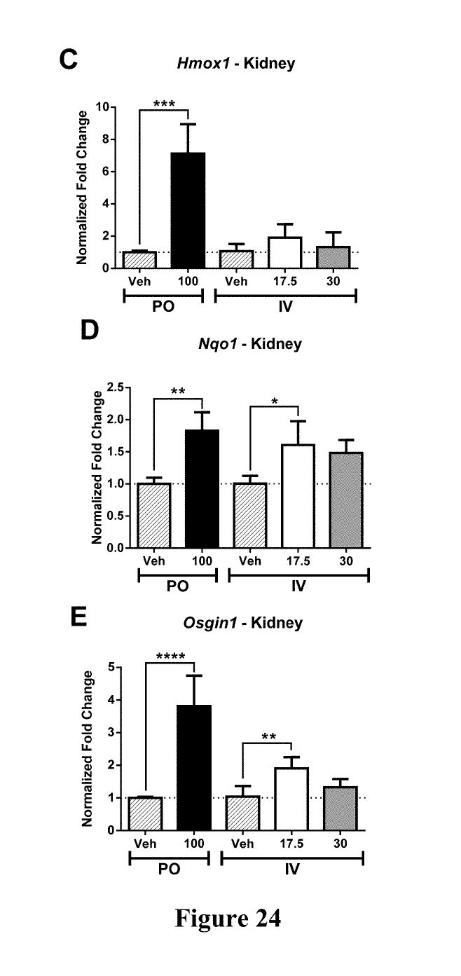

Evaluating the pharmacodynamic changes at the 2-hour time point revealed differences in the responses between the DMF PO versus IV routes of administration and also between tissues. In brain after PO dosing, 3 genes were found to be significantly modulated relative to vehicle control: Akr1b8 (1.2±0.1), Nqo1 (1.1±0.1), and Osgin1 (3.6±0.8) (FIG. 23A-E, Table 7 below). After IV dosing at 17.5 mg/kg, significant modulation was observed for Akr1b8 (1.4±0.1), Gclc (1.1±0.1), Nqo1 (1.2±0.1), and Osgin1 (3.1±0.6) and after IV dosing at 30 mg/kg, significant modulation was observed for Akr1b8 (1.3±0.1), Hmox1 (1.3±0.1), Nqo1 (1.2±0.1), and Osgin1 (5.0±0.7). A dose-response was observed for 2 genes, with significant differences (p<0.01) observed between DMF IV 17.5 versus 30 mg/kg dosing for Hmox1 and Osgin1 (FIG. 23C, E).

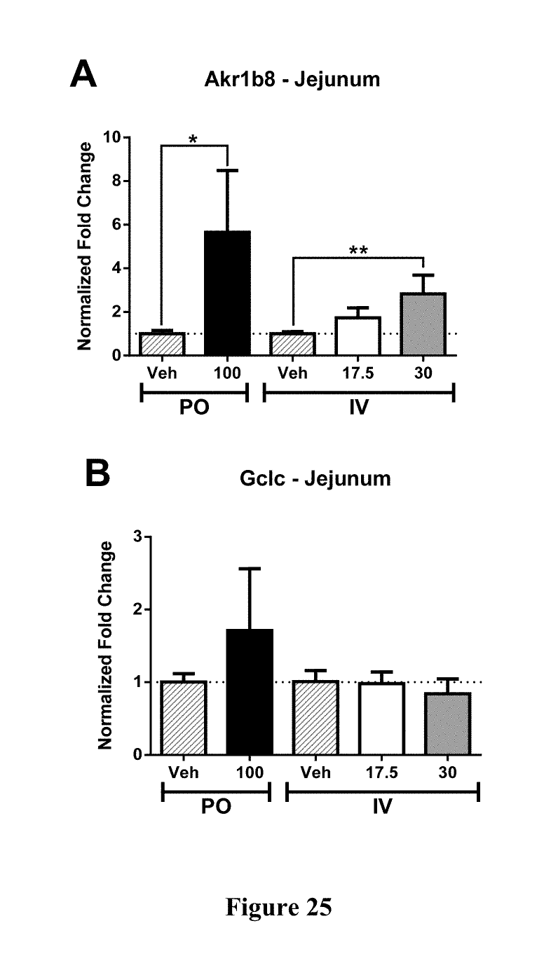

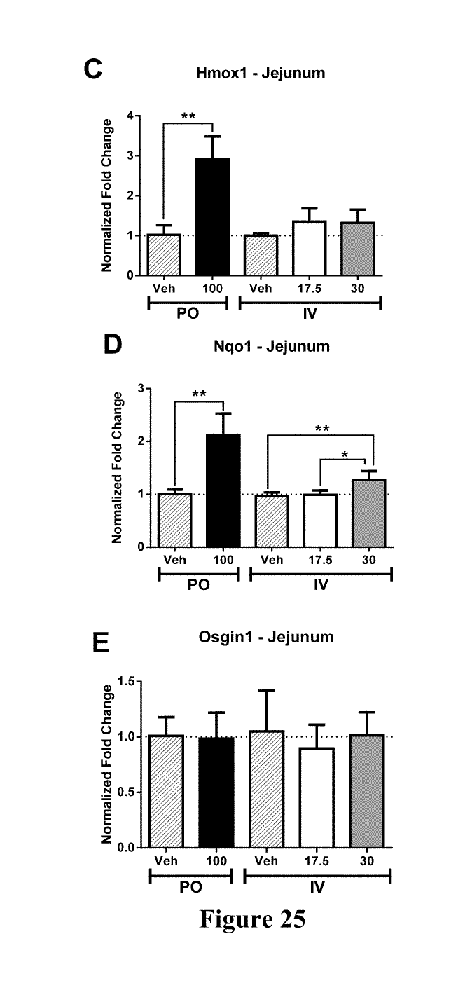

In kidney, there were significant increases after DMF PO dosing for Gclc (1.7±0.4), Hmox1 (7.1±1.8), Nqo1 (1.8±0.3), and Osgin1 (3.8±0.9) relative to vehicle controls (FIG. 25A-E, Table 7 below). The induced changes in kidney after IV dosing were more modest, with significant increases only in Akr1b8 (1.6±0.2) and Gclc (1.4±0.3) after dosing with DMF 30 mg/kg and significant increases in Nqo1 (1.6±0.4), and Osgin1 (1.9±0.4) expression with DMF 17.5 mg/kg.

In jejunum, after PO dosing for Akr1b8 (5.7±2.8), Hmox1 (2.9±0.6), and Nqo1 (2.1±0.4) were all significantly increased relative to vehicle controls (FIG. 25A-E, Table 7 below). After IV dosing of DMF at 30 mg/kg, Nqo1 (1.3±0.2) was significantly increased compared to vehicle and also compared to DMF IV at 17.5 mg/kg. No significant changes in gene expression were observed with IV DMF dosing at 17.5 mg/kg in the jejunum.

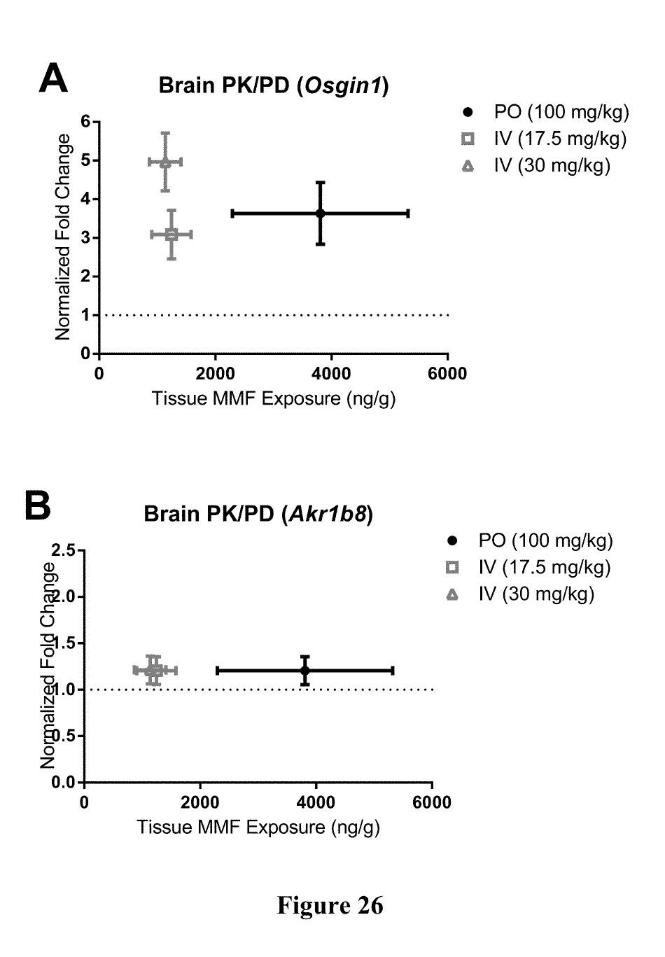

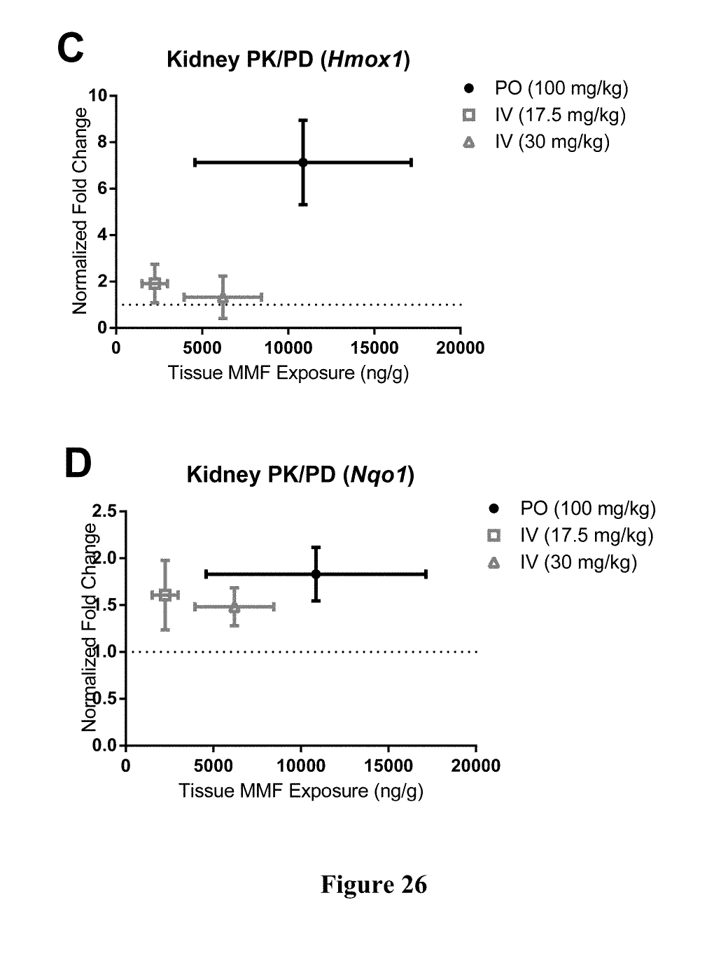

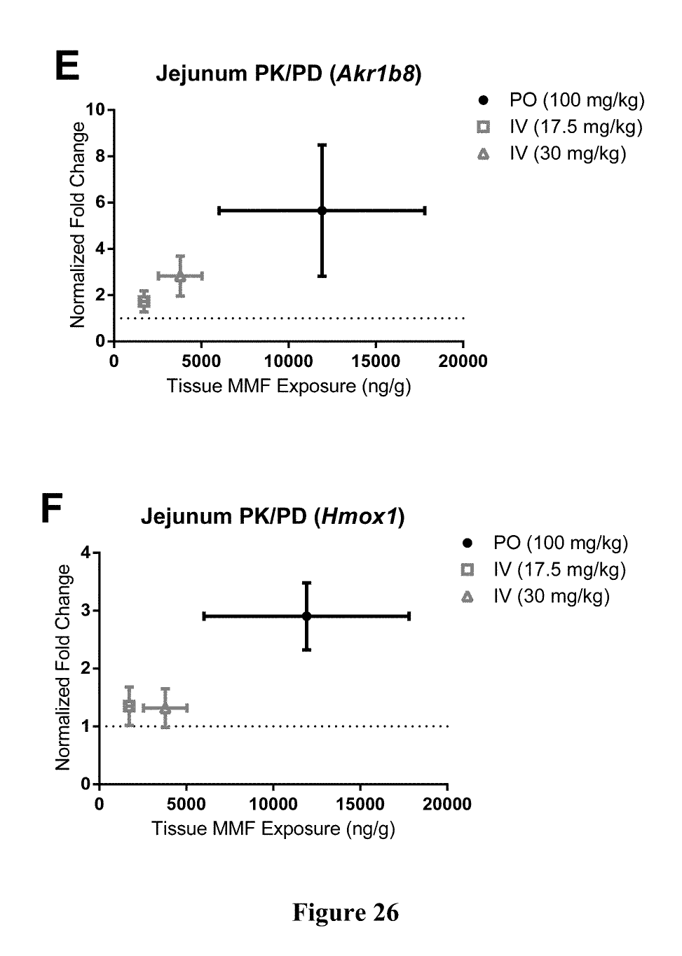

In comparing MMF exposures to pharmacodynamic responses of PO versus IV routes of DMF administration, some trends were observed. It should be noted that these exposure-pharmacodynamic relationships were not measured in the same animal due to the rapid pharmacokinetics of DMF/MMF requiring short time points (<2 hours), and the longer time points (≥2 hours) required to observe modulation of transcript levels. Therefore, ratios were calculated utilizing mean exposures at 10 minutes from one set of animals compared to transcriptional fold-changes at 2 hours in another cohort. In brain, there was not a clear exposure-response relationship for Osgin1 and Akr1b8 (FIG. 26A, B). Despite the significantly lower brain MMF exposures after IV dosing relative to DMF PO dosing, similar magnitude pharmacodynamic responses were observed. Additionally, despite similar brain MMF exposures between DMF IV 17.5 and 30 mg/kg, there was differentiation in the transcriptional responses, with Osgin1 having a significantly higher fold-change at 30 mg/kg as compared to 17.5 mg/kg. These data may indicate that other bioactive DMF-derived fumarate species are present in the brain in addition to MMF, but were not measured in the MMF-specific LC/MS/MS assay. Alternatively, there may be a more complex dose-response relationship occurring that is not captured in the single time-point exposure analysis that may be related to transport across the blood-brain barrier and metabolism of different fumaric acid ester species.

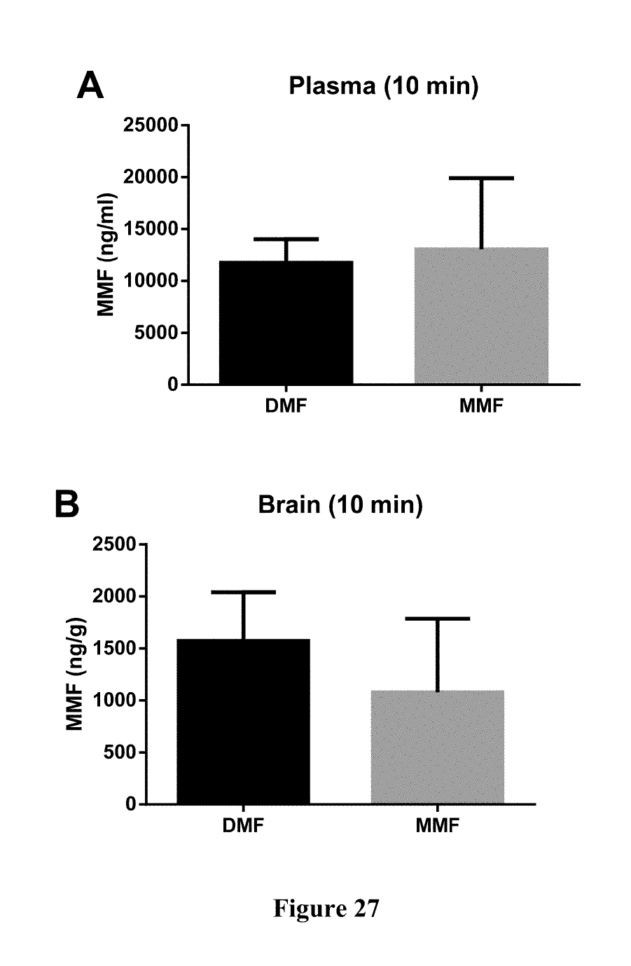

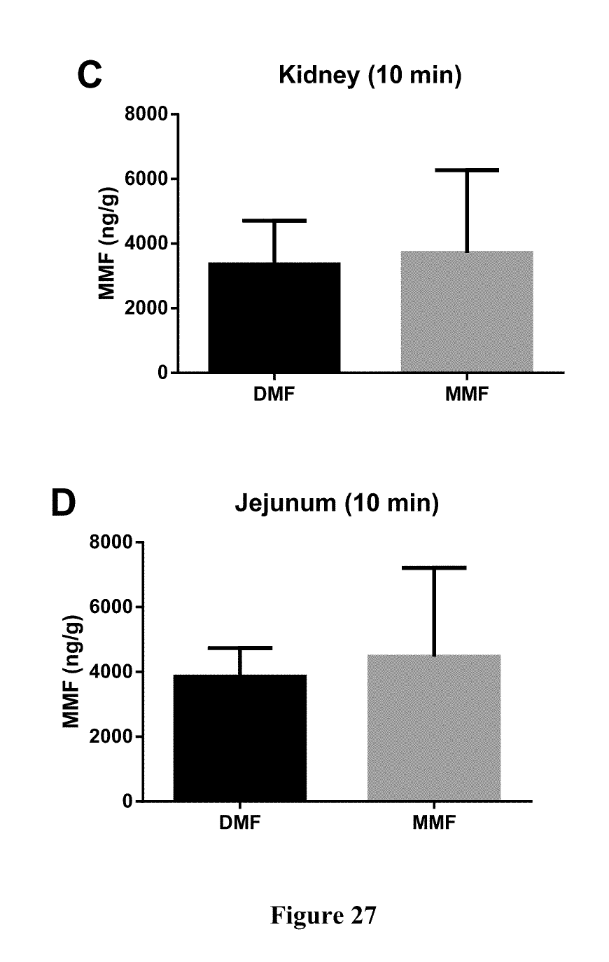

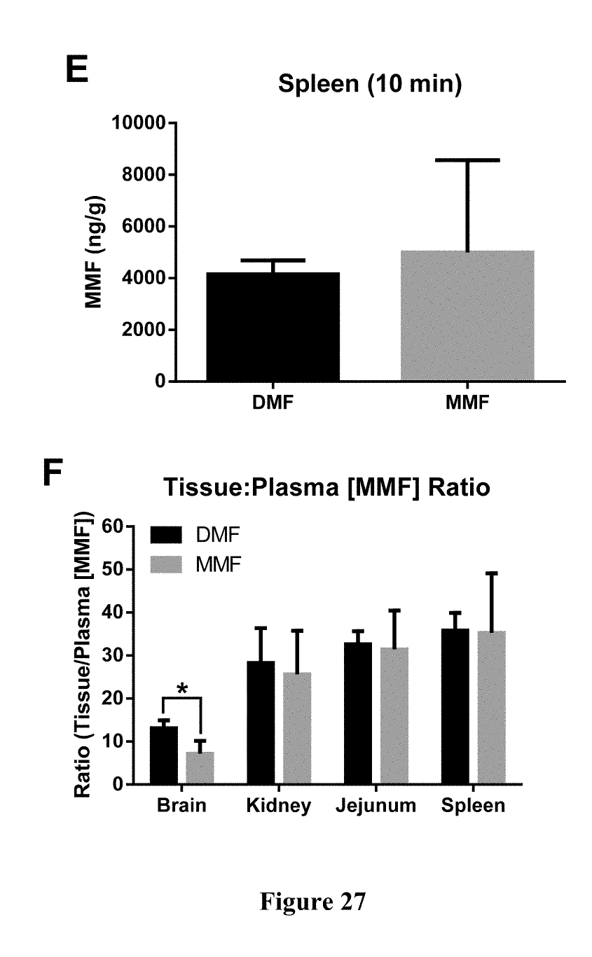

The Hmox1 responses in kidney and jejunum, and Akr1b8 in jejunum exhibited exposure-dependent transcriptional responses (FIG. 27C, E, F). However, Nqo1 responses in kidney were similar despite the significant differences in exposure, which may suggest there is maximal induction plateau for this gene that is already achieved by the lower IV dose (FIG. 26D).

Pharmacokinetics and Pharmacodynamics after Intravenous

Administration of DMF and MMF (Study 2)

To confirm and extend findings from Study 1 described above, a second study was conducted to evaluate the exposure and pharmacodynamic properties of DMF (30 mg/kg) after IV administration. As a comparator, MMF (27.08 mg/kg, equivalent “fumarate” to 30 mg/kg DMF) was also included in this study. MMF exposure levels were evaluated 10 minutes and 2 hours after dosing, while transcriptional pharmacodynamic responses were analyzed at 2 and 6 hours post-dose. A complete blood count (CBC) with differential panel was performed at 10 minutes, 2 hours, and 6 hours post-dose.

| | |
|---|---|
| Group 1: IV Vehicle | (total n = 12, 4/time point) |
| Group 2: IV DMF 30 mg/kg | (total n = 12, 4/time point) |
| Group 3: IV MMF 27.08 mg/kg | (total n = 12, 4/time point) |

TABLE 6

| Time of | | |
|---|---|---|
| Collection | MMF Exposure | Pharmacodynamics |
| 10 minutes | plasma, brain, kidney, | qRT-PCR not done |
| | jejunum, spleen | whole blood (CBC) |
| 2 hours | plasma, brain, kidney, | brain, kidney, jejunum, spleen |
| | jejunum, spleen | (qRT-PCR), whole blood (CBC) |
| 6 hours | not done | brain, kidney, jejunum, spleen |
| | | (qRT-PCR), whole blood (CBC) |

Ten minutes after dosing, 4 animals from each group were sacrificed, and plasma and tissues (brain, kidney, jejunum and spleen) were collected to quantify MMF exposure levels, as well as whole blood to perform CBC. Two hours post-dose, another 4 animals from each group were sacrificed, and plasma, whole blood, and tissues were collected. Tissue samples from the 2-hour time point were split, with half of each sample used for MMF exposure analysis, and half for RNA extraction/qRT-PCR to evaluate the expression of Nrf2 target genes. Six hours after dosing, the last 4 animals from each group were sacrificed, and whole blood and tissues were collected.

At 10 minutes post-dose, the plasma MMF exposure after DMF IV administration (11768±2261 ng/mL; 90.5±17.4 μM) was not significantly different from the plasma levels of MMF after MMF IV dosing (13050±6850 ng/mL; 100.4±52.7 μM) (FIG. 27A). In kidney, jejunum, and spleen, no significant differences were noted in MMF levels between IV DMF and MMF administration (FIG. 27C, D, E). There was a 31% difference in brain MMF levels in animals receiving MMF as compared to animals receiving DMF via IV dosing, but this difference was not significant (FIG. 27B). At 2 hours, there was no detectable MMF in plasma or in any tissues from mice that had received DMF or MMF by IV administration.

In comparing the ratio of tissue-to-plasma MMF exposure, a significantly higher brain penetration (p<0.05) was observed with IV DMF dosing (13.1±1.8) compared to MMF dosing (7.2±3) (FIG. 27F). There were no significant differences in tissue penetration ratios between DMF and MMF administration in kidney, jejunum, or spleen.

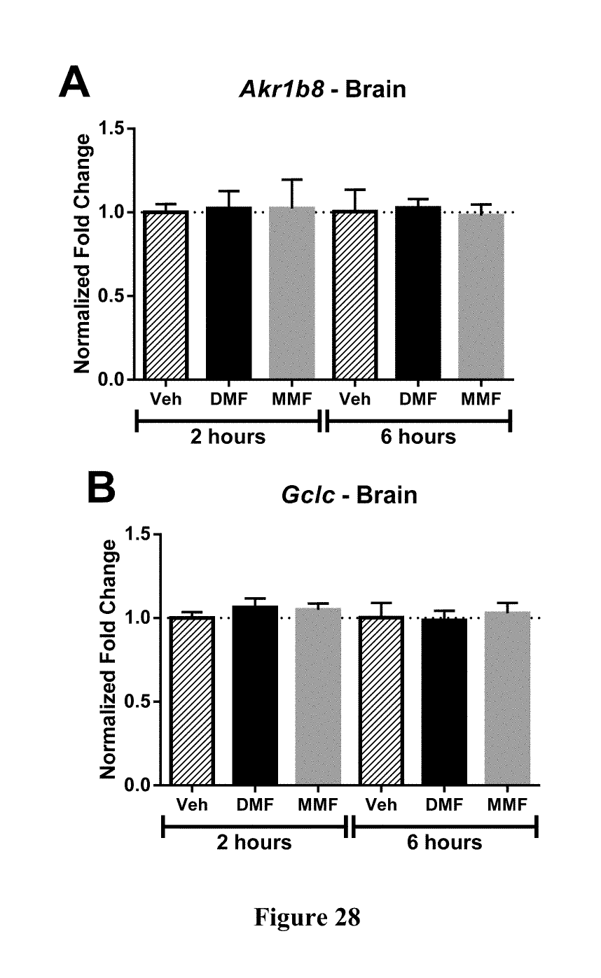

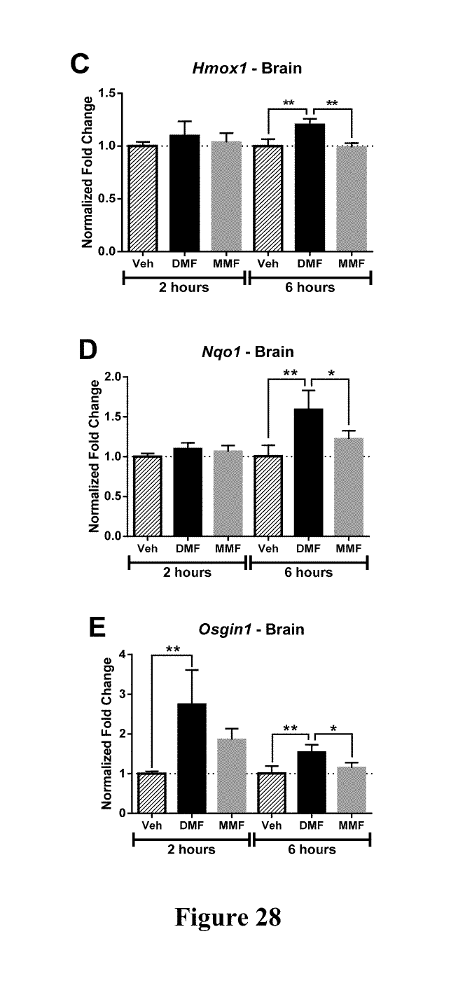

Evaluating the pharmacodynamic changes at the 2- and 6-hour time points revealed differences in the responses between IV DMF and MMF administration. In brain, 2 hours after DMF dosing, only Osgin1 was found to be significantly modulated (2.7±0.9) (FIG. 28E). At 6 hours post-dosing, Hmox1 (1.2±0.1), Nqo1 (1.6±0.2), and Osgin1 (1.5±0.2) were significantly increased in animals receiving DMF IV relative to vehicle controls (FIG. 28C, D, E, Table 7 below). The previously identified significant increases in Akr1b8 expression with DMF administered IV at 30 mg/kg (FIG. 23A) were not repeated here (FIG. 28A), but detecting significant changes of small magnitude (<1.5-fold in Study 1) depends upon the variability in individual data sets for both controls and treated groups. There was no significant increase in transcription of any gene after MMF IV administration relative to vehicle controls. The genes Hmox1, Nqo1, and Osgin1 were all significantly increased by DMF as compared to MMF IV administration at the 6 hour time point (FIG. 28C, D, E, Table 7 below).

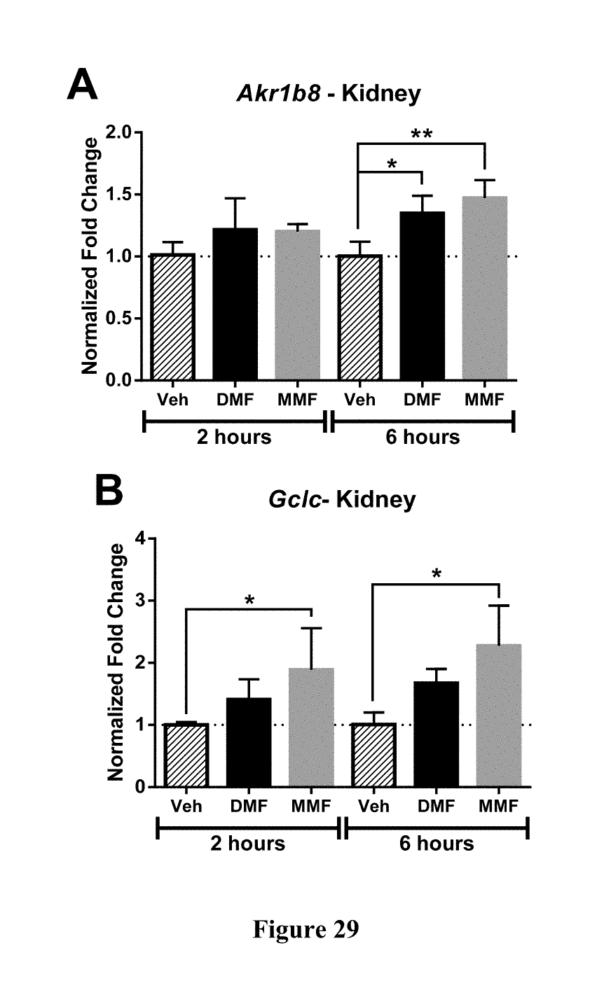

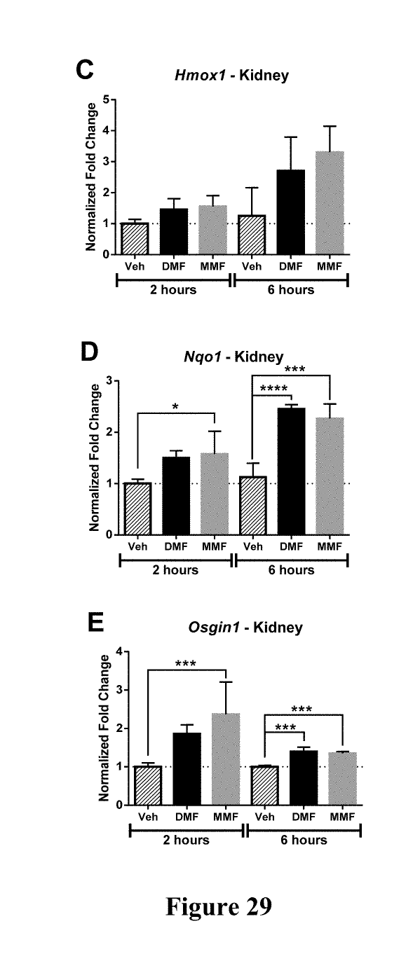

In kidney, 2 hours after dosing with MMF administer IV there were significant increase in transcript levels of Gclc (1.9±0.7), Nqo1 (1.6±0.4), and Osgin1 (2.4±0.8) all relative to vehicle controls (FIG. 29B, D, E, Table 1 below). No significant changes were observed for DMF administration at the 2 hour time point. Six hours after dosing of MMF via IV infusion, there were significant increases in Akr1b8 (1.5±0.1), Gclc (2.3±0.6), Nqo1 (2.3±0.3), and Osgin1 (1.4±0.0), relative to vehicle controls (FIG. 29A, B, D, E, Table 1 below). Six hours after DMF IV administration, there were significant increases in Akr1b8 (1.3±0.1), Nqo1 (2.5±0.1), and Osgin1 (1.4±0.1) relative to vehicle controls in the kidney (FIG. 29A, D, E, Table 1 below).

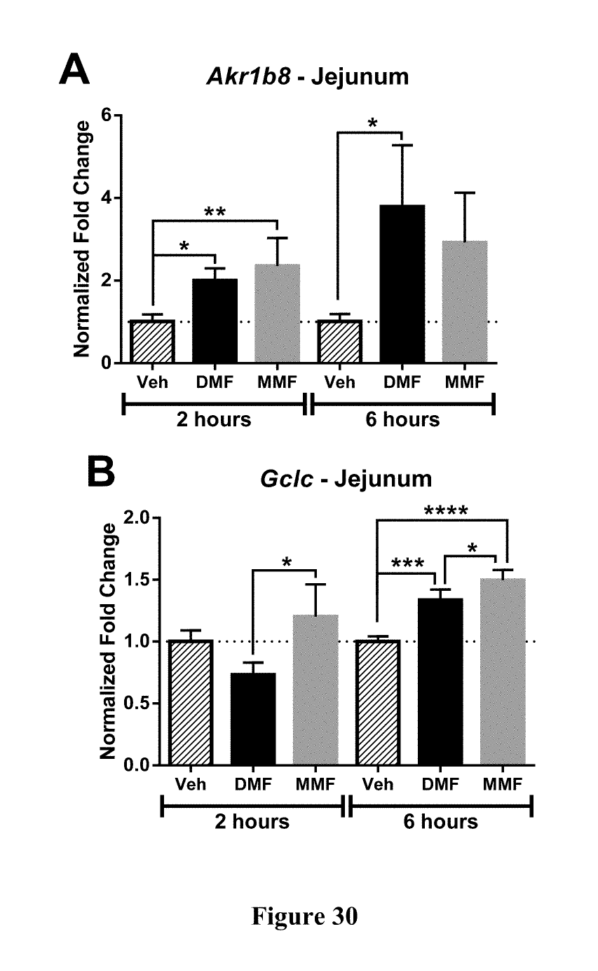

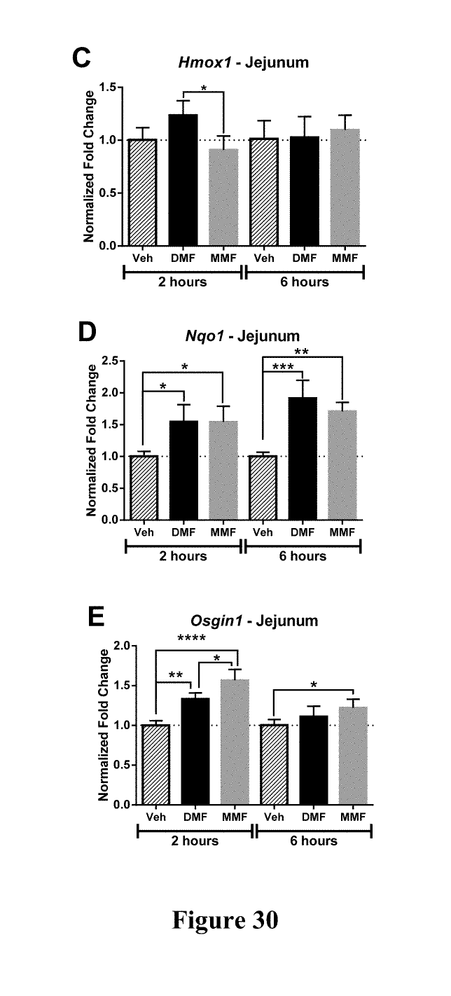

Two hours after IV dosing, in the jejunum there were significant increases observed in expression of Akr1b8 (2.4±0.7, 2.0±0.3), Nqo1 (1.5±0.2, 1.5±0.3), and Osgin1 (1.6±0.1, 1.3±0.1) for MMF and DMF, respectively, relative to vehicle controls (FIG. 30A, D, E, Table 1 below). Of these changes, only the change in Osgin1 was significant comparing MMF to DMF (p<0.05). Six hours after dosing of MMF via IV administration, there were significant increases in Gclc (1.5±0.1), Nqo1 (1.7±0.1), and Osgin1 (1.2±0.1) relative to vehicle controls (FIG. 30B, D, E, Table 1 below). Six hours after administering DMF via IV infusion, there were significant increases in Akr1b8 (3.8±1.5), Gclc (1.3±0.1), and Nqo1 (1.9±0.3) relative to vehicle controls (FIG. 30A, B, D, Table 1 below). In comparing expression levels after IV administration of DMF versus MMF, 2 hours after dosing there were significant differences in Gclc, Hmox1, and Osgin1. 6 hours after dosing there were significant differences between animals receiving DMF and MMF only for Gclc.

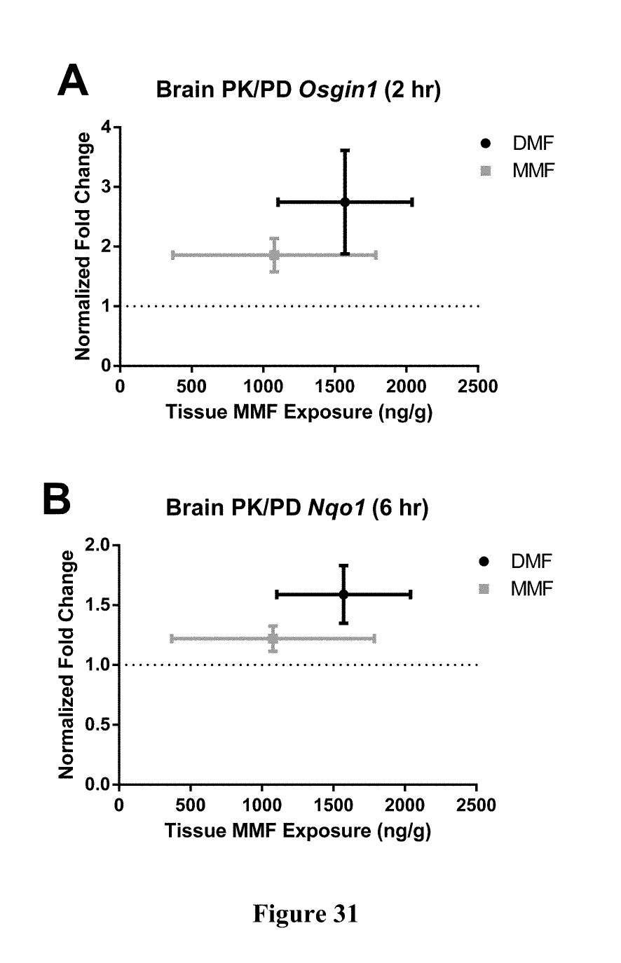

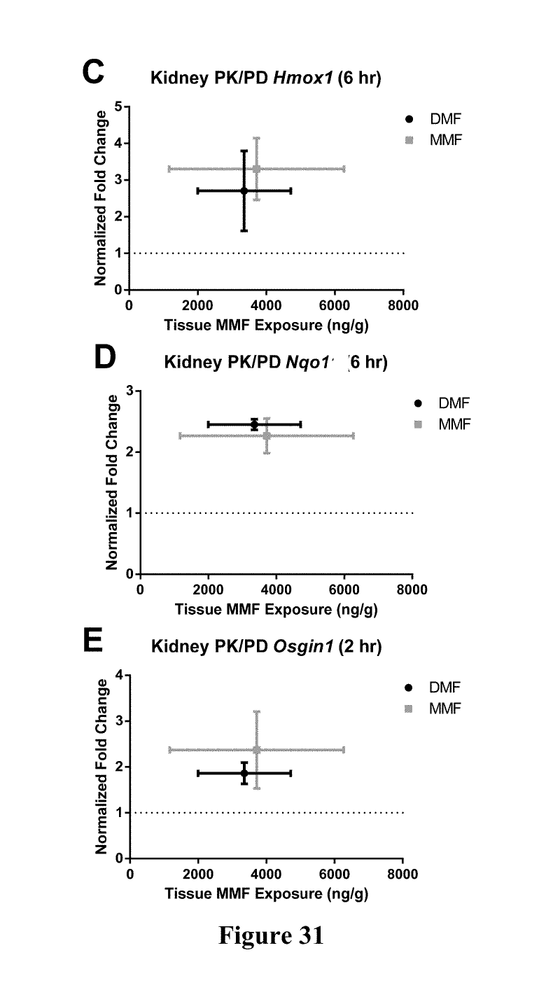

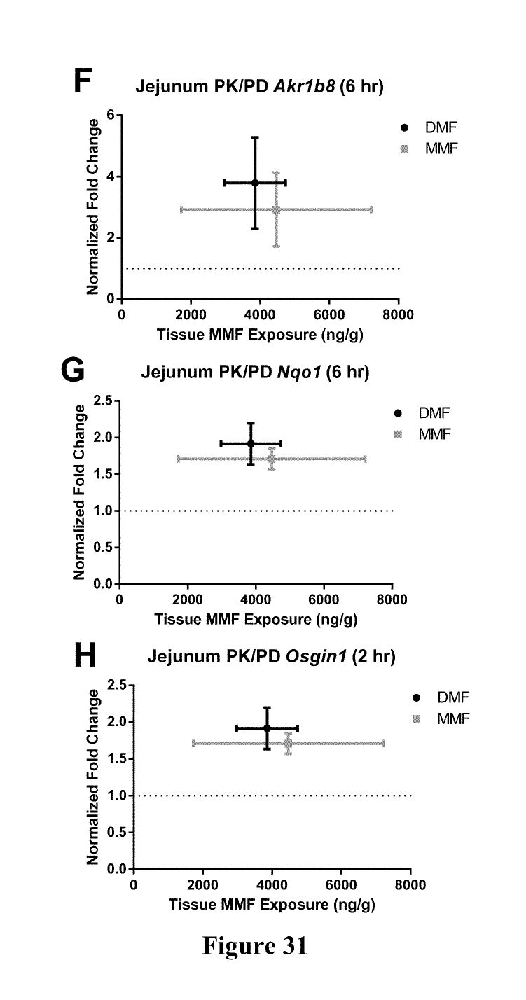

In comparing the exposure to pharmacodynamic response of IV DMF versus MMF administration, it should be again noted that these “PK/PD” relationships are not taken from the same animal due to temporal separation between short compound pharmacokinetics and the longer time required to develop pharmacodynamic transcriptional responses. Ratios were calculated utilizing mean exposures at 10 minutes and transcriptional fold-changes at 2 or 6 hours. In brain, there was a clear exposure-response relationship in comparing DMF and MMF IV administration for Osgin1 at 2 hours and Nqo1 at 6 hours (FIG. 31A, B). In both cases, DMF IV produced higher MMF exposure, as well as higher normalized fold-change in transcription relative to MMF IV. In kidney, with Hmox1 at 6 hours and Osgin1 at 2 hours, MMF IV dosing resulted in a higher exposure, as well as a larger normalized fold change relative to DMF IV (FIG. 31C, E). In kidney with Nqo1 at 6 hours, and in jejunum with Nqo1 at 6 hours and Osgin1 at 2 hours, despite the higher MMF exposures after MMF IV dosing, similar transcriptional responses were observed relative to DMF IV dosing (FIG. 31D, G, H), and in the case of Akr1b8 at 6 hours, MMF IV administration had a lower normalized fold change (FIG. 31F).

TABLE 7
Summary of Pharmacodynamic Changes for DMF and MMF IV Dosing;
Study 1 and Study 2.

| | | DMF PO | DMF IV | DMF IV | DMF IV | DMF IV | MMF IV | MMF IV |
|---|---|---|---|---|---|---|---|---|
| | | 100 mpk | 17.5 mpk | 30 mpk | 30 mpk | 30 mpk | 30 mpk | 30 mpk |
| Gene | Tissue | 2 hr | 2 hr | 2 hr | 2 hr | 6 hr | 2 hr | 6 hr |
| Akr1b8 | Brain | 1.2 ± 0.1 | 1.4 ± 0.1 | 1.3 ± 0.1 | 1.0 ± 0.1 | 1.0 ± 0.1 | 1.0 ± 0.2 | 1.0 ± 0.1 |
| Gclc | Brain | 1.1 ± 0.1 | 1.1 ± 0.1 | 1.1 ± 0.0 | 1.1 ± 0.1 | 1.0 ± 0.1 | 1.0 ± 0.0 | 1.0 ± 0.1 |
| Hmox1 | Brain | 1.2 ± 0.2 | 1.1 ± 0.1 | 1.3 ± 0.1 | 1.1 ± 0.1 | 1.2 ± 0.1 | 1.0 ± 0.1 | 1.0 ± 0.0 |
| Nqo1 | Brain | 1.1 ± 0.1 | 1.2 ± 0.1 | 1.3 ± 0.1 | 1.1 ± 0.1 | 1.6 ± 0.2 | 1.1 ± 0.1 | 1.2 ± 0.1 |
| Osgin1 | Brain | 3.6 ± 0.8 | 3.1 ± 0.6 | 5.0 ± 0.8 | 2.7 ± 0.9 | 1.5 ± 0.2 | 1.9 ± 0.3 | 1.1 ± 0.1 |
| Akr1b8 | Kidney | 1.3 ± 0.3 | 1.4 ± 0.3 | 1.6 ± 0.2 | 1.2 ± 0.3 | 1.3 ± 0.1 | 1.2 ± 0.1 | 1.5 ± 0.1 |
| Gclc | Kidney | 1.7 ± 0.4 | 1.2 ± 0.2 | 1.4 ± 0.3 | 1.4 ± 0.3 | 1.7 ± 0.2 | 1.9 ± 0.7 | 2.3 ± 0.6 |
| Hmox1 | Kidney | 7.1 ± 1.8 | 1.9 ± 0.8 | 1.3 ± 0.9 | 1.5 ± 0.4 | 2.7 ± 1.1 | 1.6 ± 0.3 | 3.3 ± 0.8 |
| Nqo1 | Kidney | 1.8 ± 0.3 | 1.6 ± 0.4 | 1.5 ± 0.2 | 1.5 ± 0.1 | 2.5 ± 0.1 | 1.6 ± 0.4 | 2.3 ± 0.3 |
| Osgin1 | Kidney | 3.8 ± 0.9 | 1.9 ± 0.4 | 1.3 ± 0.3 | 1.9 ± 0.2 | 1.4 ± 0.1 | 2.4 ± 0.8 | 1.4 ± 0.0 |
| Akr1b8 | Jejunum | 5.7 ± 2.8 | 1.7 ± 0.5 | 2.8 ± 0.9 | 2.0 ± 0.3 | 3.8 ± 1.5 | 2.4 ± 0.7 | 2.9 ± 1.2 |
| Gclc | Jejunum | 1.7 ± 0.9 | 1.0 ± 0.2 | 0.8 ± 0.2 | 0.7 ± 0.1 | 1.3 ± 0.1 | 1.2 ± 0.3 | 1.5 ± 0.1 |
| Hmox1 | Jejunum | 2.9 ± 0.6 | 1.4 ± 0.3 | 1.3 ± 0.3 | 1.2 ± 0.1 | 1.0 ± 0.2 | 0.9 ± 0.1 | 1.1 ± 0.1 |
| Nqo1 | Jejunum | 2.1 ± 0.4 | 1.0 ± 0.1 | 1.3 ± 0.2 | 1.5 ± 0.3 | 1.9 ± 0.3 | 1.5 ± 0.2 | 1.7 ± 0.1 |
| Osgin1 | Jejunum | 1.0 ± 0.2 | 0.9 ± 0.2 | 1.0 ± 0.2 | 1.3 ± 0.1 | 1.1 ± 0.1 | 1.6 ± 0.1 | 1.2 ± 0.1 |

| | |
|---|---|
| Study 1 | Study 2 |

Table 7 is a summary table of fold changes, relative to time-matched vehicle controls, for indicated genes in indicated tissues. Values indicate the mean fold-change±the standard deviation (n=4). Changes that were significant from vehicle are indicated in bold (one way ANOVA with Tukey's multiple comparison test).

Pilot Analysis of Blood Parameters after Intravenous Administration of DMF or MMF Conducted as Part of Study 2

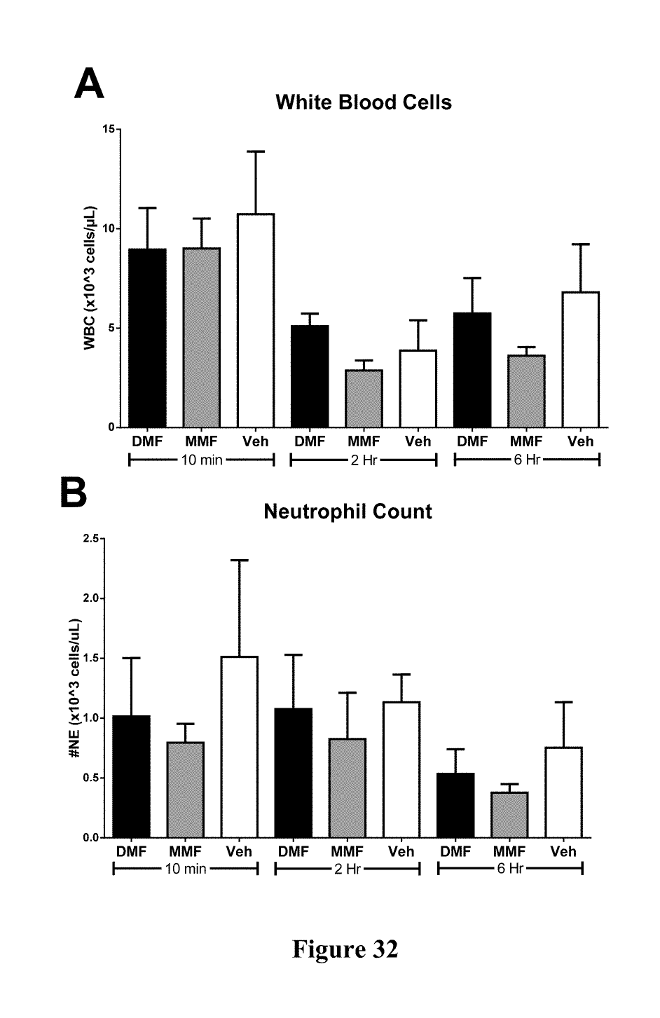

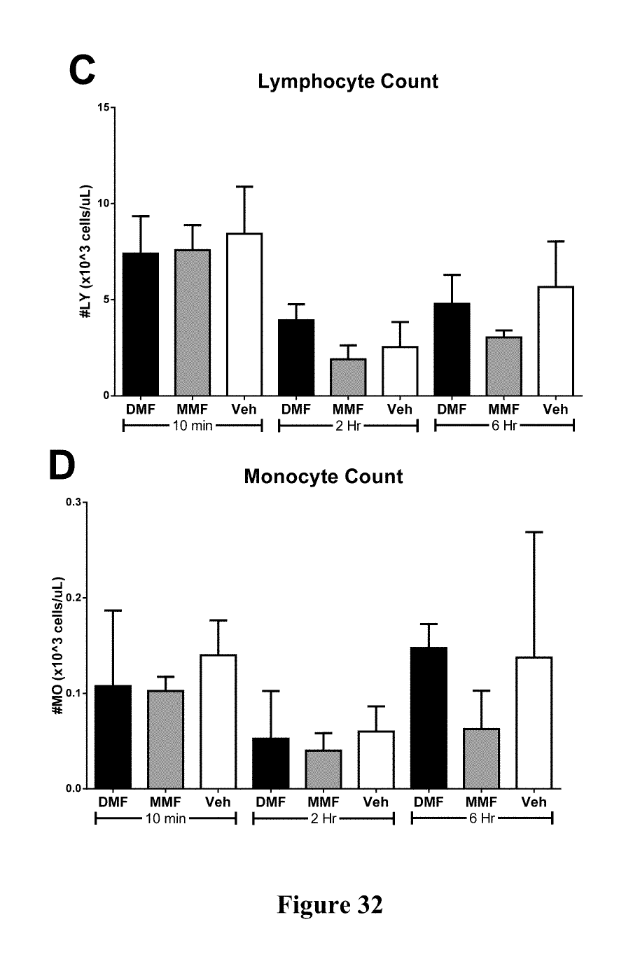

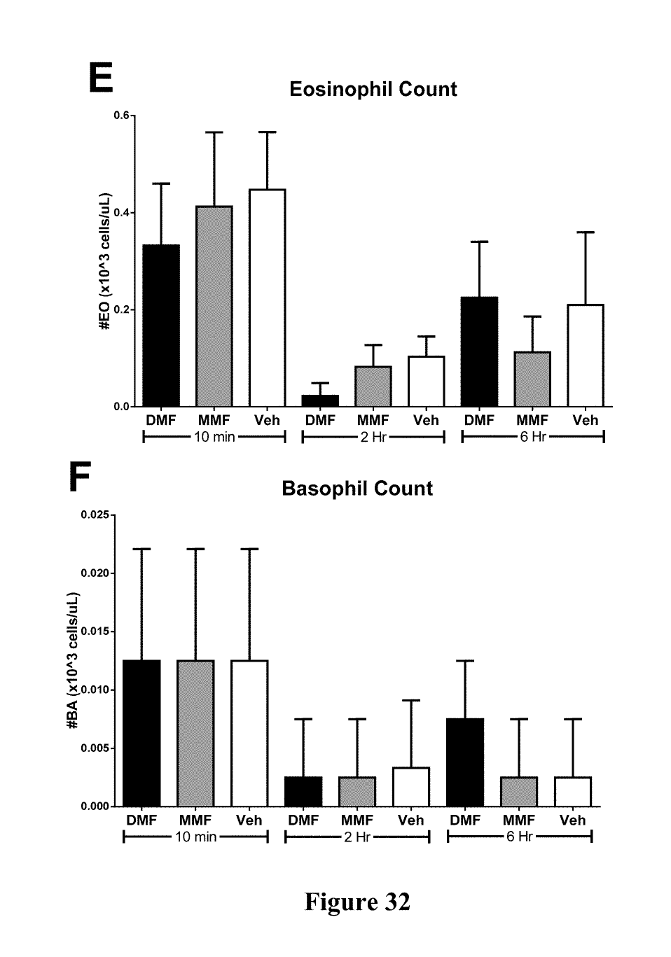

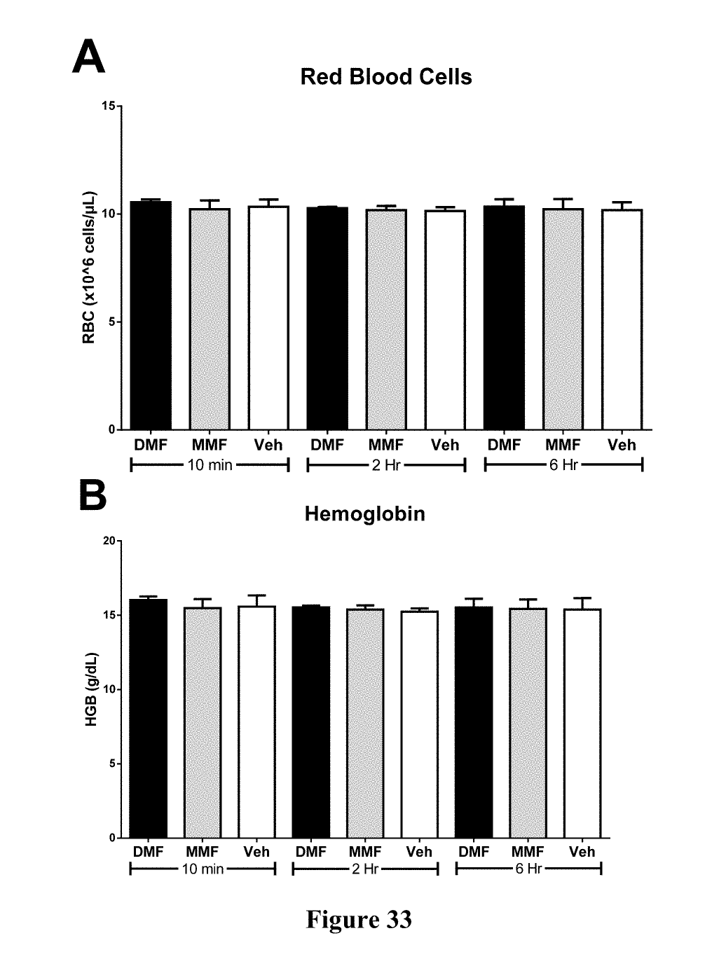

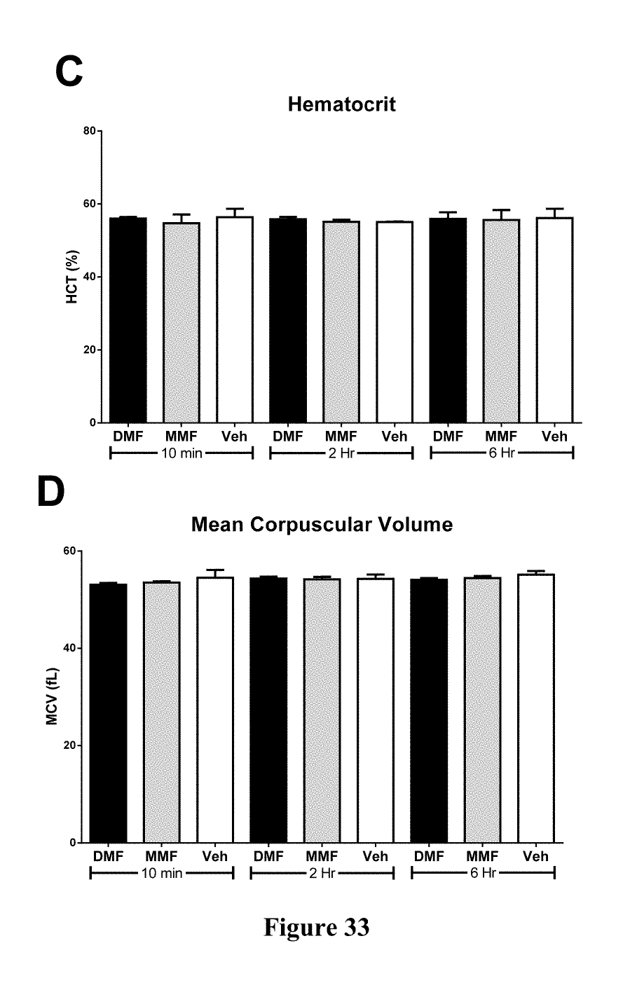

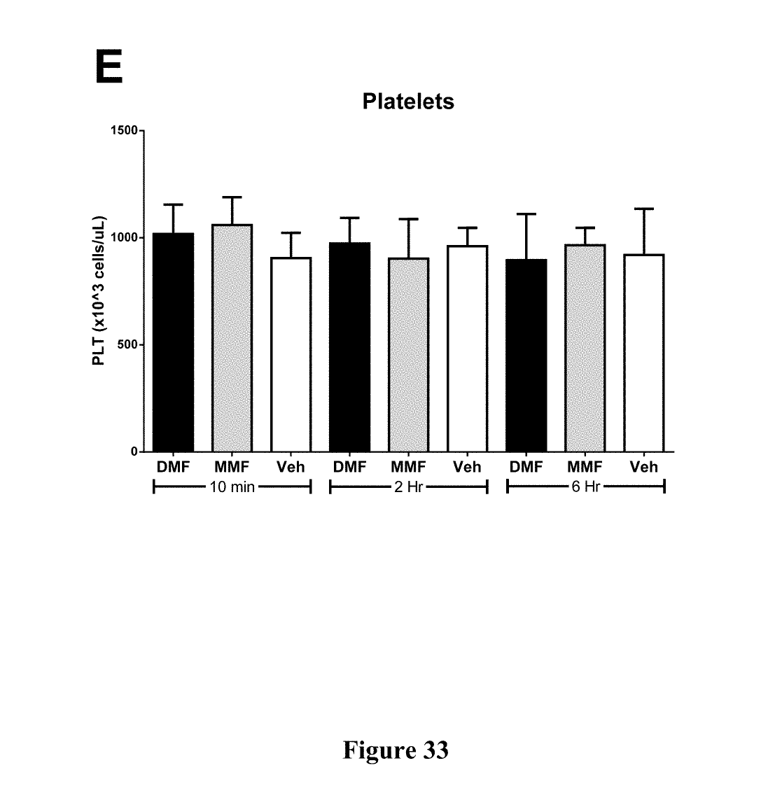

To determine if there were any deleterious effects on blood cell populations after administration of DMF 30 mg/kg or MMF 27.08 mg/kg via intravenous infusion, whole blood from 4 mice per group, collected at 10 minutes, 2 hours, and 6 hours post-dose, was sent to Charles River Laboratories for complete blood count (CBC) with differential. While it is difficult to make any significant conclusions from only 4 animals per group, there appeared to be a general reduction in white blood cells at the 2-hour time point (FIG. 32A). This reduction in cell counts for neutrophils, lymphocytes, monocytes, eosinophils and basophils did not specifically coincide with DMF or MMF treatment, since similar reductions were also observed in the vehicle control groups (FIG. 32B-F). This suggests a vehicle-related phenomenon that was not associated with DMF or MMF administration. There appeared to be some recovery in some cell populations at the 6-hour time point (lymphocytes, monocytes and eosinophils), whereas others remained significantly lower as compared to the 10 minute time point (neutrophils and basophils). There was no effect on red blood cells, hemoglobin, hematocrit, mean corpuscular volume, or platelets (FIG. 33A-E). Statistical analyses on blood cell fractions were not completed due to the overall variability and low number of replicates for the samples in this pilot study.

Multidose Intravenous DMF Administration with WBC Count (Study 3)

To determine effects and tolerability of multi-dose IV administration of DMF, Study 3 was conducted to evaluate the pharmacodynamic properties of DMF (30 mg/kg) after 5 once-daily IV doses and to investigate the occurrence of histopathological alterations in the proximity of the injection site in the tail. A CBC with differential panel was performed at 10 minutes post-last dose via facial vein bleeding, and at 2 hours after the last dose, pharmacodynamic Nrf2 transcriptional responses were analyzed in tissues from the same animals. Histopathology was also performed on regions of the tail in a pilot toxicology study.

Group 1: IV Vehicle (n=4)

Group 2: IV DMF 30 mg/kg (n=5)

TABLE 8

| Time of Tissue Collection | Pharmacodynamics |
|---|---|
| 10 minutes post-last dose | whole blood (CBC) |
| 2 hours post-last dose | brain, kidney, jejunum, spleen (qRT-PCR) |

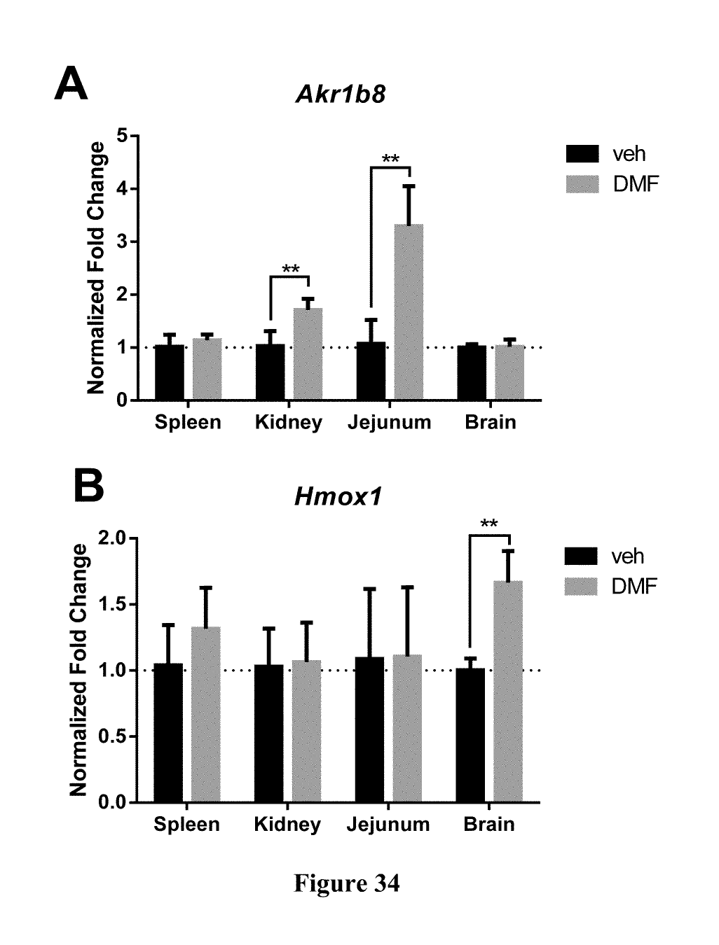

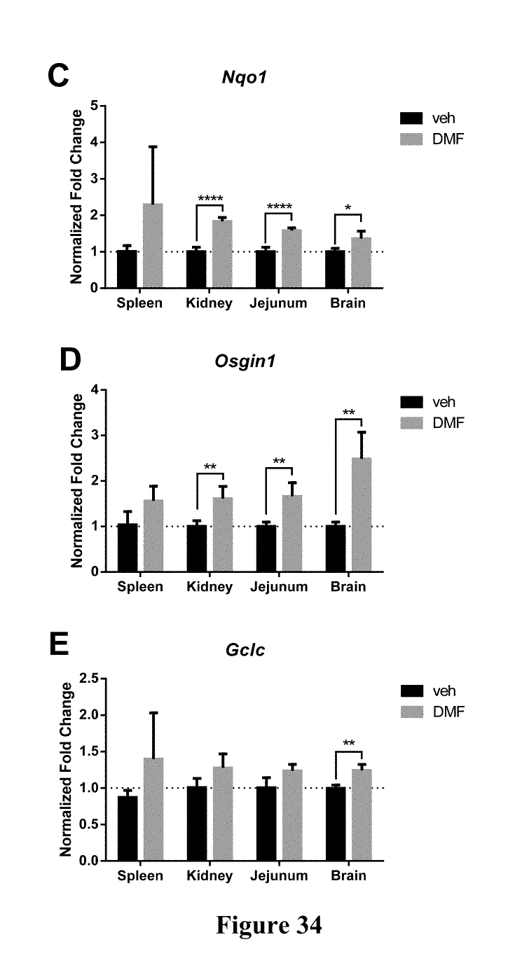

Pharmacodynamic data from this multi-dose study was consistent with single dose responses, with clear increases in transcriptional responses in the brain. In brain at 2 hours post-last dose, there were increases after IV DMF 30 mg/kg dosing for Osgin1 (2.5±0.6 normalized fold change relative to vehicle control) and Hmox1 (1.7±0.2) (FIG. 34B, D). In jejunum, there was an increase seen with Akr1b8 (3.3±0.75) (FIG. 34A). Other transcripts were generally unaffected in tissues analyzed following multi-day IV dosing of DMF (FIG. 34A-E).

Sections of mouse tail were collected for histological analysis. IV administration of DMF 30 mg/kg, once a day for 5 consecutive days, resulted in changes similar to that which was observed in vehicle control mice, consisting of minimal to mild perivascular mixed inflammatory infiltrate by neutrophils and eosinophils, acute hemorrhage, and occasional thrombosis (data not presented). The similar changes in both vehicle control and DMF treated mice were consistent with a procedure- and/or vehicle-related response.

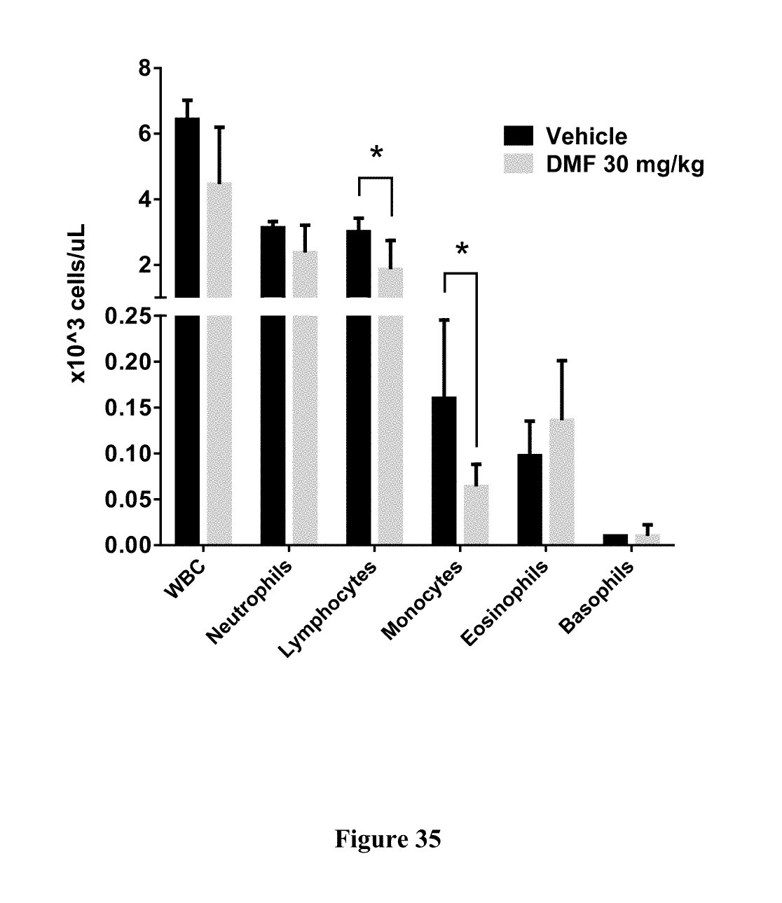

To determine if there were any deleterious effects on blood cell populations after multi-dose IV administration of DMF 30 mg/kg or vehicle, whole blood was collected at 10 minutes post-last (5^{th}) dose, and sent to Charles River Laboratories for complete blood count (CBC) with differential. While difficult to make any significant conclusions from only 4-5 animals per group, there appeared to be a general reduction in white blood cells at 2 hours post-last dose with DMF treatment (FIG. 35). Specifically, a significant decrease in lymphocytes (38%) and monocytes (60%) was observed with DMF treatment. Consistent with Study 2 discussed above, there were no effects on red blood cells, hemoglobin, hematocrit, mean corpuscular volume, or platelets in this study.

Conclusions

Several neurodegenerative diseases have inflammation and oxidative stress as central pathological components. Oral DMF has been shown to activate the Nrf2 pathway in preclinical and clinical studies, and this may mediate, at least in part, the therapeutic effects of treatment in MS (Linker R A, Lee D H, Ryan S, van Dam A M, Conrad R, Bista P, Zeng W, Hronowsky X, Buko A, Chollate S, Ellrichmann G, Bruck W, Dawson K, Goelz S, Wiese S, Scannevin R H, Lukashev M, Gold R. Fumaric acid esters exert neuroprotective effects in neuroinflammation via activation of the Nrf2 antioxidant pathway. Brain. 2011 March; 134(Pt 3):678-92; Scannevin R H, Chollate S, Jung M Y, Shackett M, Patel H, Bista P, Zeng W, Ryan S, Yamamoto M, Lukashev M, Rhodes K J. Fumarates promote cytoprotection of central nervous system cells against oxidative stress via the nuclear factor (erythroid-derived 2)-like 2 pathway. J Pharmacol. Exp. Ther. 2012 April; 341(1):274-84; L. Amaravadi, S. Gopal, R. Gold, R. J. Fox, A. Mikulskis, M. Lukashev, J. Kong, M. Stephan, K. T. Dawson. Effects of BG-12 on a marker of Nrf2 pathway activation: pharmacodynamic results from the phase 3 DEFINE and CONFIRM studies. Thursday, Oct. 11, 2012, 15:30-17:00. ECTRIMS 2012, Lyon France). Preclinical evidence, indicates that increasing MMF exposure (in the periphery and CNS) leads to higher efficacy in neurodegenerative models, however, human dosing cannot be substantially increased beyond current levels due to dose-limiting tolerability with oral administration of the current formulation. Thus, if a mechanism existed to selectively increase relative CNS exposure while maintaining the profile associated with existing peripheral exposures, this may drive enhanced efficacy in neurodegenerative disease through increased CNS cellular resistance to toxic oxidative and inflammatory stress.

The imaging data presented in Example 1 demonstrates that IV administered DMF resulted in selective partitioning of DMF or DMF-derived compounds into the CNS, while oral delivery produced a distribution more restricted to the GI tract. The results from the studies described in this report confirmed that IV DMF resulted in a greater relative partitioning of biologically active MMF into the brain compared to PO DMF dosing, which overall resulted in CNS pharmacodynamic responses that were achieved at lower total DMF doses and corresponding plasma exposures. The exposure-response relationships were somewhat unclear for all genes and doses, and potentially future studies aimed at characterizing the biodistribution, pharmacokinetics and identity of all DMF-derived metabolites may provide more insight into these relationships.

Summary of Results

Study 1: Single Dose of DMF PO (100 mg/kg) or IV (17.5 or 30 mg/kg)
Plasma exposures at 10 min are relatively linear with respect to total administered dose.
MMF levels in plasma and tissue were significantly higher in plasma, brain, kidney, and jejunum after DMF PO dosing as compared to DMF IV dosing. A post-hoc analysis of the two IV doses revealed a significant exposure dose response in plasma, kidney, and jejunum. There was no dose response of MMF brain exposure with DMF IV administration, as 17.5 and 30 mg/kg doses resulted in similar MMF exposures.
Comparing the plasma-to-brain ratios; DMF IV (17.5 mg/kg) produced roughly double the ratio produced by 100 mg/kg PO. The brain penetration ratio for DMF IV (30 mg/kg) was similar to PO. In kidney DMF IV (17.5 mg/kg) ratio was not different than PO, but DMF IV (30 mg/kg) was significantly higher than PO. In jejunum there was no difference between any of the tissue penetration ratios.
In the brain at 2 hours, DMF IV induced significant changes in the mRNAs for Akr1b8, Gclc, Hmox1, Nqo1, and Osgin1. There was a dose-response in the induction of Osgin1 and Hmox1 between 17.5 and 30 mg/kg DMF, but not for Akr1b8, Gclc, and Nqo1. PO dosing induced significant changes in the expression of Akr1b8, Nqo1, and Osgin1.
In the kidney at 2 hours, DMF administered IV induced significant changes in Akr1b8 and Gclc at 30 mg/kg and Nqo1 and Osgin1 at 17.5 mg/kg. DMF PO induced significant changes in the expression of Gclc, Hmox1, Nqo1, and Osgin1.
In the jejunum at 2 hours, DMF administered PO induced significant changes in Akr1b8, Hmox1, and Nqo1. DMF administered IV induced significant changes at 30 mg/kg for Akr1b8, and Nqo1. No significant gene modulation was observed for DMF administered IV at 17.5 mg/kg.
There were no clear exposure:pharmacodynamic relationships in brain; higher absolute tissue MMF exposures did not correlate with higher PD responses. In peripheral tissues higher exposures with PO dosing generally correlated with greater induction of PD.

Study 2: Single Dose of DMF IV (30 mg/kg) or MMF IV (27.08 mg/kg)
Absolute exposures of MMF at 10 minutes were similar between the DMF IV and MMF IV groups in all tissues. However, the ratio of brain penetration was significant higher for DMF IV as compared to MMF IV. A higher ratio of tissue penetration for DMF IV as compound to MMF IV was not observed in kidney, jejunum or spleen.
In the brain, DMF IV induced significant increases in Osgin1 2 hours after dosing, and in Nqo1, Hmox1, and Osgin1 6 hours after dosing. MMF IV did not induce any significant responses. The responses to Hmox1, Nqo1, and Osgin1 were significantly different between DMF and MMF IV administration at the 6 hour time point.
In the kidney, DMF and MMF administered via IV infusion produced similar significant increases in Akr1b8 (6 hours), Nqo1 (6 hours), and Osgin1 (6 hours). MMF administered IV induced additional significant increases in Gclc (2 and 6 hours) and Nqo1 (2 hours), and Osgin1 (2 hours).
In the jejunum, DMF and MMF administered via IV infusion produced significant increases in Akr1b8 (6 hours), Gclc (6 hours), Nqo1 (2 and 6 hours), and Osgin1 (2 hours). MMF administered via IV infusion produced an additional significant increase in Osgin1 at 6 hours.
There were no clear exposure:pharmacodynamic relationships for DMF IV or MMF IV in kidney and jejunum. There was some evidence for an exposure:pharmacodynamic relationship in the brain, as DMF IV resulted in higher MMF brain exposures compared to MMF IV, and had correspondingly greater pharmacodynamic effects.
Pilot analysis of blood cell profiles revealed substantial effects of treatment in all three groups, including vehicle. There was no consistent differentiation of DMF or MMF from the vehicle group.

Study 3: Once Daily Dosing of DMF IV (30 mg/kg) for 5 Consecutive Days
After 5 consecutive days of once-daily DMF IV dosing, a significant increase was observed in brain Hmox1, Osgin1, and Gclc levels 2 hours after dosing, similar to responses after a single dose. Significant increases were also observed in Akr1b8, Nqo1, and Osgin1 levels in the kidney and jejunum.
Pilot histopathology analysis of the tail after the final dose on day 5 did not reveal any treatment-related findings (data not presented herein).
Ten minutes after the 5th DMF IV dose, there were significant decreases in lymphocyte and monocyte counts relative to vehicle controls, while other cell populations were unchanged.

### 6.3 Example 3: Animal Models for Neurological Diseases

As shown in Examples 1 and 2 above, DMF when administered intravenously surprisingly accumulates more in the brain rather than in peripheral tissues, and in greater amounts in the brain, as compared to when DMF is administered orally. It can be predicted, based on these data, that DMF administered intravenously will have a more potent effect in vivo as opposed to DMF administered orally, with respect to effects upon the central nervous system, which are necessary for therapy of neurological diseases such as stroke, amyotrophic lateral sclerosis, Huntington's disease, Alzheimer's disease, and Parkinson's disease. Therefore, it can be expected that, when DMF is administered in animal models of neurological diseases, DMF administered intravenously will show a more pronounced pharmacodynamic effect when compared to DMF administered orally at the same dose, or that DMF administered intravenously will show a similar pharmacodynamic effect at a lower dose when compared to DMF administered orally at a higher dose.

### 6.3.1 Animal Models for Assessing Therapeutic Efficacy of DMF in Neurological Diseases

### 6.3.1.1. Stroke

The primary objective of this study is to evaluate the impact of dimethyl fumarate (DMF) delivered intravenously on a mouse and/or rat model of ischemic and hemorrhagic stroke. Animals will be subjected to pretreatment with IV DMF or placebo prior to the induction of stroke. Also, post stroke treatment with IV DMF vs placebo will also be evaluated. Ding Y, Chen M, Wang M, Li Y, Wen A. Post treatment with 11-Keto-β-Boswellic Acid Ameliorates Cerebral Ischemia-Reperfusion Injury: Nrf2/HO-1 Pathway as a Potential Mechanism. Mol. Neurobiol. 2014 Oct. 28 [epub ahead of print]; Ashabi G, Khalaj L, Khodagholi F, Goudarzvand M, Sarkaki A. Pre-treatment with metformin activates Nrf2 antioxidant pathways and inhibits inflammatory responses through induction of AMPK after transient global cerebral ischemia. Metab. Brain. Dis. 2014 Nov. 21 [epub ahead of print]; and Zhao X, Sun G, Ting S M, Song S, Zhang J, Edwards N J, Aronowski J. Cleaning up after ICH: the role of Nrf2 in modulating microglia function and hematoma clearance. J. Neurochem. 2014 Oct. 18 [epub ahead of print].

The majority of ischemic strokes occur in the territory of middle cerebral artery (MCA), and many animal stroke models focus on this artery. The intraluminal monofilament model of middle cerebral artery occlusion (MCAO) involves the insertion of a surgical filament into the external carotid artery and threading it forward into the internal carotid artery (ICA) until the tip occludes the origin of the MCA, resulting in a cessation of blood flow and subsequent brain infarction in the MCA territory. The MCAO technique will be used to model permanent or transient occlusion. For transient MCAO the suture is removed after a certain interval (30 min, 1 h, or 2 h), reperfusion is achieved. For permanent MCAO, the filament is left in place (24 h). To evaluate the extent of cerebral infarction, excised brain slices will be stained with 2,3,5-triphenyltetrazolium chloride (TTC). Image analysis of the TTC staining will be used to quantitatively determine drug effect. Both pre- and post-infarct drug treatment will be evaluated for therapeutic effect. Chiang T1, Messing R O, Chou W H. Mouse model of middle cerebral artery occlusion. J. Vis. Exp. 2011 Feb. 13; (48). pii: 2761.

### 6.3.1.2. Amyotrophic Lateral Sclerosis

The primary objective of this study is to evaluate the impact of dimethyl fumarate (DMF) delivered intravenously on a mouse model of amyotrophic lateral sclerosis (ALS). ALS is a late onset neurodegenerative disease characterized by loss of upper and lower motor neurons. Mutations in SOD1 cause a genetically inherited form of ALS (Rosen, D. R., Siddique, T., Patterson, D., Figlewicz, D. A., Sapp, P., Hentati, A., Donaldson, D., Goto, J., O'Regan, J. P., Deng, H. X., et al. (1993). Mutations in Cu/Zn superoxide dismutase gene are associated with familial amyotrophic lateral sclerosis. Nature 362, 59-62.). Expression of SOD1-G93A in the mouse leads to disease phenotype that resembles the human disease, including loss of motor neurons, paralysis, and premature mortality (Gurney, M. E., Pu, H., Chiu, A. Y., Dal Canto, M. C., Polchow, C. Y., Alexander, D. D., Caliendo, J., Hentati, A., Kwon, Y. W., Deng, H. X., et al. (1994). Motor neuron degeneration in mice that express a human Cu,Zn superoxide dismutase mutation. Science 264, 1772-1775). Currently, only one drug is approved for the treatment of ALS, and it provides only a modest survival benefit to the patients.

Animals

Female SOD1-G93A mice will be purchased from Jackson Laboratories. All mice will be transgene positive. Sixteen mice will be used per dosing group. Dosing will begin at day 50-55.

Transgene Confirmation and Group Distribution

Each SOD1-G93A mice will carry a variable number of copies of the transgene. The number of copies will influences disease onset and progression, copy number will be monitored by quantitative PCR. Groups will be balanced for copy number. In addition, littermates will be distributed across groups.

Dosing

Mice will be dosed once daily either with vehicle or DMF oral or intravenously.

Monitoring of Motor Disease Onset

Disease onset and progression in the SOD1-G93A will be monitored by three approaches. All approaches will be done in a blinded fashion. Body weight will be recorded on a daily basis. Mice will be inspected on a daily basis for disease onset, as defined by leg tremor or decreased hind limb flexion upon tail lift. Mice will also be assessed on an accelerating rotarod test at weeks 12, 14, and 16. For this test, mice will be first acclimated to a rod rotating at 2 rpm for 300 seconds once a day for two days. For the actual test, mice will be placed on rod accelerating from 2 to 40 rpm over 90 seconds. Upon reaching full speed, the test continues for an additional 30 seconds. Mice will be tested three times per day and the longest time recorded. Mice that will be unable to remain on the rod for 20 seconds will be excluded from further testing.

### Definition of Endpoint

Mice will be determined to have reached endpoint criteria for humane euthanasia when they fail to right from either side within 15 seconds.

Statistical Analysis

For rotarod and body weight, values will be compared by two-way ANOVA.

Body weight will further be evaluated by break point analysis. Break point analysis identifies the inflection point of a curve, by completing a series of linear regression fits. The intersection of the curve fits for the ascending and descending slopes with the minimal residual error is defined as the break point. For each mouse, the breakpoint will be determined. The two groups will then be compared by Student's T-test. Differences in onset and survival will be tested for using the Mantel-Cox log rank test. A further test with the Cox proportional hazards test incorporating littermate status will be completed.

DMF administered intravenously is expected to be more efficacious than DMF administered orally.

### 6.3.1.3. Huntington's Disease

A. Neuroprotective Effects in a Transgenic Mouse Model of Huntington's Disease

Transgenic HD mice of the N171-82Q strain and non-transgenic littermates will be treated with DMF administered orally, DMF administered intravenously, or a vehicle from 10 weeks of age. An IV dose of 30 mg DMF/kg will be administered and an oral dose of 100 mg DMF/kg will be administered. The mice will be placed on a rotating rod (“rotarod”). The length of time at which a mouse falls from the rotarod will be recorded as a measure of motor coordination. The total distance traveled by a mouse will also be recorded as a measure of overall locomotion. N171-82Q transgenic HD mice administered DMF intravenously are expected to remain on the rotarod for a longer period of time and travel farther than mice administered vehicle or oral DMF.

B. Malonate Model of Huntington's Disease

A series of reversible and irreversible inhibitors of enzymes involved in energy generating pathways has been used to generate animal models for neurodegenerative diseases such as Parkinson's and Huntington's diseases. In particular, inhibitors of succinate dehydrogenase, an enzyme that impacts cellular energy homeostasis, has been used to generate a model for Huntington's disease. Kumar P, Kalonia H, Kumar A. Huntington's disease: pathogenesis to animal models. Pharmacol. Rep. 2010 January-February; 62(1):1-14.

Rats will be anesthetized under Bupivacaine (2 mg/kg), Brevital (50 mg/kg, i.p.) and isoflurane anesthesia, placed in a stereotaxic frame and prepared for intrastriatal injection. A single injection of malonate will be injected at a single site in the left striatum corresponding to stereotaxic coordinates at AP: +7.0 mm from bregma; Lateral: 2.8 mm from midline; DV: −5.5 mm from surface of the skull at bregma.

Malonate (in saline) will be injected at a dose of 2.0 μmols in 2.0 μl over 4 minutes using a syringe pump. The injection needle will be left in place for an additional 1.0 min following the infusion and will be withdrawn slowly to minimize reflux of the infusate up the needle tract. Rats will be kept warm on a heating pad until waking from anesthesia (Green and Greenamyre. Characterization of the excitotoxic potential of the reversible succinate dehydrogenase inhibitor malonate. J. Neurochemistry (1995), 64(1):430-4436).

All male Sprague-Dawley rats will be dosed with DMF 24 hours before stereotaxic injection of malonate, rats will be orally or intravenously dosed daily thereafter until the conclusion of the study for a total of 6 (Experiment 1) or 7 (Experiment 2) doses of DMF. Thirty minutes after the last dose, all animals will be sacrificed by CO₂, and their brains will be quickly removed and stored in 20% sucrose in PBS for at least 1 hour, then transferred to 30% sucrose in PBS overnight. On next day, all brains will be embedded in OCT and stored in −80°. The treatment groups will be dosed as follows: vehicle, oral 30 mg/kg, oral 100 mg/kg, I.V. 15 mg/kg and I.V. 30 mg/kg.

Four days after the stereotaxic malonate injection, apomorphrine (1.0 mg/kg) will be given by subcutaneous injection, and ipsilateral turning behavior is determined at 15 min intervals for 60 min.

Cytochrome Oxidase Histochemistry and Analysis of Lesion Size

Incubation medium will consist of 200 mg cytochrome C and 200 mg of 3, 3′-diaminobenzidine tetrahydrochloride (DAB) in 500 ml of 0.1 M phosphate buffer, pH 7.4. Slides will be incubated for 90 minutes at 37° C. and washed twice with PBS (2-5 min.), then removed to 4% neutral, buffered, paraformaldehyde for 20 minutes. Sections will be rinsed twice with PBS (2-5 min.), then twice with distilled water (2-5 min.), dehydrated, cleared in xylene, and coverslipped. The lesion area on each section will be quantified using Open-Lab Image Analysis System. Sections will be taken throughout the entire striatum and a total of twenty 25 μm sections will be analyzed for each rat. Area measurements will be summed and multiplied by intersectional distance (200 μm) to determine the lesion volume for each subject.

Immunofloresence Microscopy

For tissue to be analyzed by immunofluorescence, animals will be anesthetized and transcardially perfused with 4% paraformaldehyde. Brains will be removed and post-fixed overnight at 4° C. Tissue will be then transferred to 30% sucrose in PBS. Serial 25-μM frozen sections will be cut in the coronal plane and air-dried onto gelatin-coated glass slides. All tissue sections will be washed in PBS and blocked with 5% normal goat serum in solution with 0.1% Triton X-100/PBS for 45 minutes at room temperature. Sections will then incubated with monoclonal anti-NeuN (1:500; Millipore, Temecula, Calif., USA) for neurons and polyclonal anti-glial fibrillary acidic protein (GFAP) (1:600) for astrocytes in blocking solution overnight at 4° C. Secondary antibody incubations will be done for 1 hour at room temperature. All immunofluorescence images will be acquired with Openlab and Spectrum software.

Statistical Analysis

Data for lesion volume will be analyzed by one way analysis of variance (ANOVA) followed by a Tukey's test to assess differences between the treatment and vehicle groups.

Pharmacokinetic Analyses of MMF Exposure

Tissue Sample Homogenization:

The study and blank brain samples will be transferred into Tissue Tubes (TT1) (purchased from Covaris, Inc) and attached to a borosilicate glass tube and placed on dry ice for a minimum of 15 minutes. The samples will be pulverized using the Cryoprep System (Covaris Inc.) and transferred to glass tube prior to homogenization. For every 1 part (i.e. 100 mg) of tissue, 2 parts (i.e. 200 uL) of water with 15 mg/mL NaF will be added to each glass tube. The tissues will be homogenized at 4° C. using Covaris E210 for approximately 3 minutes per sample. The Covaris settings will be as follows: Duty Cycle 20%, Intensity 10, and Cycles/Burst 1000. After homogenization 1 part (i.e. 100 uL) acetonitrile will be added to each glass tube, which will be equal to the volume of water added in previously. The homogenates will be homogenized with the E210 for an additional 30 sec per sample. The homogenized tissue will be stored on ice prior to extraction on the same day (Homogenization Dilution Factor=4). The remaining samples will be stored at −80° C.

Sample Preparation:

Study plasma samples will be kept on ice. The total period of time that samples will be exposed to room temperature is less than 4.5 hours per extraction. A 50 μL aliquot of sample (study samples, blank control, calibration standard or QC sample) will be manually transferred into a 96-well plate according to a pre-determined layout. A 5 uL aliquot of 50:50 acetonitrile: water will be added into the wells of the blanks and study samples only. 2004, of internal standard spiking solution will be added to each tube, except for the double blank to which 2004, of 100% acetonitrile will be added. The plate will then vortexed for approximately 60 seconds and centrifuged for 10 minutes at 3000 rpm. A volume of 150 μL of the supernatant will be transferred into a new 96-well injection plate and the supernatant is evaporated to dryness under nitrogen. The dried extracts will be reconstituted in 150 uL 10% acetonitrile, 0.1% formic acid in water. The plate will be vortexed for 2 min and loaded onto an autosampler for injection to determine the concentrations of MMF by LC-MS/MS.

LC-Ms/Ms Assay:

An Agilent 1200 binary pump with a Leap CTC PAL refrigerated autosampler and a Waters Atlantis dC18 column (50×2.1 mm, 3 μm) will be used to analyze samples. The mobile phases used during analysis will be: Mobile Phase A: 0.1% formic acid in water; Mobile Phase B: 0.1% formic acid in acetonitrile. The flow rate is 400-500 μL/min and the injection volume will be approximately 30 μL. The detector will be an Applied Biosystems/MDS Sciex API 4000 triple quadrupole mass spectrometer. The instrument will be equipped with TIS source in negative mode and the analyte is monitored in the MRM mode. Q1 and Q3 are operated with unit/low resolution, respectively and the MS/MS transition masses for MMF and MEF (internal standard) will be 128.8->70.9 and 142.8->70.9. The concentrations for unknown samples will be calculated against Standards.

DMF administered intravenously is expected to be more efficacious than DMF administered orally in the animal models of Huntington's disease.

### 6.3.1.4. Alzheimer's Disease

Heterozygous transgenic mice expressing the Swedish AD mutant gene, hAPPK670N, M671L (Tg2576; Hsiao, Learning & Memory 2001, 8, 301-308) will be used as an animal model of Alzheimer's disease. Animals will be housed under standard conditions with a 12:12 light/dark cycle and food and water available ad libitum. Beginning at 9 months of age, mice will be divided into three groups. The first two groups of animals will receive DMF either orally or intravenously over six weeks. The remaining control will group receive vehicle intravenously for six weeks.

Behavioral testing will be performed using the same sequence over two weeks in all experimental groups: (1) spatial reversal learning, (2) locomotion, (3) fear conditioning, and (4) shock sensitivity.

Acquisition of the spatial learning paradigm and reversal learning will be tested during the first five days of DMF administration using a water T-maze as described in Bardgett et al., Brain Res Bull 2003, 60, 131-142. Mice will be habituated to the water T-maze during days 1-3, and task acquisition begins on day 4. On day 4, mice will be trained to find the escape platform in one choice arm of the maze until 6 to 8 correct choices are made on consecutive trails. The reversal learning phase will be conducted on day 5. During the reversal learning phase, mice will be trained to find the escape platform in the choice arm opposite from the location of the escape platform on day 4. The same performance criteria and inter-trial interval will be used as during task acquisition.

Large ambulatory movements will be assessed to determine that the results of the spatial reversal learning paradigm are not influenced by the capacity for ambulation. After a rest period of two days, horizontal ambulatory movements, excluding vertical and fine motor movements, will be assessed in a chamber equipped with a grid of motion-sensitive detectors on day 8. The number of movements accompanied by simultaneous blocking and unblocking of a detector in the horizontal dimension will be measured during a one-hour period.

The capacity of an animal for contextual and cued memory will be tested using a fear conditioning paradigm beginning on day 9. Testing will take place in a chamber that contains a piece of absorbent cotton soaked in an odor-emitting solution such as mint extract placed below the grid floor. A 5-min, 3 trial 80 db, 2800 Hz tone-foot shock sequence will be administered to train the animals on day 9. On day 10, memory for context will be tested by returning each mouse to the chamber without exposure to the tone and foot shock, and recording the presence or absence of freezing behavior every 10 seconds for 8 minutes. Freezing will be defined as no movement, such as ambulation, sniffing or stereotypy, other than respiration.

On day 11, the response of the animal to an alternate context and to the auditory cue will be tested. Coconut extract is placed in a cup and the 80 dB tone is presented, but no foot shock will be delivered. The presence or absence of freezing in response to the alternate context will then determined during the first 2 minutes of the trial. The tone will then be presented continuously for the remaining 8 minutes of the trial, and the presence or absence of freezing in response to the tone will be determined.

On day 12, the animals will be tested to assess their sensitivity to the conditioning stimulus, i.e., foot shock.

Following the last day of behavioral testing, animals will be anesthetized and the brains removed, post-fixed overnight, and sections cut through the hippocampus. The sections will be stained to image β-amyloid plaques.

Data will be analyzed using appropriate statistical methods. Animals treated with DMF administered intravenously are expected to have improved learning and memory function compared with placebo animals or animals administered DMF orally.

### 6.3.1.5. Parkinson's Disease

To evaluate the effects of DMF administered intravenously (IV), DMF will be formulated in Captisol® and will be injected into rats or mice via tail vein infusion and will be compared to rats or mice receiving DMF by oral gavage (PO). Two IV doses of DMF will be tested, 17.5 and 30 mg/kg as well as a vehicle only control alongside DMF (100 mg/kg) or vehicle (HPMC) administered via PO.

Group 1: IV Vehicle

Group 2: IV DMF 17.5 mg/kg

Group 3: IV DMF 30 mg/kg

Group 4: PO Vehicle only

Group 5: PO DMF 100 mg/kg

A. MPTP Induced Neurotoxicity

MPTP, or 1-methyl-4-phenyl-1,2,3,6-tetrahydropyridine is a neurotoxin that produces a Parkinsonian syndrome in both humans and experimental animals. Studies of the mechanism of MPTP neurotoxicity show that it involves the generation of a major metabolite, MPP+, formed by the activity of monoamine oxidase on MPTP Inhibitors of monoamine oxidase block the neurotoxicity of MPTP in both mice and primates. The specificity of the neurotoxic effects of MPP+ for dopaminergic neurons appears to be due to the uptake of MPP+ by the synaptic dopamine transporter. Blockers of this transporter prevent MPP+ neurotoxicity. MPP+ has been shown to be a relatively specific inhibitor of mitochondrial complex I activity, binding to complex I at the retenone binding site and impairing oxidative phosphorylation. In vivo studies have shown that MPTP can deplete striatal ATP concentrations in mice. It has been demonstrated that MPP+ administered intrastriatally to rats produces significant depletion of ATP as well as increased lactate concentration confined to the striatum at the site of the injections. Compounds that enhance ATP production can protect against MPTP toxicity in mice. Tieu K1, Perier C, Caspersen C, Teismann P, Wu D C, Yan S D, Naini A, Vila M, Jackson-Lewis V, Ramasamy R, Przedborski S. D-beta-hydroxybutyrate rescues mitochondrial respiration and mitigates features of Parkinson disease. J. Clin. Invest. 2003 September; 112(6):892-901.

DMF will be administered orally or intravenously to animals, such as mice or rats, for three weeks before treatment with 1-methyl-4-phenyl-1,2,3,6-tetrahydropyridine (MPTP). MPTP will be administered at an appropriate dose, dosing interval, and mode of administration for 1 week before sacrifice. Control groups will receive either normal saline or MPTP hydrochloride alone. Following sacrifice the two striate will be rapidly dissected and placed in chilled 0.1 M perchloric acid. Tissue will be subsequently sonicated and aliquots analyzed for protein content using a fluorometer assay. Dopamine, 3,4-dihydroxyphenylacetic acid (DOPAC), and homovanillic acid (HVA) will also quantified. Concentrations of dopamine and metabolites will be expressed as nmol/mg protein.

Compounds that protect against DOPAC depletion induced by MPTP, HVA, and/or dopamine depletion are neuroprotective and therefore can be useful for the treatment of Parkinson's disease.

B. Haloperidol-Induced Hypolocomotion

The ability of a compound to reverse the behavioral depressant effects of dopamine antagonists, such as haloperidol, in rodents is considered a valid method for screening drugs with potential anti-Parkinsonian effects (Mandhane, et al., Eur. J. Pharmacol. 1997, 328, 135-141). Hence, the ability of compounds of the disclosure to block haloperidol-induced deficits in locomotor activity in mice can be used to assess both in vivo and potential anti-Parkinsonian efficacy.

Mice used in the experiments will be housed in a controlled environment and allowed to acclimatize before experimental use. One and one-half (1.5) hours before testing, mice will be administered 0.2 mg/kg haloperidol, a dose that reduces baseline locomotor activity by at least 50%. DMF will be administered orally or via intravenously 5-60 min prior to testing. The animals will be then placed individually into clean, clear polycarbonate cages with a flat perforated lid. Horizontal locomotor activity will be determined by placing the cages within a frame containing a 3×6 array of photocells interfaced to a computer to tabulate beam interrupts. Mice will be left undisturbed to explore for 1 h, and the number of beam interruptions made during this period serves as an indicator of locomotor activity. Data from mice administered DMF intravenously will be compared with data for control animals which will receive no DMF and with control animals which will receive DMF orally and will be evaluated for statistically significant differences.

DMF administered intravenously is expected to improve locomotor activity more than DMF administered orally.

C. 6-Hydroxydopamine Animal Model

The neurochemical deficits seen in Parkinson's disease can be reproduced by local injection of the dopaminergic neurotoxin, 6-hydroxydopamine (6-OHDA) into brain regions containing either the cell bodies or axonal fibers of the nigrostriatal neurons. By unilaterally lesioning the nigrostriatal pathway on only one-side of the brain, a behavioral asymmetry in movement inhibition is observed. Although unilaterally-lesioned animals are still mobile and capable of self-maintenance, the remaining dopamine-sensitive neurons on the lesioned side become supersensitive to stimulation. This is demonstrated by the observation that following systemic administration of dopamine agonists, such as apomorphine, animals show a pronounced rotation in a direction contralateral to the side of lesioning. The ability of compounds to induce contralateral rotations in 6-OHDA lesioned rats has been shown to be a sensitive model to predict drug efficacy in the treatment of Parkinson's disease.

Male Sprague-Dawley rats will be housed in a controlled environment and will be allowed to acclimatize before experimental use. Fifteen minutes prior to surgery, animals will be given an intraperitoneal injection of the noradrenergic uptake inhibitor desipramine (25 mg/kg) to prevent damage to nondopamine neurons. Animals will then be placed in an anesthetic chamber and anesthetized using a mixture of oxygen and isoflurane. Once unconscious, the animals will be transferred to a stereotaxic frame, where anesthesia will be maintained through a mask. The top of the head will be shaved and sterilized using an iodine solution. Once dry, a 2 cm long incision will be made along the midline of the scalp and the skin refracted and clipped back to expose the skull. A small hole will then be drilled through the skull above the injection site. In order to lesion the nigrostriatal pathway, the injection cannula will be slowly lowered to position above the right medial forebrain bundle at −3.2 mm anterior posterior, −1.5 mm medial lateral from the bregma, and to a depth of 7.2 mm below the dura mater. Two minutes after lowering the cannula, 6-OHDA will be infused at a rate of 0.5 μL/min over 4 min, to provide a final dose of 8 μg. The cannula will be left in place for an additional 5 min to facilitate diffusion before being slowly withdrawn. The skin will be then sutured shut, the animal removed from the sterereotaxic frame, and returned to its housing. The rats will be allowed to recover from surgery for two weeks before behavioral testing.

Rotational behavior will be measured using a rotameter system having stainless steel bowls (45 cm diameter×15 cm high) enclosed in a transparent Plexiglas cover around the edge of the bowl and extending to a height of 29 cm. To assess rotation, rats will be placed in a cloth jacket attached to a spring tether connected to an optical rotameter positioned above the bowl, which assesses movement to the left or right either as partial (45°) or full (360°) rotations.

To reduce stress during administration of a test compound, rats will initially be habituated to the apparatus for 15 min on four consecutive days. On the test day, rats will be given DMF either orally or intravenously. Immediately prior to testing, animals will be given a subcutaneous injection of a sub-threshold dose of apomorphine, and then placed in the harness and the number of rotations will be recorded for one hour. The total number of full contralateral rotations during the hour test period will serve as an index of anti-Parkinsonian drug efficacy.

DMF administered intravenously is expected to be more efficacious than DMF administered orally.

### 6.3.2 Efficacy of Dimethyl Fumarate in Animal Models of Amyotrophic Lateral Sclerosis and Huntington's Disease

The following experiments show the efficacy of DMF in animal models of neurological diseases upon oral administration.

### 6.3.2.1. Amyotrophic Lateral Sclerosis

Summary

The primary objective of this study was to evaluate the efficacy of orally administered dimethyl fumarate (DMF) on a mouse model of amyotrophic lateral sclerosis (ALS). DMF has been shown to activate the Nrf2 antioxidant response pathway. Genetic activation of the Nrf2 pathway has been shown to slow disease progression in an ALS mouse model.

One genetic cause of ALS is mutations in the superoxide dismutase (SOD1) gene. Mice expressing a specific mutant, SOD1-G93A, develop age-dependent motor neuron loss, which leads to paralysis and premature morbidity. The SOD1-G93A mice are a widely used model for ALS.

SOD1-G93A mice were dosed with either vehicle or 100 mg/kg/day of DMF by oral gavage starting at approximately day 50. Motor performance was assessed by rotarod at day 85, 100, and 115. Health was monitored by clinical observation and body weight. Rotarod performance was not significantly different between vehicle and DMF dosed groups at any of the three time points. Body weight was modestly impacted by DMF. While there was no shift in date of peak body weight, breakpoint analysis showed a significant shift in the inflection point between weight gain and weight loss. DMF had no significant impact on disease on-set or survival in these mice.

Material and Methods

Animals

Thirty-two female SOD1-G93A mice were purchased from Jackson Laboratories (Line 2726). All mice were transgene positive. Sixteen mice were used per dosing group. Dosing began at day 50-55. All procedures were conducted in accordance with the NIH Guide for the Care and Use of Laboratory Animals and were approved by the Biogen Idec Institutional Animal Care and Use Committee (IACUC Protocol No. 0408-2011).

Transgene Confirmation and Group Distribution

Each SOD1-G93A mice carries a variable number of copies of the transgene. The number of copies influences disease onset and progression, copy number was monitored by quantitative PCR. Groups were balanced for copy number. In addition, littermates were distributed across groups.

Test Article and Dosing

Dimethyl fumarate was suspended in 0.8% hydroxypropylmethylcellulose (HPMC) at 10 mg/ml. Mice were dosed once daily either with vehicle or DMF. Dosing volume was 10 ml/kg, introduced by oral gavage.

Monitoring of Motor Disease Onset

Disease onset and progression in the SOD1-G93A was monitored by three approaches. All approaches were done in a blinded fashion. Body weight was recorded on a daily basis. Mice were also inspected on a daily basis for disease onset, as defined by leg tremor or decreased hind limb flexion upon tail lift. Mice were also assessed on an accelerating rotarod test at weeks 12, 14, and 16. For this test, mice were first acclimated to a rod rotating at 2 rpm for 300 seconds once a day for two days. For the actual test, mice are placed on rod accelerating from 2 to 40 rpm over 90 seconds. Upon reaching full speed, the test continues for an additional 30 seconds. Mice were tested three times per day and the longest time recorded. Mice that were unable to remain on the rod for 20 seconds were excluded from further testing.

### Definition of Endpoint

Mice were determined to have reached endpoint criteria for humane euthanasia when then when they failure to right from either side within 15 seconds.

Statistical Analysis

For rotarod and body weight, values were compared by two-way ANOVA. Body weight was further evaluated by break point analysis. Break point analysis identifies the inflection point of a curve, by completing a series of linear regression fits. The intersection of the curve fits for the ascending and descending slopes with the minimal residual error is defined as the break point. For each mouse, the breakpoint was determined. The two groups were then compared by Student's T-test. Differences in onset and survival were tested for using the Mantel-Cox log rank test. A further test with the Cox proportional hazards test incorporating littermate status was completed.

Results

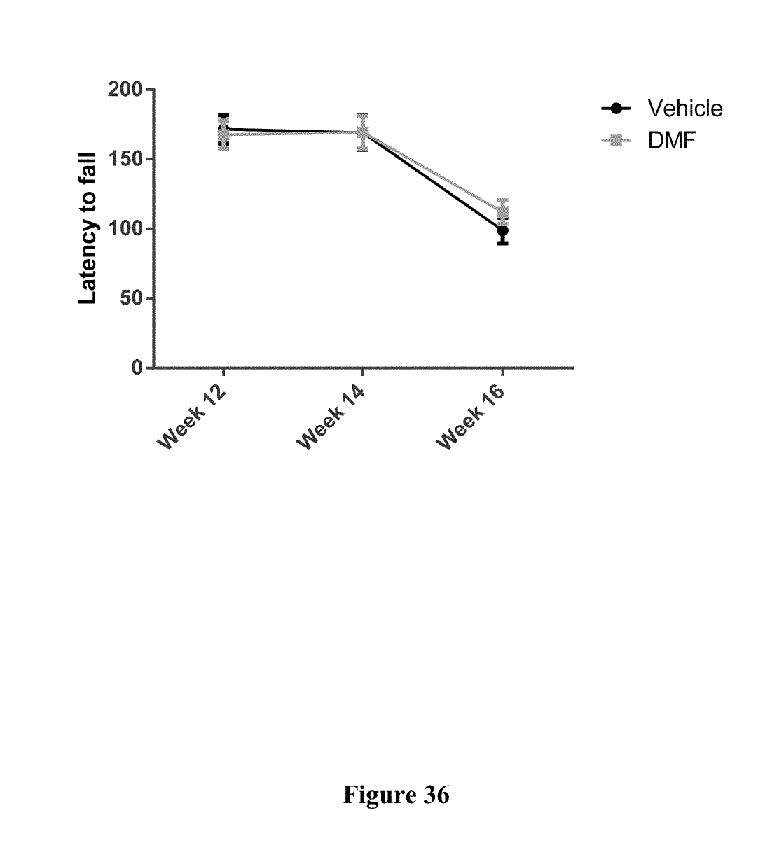

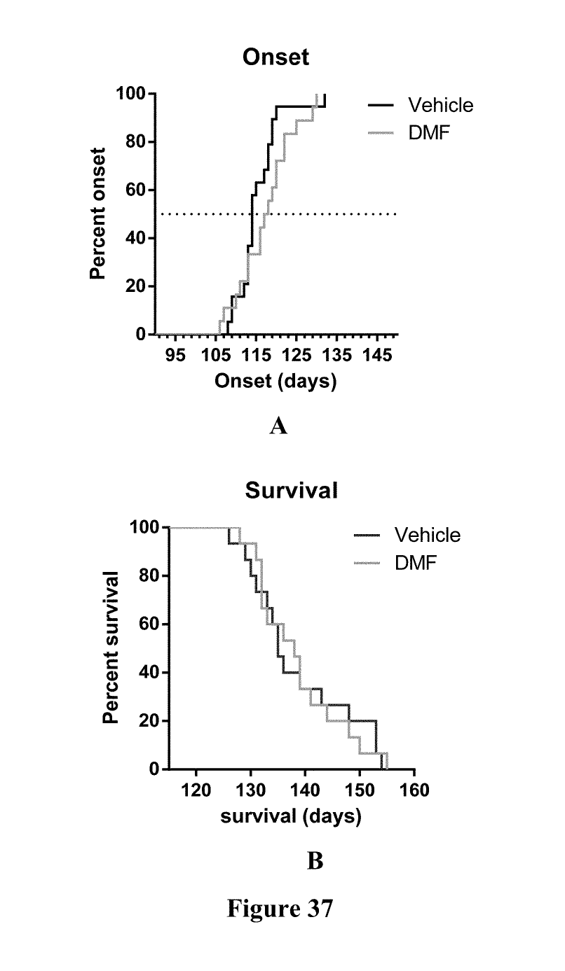

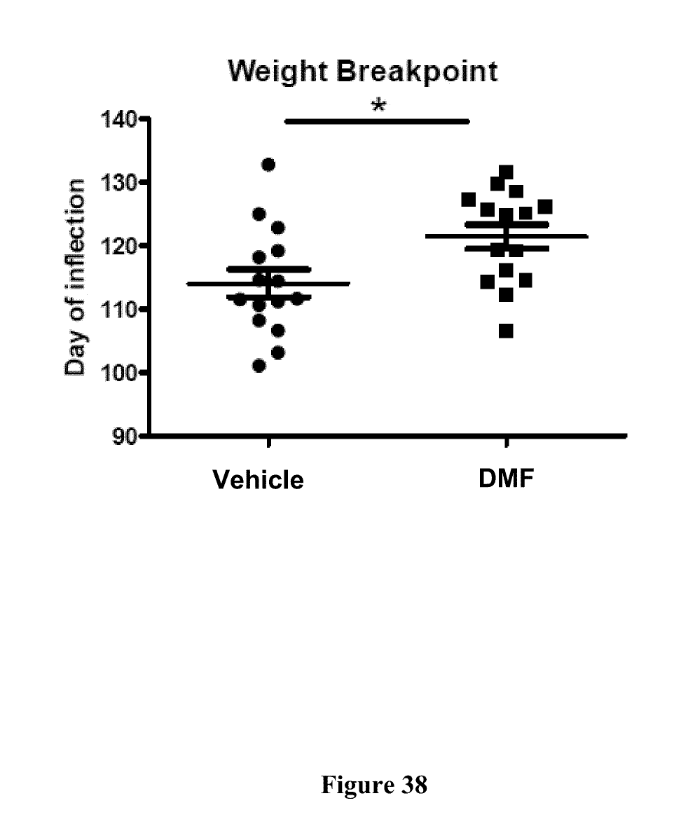

Treatment of SOD1-G93A mice with DMF (p.o., 100 mg/kg daily) did not alter rotarod performance. Performance in both, the vehicle and the DMF group, decreased between week 14 and week 16, consistent with disease onset (FIG. 36). DMF (p.o. 100 mg/kg daily) did not significantly change onset as assessed by limb splay (FIG. 37A). DMF (p.o. 100 mg/kg daily) did not prolong survival of the SOD1-G93A mice (FIG. 37B). Accounting for littermate by Cox proportional hazard analysis did not reveal any compound effects, although littermate matching was found to be a significant contributor to survival (P<0.001). Breakpoint analysis indicates the transition from weight gain to weight loss is significantly delayed (p<0.05) (FIG. 38). Body weight loss is reflective of muscle mass loss.

In conclusion, DMF at the dose level and frequency tested did not improve motor performance or prolong survival in the SOD1-G93A mice. DMF did delay body weight loss, but the benefit was marginal. As discussed above, DMF administered intravenously is expected to be more efficacious in this animal model than DMF administered orally.

### REFERENCES

Gurney, M. E., Pu, H., Chiu, A. Y., Dal Canto, M. C., Polchow, C. Y., Alexander, D. D., Caliendo, J., Hentati, A., Kwon, Y. W., Deng, H. X., et al. (1994). Motor neuron degeneration in mice that express a human Cu,Zn superoxide dismutase mutation. Science 264, 1772-1775.
Neymotin, A., Calingasan, N. Y., Wille, E., Naseri, N., Petri, S., Damiano, M., Liby, K. T., Risingsong, R., Sporn, M., Beal, M. F., et al. (2011). Neuroprotective effect of Nrf2/ARE activators, CDDO ethylamide and CDDO trifluoroethylamide, in a mouse model of amyotrophic lateral sclerosis. Free radical biology & medicine 51, 88-96.
Rosen, D. R., Siddique, T., Patterson, D., Figlewicz, D. A., Sapp, P., Hentati, A., Donaldson, D., Goto, J., O'Regan, J. P., Deng, H. X., et al. (1993). Mutations in Cu/Zn superoxide dismutase gene are associated with familial amyotrophic lateral sclerosis. Nature 362, 59-62.
Scott, S., Kranz, J., E., Cole, J., Lincecum, J., M., Thompson, K., Kelly, N., Bostrom, A., Theodoss, J., Al-Nakhala, B. M., Vieira, F. G., et al. (2008). Design, power, and interpretation of studies in the standard murine model of ALS. Amyotrophic Lateral Sclerosis 9.
Thierry, B., Patrick, B., Jean-Louis, A., and Rebecca, M. P. (2010). Olesoxime (TRO19622): A Novel Mitochondrial-Targeted Neuroprotective Compound. Pharmaceuticals 3.
Vargas, M. R., Johnson, D. A., Sirkis, D. W., Messing, A., and Johnson, J. A. (2008). Nrf2 activation in astrocytes protects against neurodegeneration in mouse models of familial amyotrophic lateral sclerosis. The Journal of neuroscience: the official journal of the Society for Neuroscience 28, 13574-13581.

### 6.3.2.2. Huntington's Disease

Summary

The primary aim of the current studies was to assess the potential neuroprotective effects of dimethyl fumarate (DMF) on a malonate-induced striatal lesion in rats, which is an animal model for Huntington's disease. DMF has been shown to activate the Nrf2 antioxidant response pathway and is the active ingredient in BG-12, an oral formulation currently under clinical investigation for the treatment of Multiple Sclerosis.

Two separate experiments were conducted and are reported here. The first experiment investigated the neuroprotective effect of two doses of DMF (30 mg/kg and 100 mg/kg) on striatal lesion size produced by an intra-striatal administration of malonate. The second experiment was conducted to expand the dose-response function of the neuroprotective effect of DMF on striatal lesion size and to explore functional recovery in the malonate-induced striatal lesion animals. This second experiment included four treatment groups: vehicle, 50 mg/kg, 75 mg/kg and 100 mg/kg of DMF dosed once daily via oral gavage.

All male Sprague-Dawley rats received their first dose of DMF 24 hours before a single stereotaxic injection of malonate in the left striatum under isoflurane anesthesia, rats were dosed daily thereafter until the conclusion of the study for a total of 4 (Experiment 1) or five (Experiment 2) doses of DMF. Three days after the malonate injection and 30 min after the last dose, all animals were sacrificed, and their brains were removed and stored in 20% sucrose for at least one hour, then transferred to 30% sucrose overnight, on the next day, all brains were embedded in OCT and stored in −80°. Striatal lesion volume was determined by histochemical methods for cytochrome C oxidase. In the second experiment, functional recovery was also measured by challenging animals with a dose of apomorphine to induce rotational behavior. Animals were sacrificed 30 minutes after their last dose of DMF and four days after the malonate injection.

Animals dosed with either 75 or 100 mg/kg of DMF significantly reduced malonateinduced lesion volume by 44 and 61%, respectively. Additionally, a significant 41% reduction in rotational behavior was also seen in the animals treated with 100 mg/kg of DMF. There was also a significant protection of neurons in animals treated with DMF, as evidenced by neuron-specific immunostaining in the malonate-lesioned animals. These findings suggest in vivo neuroprotection by DMF as this compound was able to reduce the size of the malonate-induced lesions in rat striatum, and preserve neuronal and behavioral function.

Material and Methods

Animals:

Forty-five adult male Sprague-Dawley rats (Charles River, Wilmington, Mass.) weighing 275-300 grams at the beginning of the experiment were used. Twenty-one rats for experiment #1 (7 rats per group for three treatments) and fifty six rats for experiment #2 (14 rats per group for four treatments). All procedures were conducted in accordance with the NIH Guide for the Care and Use of Laboratory Animals and were approved by the Biogen Idec Institutional Animal Care and Use Committee (IACUC Protocol No. 0349-2010).

Malonate-Induced Striatal Lesion in Rodents:

Rats were anesthetized under Bupivacaine (2 mg/kg), Brevital (50 mg/kg, i.p.) and isoflurane anesthesia, placed in a stereotaxic frame and prepared for intrastriatal injection. A single injection of malonate was injected at a single site in the left striatum corresponding to stereotaxic coordinates at AP: +7.0 mm from bregma; Lateral: 2.8 mm from midline; DV: −5.5 mm from surface of the skull at bregma. Malonate (in saline) was injected at a dose of 2.0 μmols in 2.0 μl over 4 minutes using a syringe pump. The injection needle was left in place for an additional 1.0 min following the infusion and was withdrawn slowly to minimize reflux of the infusate up the needle tract. Rats were kept warm on a heating pad until waking from anesthesia (Green and Greenamyre, 1995).

All male Sprague-Dawley rats received DMF 24 hours before stereotaxic injection of malonate, rats were orally dosed daily thereafter until the conclusion of the study for a total of 6 (Experiment 1) or 7 (Experiment 2) doses of DMF. Three (Experiment 1) or four (Experiment 2) days after the malonate injection and 30 min after the last dose, all animals were sacrificed by CO₂, and their brains were quickly removed and stored in 20% sucrose in PBS for at least 1 hour, then transfer them to 30% sucrose in PBS overnight. On next day, all brains were embedded in OCT and stored in −80° C. The first experiment included three treatment groups: vehicle, 30 mg/kg and 100 mg/kg, and the second experiment included four treatment groups: vehicle, 50 mg/kg, 75 mg/kg and 100 mg/kg.

Rotational Behavior Induced by Apomorphine:

Four days after the stereotaxic malonate injection, apomorphine (1.0 mg/kg) was given by subcutaneous injection, and ipsilateral turning behavior was determined at 15 min intervals for 60 min.

Cytochrome Oxidase Histochemistry and Analysis of Lesion Size:

Incubation medium consisted of 200 mg cytochrome C and 200 mg of 3, 3′-Diaminobenzidine tetrahydrochloride (DAB) in 500 ml of 0.1 M phosphate buffer, pH 7.4. Slides were incubated for 90 minutes at 37° C. and wash twice with PBS (2-5 min.), then removed to 4% neutral, buffered, paraformaldehyde for 20 minutes. Sections were rinsed twice with PBS (2-5 min.), then twice with distilled water (2-5 min.), dehydrated, cleared in xylene, and coverslipped.

The lesion area on each section was quantified using Open-Lab Image Analysis System. Sections were taken throughout the entire striatum and a total of twenty 25 μm sections were analyzed for each rat. Area measurements were summed and multiplied by intersectional distance (200 μm) to determine the lesion volume for each subject.

Immunofloresence Microscopy

For tissue to be analyzed by immunofluorescence, animals were anesthetized and transcardially perfused with 4% paraformaldehyde. Brains were removed and post-fixed overnight at 4° C. Tissue was then transferred to 30% sucrose in PBS. Serial 25-μM frozen sections were cut in the coronal plane and air-dried onto gelatin-coated glass slides. All tissue sections were washed in PBS and blocked with 5% normal goat serum in solution with 0.1% Triton X-100/PBS for 45 minutes at room temperature. Sections were then incubated with monoclonal anti-NeuN (1:500; Millipore, Temecula, Calif., USA) for neurons and polyclonal anti-glial fibrillary acidic protein (GFAP) (1:600) for astrocytes in blocking solution overnight at 4° C. Secondary antibody incubations were for 1 hour at room temperature. All immunofluorescence images were acquired with Openlab and Spectrum software.

Test Articles, Controls, and Histology Chemical Supplies:

DMF (Experiment #1: Lot 16293-33; Experiment #2: lot 16275-15) and formulation vehicle (0.8% hypromellose). Sodium malonate dibasic monohydrate (Sigma-Aldrich, lot 108k5314). Apomorphrine (Sigma-Aldrich, batch#096k1414, M.W.=312.8). All test articles were formulated to be injected at a volume of 1.0 ml/kg body weight except malonate. For histology, 3, 3′-Diaminobenzidin (DAB) (Sigma-Aldrich, lot#588505, M.W.=214.27), Cytochrome C from bovine heart (Sigma-Aldrich, lot#070m7001v, M.W.=12,327 basis).

Statistical Analysis:

Data for lesion volume was analyzed by one way analysis of variance (ANOVA) followed by a Tukey's test to assess differences between the treatment and vehicle groups.

Pharmacokinetic Analyses of MMF Exposure:

Tissue Sample Homogenization

The study and blank brain samples was transferred into Tissue Tubes (TT1) (purchased from Covaris, Inc) and attached to a borosilicate glass tube and placed on dry ice for a minimum of 15 minutes. The samples were pulverized using the Cryoprep System (Covaris Inc.) and transferred to glass tube prior to homogenization. For every 1 part (i.e. 100 mg) of tissue, 2 parts (i.e. 200 μL) of water with 15 mg/mL NaF was added to each glass tube. The tissues were homogenized at 4° C. using Covaris E210 for approximately 3 minutes per sample. The Covaris settings were as follows: Duty Cycle 20%, Intensity 10, and Cycles/Burst 1000. After homogenization 1 part (i.e. 100 uL) acetonitrile was added to each glass tube, which was equal to the volume of water added in previously. The homogenates were homogenized with the E210 for an additional 30 sec per sample. The homogenized tissue stored on ice prior to extraction on the same day (Homogenization Dilution Factor=4). The remaining samples were stored at −80° C.

Sample Preparation

Study plasma samples were on ice. The total period of time that samples were exposed to room temperature was less than 4.5 hours per extraction. A 50 μL aliquot of sample (study samples, blank control, calibration standard or QC sample) was manually transferred into a 96-well plate according to a pre-determined layout. A 5 μL aliquot of 50:50 acetonitrile: water was added into the wells of the blanks and study samples only. 200 μL of internal standard spiking solution was added to each tube, except for the double blank to which 2004, of 100% acetonitrile was added. The plate was then vortexed for approximately 60 seconds and centrifuged for 10 minutes at 3000 rpm. A volume of 150 μL of the supernatant was transferred into a new 96-well injection plate and evaporated the supernatant to dryness under nitrogen. The dried extracts were reconstituted in 1504, 10% acetonitrile, 0.1% formic acid in water. The plate was vortexed for 2 min and loaded onto an autosampler for injection to determine the concentrations of BIO-022817 by LC-MS/MS.

LC-MS/MS Assay

The HPLC system used consisted of an Agilent 1200 binary pump with a Leap CTC PAL refrigerated autosampler. A Waters Atlantis dC18 column (50×2.1 mm, 3 μm) was used. The mobile phases used during analysis are: Mobile Phase A: 0.1% formic acid in water; Mobile Phase B: 0.1% formic acid in acetonitrile. The flow rate was 400-500 μL/min and the injection volume was approximately 30 μL. The detector was an Applied Biosystems/MDS Sciex API 4000 triple quadrupole mass spectrometer. The instrument was equipped with TIS source in negative mode and the analyte was monitored in the MRM mode. Q1 and Q3 were operated with unit/low resolution, respectively and the MS/MS transition masses for BIO-022817 and MEF (IS) are 128.8->70.9 and 142.8->70.9. The concentrations for unknown samples were calculated against Standards.

Results

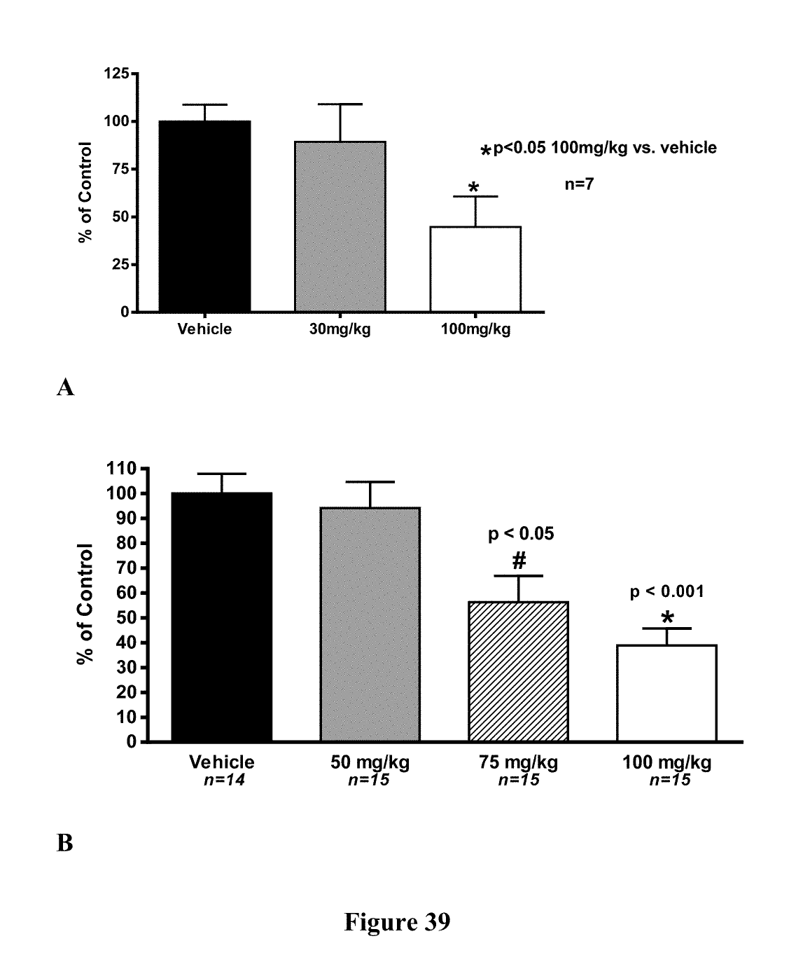

Experiment 1 shows that treatment with 100 mg/kg of DMF reduced malonate induced lesion volume by 55% (FIG. 39A). The values in FIG. 39A represent mean % of control and the error bars denote SEM. n=7 per group. Experiment 2 shows treatment with 75 or 100 mg/kg of DMF reduced malonate-induced lesion volume by 44 and 61%, respectively (FIG. 39B). The values in FIG. 39B represent mean % of control and the error bars denote SEM.

Experiment 2 shows that the treatment with 100 mg/kg of DMF results in a significant 41% decrease in apomorphrine-induced rotational behavior relative to the vehicle treated group (FIG. 40). The bars in FIG. 40 represent mean rotations over a 60 minute period and the error bars denote SEM.

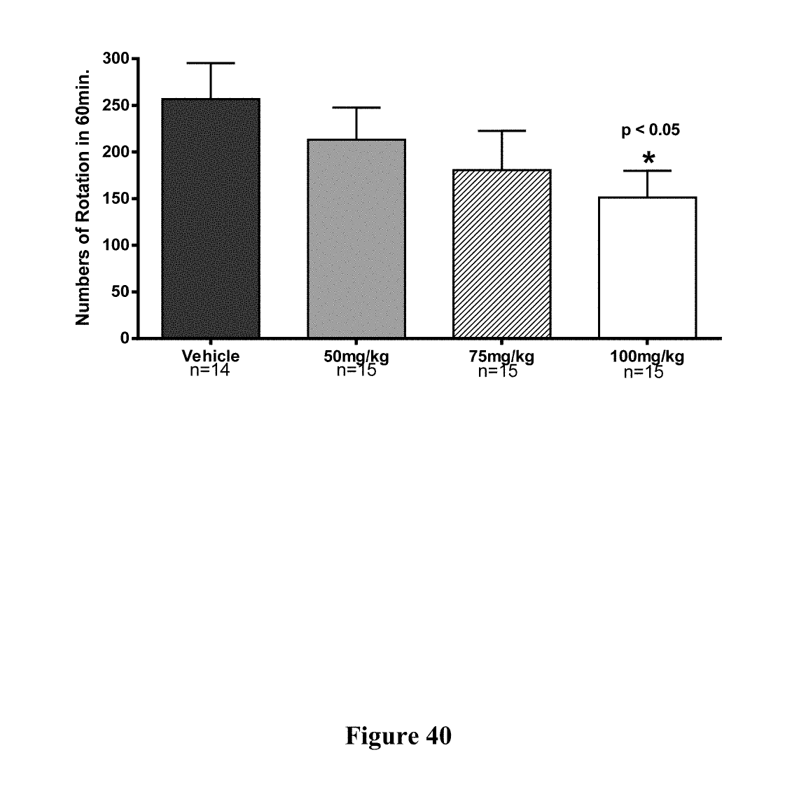

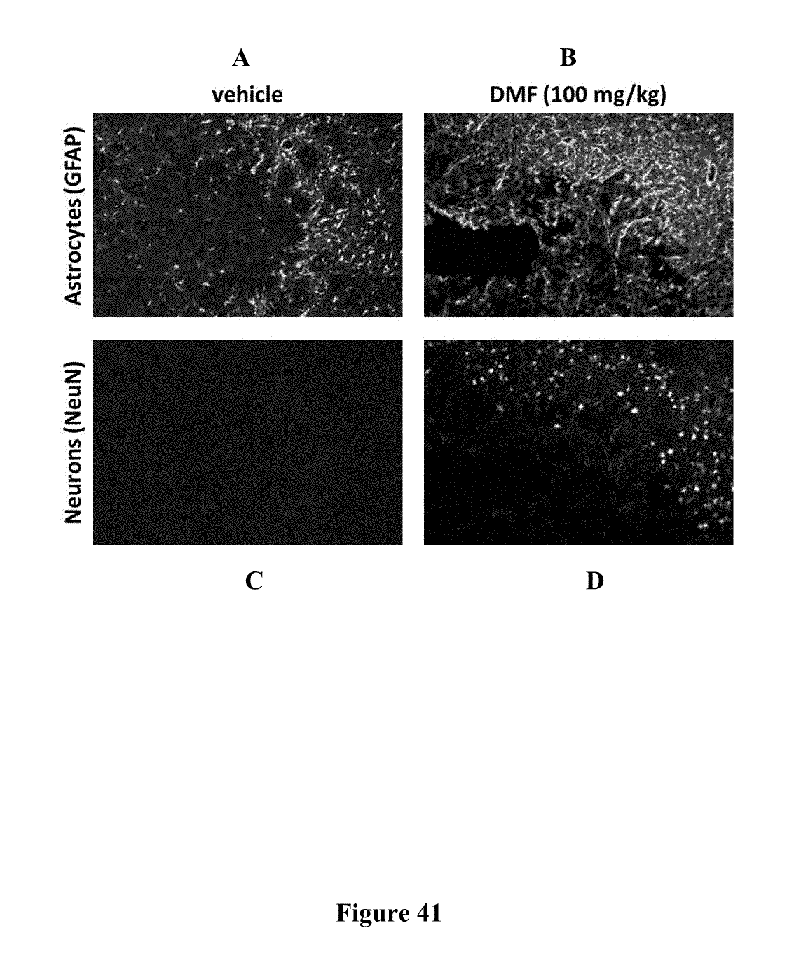

FIG. 41 shows representative images of lesioned rat brain sections staining for immunofluorescence. Proximal to the injection region there is an increase in the number of surviving neurons in animals that were administered vehicle (FIG. 41A, C) as compared to the animals treated with 100 mg/kg DMF (FIG. 41B, D). Astrocytes appear to survive in both vehicle and DMF treated animals near the lesion border. The images are 10× magnified.

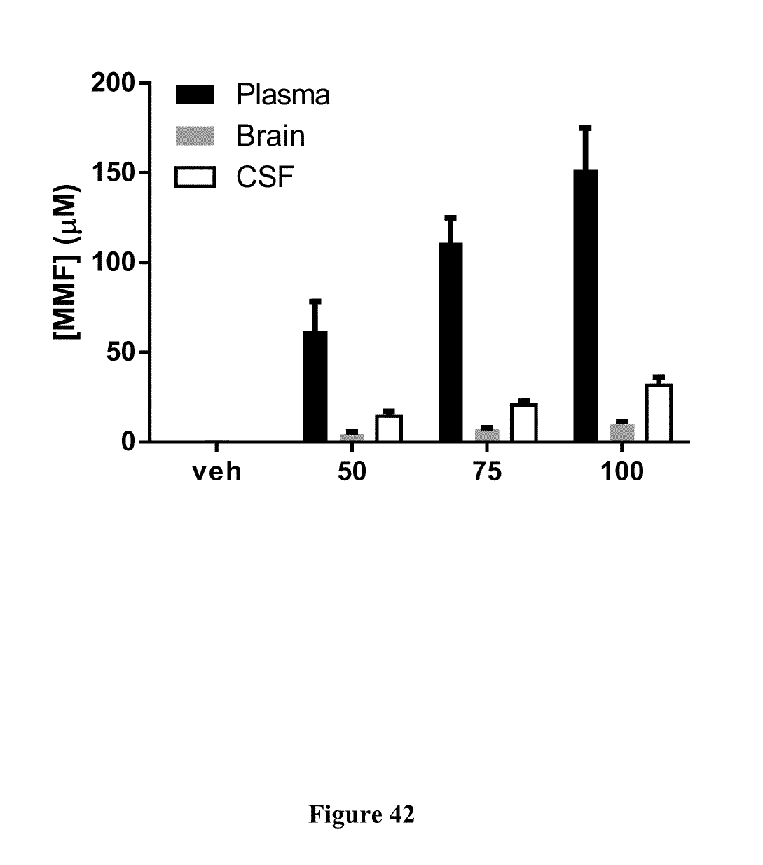

30 minutes after the last dose of DMF, all animals had plasma, cerebrospinal fluid (CSF) and brain tissue (cerebellum) collected to determine the levels of monomethyl fumarate (MMF, primary metabolite of DMF) in each compartment (FIG. 42). Mean values for all animals in each group are presented in ng/ml MMF. Error bars indicate standard error. Groups had 14 or 15 animals, as indicated previously. FIG. 42 shows that the concentration of MMF at all doses of DMF administered is significantly higher in the plasma as compared to both, the Brain and the CSF.

In conclusion, both experiments indicated that treatment with 100 mg/kg of DMF significantly reduced malonate-induced lesion volume by at least 55%. Increasing the number of animals in the second study revealed a significant 44% reduction in lesion volume with 75 mg/kg DMF treatment. Further, rotational behavior was significantly reduced 41% in animals treated with 100 mg/kg of DMF, suggesting a preservation of behavioral/motor function with treatment. Malonate-lesioned animals treated with 100 mg/kg DMF evidenced significant neuroprotection, as using a neuron-specific antibody (NeuN) for immunofluorescence microscopy revealed a preservation of neurons in the region proximal to the malonate injection site. Animals dosed with DMF had a dose-dependent increase in plasma, CSF and brain concentration of MMF, indicating the compound was reaching the target tissue, i.e., the brain.

As discussed above, DMF administered intravenously is expected to be more efficacious in this animal model than DMF administered orally.

### REFERENCES

Green and Greenamyre. Characterization of the excitotoxic potential of the reversible succinate dehydrogenase inhibitor malonate. J. Neurochemistry 1995:64(1): 430-4436.
Thatcher G R, et al. Novel nitrates as NO mimetics directed at Alzheimer's disease. J. Alzheimer's Disease 2004: 6: S75-S84.
Fancellu R, et al. Neuroprotective effects mediated by dopamine receptor agonists against malonate-induced lesion in the rat striatum. Neuro Sci. 2003:24:180-181.
Xia X G, et al. Dopamine mediates striatal malonate toxicity via dopamine transporter-dependent generation of reactive oxygen species and D2 but not D1 receptor activation. Journal of Neurochemistry 2001: 79: 63-70.
Linker R, et al. Fumaric acid esters exert neuroprotective effects in neuroinflammation via activation of Nrf2 antioxidant pathway. Brain 2011: 134: 678-692.

### 6.4 Example 4: Formulations for Intravenous Administration of DMF

### 6.4.1 Nano-Suspension of DMF

Summary

The nano suspension (150 mg/ml, D₅₀ around 180 nm) of DMF was made by overnight milling the mixture of DMF, hydroxylpropyl methylcellulose (HPMC), and sodium dodecyl sulfate (SDS) in pH 5.0 phosphate buffer. Chemical and physical stability of the nano suspension at ambient temperature was examined using high-performance liquid chromatography (HPLC), microscopy and particle size distribution (PSD). The results obtained indicated that the nano suspension was both chemically and physically stable up to 7 days at ambient temperature. A nano suspension is suitable, e.g., for IV formulations requiring >5 mg/ml of DMF.

Preparation of the DMF Nano Suspension

The nano suspension was prepared as follows:

1.5 g of DMF, 100 mg of HPMC E6 (Dow Chemical, Lot No. VL31012N22), and 20 mg of SDS (Fisher Scientific, Lot No. 090217) were added to a 50 ml Corning 430290 centrifuge tube. 5.0 ml of pH5.0 phosphate buffer (Fisher Scientific, Lot No. 100498) were added to the tube. Further, ZrO₂ beads (˜10 ml) were also added to the tube. The sample was vortexed in a Fisher Scientific multi-tube vortexer at 2500 rpm for 60 min and the particle size was checked via optical microscopic image. An additional 5.0 ml of pH5 phosphate buffer was added and the sample was further milled overnight. The temperature of the sample was ˜60° C. due to the overnight milling. The particle size was determined via microscopic image and Mastersizer 2000.

Particle Size

Particle size analysis was performed by laser diffraction techniques with a Malvern Mastersizer Hydro 2000S particle size analyzer (Malvern Instruments, England). The particle size was determined with the compound dispersed in 0.2% Teepol 610S (Aldrich) aqueous solution that was pre-saturated with DMF for 4 hours at ambient temperature.

Stability

The suspension was stored at room temperature and samples were taken at day 1 and day 7. The stability of the samples was examined using HPLC method for chemical stability, PSD and microscopic image (data not provided) for physical stability.

Chemical Stability:

The chemical stability of the DMF nanosuspension is shown in

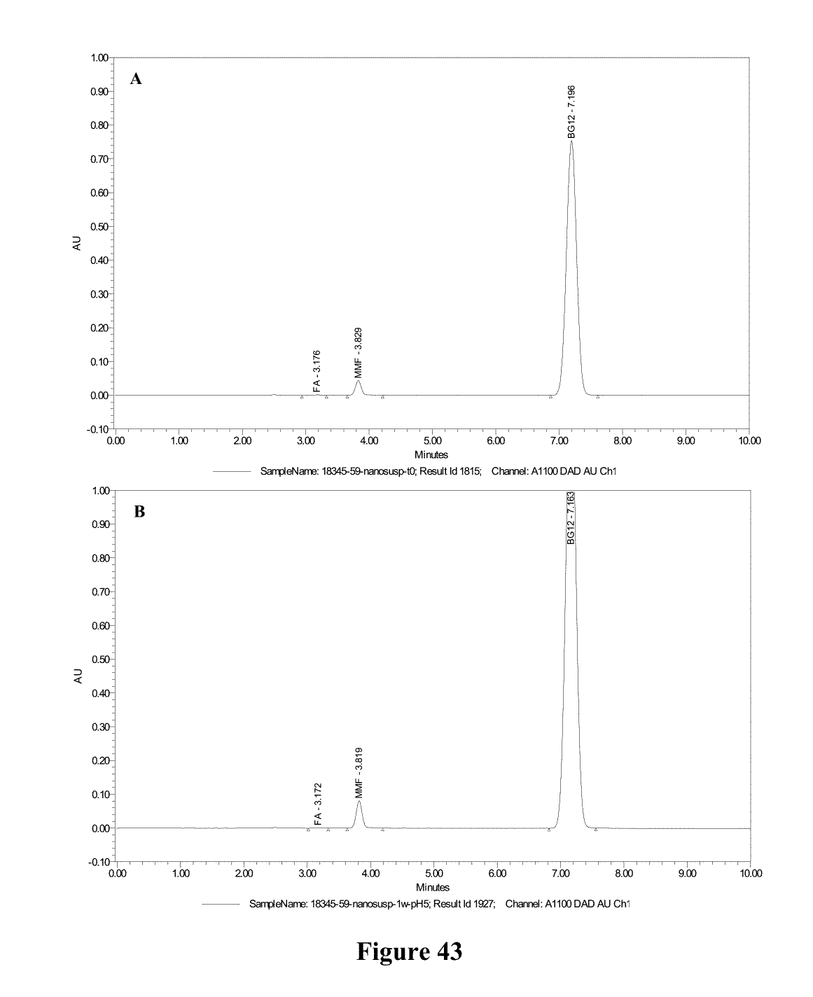

Table 9 and FIG. 43. The results indicated that the nano suspension was chemically stable up to 7 days.

TABLE 9
Stability of DMF nano suspension (area %)
Area % of DMF remaining^{a}

| Sample name | Day 1 | Day 7 |
|---|---|---|
| DMF | 99.9 | n/a |
| 150 mg/ml DMF | 96.6 | 96.6 |
| nano suspension^{b} | | |

^{a}An isocratic HPLC method was used for DMF chemical stability studies; Column: Variane Lichrosorb RP18, 250 mm × 4.6 mm; Mobile phase A: 0.1% phosphate acid in water (70%); Mobile phase B: Methanol (30%); Run time: 10 min; Flow rate: 1.0 mL/min; Injection volume: 10 μL; Column temperature: 35° C.; Detector wavelength: 210 nm with 4 nm bandwith.
^{b}about 3% of MMF, a DMF degradation product, was formed after overnight milling. Degradation may have been due to the higher temperature during the milling process.

Physical Stability:

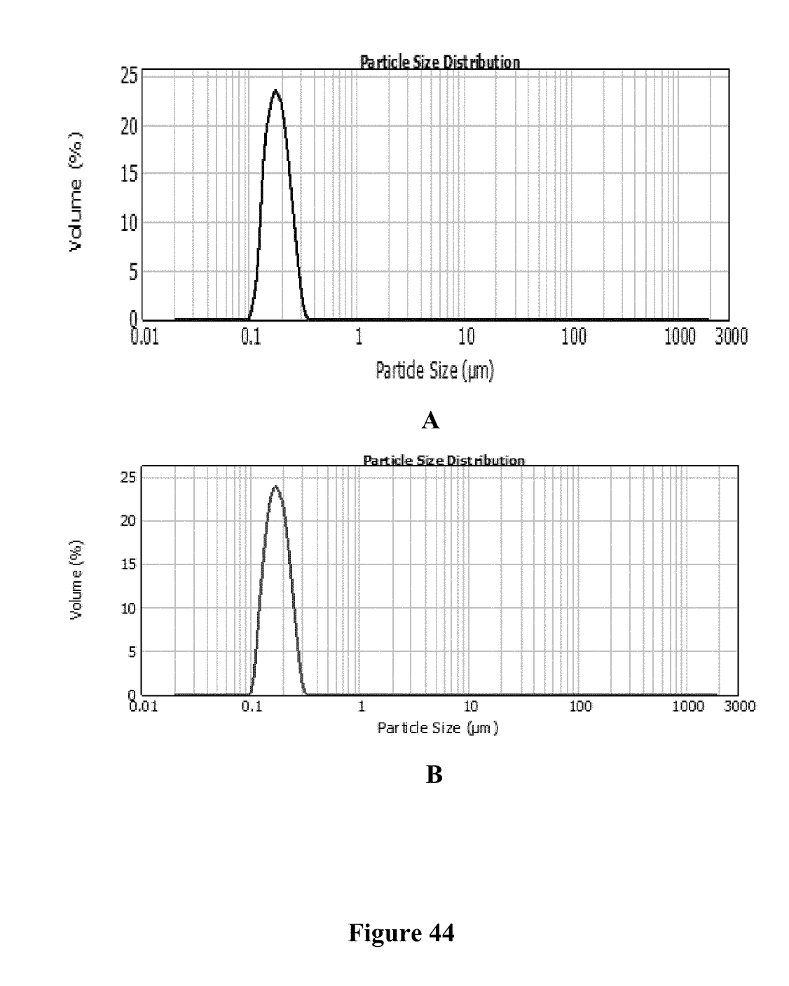

No size changes were observed for the nano suspension from both PSD (FIG. 44A (Day 1) (150 mg/ml, D₅₀ about 180 nm) and FIG. 44B (Day 7) (D₅₀ about 175 nm) and microscopic image (data not provided) when the sample was stored at ambient temperature up to 7 days. Hence, the nano suspension was physically stable for at least 7 days.

### 6.4.2 Formulation with DMF in Solution

Summary

The solubility of DMF in water with or without various excipients at ambient temperature was investigated and is summarized in Table 10:

TABLE 10

| Solution | | Solubility |
|---|---|---|
| No. | Media | (mg/ml) |
| 1 | water | 2.7 |
| 2 | D5W (5% Dextrose in water) | 2.39 |
| 3 | Saline (0.9% NaCl) | 2.08 |
| 4 | 5% HPbCD (Hydroxypropyl-β- | 4.03 |
| | cyclodextrin) | |
| 5 | 40% HPbCD | 15.13 |
| 6 | 5% Captisol (sulfobutyl ether | 3.18 |
| | cyclodextrin) | |
| 7 | 20% Captisol | 4.11 |
| 8 | 40% Captisol | 7.90 |

The solubility of DMF in cyclodextrin vehicles (solutions 4-8) was found to increase with the percentage of cyclodextrin increasing. Further, the solubility of DMF in water was found to increase with study temperatures (2.7 mg/ml at 20° C.). At 37° C. the aqueous solubility of DMF in water, simulated gastric fluid (SGF) and simulated intestinal fluid (SIF) was 2.84, 2.95, and 3.11 mg/ml, respectively. The solubility of DMF varied from 3.32 mg/ml in pH4 citrate buffer to 4.02 mg/ml in pH8 borate buffer.

The stability of DMF in aqueous media was pH dependent. At 37° C., DMF in solution was found stable in weak acidic conditions, with the maximum stability at around pH 4. The stability of DMF in solution decreased rapidly as the solution pH went above 7 at 37° C. Major degradation products of DMF observed were methyl hydrogen fumarate and fumaric acid.

DMF in 20% Captisol solution was stable up to 2 days at 0.2 mg/ml and up to 7 days at 4.0 mg/ml when the solution was stored at ambient temperature. An IV formulation comprising 20% Captisol is suitable, e.g., for IV formulations requiring <4 mg/ml of DMF. Saline and D5W formulations are useful, e.g., for IV formulations requiring <2 mg/ml. In certain embodiments, such Saline and D5W formulations are freshly prepared.

Solubility in Water

A 24-hour equilibrium solubility of DMF in water was determined at 20, 35, and 50° C., using a suspension-equilibrium method. Suspensions of about 20 mg of DMF in 1 mL water were agitated at the measuring temperatures for 24 hours. Samples were then filtrated at the measuring temperatures through a 0.2 μm filter unit, and the concentrations of the filtrate were determined using HPLC. The solids in equilibrium were confirmed, by FT-Raman technique, to be the same form as the starting material. The solubility of DMF was found to increase with study temperatures (Table 11).

TABLE 11
Solubility of DMF in water

| Temperature (° C.) | Solubility (mg/mL) |
|---|---|
| 20 | 2.7 |
| 35 | 3.5 |
| 50 | 5.5 |

### Solubility in Aqueous Media at 37° C.

Solubility of DMF in aqueous media was determined at 37° C. by preparing a saturated solution in water, simulated gastric fluid (SGF, without pepsin), simulated intestinal fluid (SIF, without pancreatin) or pH 2, 4, 6, 8, and 10 buffers. The preparations were rotated at 37° C. in an Isotemp Oven for 24 hours, followed by filtration through a 0.2 μm filter and determination of the amount of DMF in the solution by HPLC. At 37° C. the aqueous solubility of DMF in water, SGF and SIF was 2.84, 2.95 and 3.11 mg/ml, respectively. And the solubility of DMF varied from 3.32 mg/ml in pH4 citrate butter to 4.02 mg/ml in pH8 borate buffer (Table 12)

TABLE 12
The solubility of DMF in different aqueous media at 37° C.

| | | Solubility | |
|---|---|---|---|
| Solution | pH | (mg/ml) | Solubility Description** |
| Water | 5.20 | 2.84 | Slightly soluble |
| SGF (without pepsin) | 6.90 | 2.95 | Slightly soluble |
| SIF (without pancreatin) | 1.20 | 3.11 | Slightly soluble |
| pH 2 Citrate Buffer | 1.93 | 3.77 | Slightly soluble |
| pH 4 Citrate Buffer | 3.89 | 3.32 | Slightly soluble |
| pH 6 Citrate Buffer | 6.01 | 3.47 | Slightly soluble |
| pH 8 Borate Buffer | 5.44 | 4.02 | Slightly soluble |
| pH 10 Borate Buffer | 5.49 | 3.57 | Slightly soluble |

| Descriptive | Parts of Solvent Required |
|---|---|
| Term | for 1 Part of Solute (mg/ml) |
| Very soluble | Less than 1 (>1000) |
| Freely soluble | From 1 to 10 (100~1000) |
| Soluble | From 10 to 30 (33.3~100) |
| Sparingly soluble | From 30 to 100 (10~33.3) |
| Slightly soluble | From 100 to 1000 (1~10) |
| Very slightly soluble | From 1000 to 10,000 (0.1~1) |
| Practically insoluble, or Insoluble | Greater than or equal to 10,000 |
| | (<0.1) |

**The solubility is described as defined in the U.S. Pharmacopeia 27:

Solution Stability in Aqueous Solution

Stock DMF solutions in water were prepared at 0.4 mg/ml concentration. Solution stability studies were conducted using 0.2 mg/ml concentration solutions, which were achieved by mixing equal volumes of the stock solution and individual study media. Solution was studied in water, SIF, SGF, aqueous buffers of pH 2, 4, 6, and 10. Samples stored at 37° C. or ambient temperature were pulled at predetermined time points, and analyzed by HPLC.

The experimental results indicated the stability of DMF in aqueous media was pH dependent. At 37° C., DMF in solution was found stable in weak acidic conditions, with the maximum stability at around pH 4 (Table 13). The stability of DMF in solution decreased rapidly as the solution pH went above 7 at 37° C. In the pH 10 borate buffer solution, more than half of DMF degraded before an initial concentration was able to be determined by HPLC. Major degradants observed from DMF were methyl hydrogen fumarate and fumaric acid.

TABLE 13
Stability of DMF in different solutions at 37° C. and ambient
temperature.

| | area % (assay) | | | | |
|---|---|---|---|---|---|
| | | 37° C. | Ambient Temperature | | |
| Solution | initial | 24 hr | 24 hr | 48 hr | 72 hr |
| Water | 96.6 (98.9) | 77.4 (73.9) | 84.8 | 85.1 | 83.5 |
| | | | (81.6) | (80.2) | (79.3) |
| SGF (without | 99.3 (102.9) | 92.2 (91.1) | 96.6 | 95.6 | 95.4 |
| pepsin) | | | (95.2) | (92.5) | (91.6) |
| SIF (without | 99.3 (100.9) | 29.5 (27.8) | 68.7 | 64.5 | 62.2 |
| pancreatin) | | | (68.3) | (60.3) | (59.0) |
| pH 2 Citrate | 99.5 (101.0) | 97.9 (96.0) | 99.1 | 98.9 | 98.7 |
| Buffer | | | (94.7) | (93.2) | (92.0) |
| pH 4 Citrate | 99.8 (101.9) | 99.6 (100.4) | 99.6 | 99.2 | 99.6 |
| Buffer | | | (97.2) | (98.5) | (93.9) |
| pH 6 Citrate | 100 (101.4) | 92.6 (91.8) | 97.5 | 95.3 | 96.1 |
| Buffer | | | (90.7) | (92.5) | (88.4) |
| pH 8 Borate | 98.6 (101.0) | 17.2 (15.9) | 46.5 | n/a | n/a |
| Buffer | | | (44.8) | | |
| pH 10 Borate | 37.1 (36.1) | 0/0 | 0/0 | n/a | n/a |
| Buffer | | | | | |

n/a = not available

DMF IV Formulation Development for Preclinical Studies

A DMF IV formulation, which was suitable for preclinical studies with a target concentration of about 5 mg/ml was developed in accordance with the following procedure:

1. Testing the solubility of DMF in several pure solvents (DMSO, Ethanol, propylene glycolm and PEG 400), which are suitable for IV dosage formulation (Table 14). The results are presented in Table 15.

2. Testing whether 5 mg/ml clear solution could be achieved based on the typical maximum used levels of excipients in various animal species (Table 14). For example, 2% DMSO (0.1 ml/kg/5 ml/kg*100%=2.0%), 10% PEG400 (1 ml/kg/5 ml/kg*50%=10%) and 20% Captisol (sulfobutyl ether cyclodextrin) (5 ml/kg/5 ml/kg*20%=20%) could be used for rodent IV formulation assumed the dosage of 5 ml/kg.

3. Testing the solubility of DMF in 10% PEG400 with different excipients since PEG400 provided the best solubility for DMF. The studies were performed by making the PEG400 stock solution and diluting the solution with different vehicles (Table 16). The results indicated that even 3.0 mg/ml clear solution could not be achieved by using 10% PEG400 with most excipients, but 20% Captisol and 15% HPbCD might be the better selections since both of them generated clear solutions at 2.8 mg/ml.

4. Testing the solubility of DMF in 15% HPbCD and 20% Captisol. The results indicated that about 5 mg/ml DMF clear solution could be achieved by using 20% Captisol (Table 17).

TABLE 14
Typical maximum used levels of excipients in various animal species.

| | Rodents | | | | | | | | | | | |
|---|---|---|---|---|---|---|---|---|---|---|---|---|
| | (rat and mouse) | | | | Dogs | | | | Monkeys | | | |
| | Oral | | IV | | Oral | | IV | | Oral | | IV | |
| | % w/w | | % w/w | | % w/w | | % w/w | | % w/w | | % w/w | |
| | (ml/kg) | | (ml/kg) | | (ml/kg) | | (ml/kg) | | (ml/kg) | | (ml/kg) | |
| DMSO | 50 | (0.5) | 100 | (0.1) | 50 | (0.5) | 100 | (0.05) | 50 | (0.5) | Do not use | |
| PEG-400 | 100 | (2) | 50 | (1) | 80 | (2) | 30 | (0.5) | 25 | (2) | 30 | (0.5) |
| Propylene | 80 | (2) | 50 | (0.5) | 50 | (2) | 30 | (0.5) | 50 | (0.5) | 10 | (0.5) |
| glycol | | | | | | | | | | | | |
| Ethanol | 50 | (0.5) | 20 | (0.5) | 50 | (2) | 20 | (0.5) | 25 | (2) | 20 | (0.5) |
| Tween 80 | 50 | (2.5) | 2 | (0.25) | 25 | (1) | Do not use | | 25 | (1) | 0.5 | (0.005) |
| Polaxamer | 15 | (1.5) | 15 | (0.5) | 15 | (1.5) | 15 | (0.5) | 15 | (1) | 15 | (0.3) |
| Cremophor EL | 10 | (0.5) | 10 | (0.5)* | 10 | (0.5) | 10 | (0.1)* | 10 | (0.5) | Do not use | |
| Oils | 100 | (5) | 15 | (1.5)‡ | 100 | (2) | 15 | (1.5)‡ | 100 | (2) | 15 | (1.5)‡ |
| Sulfobutyl | 40 | (5) | 20 | (5)§ | 40 | (2) | 20 | (2)§ | 40 | (2) | 20 | (2)§ |
| ether | | | | | | | | | | | | |
| cyclodextrin | | | | | | | | | | | | |

S. Neervannan; Expert Opin. Drug Metab. Toxicol. 2006 2(5) p715-731

TABLE 15
Solubility of DMF in some solvents, which are suitable for IV
formulation.

| Excipients | Target vehicle | Solubility (mg/ml) | Suitable for 5 mg/ml dosage? |
|---|---|---|---|
| DMSO | | 23-29 | |
| | 2% DMSO in | | No, 5 mg/ml solution could be |
| | DIW | | achieved by heating the sample; |
| | | | but precipitate formed when the |
| | | | sample was cooled to room |
| | | | temperature. |
| PEG400 | | 38-46 | |
| | 10% PEG400 in | | Might be, further studies in Table |
| | DIW | | 5b |
| propylene | | <20 | |
| Glycol (PG) | 5% propylene | | No, 5 mg/ml solution could be |
| | Glycol (PG) | | achieved by heating the sample; |
| | | | but precipitate formed when the |
| | | | sample was cooled to room |
| | | | temperature. |
| Ethanol | | <20 | |
| | 2% Ethanol | | No, 5 mg/ml solution could be |
| | | | achieved by heating the sample; |
| | | | but precipitate formed when the |
| | | | sample was cooled to room |
| | | | temperature. |

TABLE 16
Solubility of DMF in 10% PEG400 with different vehicles.
DMF stock solution: 71.88 mg dissolved in 2.5 ml PEG400 (28.7 mg/ml)
100 μl of stock solution was diluted with 900 μl the
solvents below (final concentration 2.8 mg/ml)

| | Solution or | Suitable for |
|---|---|---|
| vehicles added | precipitated | 5 mg/ml dosage? |
| 0.5% HPMC | precipitated | No |
| 0.5% HPMC + 0.2% SDS | precipitated | No |
| 2.0% Tween 80 | precipitated | No |
| 1.5% Poloxamer 188 | precipitated | No |
| 1.5% Poloxamer 237 | precipitated | No |
| D5W | precipitated | No |
| D5W phosphate buffer (pH 6.0) | precipitated | No |
| Saline (pH 7) | precipitated | No |
| H₂O | precipitated | No |
| 20% Captisol | Clear solution | Might be |
| 15% HPbCD | Clear solution | Might be |

TABLE 17
Solubility of DMF in 15% HPbCD and 20% Captisol.
5 mg/ml DMF in 15% HPbCD or 20% Captisol was made
by heating then cooling the sample to room temperature

| | Final | Solution or | |
|---|---|---|---|
| vehicles | pH | precipitated | Suitable for 5 mg/ml dosage? |
| 15% HPbCD | 3.51 | solution | No, 5 mg/ml solution could be |
| | | | achieved by heating the sample; but |
| | | | precipitate formed when the sample |
| | | | was cooled to room temperature. |
| 20% | 4.71 | solution | Might be, 5 mg/ml solution could be |
| Captisol | | | achieved by heating the sample; the |
| | | | sample remained as clear solution |
| | | | after cooling to room temperature |

Solubility Test of DMF in HPbCD, Captisol, Saline and D5W

The solubility of DMF in 5% HPbCD, 40% HPbCD, 5% Captisol, and 40% Captisol was tested to investigate the effect of percentage of cyclodextrin on the solubility of DMF. The solubility of DMF in D5W (5% Dextrose in water) and Saline (0.9% NaCl solution) was also tested for the possibility of low concentration DMF IV formulations.

Solubility of DMF in these media was determined at ambient temperature by preparing a saturated solution. The preparations were rotated at ambient temperature for 24 hours, followed by filtration through a 0.45 μm filter and determination of the amount of DMF in the solution by HPLC.

TABLE 18
The solubility of DMF in HPbCD, Captisol, D5W and Saline.

| | | Solubility |
|---|---|---|
| Solution | pH | (mg/ml) |
| 5% HPbCD | 4.30 | 4.03 |
| 40% HPbCD | 3.90 | 15.13 |
| 5% Captisol | 4.80 | 3.18 |
| 40% Captisol | 4.42 | 7.90 |
| D5W (5% Dextrose in water) | 6.32 | 2.39 |
| saline | 6.67 | 2.08 |

Stability and Solubility Test of DMF in 20% Captisol

Based on the information discussed above, 20% Captisol was selected for further investigation.

The solubility of DMF in 20% Captisol was determined at room temperature by preparing a saturated solution in 20% Captisol. The preparations were heated at 50° C. for 5-10 min, followed by cooled to room temperature. At this time precipitate was formed and the sample was filtered through a 0.2 μm filter and determination of the amount of DMF in the solution by HPLC.

The solubility of DMF in 20% Captisol was 4.11 mg/ml.

The stability of 0.2 and 4.0 mg/ml of DMF 20% Captisol solutions was studied. Samples stored at ambient temperature were pulled at predetermined time points (0, 2 and 7 days), and analyzed by HPLC.

The results indicated that DMF 20% Captisol solution was stable up to 2 d at 0.2 mg/ml and up to 7 days at 4.0 mg/ml when the solution was stored at ambient temperature.

TABLE 19
Stability of DMF 20% Captisol at ambient temperature.

| | area %^{a} | | |
|---|---|---|---|
| Solution | initial | 2 d | 7 d |
| 0.2 mg/ml | 99.7 | 99.5 | 97.2 |
| 4 mg/ml | 99.7 | | 99.4 |

^{a}An isocratic HPLC method was used for DMF chemical stability studies; Column: Variane Lichrosorb RP18, 250 mm × 4.6 mm; Mobile phase A: 0.1% phosphate acid in water (70%); Mobile phase B: Methanol (30%); Run time: 10 min; Flow rate: 1.0 mL/min; Injection volume: 10 μL; Column temperature: 35° C.; Detector wavelength: 210 nm with 4 nm bandwith.

Exemplary Protocol for Preparing a Solution of DMF for Intravenous Administration

Shown below is an exemplary protocol for preparing a solution of DMF suitable for intravenous administration.

Conditions of Handling and Use
For Preclinical Development

| | |
|---|---|
| Product Code | Dimethyl Fumarate (DMF) |
| Formulation code | DMF IV formulation |
| Drug Substance | Dimethyl fumarate (DMF) |
| Drug Substance | Biogen Idec |
| Manufacturer | |
| Dosage Form | Liquid |
| Route of administration | IV |
| Formulation: | Solution |
| Formulation | 20% Captisol (β-Cyclodextrin Sulfobutyl Ethers, Sodium Salts) |
| Composition | |
| Storage Condition | 22° C. ± 5° C. |
| Stability | DMF in Captisol solution at 4.0 mg/mL at ambient temperature |
| | are chemically and physically stable for 7 days. |
| | DMF in Captisol solution at 0.2 mg/mL at ambient temperature |
| | are chemically and physically stable for 2 days. (about 2.5% |
| | degradents were detected in the 7 day solution) |
| Vehicle/Diluent | β-Cyclodextrin Sulfobutyl Ethers, Sodium Salts (Captisol - |
| | Research grades, CyDex) |
| | If specific product number of Captisol, listed above, cannot be |
| | used, please contact sponsor. For all other materials equivalent |
| | grades can be used |

### INCORPORATION BY REFERENCE

Various references such as patents, patent applications, and publications are cited herein, the disclosures of which are hereby incorporated by reference herein in their entireties.

1. A method of treating a neurological disease in a human patient in need thereof comprising administering intravenously to the patient a pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing.

2-262. (canceled)

263. The method of claim 1, wherein the disease is stroke, amyotrophic lateral sclerosis, Huntington's disease, Alzheimer's disease, Parkinson's disease, or multiple sclerosis.

264. The method of claim 263, wherein the disease is stroke.

265. The method of claim 264, wherein the at least one fumarate is selected from the group consisting of dialkyl fumarate, monoalkyl fumarate, a combination of dialkyl fumarate and monoalkyl fumarate, a deuterated form of any of the foregoing, and a pharmaceutically acceptable salt, tautomer, or stereoisomer of any of the foregoing.

266. The method of claim 265, wherein the fumarate is dimethyl fumarate.

267. The method of claim 266, wherein the amount of dimethyl fumarate that is administered in said step of administering intravenously is in the range of 1 to 1000 milligrams.

268. The method of claim 267, wherein the amount of dimethyl fumarate that is administered in said step of administering intravenously is in the range of 10 to 750 milligrams.

269. The method of claim 267, wherein the amount of dimethyl fumarate that is administered in said step of administering intravenously is in the range of 48 to 240 milligrams.

270. The method of claim 266, wherein a therapeutically effective amount of dimethyl fumarate is administered in said step of administering intravenously, said amount being less than 480 milligrams.

271. The method of claim 264, wherein the method consists essentially of said administering step.

272. The method of claim 264, wherein the at least one fumarate is the only active agent(s) administered to the patient for said treating.

273. The method of claim 264, wherein the only active agent(s) in the pharmaceutical composition is the at least one fumarate.

274. The method of claim 273, wherein the only active agents in the pharmaceutical composition are dimethyl fumarate and monomethyl fumarate.

275. The method of claim 273, wherein the only active agent in the pharmaceutical composition is one fumarate selected from said group.

276. The method of claim 264, wherein the only active agent(s) in the pharmaceutical composition is dimethyl fumarate and optionally one or more compounds produced by degradation from dimethyl fumarate in said pharmaceutical composition prior to said administering.

277. The method of claim 272, wherein the only active agent in the pharmaceutical composition is dimethyl fumarate.

278. The method of claim 264, wherein the pharmaceutical composition consists essentially of the at least one fumarate.

279. The method of claim 278, wherein the pharmaceutical composition consists essentially of dimethyl fumarate.

280. The method of claim 264, wherein said administering is performed daily.

281. The method of claim 264, wherein said administering is performed once per week.

282. The method of claim 264, wherein said administering is performed every other week.

283. The method of claim 264, wherein said administering is performed once per month.

284. The method of claim 280, wherein the step of administering intravenously is repeated over a time period of at least two weeks.

285. The method of claim 280, wherein the step of administering intravenously is repeated over a time period of at least one month.

286. The method of claim 280, wherein the step of administering intravenously is repeated over a time period of at least six months.

287. The method of claim 280, wherein the step of administering intravenously is repeated over a time period of at least one year.

288. The method of claim 264, wherein said administering is part of a treatment regimen wherein said administering intravenously to the patient alternates with one or more steps of administering the fumarate orally to the patient.

289. The method of claim 288, wherein the fumarate is dimethyl fumarate, and the amount of dimethyl fumarate administered orally is 480 mg daily.

290. The method of claim 264, wherein the patient does not have a known hypersensitivity to the fumarate.

291. The method of claim 264, wherein the patient is not treated simultaneously with a fumarate and any immunosuppressive or immunomodulatory medications or natalizumab.

292. The method of claim 264, wherein the patient is not treated simultaneously with a fumarate and any medications carrying a known risk of causing progressive multifocal leukoencephalopathy (PML).

293. The method of claim 264, wherein the patient has no identified systemic medical condition resulting in a compromised immune system function.

294. The method of claim 264, wherein the disease is hemorrhagic stroke.

295. The method of claim 264, wherein the disease is ischemic stroke.

296. The method of claim 264, wherein the pharmaceutical composition is a sterile isotonic solution.

297. The method of claim 264, wherein the pharmaceutical composition is a nanosuspension.

298. The method of claim 264, wherein the pharmaceutical composition is an aqueous solution, wherein the aqueous solution comprises a cyclodextrin and said at least one fumarate, wherein the cyclodextrin is an alpha cyclodextrin or a substituted beta cyclodextrin.

299. A pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is a nanosuspension.

300. A pharmaceutical composition comprising at least one fumarate selected from the group consisting of dialkyl fumarate, monoalkyl fumarate, a combination of dialkyl fumarate and monoalkyl fumarate, a prodrug of monoalkyl fumarate, a deuterated form of any of the foregoing, and a pharmaceutically acceptable salt, tautomer, or stereoisomer of any of the foregoing, wherein the pharmaceutical composition is an aqueous solution, wherein the aqueous solution further comprises a cyclodextrin, wherein the cyclodextrin is an alpha cyclodextrin or a substituted beta cyclodextrin.
